(19) United States
(12) Patent Application Publication
(10) Pub. No.: US 2006/0142192 A1
(43) Pub. Date: Jun. 29, 2006

(54) Soluble ZcytoR21, anti-ZcytoR21 antibodies and binding partners and methods of using in inflammation

(76) Inventors: Zeren Gao, Redmond, WA (US); Rolf E. Kuestner, Bothell, WA (US); Mark W. Appleby, Shoreline, WA (US); Katherine E. Lewis, Lake Forest Park, WA (US); Patricia A. McKernan, Seattle, WA (US); Shannon L. Okada, Seattle, WA (US); David W. Taft, Kirkland, WA (US); Joseph L. Kuijper, Kenmore, WA (US); Stephen R. Jaspers, Edmonds, WA (US); Steven D. Levin, Seattle, WA (US)

Correspondence Address: ZymoGenetics, Inc., 1201 Eastlake Avenue East, Seattle, WA, 98102, US

(21) Appl. No.: 11/253,200

(22) Filed: Oct. 18, 2005

Related U.S. Application Data
(60) Provisional application No. 60/619,651, filed on Oct. 18, 2004.
(60) Provisional application No. 60/622,207, filed on Oct. 25, 2004.

(51) Int. Cl. A61K 38/17; C07K 14/715

(52) U.S. Cl. 514/12; 530/350

(57) ABSTRACT

The present invention relates ZcytoR21 antagonists, such as soluble receptors and anti-ZcytoR21 antibodies, that are useful in blocking, inhibiting, reducing, antagonizing or neutralizing the activity of IL-17C. IL-17C is a cytokine that is involved in inflammatory processes and human disease. ZcytoR21 is a receptor for IL-17C. The present invention includes soluble ZcytoR21, anti-ZcytoR21 antibodies and binding partners, as well as methods for antagonizing IL-17C using such soluble receptors, antibodies and binding partners.

### BACKGROUND OF THE INVENTION

Cytokines are soluble, small proteins that mediate a variety of biological effects, including the regulation of the growth and differentiation of many cell types (see, for example, Arai et al., Annu. Rev. Biochem. 59:783 (1990); Mosmann, Curr. Opin. Immunol. 3:311 (1991); Paul and Seder, Cell 76:241 (1994)). Proteins that constitute the cytokine group include interleukins, interferons, colony stimulating factors, tumor necrosis factors, and other regulatory molecules. For example, human interleukin-17 is a cytokine which stimulates the expression of interleukin-6, intracellular adhesion molecule 1, interleukin-8, granulocyte macrophage colony-stimulating factor, and prostaglandin E2 expression, and plays a role in the preferential maturation of CD34+ hematopoietic precursors into neutrophils (Yao et al., J. Immunol. 155:5483 (1995); Fossiez et al., J. Exp. Med. 183:2593 (1996)).

Receptors that bind cytokines are typically composed of one or more integral membrane proteins that bind the cytokine with high affinity and transduce this binding event to the cell through the cytoplasmic portions of the certain receptor subunits. Cytokine receptors have been grouped into several classes on the basis of similarities in their extracellular ligand binding domains.

The demonstrated in vivo activities of cytokines and their receptors illustrate the clinical potential of, and need for, other cytokines, cytokine receptors, cytokine agonists, and cytokine antagonists. For example, demonstrated in vivo activities of the pro-inflammatory cytokine family illustrates the enormous clinical potential of, and need for antagonists of pro-inflammatory molecules.

### DETAILED DESCRIPTION OF THE INVENTION

Genome-wide homology comparisons led to identification of five ligands and four receptor paralogs within the IL-17/IL-17R family. Most of these remain un-paired orphans. Establishment of receptor-ligand pairs in this family has been complicated because nearly all IL-17R homologs are represented by multiple splice variants, resulting in alternative extracellular domains. Emerging data suggests that IL-17C, like IL-17, IL-17A and IL-17F, is a pro-inflammatory cytokine causing neutrophilia when expressed by intranasal administration and adenoviral infection in mouse lungs. Specifically, the pro-inflammatory cytokine IL-17C has a high degree of sequence similarity to IL-17. IL-17 is a T cell-derived cytokine that plays an important role in the initiation or maintenance of the proinflammatory response. Whereas expression of IL-17 is restricted to activated T cells, the IL-17 receptor (IL-17R) is found to be widely expressed, a finding consistent with the pleiotropic activities of IL-17. IL-17C is related to IL-17, having approximately 27% amino acid identity. See e.g Li H et al, “Cloning and characterization of IL-17B and IL-17C, two new members of the IL-17 cytokine family” PNAS 97(2): 773-8 (2000). Although no expression of IL-17C mRNA is found in activated T cells, in a survey of cytokine induction, IL-17C does stimulate the release of tumor necrosis factor a and IL-1b from the monocytic cell line, THP-1, whereas IL-17 has only a weak effect in this system. Further, fluorescence activated cell sorter analysis shows that IL-17C binds to THP-1 cells. IL-17C is not active in an IL-17 assay, nor does it stimulate IL-6 release from human fibroblasts or bind to the human IL-17 receptor extracellular domain. This data shows that there is a family of IL-17-related cytokines differing in patterns of expression and proinflammatory responses that may be transduced through a cognate set of cell surface receptors. Members of the IL-17 family have been implicated as factors that contribute to the progression of various autoimmune and inflammatory diseases including rheumatoid arthritis and asthma.

IL-17C's ability to bind to members of the IL-17R family has been investigated. It has been discovered that IL-17C binds specifically to ZcytoR21 (also known as Il-17RE). Accordingly, we now report that we have identified ZcytoR21 as the receptor for IL-17C. Since intervention of other IL-17 family members has been proposed as an effective therapy for several auto-immune diseases, using the soluble receptors and antibodies of the present invention as immunomodulators, such as agonists or antagonists, to enhance, stimulate, agonize, block, inhibit, reduce, antagonize or neutralize the activity of IL-17C or ZcytoR21, may be advantageous. The present invention addresses these needs by providing antagonists to pro-inflammatory cytokine IL-17C. The invention further provides uses therefor in inflammatory disease, as well as related compositions and methods.

A) Overview

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multi-step pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, etc.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

A central event in both humoral and cell mediated immune responses is the activation and clonal expansion of helper T cells. Helper T cell activation is initiated by the interaction of the T cell receptor (TCR)-CD3 complex with an antigen-MHC on the surface of an antigen presenting cell. This interaction mediates a cascade of biochemical events that induce the resting helper T cell to enter a cell cycle (the G0 to G1 transition) and results in the expression of a high affinity receptor for IL-2 and sometimes IL-4. The activated T cell progresses through the cycle proliferating and differentiating into memory cells or effector cells.

In addition to the signals mediated through the TCR, activation of T cells involves additional costimulation induced by cytokines released by the antigen presenting cell or through interactions with membrane bound molecules on the antigen presenting cell and the T cell. The cytokines IL-1 and IL-6 have been shown to provide a costimulatory signal. Also, the interaction between the B7 molecule expressed on the surface of an antigen presenting cell and CD28 and CTLA-4 molecules expressed on the T cell surface effect T cell activation. Activated T cells express an increased number of cellular adhesion molecules, such as ICAM-1, integrins, VLA-4, LFA-1, CD56, etc.

T-cell proliferation in a mixed lymphocyte culture or mixed lymphocyte reaction (MLR) is an established indication of the ability of a compound to stimulate the immune system. In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Immune related diseases could be treated by suppressing the immune response. Using soluble receptors and/or neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The IL-17 cytokine/receptor families appear to represent a unique signaling system within the cytokine network that will offer innovative approaches to the manipulation of immune and inflammatory responses. Accordingly, the present invention is based on the pairing of IL-17C with its orphan receptor, ZcytoR21.

As such, antagonists to IL-17C activity, such as ZcytoR21 soluble receptors and antibodies thereto, are useful in therapeutic treatment of inflammatory diseases, particularly as antagonists to IL-17C in the treatment of asthma or psoriasis. Moreover, antagonists to IL-17C activity, such as ZcytoR21 soluble receptors and antibodies thereto including the anti-human-ZcytoR21 monoclonal and neutralizing antibodies of the present invention, are useful in therapeutic treatment of other inflammatory diseases for example as bind, block, inhibit, reduce, antagonize or neutralize IL-17C in the treatment of atopic and contact dermatitis, IBD, colitis, endotoxemia, arthritis, rheumatoid arthritis, psoriatic arthritis, adult respiratory disease (ARD), septic shock, multiple organ failure, inflammatory lung injury such as asthma, chronic obstructive pulmonary disease (COPD), airway hyper-responsiveness, chronic bronchitis, allergic asthma, bacterial pneumonia, psoriasis, eczema, and inflammatory bowel disease such as ulcerative colitis and Crohn's disease, helicobacter pylori infection, intraabdominal adhesions and/or abscesses as results of peritoneal inflammation (i.e. from infection, injury, etc.), systemic lupus erythematosus (SLE), multiple sclerosis, systemic sclerosis, nephrotic syndrome, organ allograft rejection, graft vs. host disease (GVHD), kidney, lung, heart, etc. transplant rejection, streptococcal cell wall (SCW)-induced arthritis, osteoarthritis, gingivitis/periodontitis, herpetic stromal keratitis, cancers including prostate, renal, colon, ovarian, cervical, leukemia, angiogenesis, restenosis and kawasaki disease.

Cytokine receptors subunits are characterized by a multi-domain structure comprising a ligand-binding domain and an effector domain that is typically involved in signal transduction. Multimeric cytokine receptors include monomers, homodimers (e.g., PDGF receptor αα and ββ isoforms, erythropoietin receptor, MPL [thrombopoietin receptor], and G-CSF receptor), heterodimers whose subunits each have ligand-binding and effector domains (e.g., PDGF receptor αβ isoform), and multimers having component subunits with disparate functions (e.g., IL-2, IL-3, IL-4, IL-5, IL-6, IL-7, and GM-CSF receptors). Some receptor subunits are common to a plurality of receptors. For example, the AIC2B subunit, which cannot bind ligand on its own but includes an intracellular signal transduction domain, is a component of IL-3 and GM-CSF receptors. Many cytokine receptors can be placed into one of four related families on the basis of their structures and functions. Class I hematopoietic receptors, for example, are characterized by the presence of a domain containing conserved cysteine residues and the WSXWS motif. Additional domains, including protein kinase domains; fibronectin type III domains; and immunoglobulin domains, which are characterized by disulfide-bonded loops, are present in certain hematopoietic receptors. Cytokine receptor structure has been reviewed by Urdal, Ann. Reports Med. Chem. 26:221-228, 1991 and Cosman, Cytokine 5:95-106, 1993. It is generally believed that under selective pressure for organisms to acquire new biological functions, new receptor family members arose from duplication of existing receptor genes leading to the existence of multi-gene families. Family members thus contain vestiges of the ancestral gene, and these characteristic features “an be exploited in the isolation and identification of additional family members.

Amongst other inventions, the present invention provides novel uses for a soluble receptor, designated “ZcytoR21” or “soluble ZcytoR21” or “sZcytoR21”, all of which may be used herein interchangeably, and neutralizing antibodies to ZcytoR21 cytokine receptors. The present invention also provides soluble ZcytoR21 polypeptide fragments and fusion proteins, for use in human inflammatory and autoimmune diseases. The anti-ZcytoR21 antibodies and soluble ZcytoR21 receptors of the present invention, including the neutralizing anti-ZcytoR21 antibodies of the present invention, can be used to block, inhibit, reduce, antagonize or neutralize the activity of IL-17C in the treatment of inflammation and inflammatory diseases such as psoriasis, psoriatic arthritis, rheumatoid arthritis, endotoxemia, inflammatory bowel disease (IBD), colitis, asthma, allograft rejection, immune mediated renal diseases, hepatobiliary diseases, multiple sclerosis, atherosclerosis, promotion of tumor growth, or degenerative joint disease and other inflammatory conditions disclosed herein.

An illustrative nucleotide sequence that encodes human ZcytoR21x1 is provided by SEQ ID NO:1; the encoded polypeptide is shown in SEQ ID NO:2. Another illustrative nucleotide sequence that encodes human ZcytoR21x2 is provided by SEQ ID NO:4; the encoded polypeptide is shown in SEQ ID NO:5. Another illustrative nucleotide sequence that encodes human ZcytoR21x3 is provided by SEQ ID NO:7; the encoded polypeptide is shown in SEQ ID NO:8. Another illustrative nucleotide sequence that encodes human ZcytoR21x4 is provided by SEQ ID NO:10; the encoded polypeptide is shown in SEQ ID NO:11. Another illustrative nucleotide sequence that encodes human ZcytoR21x6 is provided by SEQ ID NO:20 the encoded polypeptide is shown in SEQ ID NO:21. Yet another illustrative nucleotide sequence that encodes human ZcytoR21x13 is provided by SEQ ID NO:106; the encoded polypeptide is shown in SEQ ID NO:107. Yet another illustrative nucleotide sequence that encodes: human ZcytoR21x14 is provided by SEQ ID NO:108; the encoded polypeptide is shown in SEQ ID NO:109. Yet another illustrative nucleotide sequence that encodes a variant ZcytoR21s2 is provided by SEQ ID NO:112; the encoded polypeptide is shown in SEQ ID NO:113.

ZcytoR21 functions as a receptor for IL-17C (SEQ ID NOs:16 & 17). ZcytoR21 can act as a monomer, a homodimer or a heterodimer. Preferably, ZcytoR21 acts as a homodimeric receptor for IL-17C. ZcytoR21 can also act as a heterodimeric receptor subunit for a IL-17-related cytokine. Including IL-17A, IL-17B, IL-17C, IL-17D, IL-17E and IL-17F. ZcytoR21 is disclosed in commonly owned U.S. patent application Ser. No. 10/192,434, and commonly owned WIPO publication WO 03/006,609, both of which are incorporated herein in their entirety by reference. Analysis of a human cDNA clone encoding ZcytoR21x1 (SEQ ID NO:1) revealed an open reading frame encoding 667 amino acids comprising a putative signal sequence of approximately 23 amino acid residues (amino acid residues 1 to 23 of SEQ ID NO:2), an extracellular ligand-binding domain of approximately 431 amino acid residues (amino acid residues 24-454 of SEQ ID NO:2; SEQ ID NO:3), a transmembrane domain of approximately 23 amino acid residues (amino acid residues 455-477 of SEQ ID NO:2), and an intracellular domain of approximately 190 amino acid residues (amino acid residues 478 to 667 of SEQ ID NO:2).

Yet another illustrative nucleotide sequence that encodes a variant human ZcytoR21, designated as “ZcytoR21x2” is provided by SEQ ID NO:4, the encoded polypeptide is shown in SEQ ID NO:5. Analysis of a human cDNA clone encoding ZcytoR21x2 revealed an open reading frame encoding 589 amino acids (SEQ ID NO:5) comprising a putative signal sequence of approximately 23 amino acid residues (amino acid residues 1 to 23 of SEQ ID NO:5), an extracellular ligand-binding domain of approximately 353 amino acid residues (amino acid residues 24-376 of SEQ ID NO:5; SEQ ID NO:6), a transmembrane domain of approximately 23 amino acid residues (amino acid residues 377-399 of SEQ ID NO:5), and an intracellular domain of approximately 190 amino acid residues (amino acid residues 400 to 589 of SEQ ID NO:5).

Yet another illustrative nucleotide sequence that encodes a variant human ZcytoR21, designated as “ZcytoR21x3” is provided by SEQ ID NO:7, the encoded polypeptide is shown in SEQ ID NO:8. Analysis of a human cDNA clone encoding ZcytoR21x3 revealed an open reading frame encoding 609 amino acids (SEQ ID NO:8) comprising a putative signal sequence of approximately 23 amino acid residues (amino acird residues 1 to 23 of SEQ ID NO:8), an extracellular ligand-binding domain of approximately 373 amino acid residues (amino acid residues 24-396 of SEQ ID NO:8; SEQ ID NO:9), a transmembrane domain of approximately 23 amino acid residues (amino acid residues 397-419 of SEQ ID NO:8), and an intracellular domain of approximately 190 amino acid residues (amino acid residues 420 to 609 of SEQ ID NO:8).

Yet another illustrative nucleotide sequence that encodes a variant human ZcytoR21 which may be a naturally occurring soluble receptor, designated as “ZcytoR21x4” is provided by SEQ ID NO:10, the encoded polypeptide is shown in SEQ ID NO:11. Analysis of a human cDNA clone encoding ZcytoR21x4 revealed an open reading frame encoding 533 amino acids (SEQ ID NO:11) comprising a putative signal sequence of approximately 23 amino acid residues (amino acid residues 1 to 23 of SEQ ID NO:11), and an extracellular ligand-binding domain of approximately 510 amino acid residues (amino acid residues 24-533 of SEQ ID NO:11; SEQ ID NO:12).

Yet another illustrative nucleotide sequence that encodes a variant human ZcytoR21, designated as “ZcytoR21x6” is provided by SEQ ID NO:20, the encoded polypeptide is shown in SEQ ID NO:21. Analysis of a human cDNA clone encoding ZcytoR21x6 revealed an open reading frame encoding 627 amino acids (SEQ ID NO:21) comprising a putative signal sequence of approximately 23 amino acid residues (amino acid residues 1 to 23 of SEQ ID NO:21), a cytoplasmic domain of approximately 192 amino acid residues (amino acid residues 436 to 627 of SEQ ID NO:21), a transmembrane domain of approximately 21 amino acid residues (amino acid residues 415 ot 435 of SEQ ID NO:21) and an extracellular ligand-binding domain of approximately 391 amino acid residues (amino acid residues 24-414 of SEQ ID NO:21). The IL-17C binding domain (or ligand binding domain) comprises approximately 279 amino acid residues (amino acid residues 136 to 414 of SEQ ID NO:21).

Yet another illustrative nucleotide sequence that encodes a variant human ZcytoR21 which may be a naturally occurring soluble receptor, designated as “ZcytoR21x7” is provided by SEQ ID NO:22, the encoded polypeptide is shown in SEQ ID NO:23.

Yet another illustrative nucleotide sequence that encodes a variant human ZcytoR21, designated as “ZcytoR21x13” is provided by SEQ ID NO:106, the encoded polypeptide is shown in SEQ ID NO:107. Analysis of a human cDNA clone encoding ZcytoR21x13 revealed an open reading frame encoding 650 amino acids (SEQ ID NO:107) comprising a putative signal sequence of approximately 23 amino acid residues (amino acid residues 1 to 23 of SEQ ID NO:107), a cytoplasmic domain of approximately 192 amino acid residues (amino acid residues 459 to 650 of SEQ ID NO:107), a transmembrane domain of approximately 27 amino acid residues (amino acid residues 459 to 458 of SEQ ID NO:107) and an extracellular ligand-binding domain of approximately 414 amino acid residues (amino acid residues 24-437 of SEQ ID NO:107; SEQ ID NO:122). The IL-17C binding domain (or ligand binding domain) comprises approximately 279 amino acid residues (amino acid residues 159 to 437 of SEQ ID NO:107).

Yet another illustrative nucleotide sequence that encodes a variant human ZcytoR21 soluble receptor, designated as “ZcytoR21x14” is provided by SEQ ID NO:108, the encoded polypeptide is shown in SEQ ID NO:109. Analysis of a human cDNA clone encoding ZcytoR21x14 revealed an open reading frame encoding 414 amino acids (SEQ ID NO:109) comprising a putative signal sequence of approximately 23 amino acid residues (amino acid residues 1 to 23 of SEQ ID NO:109), and an extracellular ligand-binding domain of approximately 391 amino acid residues (amino acid residues 24-414 of SEQ ID NO:109). The IL-17C binding domain (or ligand binding domain) comprises approximately 279 amino acid residues (amino acid residues 136 to 414 of SEQ ID NO:109).

Yet another illustrative nucleotide sequence that encodes an engineered soluble human ZcytoR21, designated as “ZcytoR21s2” is provided by SEQ ID NO:112, the encoded polypeptide is shown in SEQ ID NO:113.

The present invention also includes preferred IL-17C binding regions. An illustrative example of a preferred binding region is provided by SEQ ID NO:114; the encoded polypeptide is shown in SEQ ID NO:115.

Another illustrative example of a preferred binding region is provided by SEQ ID NO:116; the encoded polypeptide is shown in SEQ ID NO:117.

Yet another illustrative example of a preferred binding region is provided by SEQ ID NO:118; the encoded polypeptide is shown in SEQ ID NO:119.

An illustrative nucleotide sequence that encodes a murine ZcytoR21 is provided by SEQ ID NO:13; the encoded polypeptide is shown in SEQ ID NO:14. Analysis of murine ZcytoR21 revealed an extracellular ligand-binding domain of approximately 638 amino acid residues (amino acid residues 26-663 of SEQ ID NO:14; SEQ ID NO:15). Murine ZcytoR21 functions as a receptor for murine IL-17C (SEQ ID NOs:18 & 19).

An illustrative nucleotide sequence that encodes a murine ZcytoR21 variant is provided by SEQ ID NO:160; the encoded polypeptide is shown in SEQ ID NO:161. Analysis of murine ZcytoR21 revealed an extracellular ligand-binding domain of approximately 568 amino acid residues (amino acid residues 24-591 of SEQ ID NO:161).

Another illustrative nucleotide sequence that encodes a murine ZcytoR21 is provided by SEQ ID NO:110; the encoded polypeptide is shown in SEQ ID NO:111. Analysis of murine ZcytoR21 revealed a cytoplasmic domain of 201 amino acid residues (amino acid residues 461 to 661 of SEQ ID NO:111), a transmembrane domain of 22 amino acid residues (amino acid residues 439 to 460 of SEQ ID NO:111), an extracellular ligand-binding domain of approximately 415 amino acid residues (amino acid residues 24 to 438 of SEQ ID NO:111). The murine IL-17C binding domain (or ligand binding domain) comprises approximately 275 amino acid residues (amino acid residues 136 to 410 of SEQ ID NO:111).

Yet another illustrative nucleotide sequence that encodes an engineered soluble murine ZcytoR21, designated as “mZcytoR21s2” is provided by SEQ ID NO:120, the encoded polypeptide is shown in SEQ ID NO:121.

The ZcytoR21 gene resides in human chromosome 3p25.3.

As described below, the present invention provides isolated polypeptides comprising an amino acid sequence that is at least 70%, at least 80%, or at least 90%, or greater than 95%, such as 96%, 97%, 98%, or greater than 99% or more identical to a reference amino acid sequence of any of SEQ ID NOs:2, 5, 8, 11, 14, 21, 23, 107, 109, 111 or 113 wherein the isolated polypeptide specifically binds with an antibody that specifically binds with a polypeptide comprising the amino acid sequence of any of SEQ ID NOs: 2, 5, 8, 11, 14, 21, 23, 107, 109, 111, 113, 115, 117 or 119. The present invention provides isolated polypeptides comprising an amino acid sequence that is at least 70%, at least 80%, or at least 90%, or greater than 95%, such as 96%, 97%, 98%, or greater than 99% or more identical to a reference amino acid sequence of 24-589 of SEQ ID NO:5, wherein the isolated polypeptide specifically binds with an antibody that specifically binds with a polypeptide comprising the amino acid sequence of SEQ ID NO:5. The present invention provides isolated polypeptides comprising an amino acid sequence that is at least 70%, at least 80%, or at least 90%, or greater than 95%, such as 96%, 97%, 98%, or greater than 99% or more identical to a reference amino acid sequence of 24-609 of SEQ ID NO:8, wherein the isolated polypeptide specifically binds with an antibody that specifically binds with a polypeptide comprising the amino acid sequence of SEQ ID NO:8. The present invention provides isolated polypeptides comprising an amino acid sequence that is at least 70%, at least 80%, or at least 90%, or greater than 95%, such as 96%, 97%, 98%, or greater than 99% or more identical to a reference amino acid sequence of 24-533 of SEQ ID NO:1, wherein the isolated polypeptide specifically binds with an antibody that specifically binds with a polypeptide comprising the amino acid sequence of SEQ ID NO:11. The present invention provides isolated polypeptides comprising an amino acid sequence that is at least 70%, at least 80%, or at least 90%, or greater than 95%, such as 96%, 97%, 98%, or greater than 99% or more identical to any of SEQ ID NOs: 2, 5, 8, 11, 14, 21, 23, 107, 109, 111, 113, 115, 117 or 119, wherein the isolated polypeptide specifically binds with an antibody that specifically binds with a polypeptide comprising the amino acid sequence of any of SEQ ID NOs: 2, 5, 8, 11, 14, 21, 23, 107, 109, 111, 113, 115, 117 or 119. The present invention provides isolated polypeptides comprising an amino acid sequence that is at least 70%, at least 80%, or at least 90%, or greater than 95%, such as 96%, 97%, 98%, or greater than 99% or more identical to any of SEQ ID NOs: 2, 5, 8, 11, 14, 21, 23, 107, 109, 111, 113, 115, 117 or 119, wherein the isolated polypeptide specifically binds with an antibody that specifically binds with a polypeptide comprising the amino acid sequence of any of SEQ ID NOs: 2, 5, 8, 11, 14, 21, 23, 107, 109, 111, 113, 115, 117 or 119. The present invention provides isolated polypeptides comprising an amino acid sequence that is at least 70%, at least 80%, or at least 90%, or greater than 95%, such as 96%, 97%, 98%, or greater than 99% or more identical to a reference amino acid sequence of 26-663 of SEQ ID NO:17, wherein the isolated polypeptide specifically binds with an antibody that specifically binds with a polypeptide comprising the amino acid sequence of SEQ ID NO:17.

The present invention also provides isolated polypeptides comprising an extracellular domain, wherein the extracellular domain comprises an amino acid sequence selected from the group consisting of: (a) amino acid residues 24 to 454 of SEQ ID NO:2, (b) SEQ ID NO:3; (c) amino acid residues 24-376 of SEQ ID NO:5; (d) SEQ ID NO:6; (e) amino acid residues 24-396 of SEQ ID NO:8; (f) SEQ ID NO:9; (g) amino acid residues 24-533 of SEQ ID NO:11; (h) SEQ ID NO:12; (i) amino acid residues 26-663 of SEQ ID NO:14; or (O) SEQ ID NO:15, wherein the isolated polypeptide specifically binds with an antibody that specifically binds with a polypeptide consisting of either the amino acid sequence of any of SEQ ID NOs: 2, 5, 8, 11, 14, 21, 23, 107, 109, 111, 113, 115, 117 or 119. Such polypeptides may further comprise a transmembrane domain that resides in a carboxyl-terminal position relative to the extracellular domain, wherein the transmembrane domain comprises an amino acid sequence selected from the group consisting of: (a) amino acid residues 455 to 477 of SEQ ID NO:2; (b) amino acid residues 377 to 399 of SEQ ID NO:5; or (c) amino acid residues 397 to 419 of SEQ ID NO:8. These polypeptides may also comprise an intracellular domain that resides in a carboxyl-terminal position relative to the transmembrane domain, and optionally, a signal secretory sequence that resides in an amino-terminal position relative to the extracellular domain.

The present invention also includes variant ZcytoR21 polypeptides, wherein the amino acid sequence of the variant polypeptide shares an identity with the amino acid sequence of SEQ ID NOs: 2, 5, 8, 11, 14, 21, 23, 107, 109, 111, 113, 115, 117 or 119, selected from the group consisting of at least 70% identity, at least 80% identity, at least 90% identity, at least 95% identity, or greater than 95% identity, and wherein any difference between the amino acid sequence of the variant polypeptide and the amino acid sequence of SEQ ID NOs: 2, 5, 8, 11, 14, 21, 23, 107, 109, 111, 113, 115, 117 or 119 is due to one or more conservative amino acid substitutions.

Moreover, the present invention also provides isolated polypeptides as disclosed above that bind IL-17C (e.g., human IL-17C polypeptide sequence as shown in SEQ ID NO: 17). The human IL-17C polynucleotide sequence is shown in SEQ ID NO:16. The mouse IL-17C polynucleotide sequence is shown in SEQ ID NO:18, and corresponding polyepeptide is shown in SEQ ID NO:19.

The present invention also provides isolated polypeptides and epitopes comprising at least 15 contiguous amino acid residues of an amino acid sequence of SEQ ID NOs: 2, 5, 8, 11, 14, 21, 23, 107, 109, 111, 113, 115, 117 or 119. Illustrative polypeptides include polypeptides that either comprise, or consist of SEQ ID NOs: 2, 5, 8, 11, 14, 21, 23, 107, 109, 111, 113, 115, 117 or 119, an antigenic epitope thereof, or a functional IL-17C binding fragment thereof. Moreover, the present invention also provides isolated polypeptides as disclosed above that bind to, block, inhibit, reduce, antagonize or neutralize the activity of IL-17C.

The present invention also includes variant ZcytoR21 polypeptides, wherein the amino acid sequence of the variant polypeptide shares an identity with the amino acid residues of SEQ ID NO: SEQ ID NOs: 2, 5, 8, 11, 14, 21, 23, 107, 109, 111, 113, 115, 117 or 119 selected from the group consisting of at least 70% identity, at least 80% identity, at least 90% identity, at least 95% identity, or greater than 95% identity, such as 96%, 97%, 98%, or greater than 99% or more identity, and wherein any difference between the amino acid sequence of the variant polypeptide and the corresponding amino acid sequence is due to one or more conservative amino acid substitutions. Such conservative amino acid substitutions are described herein. Moreover, the present invention also provides isolated polypeptides as disclosed above that bind to, block, inhibit, reduce, antagonize or neutralize the activity of IL-17C.

The present invention further provides antibodies and antibody fragments that specifically bind with such polypeptides. Exemplary antibodies include neutralizing antibodies, polyclonal antibodies, murine monoclonal antibodies, humanized antibodies derived from murine monoclonal antibodies, and human monoclonal antibodies. Illustrative antibody fragments include F(ab′)₂, F(ab)₂, Fab′, Fab, Fv, scFv, and minimal recognition units. Neutralizing antibodies preferably bind ZcytoR21 such that the interaction of IL-17C with ZcytoR21 is blocked, inhibited, reduced, antagonized or neutralized; anti-ZcytoR21 neutralizing antibodies such that the binding of either IL-17C to ZcytoR21 is blocked, inhibited, reduced, antagonized or neutralized are also encompassed by the present invention. That is, the neutralizing anti-ZcytoR21 antibodies of the present invention can either either bind, block, inhibit, reduce, antagonize or neutralize IL-17C singly, or bind, block, inhibit, reduce, antagonize or neutralize IL-17C and another cytokine, such as together. The present invention further includes compositions comprising a carrier and a peptide, polypeptide, or antibody described herein.

In addition, the present invention provides pharmaceutical compositions comprising a pharmaceutically acceptable carrier and at least one of such an expression vector or recombinant virus comprising such expression vectors. The present invention further includes pharmaceutical compositions, comprising a pharmaceutically acceptable carrier and a polypeptide or antibody described herein.

The present invention also contemplates anti-idiotype antibodies, or anti-idiotype antibody fragments, that specifically bind an antibody or antibody fragment that specifically binds a polypeptide comprising the amino acid sequence of SEQ ID NOs: 2, 5, 8, 11, 14, 21, 23, 107, 109, 111, 113, 115, 117 or 119 or a fragment thereof. An exemplary anti-idiotype antibody binds with an antibody that specifically binds a polypeptide consisting of any of SEQ ID NOs: 2, 5, 8, 11, 14, 21, 23, 107, 109, 111, 113, 115, 117 or 119.

The present invention also provides fusion proteins, comprising a ZcytoR21 polypeptide and an immunoglobulin moiety. In such fusion proteins, the immunoglobulin moiety may be an immunoglobulin heavy chain constant region, such as a human F_{C} fragment. The present invention further includes isolated nucleic acid molecules that encode such fusion proteins (e.g. SEQ ID NO:123).

The present invention also provides polyclonal and monoclonal antibodies that bind to polypeptides comprising an ZcytoR21 extracellular domain such as monomeric, homodimeric, heterodimeric and multimeric receptors, including soluble receptors. Moreover, such antibodies can be used antagonize the binding of ZcytoR21 ligands, such as IL-17C (SEQ ID NO:17), to the ZcytoR21 receptor.

These and other aspects of the invention will become evident upon reference to the following detailed description. In addition, various references are identified below and are incorporated by reference in their entirety.

B) Definitions

In the description that follows, a number of terms are used extensively. The following definitions are provided to facilitate understanding of the invention.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

The term “complement of a nucleic acid molecule” refers to a nucleic acid molecule having a complementary nucleotide sequence and reverse orientation as compared to a reference nucleotide sequence. For example, the sequence 5′ATGCACGGG 3′ is complementary to 5′CCCGTGCAT 3′.

The term “degenerate nucleotide sequence” denotes a sequence of nucleotides that includes one or more degenerate codons as compared to a reference nucleic acid molecule that encodes a polypeptide. Degenerate codons contain different triplets of nucleotides, but encode the same amino acid residue (i.e., GAU and GAC triplets each encode Asp).

The term “structural gene” refers to a nucleic acid molecule that is transcribed into messenger RNA (mRNA), which is then translated into a sequence of amino acids characteristic of a specific polypeptide.

An “isolated nucleic acid molecule” is a nucleic acid molecule that is not integrated in the genomic DNA of an organism. For example, a DNA molecule that encodes a growth factor that has been separated from the genomic DNA of a cell is an isolated DNA molecule. Another example of an isolated nucleic acid molecule is a chemically-synthesized nucleic acid molecule that is not integrated in the genome of an organism. A nucleic acid molecule that has been isolated from a particular species is smaller than the complete DNA molecule of a chromosome from that species.

A “nucleic acid molecule construct” is a nucleic acid molecule, either single- or double-stranded, that has been modified through human intervention to contain segments of nucleic acid combined and juxtaposed in an arrangement not existing in nature.

“Linear DNA” denotes non-circular DNA molecules having free 5′ and 3′ ends. Linear DNA can be prepared from closed circular DNA molecules, such as plasmids, by enzymatic digestion or physical disruption.

“Complementary DNA (cDNA)” is a single-stranded DNA molecule that is formed from an mRNA template by the enzyme reverse transcriptase. Typically, a primer complementary to portions of mRNA is employed for the initiation of reverse transcription. Those skilled in the art also use the term “cDNA” to refer to a double-stranded DNA molecule consisting of such a single-stranded DNA molecule and its complementary DNA strand. The term “cDNA” also refers to a clone of a cDNA molecule synthesized from an RNA template.

A “promoter” is a nucleotide sequence that directs the transcription of a structural gene. Typically, a promoter is located in the 5′ non-coding region of a gene, proximal to the transcriptional start site of a structural gene. Sequence elements within promoters that function in the initiation of transcription are often characterized by consensus nucleotide sequences. These promoter elements include RNA polymerase binding sites, TATA sequences, CAAT sequences, differentiation-specific elements (DSEs; McGehee et al., Mol. Endocrinol. 7:551 (1993)), cyclic AMP response elements (CREs), serum response elements (SREs; Treisman, Seminars in Cancer Biol. 1:47 (1990)), glucocorticoid response elements (GREs), and binding sites for other transcription factors, such as CRE/ATF (O'Reilly et al., J. Biol. Chem. 267:19938 (1992)), AP2 (Ye et al., J. Biol. Chem. 269:25728 (1994)), SP1, cAMP response element binding protein (CREB; Loeken, Gene Expr. 3:253 (1993)) and octamer factors (see, in general, Watson et al., eds., Molecular Biology of the Gene, 4th ed. (The Benjamin/Cummings Publishing Company, Inc. 1987), and Lemaigre and Rousseau, Biochem. J. 303:1 (1994)). If a promoter is an inducible promoter, then the rate of transcription increases in response to an inducing agent. In contrast, the rate of transcription is not regulated by an inducing agent if the promoter is a constitutive promoter. Repressible promoters are also known.

A “core promoter” contains essential nucleotide sequences for promoter function, including the TATA box and start of transcription. By this definition, a core promoter may or may not have detectable activity in the absence of specific sequences that may enhance the activity or confer tissue specific activity.

A “regulatory element” is a nucleotide sequence that modulates the activity of a core promoter. For example, a regulatory element may contain a nucleotide sequence that binds with cellular factors enabling transcription exclusively or preferentially in particular cells, tissues, or organelles. These types of regulatory elements are normally associated with genes that are expressed in a “cell-specific,” “tissue-specific,” or “organelle-specific” manner.

An “enhancer” is a type of regulatory element that can increase the efficiency of transcription, regardless of the distance or orientation of the enhancer relative to the start site of transcription.

“Heterologous DNA” refers to a DNA molecule, or a population of DNA molecules, that does not exist naturally within a given host cell. DNA molecules heterologous to a particular host cell may contain DNA derived from the host cell species (i.e., endogenous DNA) so long as that host DNA is combined with non-host DNA (i.e., exogenous DNA). For example, a DNA molecule containing a non-host DNA segment encoding a polypeptide operably linked to a host DNA segment comprising a transcription promoter is considered to be a heterologous DNA molecule. Conversely, a heterologous DNA molecule can comprise an endogenous gene operably linked with an exogenous promoter. As another illustration, a DNA molecule comprising a gene derived from a wild-type cell is considered to be heterologous DNA if that DNA molecule is introduced into a mutant cell that lacks the wild-type gene.

A “polypeptide” is a polymer of amino acid residues joined by peptide bonds, whether produced naturally or synthetically. Polypeptides of less than about 10 amino acid residues are commonly referred to as “peptides.”

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

A peptide or polypeptide encoded by a non-host DNA molecule is a “heterologous” peptide or polypeptide.

A “cloning vector” is a nucleic acid molecule, such as a plasmid, cosmid, or bacteriophage, that has the capability of replicating autonomously in a host cell. Cloning vectors typically contain one or a small number of restriction endonuclease recognition sites that allow insertion of a nucleic acid molecule in a determinable fashion without loss of an essential biological function of the vector, as well as nucleotide sequences encoding a marker gene that is suitable for use in the identification and selection of cells transformed with the cloning vector. Marker genes typically include genes that provide tetracycline resistance or ampicillin resistance.

An “expression vector” is a nucleic acid molecule encoding a gene that is expressed in a host cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter.

A “recombinant host” is a cell that contains a heterologous nucleic acid molecule, such as a cloning vector or expression vector. In the present context, an example of a recombinant host is a cell that produces ZcytoR21 from an expression vector. In contrast, ZcytoR21 can be produced by a cell that is a “natural source” of ZcytoR21, and that lacks an expression vector.

“Integrative transformants” are recombinant host cells, in which heterologous DNA has become integrated into the genomic DNA of the cells.

A “fusion protein” is a hybrid protein expressed by a nucleic acid molecule comprising nucleotide sequences of at least two genes. For example, a fusion protein can comprise at least part of a ZcytoR21 polypeptide fused with a polypeptide that binds an affinity matrix. Such a fusion protein provides a means to isolate large quantities of ZcytoR21 using affinity chromatography.

The term “receptor” denotes a cell-associated protein that binds to a bioactive molecule termed a “ligand.” This interaction mediates the effect of the ligand on the cell. Receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor). Membrane-bound receptors are characterized by a multi-domain structure comprising an extracellular ligand-binding domain and an intracellular effector domain that is typically involved in signal transduction. In certain membrane-bound receptors, the extracellular ligand-binding domain and the intracellular effector domain are located in separate polypeptides that comprise the complete functional receptor.

In general, the binding of ligand to receptor results in a conformational change in the receptor that causes an interaction between the effector domain and other molecule(s) in the cell, which in turn leads to an alteration in the metabolism of the cell. Metabolic events that are often linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids.

A “soluble receptor” is a receptor polypeptide that is not bound to a cell membrane. Soluble receptors are most commonly ligand-binding receptor polypeptides that lack transmembrane and cytoplasmic domains, and other linkage to the cell membrane such as via glycophosphoinositol (gpi). Soluble receptors can comprise additional amino acid residues, such as affinity tags that provide for purification of the polypeptide or provide sites for attachment of the polypeptide to a substrate, or immunoglobulin constant region sequences. Many cell-surface receptors have naturally occurring, soluble counterparts that are produced by proteolysis or translated from alternatively spliced mRNAs. Soluble receptors can be monomeric, homodimeric, heterodimeric, or multimeric, with multimeric receptors generally not comprising more than 9 subunits, preferably not comprising more than 6 subunits, and most preferably not comprising more than 3 subunits. Receptor polypeptides are said to be substantially free of transmembrane and intracellular polypeptide segments when they lack sufficient portions of these segments to provide membrane anchoring or signal transduction, respectively. Soluble receptors of cytokine receptors generally comprise the extracellular cytokine binding domain free of a transmembrane domain and intracellular domain. For example, representative soluble receptors include soluble receptors for IL-17R as shown in SEQ ID NOs:3, or 113. It is well within the level of one of skill in the art to delineate what sequences of a known cytokine receptor sequence comprise the extracellular cytokine binding domain free of a transmembrane domain and intracellular domain. Moreover, one of skill in the art using the genetic code can readily determine polynucleotides that encode such soluble receptor polyptides.

The term “secretory signal sequence” denotes a DNA sequence that encodes a peptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

An “isolated polypeptide” is a polypeptide that is essentially free from contaminating cellular components, such as carbohydrate, lipid, or other proteinaceous impurities associated with the polypeptide in nature. Typically, a preparation of isolated polypeptide contains the polypeptide in a highly purified form, i.e., at least about 80% pure, at least about 90% pure, at least about 95% pure, greater than 95% pure, such as 96%, 97%, or 98% or more pure, or greater than 99% pure. One way to show that a particular protein preparation contains an isolated polypeptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide in alternative physical forms, such as dimers or alternatively glycosylated or derivatized forms.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “expression” refers to the biosynthesis of a gene product. For example, in the case of a structural gene, expression involves transcription of the structural gene into mRNA and the translation of mRNA into one or more polypeptides.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a polypeptide encoded by a splice variant of an mRNA transcribed from a gene.

As used herein, the term “immunomodulator” includes cytokines, stem cell growth factors, lymphotoxins, co-stimulatory molecules, hematopoietic factors, an dthe like, and synthetic analogs of these molecules.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of less than 10⁹ M⁻¹.

An “anti-idiotype antibody” is an antibody that binds with the variable region domain of an immunoglobulin. In the present context, an anti-idiotype antibody binds with the variable region of an anti-ZcytoR21 antibody, and thus, an anti-idiotype antibody mimics an epitope of ZcytoR21.

An “antibody fragment” is a portion of an antibody such as F(ab′)₂, F(ab)₂, Fab′, Fab, and the like. Regardless of structure, an antibody fragment binds with the same antigen that is recognized by the intact antibody. For example, an anti-ZcytoR21 monoclonal antibody fragment binds with an epitope of ZcytoR21.

The term “antibody fragment” also includes a synthetic or a genetically engineered polypeptide that binds to a specific antigen, such as polypeptides consisting of the light chain variable region, “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.

A “chimeric antibody” is a recombinant protein that contains the variable domains and, complementary determining regions derived from a rodent antibody, while the remainder of the antibody molecule is derived from a human antibody.

“Humanized antibodies” are recombinant proteins in which murine complementarity determining regions of a monoclonal antibody have been transferred from heavy and light variable chains of the murine immunoglobulin into a human variable domain. Construction of humanized antibodies for therapeutic use in humans that are derived from murine antibodies, such as those that bind to or neutralize a human protein, is within the skill of one in the art.

As used herein, a “therapeutic agent” is a molecule or atom which is conjugated to an antibody moiety to produce a conjugate which is useful for therapy. Examples of therapeutic agents include drugs, toxins, immunomodulators, chelators, boron compounds, photoactive agents or dyes, and radioisotopes.

A “detectable label” is a molecule or atom which can be conjugated to an antibody moiety to produce a molecule useful for diagnosis. Examples of detectable labels include chelators, photoactive agents, radioisotopes, fluorescent agents, paramagnetic ions, or other marker moieties.

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075 (1985); Nilsson et al., Methods Enzymol. 198:3 (1991)), glutathione S transferase (Smith and Johnson, Gene 67:31 (1988)), Glu-Glu affinity tag (Grussenmeyer et al., Proc. Natl. Acad. Sci. USA 82:7952 (1985)), substance P, FLAG peptide (Hopp et at., Biotechnology 6:1204 (1988)), streptavidin binding peptide, or other antigenic epitope or binding domain. See, in general, Ford et al., Protein Expression and Purification 2:95 (1991). DNA molecules encoding affinity tags are available from commercial suppliers (e.g., Pharmacia Biotech, Piscataway, N.J.).

A “naked antibody” is an entire antibody, as opposed to an antibody fragment, which is not conjugated with a therapeutic agent. Naked antibodies include both polyclonal and monoclonal antibodies, as well as certain recombinant antibodies, such as chimeric and humanized antibodies.

As used herein, the term “antibody component” includes both an entire antibody and an antibody fragment.

An “immunoconjugate” is a conjugate of an antibody component with a therapeutic agent or a detectable label.

As used herein, the term “antibody fusion protein” refers to a recombinant molecule that comprises an antibody component and a ZcytoR21 polypeptide component. Examples of an antibody fusion protein include a protein that comprises a ZcytoR21 extracellular domain, and either an Fc domain or an antigen-binding region (e.g. SEQ ID NO:123).

A “target polypeptide” or a “target peptide” is an amino acid sequence that comprises at least one epitope, and that is expressed on a target cell, such as a tumor cell, or a cell that carries an infectious agent antigen. T cells recognize peptide epitopes presented by a major histocompatibility complex molecule to a target polypeptide or target peptide and typically lyse the target cell or recruit other immune cells to the site of the target cell, thereby killing the target cell.

An “antigenic peptide” is a peptide which will bind a major histocompatibility complex molecule to form an MHC-peptide complex which is recognized by a T cell, thereby inducing a cytotoxic lymphocyte response upon presentation to the T cell. Thus, antigenic peptides are capable of binding to an appropriate major histocompatibility complex molecule and inducing a cytotoxic T cells response, such as cell lysis or specific cytokine release against the target cell which binds or expresses the antigen. The antigenic peptide can be bound in the context of a class I or class II major histocompatibility complex molecule, on an antigen presenting cell or on a target cell.

In eukaryotes, RNA polymerase II catalyzes the transcription of a structural gene to produce mRNA. A nucleic acid molecule can be designed to contain an RNA polymerase II template in which the RNA transcript has a sequence that is complementary to that of a specific mRNA. The RNA transcript is termed an “anti-sense RNA” and a nucleic acid molecule that encodes the anti-sense RNA is termed an “anti-sense gene.” Anti-sense RNA molecules are capable of binding to mRNA molecules, resulting in an inhibition of mRNA translation.

An “anti-sense oligonucleotide specific for ZcytoR21” or a “ZcytoR21 anti-sense oligonucleotide” is an oligonucleotide having a sequence (a) capable of forming a stable triplex with a portion of the ZcytoR21 gene, or (b) capable of forming a stable duplex with a portion of an mRNA transcript of the ZcytoR21 gene.

A “ribozyme” is a nucleic acid molecule that contains a catalytic center. The term includes RNA enzymes, self-splicing RNAs, self-cleaving RNAs, and nucleic acid molecules that perform these catalytic functions. A nucleic acid molecule that encodes a ribozyme is termed a “ribozyme gene.”

An “external guide sequence” is a nucleic acid molecule that directs the endogenous ribozyme, RNase P, to a particular species of intracellular mRNA, resulting in the cleavage of the mRNA by RNase P. A nucleic acid molecule that encodes an external guide sequence is termed an “external guide sequence gene.”

The term “variant ZcytoR21 gene” refers to nucleic acid molecules that encode a polypeptide having an amino acid sequence that is a modification of SEQ ID NOs: 2, 5, 8, 11, 14, 21, 23, 107, 109, 111, 113, 115, 117 or 119. Such variants include naturally-occurring polymorphisms of ZcytoR21 genes, as well as synthetic genes that contain conservative amino acid substitutions of the amino acid sequence of SEQ ID NOs: 2, 5, 8, 11, 14, 21, 23, 107, 109, 111, 113, 115, 117 or 119. Additional variant forms of ZcytoR21 genes are nucleic acid molecules that contain insertions or deletions of the nucleotide sequences described herein. A variant ZcytoR21 gene can be identified, for example, by determining whether the gene hybridizes with a nucleic acid molecule having the nucleotide sequence of SEQ ID NOs:1, 4, 7, 10, 13, 20, 22, 106, 108, 110 or 112, or any of their complements, under stringent conditions.

Alternatively, variant ZcytoR21 genes can be identified by sequence comparison. Two amino acid sequences have “100% amino acid sequence identity” if the amino acid residues of the two amino acid sequences are the same when aligned for maximal correspondence. Similarly, two nucleotide sequences have “100% nucleotide sequence identity” if the nucleotide residues of the two nucleotide sequences are the same when aligned for maximal correspondence. Sequence comparisons can be performed using standard software programs such as those included in the LASERGENE bioinformatics computing suite, which is produced by DNASTAR (Madison, Wis.). Other methods for comparing two nucleotide or amino acid sequences by determining optimal alignment are well-known to those of skill in the art (see, for example, Peruski and Peruski, The Internet and the New Biology: Tools for Genomic and Molecular Research (ASM Press, Inc. 1997), Wu et al. (eds.), “Information Superhighway and Computer Databases of Nucleic Acids and Proteins,” in Methods in Gene Biotechnology, pages 123-151 (CRC Press, Inc. 1997), and Bishop (ed.), Guide to Human Genome Computing, 2nd Edition (Academic Press, Inc. 1998)). Particular methods for determining sequence identity are described below.

Regardless of the particular method used to identify a variant ZcytoR21 gene or variant ZcytoR21 polypeptide, a variant gene or polypeptide encoded by a variant gene may be functionally characterized the ability to bind specifically to an anti-ZcytoR21 antibody. A variant ZcytoR21 gene or variant ZcytoR21 polypeptide may also be functionally characterized the ability to bind to its ligand, for example, IL-17C, using a biological or biochemical assay described herein.

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The term “ortholog” denotes a polypeptide or protein obtained from one species that is the functional counterpart of a polypeptide or protein from a different species. Sequence differences among orthologs are the result of speciation.

“Paralogs” are distinct but structurally related proteins made by an organism. Paralogs are believed to arise through gene duplication. For example, α-globin, β-globin, and myoglobin are paralogs of each other.

The present invention includes functional fragments of ZcytoR21 genes. Within the context of this invention, a “functional fragment” of a ZcytoR21 gene refers to a nucleic acid molecule that encodes a portion of a ZcytoR21 polypeptide which is a domain described herein or at least specifically binds with an anti-ZcytoR21 antibody.

Due to the imprecision of standard analytical methods, molecular weights and lengths of polymers are understood to be approximate values. When such a value is expressed as “about” X or “approximately” X, the stated value of X will be understood to be accurate to ±10%.

C) Production of ZcytoR21 Polynucleotides or Genes

Nucleic acid molecules encoding a human ZcytoR21 gene can be obtained by screening a human cDNA or genomic library using polynucleotide probes based upon any of SEQ ID NOs:1, 4, 7, 10, 13, 20, 22, 106, 108, or 112. These techniques are standard and well-established, and may be accomplished using cloning kits available by commercial suppliers. See, for example, Ausubel et al. (eds.), Short Protocols in Molecular Biology, 3rd Edition, John Wiley & Sons 1995; Wu et al., Methods in Gene Biotechnology, CRC Press, Inc. 1997; Aviv and Leder, Proc. Nat'l Acad. Sci. USA 69:1408 (1972); Huynh et al., “Constructing and Screening cDNA Libraries in λgt10 and λgt11,” in DNA Cloning: A Practical Approach Vol. I, Glover (ed.), page 49 (IRL Press, 1985); Wu (1997) at pages 47-52.

Nucleic acid molecules that encode a human ZcytoR21 gene can also be obtained using the polymerase chain reaction (PCR) with oligonucleotide primers having nucleotide sequences that are based upon the nucleotide sequences of the ZcytoR21 gene or cDNA. General methods for screening libraries with PCR are provided by, for example, Yu et al., “Use of the Polymerase Chain Reaction to Screen Phage Libraries,” in Methods in Molecular Biology, Vol. 15: PCR Protocols: Current Methods and Applications, White (ed.), Humana Press, Inc., 1993. Moreover, techniques for using PCR to isolate related genes are described by, for example, Preston, “Use of Degenerate Oligonucleotide Primers and the Polymerase Chain Reaction to Clone Gene Family Members,” in Methods in Molecular Biology, Vol. 15: PCR Protocols: Current Methods and Applications, White (ed.), Humana Press, Inc. 1993. As an alternative, a ZcytoR21 gene can be obtained by synthesizing nucleic acid molecules using mutually priming long oligonucleotides and the nucleotide sequences described herein (see, for example, Ausubel (1995)). Established techniques using the polymerase chain reaction provide the ability to synthesize DNA molecules at least two kilobases in length (Adang et al., Plant Molec. Biol. 21:1131 (1993), Bambot et al., PCR Methods and Applications 2:266 (1993), Dillon et al., “Use of the Polymerase Chain Reaction for the Rapid Construction of Synthetic Genes,” in Methods in Molecular Biology, Vol. 15: PCR Protocols: Current Methods and Applications, White (ed.), pages 263-268, (Humana Press, Inc. 1993), and Holowachuk et al., PCR Methods Appl. 4:299 (1995)). For reviews on polynucleotide synthesis, see, for example, Glick and Pasternak, Molecular Biotechnology, Principles and Applications of Recombinant DNA (ASM Press 1994), Itakura et al., Annu. Rev. Biochem. 53:323 (1984), and Climie et al., Proc. Nat'l Acad. Sci. USA 87:633 (1990).

D) Production of ZcytoR21 Gene Variants

The present invention provides a variety of nucleic acid molecules, including DNA and RNA molecules, that encode the ZcytoR21 polypeptides disclosed herein. Those skilled in the art will readily recognize that, in view of the degeneracy of the genetic code, considerable sequence variation is possible among these polynucleotide molecules. Moreover, the present invention also provides isolated soluble monomeric, homodimeric, heterodimeric and multimeric receptor polypeptides that comprise at least one ZcytoR21 receptor subunit that is substantially homologous to the receptor polypeptide of any of SEQ ID NOs: 2, 5, 8, 11, 14, 21, 23, 107, 109, 111, 113, 115, 117 or 119. Thus, the present invention contemplates ZcytoR21 polypeptide-encoding nucleic acid molecules comprising degenerate nucleotides of SEQ ID NOs:1, 4, 7, 10, 13, 20, 22, 106, 108, 110, or 112, and their RNA equivalents.

Those skilled in the art will readily recognize that, in view of the degeneracy of the genetic code, considerable sequence variation is possible among these polynucleotide molecules. SEQ ID NO:7 is a degenerate nucleotide sequence that encompasses all nucleic acid molecules that encode the ZcytoR21 polypeptide of any of SEQ ID NOs: 2, 5, 8, 11, 14, 21, 23, 107, 109, 111, 113, 115, 117 or 119. Those skilled in the art will recognize that the degenerate sequence of SEQ ID NO:7 also provides all RNA sequences encoding any of SEQ ID NOs: 2, 5, 8, 11, 14, 21, 23, 107, 109, 111, 113, 115, 117 or 119, by substituting U for T. Thus, the present invention contemplates ZcytoR21 polypeptide-encoding nucleic acid molecules comprising nucleotide 154 to nucleotide 2229 of SEQ ID NO:1, and their RNA equivalents. Similarly, the ZcytoR21 degenerate sequence of SEQ ID NO:6 also provides all RNA sequences encoding SEQ ID NO:5, by substituting U for T.

Table 1 sets forth the one-letter codes to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 1

| | | | |
|---|---|---|---|
| Nucleotide | Resolution | Complement | Resolution |

| | | | |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons, encompassing all possible codons for a given amino acid, are set forth in Table 2.

TABLE 2

| | One | | |
|---|---|---|---|
| Amino | Letter | | Degenerate |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding an amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequences of SEQ ID NO:3. Variant sequences can be readily tested for functionality as described herein.

Different species can exhibit “preferential codon usage.” In general, see, Grantham et al., Nucl. Acids Res. 8:1893 (1980), Haas et al. Curr. Biol. 6:315 (1996), Wain-Hobson et al., Gene 13:35% (1981), Grosjean and Fiers, Gene 18:199 (1982), Holm, Nuc. Acids Res. 14:3075 (1986), Ikemura, J. Mol. Biol. 158:573 (1982), Sharp and Matassi, Curr. Opin. Genet. Dev. 4:851 (1994), Kane, Curr. Opin. Biotechnol. 6:494 (1995), and Makrides, Microbiol. Rev. 60:512 (1996). As used herein, the term “preferential codon usage” or “preferential codons” is a term of art referring to protein translation codons that are most frequently used in cells of a certain species, thus favoring one or a few representatives of the possible codons encoding each amino acid (See Table 2). For example, the amino acid threonine (Thr) may be encoded by ACA, ACC, ACG, or ACT, but in mammalian cells ACC is the most commonly used codon; in other species, for example, insect cells, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequences disclosed herein serve as a template for optimizing expression of polynucleotides in various cell types and species commonly used in the art and disclosed herein. Sequences containing preferential codons can be tested and optimized for expression in various species, and tested for functionality as disclosed herein.

A ZcytoR21-encoding cDNA can be isolated by a variety of methods, such as by probing with a complete or partial human cDNA or with one or more sets of degenerate probes based on the disclosed sequences. A cDNA can also be cloned using the polymerase chain reaction with primers designed from the representative human ZcytoR21 sequences disclosed herein. In addition, a cDNA library can be used to transform or transfect host cells, and expression of the cDNA of interest can be detected with an antibody to ZcytoR21 polypeptide.

Those skilled in the art will recognize that the sequence disclosed in SEQ ID NO:1 represents a single allele of human ZcytoR21, and that allelic variation and alternative splicing are expected to occur. Allelic variants of this sequence can be cloned by probing cDNA or genomic libraries from different individuals according to standard procedures. Allelic variants of the nucleotide sequences disclosed herein, including those containing silent mutations and those in which mutations result in amino acid sequence changes, are within the scope of the present invention, as are proteins which are allelic variants of the amino acid sequences disclosed herein. cDNA molecules generated from alternatively spliced mRNAs, which retain the properties of the ZcytoR21 polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art.

Using the methods discussed above, one of ordinary skill in the art can prepare a variety of polypeptides that comprise a soluble ZcytoR21 receptor subunit that is substantially homologous to either SEQ ID NOs:1, 4, 7, 10, 13, 20, 22, 106, 108, 110 or 112 or that encodes amino acids of either SEQ ID NOs: 2, 5, 8, 11, 14, 21, 23, 107, 109, 111, 113, 115, 117 or 119, or allelic variants thereof and retain the ligand-binding properties of the wild-type ZcytoR21 receptor. Such polypeptides may also include additional polypeptide segments as generally disclosed herein.

Within certain embodiments of the invention, the isolated nucleic acid molecules can hybridize under stringent conditions to nucleic acid molecules comprising nucleotide sequences disclosed herein. For example, such nucleic acid molecules can hybridize under stringent conditions to nucleic acid molecules comprising the nucleotide sequence of any of SEQ ID NOs: 1, 4, 7, 10, 13, 20, 22, 106, 108, 110 or 112, or to nucleic acid molecules comprising a nucleotide sequence complementary to any of SEQ ID NOs: 1, 4, 7, 10, 13, 20, 22, 106, 108, 110 or 112, or fragments thereof.

In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Following hybridization, the nucleic acid molecules can be washed to remove non-hybridized nucleic acid molecules under stringent conditions, or under highly stringent conditions. See, for example, Sambrook et al., Molecular Cloning: A Laboratory Manual, Second Edition (Cold Spring Harbor Press 1989); Ausubel et al., (eds.), Current Protocols in Molecular Biology (John Wiley and Sons, Inc. 1987); Berger and Kimmel (eds.), Guide to Molecular Cloning Techniques, (Academic Press, Inc. 1987); and Wetmur, Crit. Rev. Biochem. Mol. Biol. 26:227 (1990)). Sequence analysis software such as OLIGO 6.0 (LSR; Long Lake, Minn.) and Primer Premier 4.0 (Premier Biosoft International; Palo Alto, Calif.), as well as sites on the Internet, are available tools for analyzing a given sequence and calculating Tₘ based on user-defined criteria. It is well within the abilities of one skilled in the art to adapthybridization and wash conditions for use with a particular polynucleotide hybrid.

The present invention also provides isolated ZcytoR21 polypeptides that have a substantially similar sequence identity to the polypeptides of any of SEQ ID NOs: 2, 5, 8, 11, 14, 21, 23, 107, 109, 111, 113, 115, 117 or 119, or their orthologs. The term “substantially similar sequence identity” is used herein to denote polypeptides having at least 70%, at least 80%, at least 90%, at least 95%, such as 96%, 97%, 98%, or greater than 95% sequence identity to the sequences shown in any of SEQ ID NOs: 2, 5, 8, 11, 14, 21, 23, 107, 109, 111, 113, 115, 117 or 119, or their orthologs. For example, variant and orthologous ZcytoR21 receptors can be used to generate an immune response and raise cross-reactive antibodies to human ZcytoR21. Such antibodies can be humanized, and modified as described herein, and used therauputically to treat psoriasis, psoriatic arthritis, IBD, colitis, endotoxemia as well as in other therapeutic applications described herein.

The present invention also contemplates ZcytoR21 variant nucleic acid molecules that can be identified using two criteria: a determination of the similarity between the encoded polypeptide with the amino acid sequence of any of SEQ ID NOs: 2, 5, 8, 11, 14, 21, 23, 107, 109, 111, 113, 115, 117 or 119, and a hybridization assay. Such ZcytoR21 variants include nucleic acid molecules (1) that remain hybridized with a nucleic acid molecule having the nucleotide sequence of SEQ ID NOs:1, 4, 7, 10, 13, 20, 22, 106, 108, 110 or 112 (or its complement) under stringent washing conditions, in which the wash stringency is equivalent to 0.5×-2×SSC with 0.1% SDS at 55-65° C., and (2) that encode a polypeptide having at least 70%, at least 80%, at least 90%, at least 95%, or greater than 95% such as 96%, 97%, 98%, or 99%, sequence identity to the amino acid sequence of any of SEQ ID NOs: 2, 5, 8, 11, 14, 21, 23, 107, 109, 111, 113, 115, 117 or 119. Alternatively, ZcytoR21 variants can be characterized as nucleic acid molecules that: (1) remain hybridized with a nucleic acid molecule having the nucleotide sequence of SEQ ID NOs:1, 4, 7, 10, 13, 20, 22, 106, 108, 110 or 112 (or its complement) under highly stringent washing conditions, in which the wash stringency is equivalent to 0.1×-0.2×SSC with 0.1% SDS at 50-65° C., and (2) encode a polypeptide having at least 70%, at least 80%, at least 90%, at least 95% or greater than 95%, such as 96%, 97%, 98%, or 99% or greater, sequence identity to the amino acid sequence of any of SEQ ID NOs: 2, 5, 8, 11, 14, 21, 23, 107, 109, 111, 113, 115, 117 or 119.

Percent sequence identity is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.) as shown in Table 3 (amino acids are indicated by the standard one-letter codes). The percent identity is then calculated as: ([Total number of identical matches]/[length of the longer sequence plus the number of gaps introduced into the longer sequence in order to align the two sequences])(100).

TABLE 3

| | | | | | | | | | | | | | | | | | | | |
|---|---|---|---|---|---|---|---|---|---|---|---|---|---|---|---|---|---|---|---|
| A | R | N | D | C | Q | E | G | H | I | L | K | M | F | P | S | T | W | Y | V |

| | | | | | | | | | | | | | | | | | | | | |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative ZcytoR21 variant. The FASTA algorithm is described by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., any of SEQ ID NOs: 2, 5, 8, 11, 14, 21, 23, 107, 109, 111, 113, 115, 117 or 119) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from three to six, most preferably three, with other parameters set as described above.

The present invention includes nucleic acid molecules that encode a polypeptide having a conservative amino acid change, compared with an amino acid sequence disclosed herein. For example, variants can be obtained that contain one or more amino acid substitutions of any of SEQ ID NOs: 2, 5, 8, 11, 14, 21, 23, 107, 109, 111, 113, 115, 117 or 119, in which an alkyl amino acid is substituted for an alkyl amino acid in a ZcytoR21 amino acid sequence, an aromatic amino acid is substituted for an aromatic amino acid in a ZcytoR21 amino acid sequence, a sulfur-containing amino acid is substituted for a sulfur-containing amino acid in a ZcytoR21 amino acid sequence, a hydroxy-containing amino acid is substituted for a hydroxy-containing amino acid in a ZcytoR21 amino acid sequence, an acidic amino acid is substituted for an acidic amino acid in a ZcytoR21 amino acid sequence, a basic amino acid is substituted for a basic amino acid in a ZcytoR21 amino acid sequence, or a dibasic monocarboxylic amino acid is substituted for a dibasic monocarboxylic amino acid in a ZcytoR21 amino acid sequence. Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3). Particular variants of ZcytoR21 are characterized by having at least 70%, at least 80%, at least 90%, at least 95% or greater than 95% such as 96%, 97%, 98%, or 99% or greater sequence identity to the corresponding amino acid sequence (e.g., any of SEQ ID NOs: 2, 5, 8, 11, 14, 21, 23, 107, 109, 111, 113, 115, 117 or 119), wherein the variation in amino acid sequence is due to one or more conservative amino acid substitutions.

Conservative amino acid changes in a ZcytoR21 gene can be introduced, for example, by substituting nucleotides for the nucleotides recited in SEQ ID NOs:1, 4, 7, 10, 13, 20, 22, 106, 108, 110 or 112. Such “conservative amino acid” variants can be obtained by oligonucleotide-directed mutagenesis, linker-scanning mutagenesis, mutagenesis using the polymerase chain reaction, and the like (see Ausubel (1995); and McPherson (ed.), Directed Mutagenesis: A Practical Approach (IRL Press 1991)). A variant ZcytoR21 polypeptide can be identified by the ability to specifically bind anti-ZcytoR21 antibodies.

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is typically carried out in a cell-free system comprising an E. coli S30 extract and commercially available enzymes and other reagents. Proteins are purified by chromatography. See, for example, Robertson et al., J. Am. Chem. Soc. 113:2722 (1991), Ellman et al., Methods Enzymol. 202:301 (1991), Chung et al., Science 259:806 (1993), and Chung et al., Proc. Nat'l Acad. Sci. USA 90:10145 (1993).

In a second method, translation is carried out in Xenopus oocytes by microinjection of mutated mRNA and chemically aminoacylated suppressor tRNAs (Turcatti et al., J. Biol. Chem. 271:19991 (1996)). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470 (1994). Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395 (1993)).

A limited number of non-conservative amino acids, amino acids that are not encoded by the genetic code, non-naturally occurring amino acids, and unnatural amino acids may be substituted for ZcytoR21 amino acid residues.

Essential amino acids in the polypeptides of the present invention can be identified according to procedures known in the art, such as site-directed mutagenesis or alanine-scanning mutagenesis (Cunningham and Wells, Science 244:1081 (1989), Bass et al., Proc. Nat'l Acad. Sci. USA 88:4498 (1991), Coombs and Corey, “Site-Directed Mutagenesis and Protein Engineering,” in Proteins: Analysis and Design, Angeletti (ed.), pages 259-311 (Academic Press, Inc. 1998)). In the latter technique, single alanine mutations are introduced at every residue in the molecule, and the resultant mutant molecules are tested for biological activity to identify amino acid residues that are critical to the activity of the molecule. See also, Hilton et al., J. Biol. Chem. 271:4699 (1996).

Although sequence analysis can be used to further define the ZcytoR21 ligand binding region, amino acids that play a role in ZcytoR21 binding activity (such as binding of ZcytoR21 to Il-17C, or to an anti-ZcytoR21 antibody) can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306 (1992), Smith et al., J. Mol. Biol. 224:899 (1992), and Wlodaver et al., FEBS Lett. 309:59 (1992).

Multiple amino acid substitutions can be made and tested using known methods of mutagenesis and screening, such as those disclosed by Reidhaar-Olson and Sauer (Science 241:53 (1988)) or Bowie and Sauer (Proc. Nat'l Acad. Sci. USA 86:2152 (1989)). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832 (1991), Ladner et al., U.S. Pat. No. 5,223,409, Huse, international publication No. WO 92/06204, and region-directed mutagenesis (Derbyshire et al., Gene 46:145 (1986), and Ner et al., DNA 7:127, (1988)). Moreover, ZcytoR21 labeled with biotin or FITC can be used for expression cloning of ZcytoR21 ligands.

Variants of the disclosed ZcytoR21 nucleotide and polypeptide sequences can also be generated through DNA shuffling as disclosed by Stemmer, Nature 370:389 (1994), Stemmer, Proc. Nat'l Acad. Sci. USA 91:10747 (1994), and international publication No. WO 97/20078. Briefly, variant DNA molecules are generated by in vitro homologous recombination by random fragmentation of a parent DNA followed by reassembly using PCR, resulting in randomly introduced point mutations. This technique can be modified by using a family of parent DNA molecules, such as allelic variants or DNA molecules from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed herein can be combined with high-throughput, automated screening methods to detect activity of cloned, mutagenized polypeptides in host cells. Mutagenized DNA molecules that encode biologically active polypeptides, or polypeptides that bind with anti-ZcytoR21 antibodies, can be recovered from the host cells and rapidly sequenced using modern equipment. These methods allow the rapid determination of the importance of individual amino acid residues in a polypeptide of interest, and can be applied to polypeptides of unknown structure.

The present invention also includes “functional fragments” of ZcytoR21 polypeptides and nucleic acid molecules encoding such functional fragments. Routine deletion analyses of nucleic acid molecules can be performed to obtain functional fragments of a nucleic acid molecule that encodes a ZcytoR21 polypeptide. As an illustration, DNA molecules having the nucleotide sequence of SEQ ID NOs:1, 4, 7, 10, 13, 20, 22, 106, 108, 110 or 112 can be digested with Bal31 nuclease to obtain a series of nested deletions. The fragments are then inserted into expression vectors in proper reading frame, and the expressed polypeptides are isolated and tested for the ability to bind anti-ZcytoR21 antibodies. One alternative to exonuclease digestion is to use oligonucleotide-directed mutagenesis to introduce deletions or stop codons to specify production of a desired fragment. Alternatively, particular fragments of a ZcytoR21 gene can be synthesized using the polymerase chain reaction.

This general approach is exemplified by studies on the truncation at either or both termini of interferons have been summarized by Horisberger and Di Marco, Pharmac. Ther. 66:507 (1995). Moreover, standard techniques for functional analysis of proteins are described by, for example, Treuter et al., Molec. Gen. Genet. 240:113 (1993), Content et al., “Expression and preliminary deletion analysis of the 42 kDa 2-5A synthetase induced by human interferon,” in Biological Interferon Systems, Proceedings of ISIR-TNO Meeting on Interferon Systems, Cantell (ed.), pages 65-72 (Nijhoff 1987), Herschman, “The EGF Receptor,” in Control of Animal Cell Proliferation, Vol. 1, Boynton et al., (eds.) pages 169-199 (Academic Press 1985), Coumailleau et al., J. Biol. Chem. 270:29270 (1995); Fukunaga et al., J. Biol. Chem. 270:25291 (1995); Yamaguchi et al., Biochem. Pharmacol. 50:1295 (1995), and Meisel et al., Plant Molec. Biol. 30:1 (1996).

The present invention also contemplates functional fragments of a ZcytoR21 gene that have amino acid changes, compared with an amino acid sequence disclosed herein. A variant ZcytoR21 gene can be identified on the basis of structure by determining the level of identity with disclosed nucleotide and amino acid sequences, as discussed above. An alternative approach to identifying a variant gene on the basis of structure is to determine whether a nucleic acid molecule encoding a potential variant ZcytoR21 gene can hybridize to a nucleic acid molecule comprising a nucleotide sequence, such as SEQ ID NOs:1, 4, 7, 10, 13, 20, 22, 106, 108, 110, or 112.

The present invention also includes using functional fragments of ZcytoR21 polypeptides, antigenic epitopes, epitope-bearing portions of ZcytoR21 polypeptides, and nucleic acid molecules that encode such functional fragments, antigenic epitopes, epitope-bearing portions of ZcytoR21 polypeptides. For example, such ZcytoR21 fragments include polypeptides encoded by SEQ ID NOs:115, 117 or 119. These fragments encode binding domains of ZcytoR21 and are used to generate polypeptides for use in generating antibodies and binding partners that bind, block, inhibit, reduce, antagonize or neutralize activity of IL-17C. A “functional” ZcytoR21 polypeptide or fragment thereof as defined herein is characterized by its ability to block, inhibit, reduce, antagonize or neutralize IL-17C inflammatory, proliferative or differentiating activity, by its ability to induce or inhibit specialized cell functions, or by its ability to bind specifically to an anti-ZcytoR21 antibody, cell, or IL-17C. As previously described herein, ZcytoR21 is characterized by a unique cytokine receptor structure and domains as described herein. Thus, the present invention further contemplates using fusion proteins encompassing: (a) polypeptide molecules comprising one or more of the domains described above; and (b) functional fragments comprising one or more of these domains. The other polypeptide portion of the fusion protein may be contributed by another cytokine receptor, such as IL-17RA, IL-17RB, IL-17RC, IL-17RD, IL-17RE, or by a non-native and/or an unrelated secretory signal peptide that facilitates secretion of the fusion protein.

The present invention also provides polypeptide fragments or peptides comprising an epitope-bearing portion of a ZcytoR21 polypeptide described herein. Such fragments or peptides may comprise an “immunogenic epitope,” which is a part of a protein that elicits an antibody response when the entire protein is used as an immunogen. Immunogenic epitope-bearing peptides can be identified using standard methods (see, for example, Geysen et al., Proc. Nat'l Acad. Sci. USA 81:3998 (1983)).

In contrast, polypeptide fragments or peptides may comprise an “antigenic epitope,” which is a region of a protein molecule to which an antibody can specifically bind. Certain epitopes consist of a linear or contiguous stretch of amino acids, and the antigenicity of such an epitope is not disrupted by denaturing agents. It is known in the art that relatively short synthetic peptides that can mimic epitopes of a protein can be used to stimulate the production of antibodies against the protein (see, for example, Sutcliffe et al., Science 219:660 (1983)). Accordingly, antigenic epitope-bearing peptides, antigenic peptides, epitopes, and polypeptides of the present invention are useful to raise antibodies that bind with the polypeptides described herein, as well as to identify and screen anti-ZcytoR21 monoclonal antibodies that are neutralizing, and that may bind, block, inhibit, reduce, antagonize or neutralize the activity of IL-17C. Such neutralizing monoclonal antibodies of the present invention can bind to an ZcytoR21 antigenic epitope. Hopp/Woods hydrophilicity profiles can be used to determine regions that have the most antigenic potential within any of SEQ ID NOs: 2, 5, 8, 11, 14, 21, 23, 107% 109, 111, 113, 115, 117 or 119 (Hopp et al., Proc. Natl. Acad. Sci. 78:3824-3828, 1981; Hopp, J. Immun. Meth. 88:1-18, 1986 and Triquier et al., Protein Engineering 11:153-169, 1998). The profile is based on a sliding six-residue window. Buried G, S, and T residues and exposed H, Y, and W residues were ignored. In ZcytoR21 these regions can be determined by one of skill in the art. Moreover, ZcytoR21 antigenic epitopes within any of SEQ ID NOs: 2, 5, 8, 11, 14, 21, 23, 107, 109, 111, 113, 115, 117 or 119 as predicted by a Jameson-Wolf plot, e.g., using DNASTAR Protean program (DNASTAR, Inc., Madison, Wis.) serve as preferred antigenic epitpoes, and can be determined by one of skill in the art. The results of this analysis indicated that SEQ ID NOs: 115 (“antigenic peptide 1”), 117 (“antigenic peptide 2”), 119 (“antigenic peptide 3”), and the following amino acid sequences of SEQ ID NO:6 would provide suitable antigenic peptides: amino acids 51 to 59 (“antigenic peptide 4”), amino acids 72 to 83 (“antigenic peptide 5”), 91 to 97 (“antigenic peptide 6”), amino acids 174 to 180 (“antigenic peptide 7”), and amino acids 242 to 246 (“antigenic peptide 8”). The present invention contemplates the use of any one of, or any sub-combinations thereof, of antigenic peptides 1 to 8 to generate antibodies to ZcytoR21. The present invention also contemplates polypeptides comprising at least one of antigenic peptides 1 to 8. For instance, antigenic peptides 1 and 2 may be combined to generate a polypeptide useful in generating an antibody antagonist of the the present invention.

In preferred embodiments, antigenic epitopes to which neutralizing antibodies of the present invention bind would contain residues of any of SEQ ID NOs:2, 3, 5, 6, 8, 9, 11, 12, 14, 15, 21, 23, 107, 109, 111, 113, 115, 117, or 119 that are important to ligand-receptor binding, for example, with ZcytoR21 and IL-17C. Most preferably, antigenic epitopes to which neutralizing antibodies of the present invention bind would contain residues of any of SEQ ID NOs: 115, 117, or 119.

Antigenic epitope-bearing peptides and polypeptides can contain at least four to ten amino acids, at least ten to fifteen amino acids, or about 15 to about 30 amino acids of an amino acid sequence disclosed herein. Such epitope-bearing peptides and polypeptides can be produced by fragmenting a ZcytoR21 polypeptide, or by chemical peptide synthesis, as described herein. Moreover, epitopes can be selected by phage display of random peptide libraries (see, for example, Lane and Stephen, Curr. Opin. Immunol. 5:268 (1993), and Cortese et al., Curr. Opin. Biotechnol. 7:616 (1996)). Standard methods for identifying epitopes and producing antibodies from small peptides that comprise an epitope are described, for example, by Mole, “Epitope Mapping,” in Methods in Molecular Biology, Vol. 10, Manson (ed.), pages 105-116 (The Humana Press, Inc. 1992), Price, “Production and Characterization of Synthetic Peptide-Derived Antibodies,” in Monoclonal Antibodies: Production, Engineering, and Clinical Application, Ritter and Ladyman (eds.), pages 60-84 (Cambridge University Press 1995), and Coligan et al. (eds.), Current Protocols in Immunology, pages 9.3.1-9.3.5 and pages 9.4.1-9.4.11 (John Wiley & Sons 1997).

For any ZcytoR21 polypeptide, including variants and fusion proteins, one of ordinary skill in the art can readily generate a fully degenerate polynucleotide sequence encoding that variant using the information set forth in Tables 1 and 2 above. Moreover, those of skill in the art can use standard software to devise ZcytoR21 variants based upon the nucleotide and amino acid sequences described herein.

E) Production of ZcytoR21 Polypeptides

The polypeptides of the present invention, including full-length polypeptides; soluble monomeric, homodimeric, heterodimeric and multimeric receptors; full-length receptors; receptor fragments (e.g. ligand-binding fragments and antigenic epitopes), functional fragments, and fusion proteins, can be produced in recombinant host cells following conventional techniques. To express a ZcytoR21 gene, a nucleic acid molecule encoding the polypeptide must be operably linked to regulatory sequences that control transcriptional expression in an expression vector and then, introduced into a host cell. In addition to transcriptional regulatory sequences, such as promoters and enhancers, expression vectors can include translational regulatory sequences and a marker gene which is suitable for selection of cells that carry the expression vector.

Expression vectors that are suitable for production of a foreign protein in eukaryotic cells typically contain (1) prokaryotic DNA elements coding for a bacterial replication origin and an antibiotic resistance marker to provide for the growth and selection of the expression vector in a bacterial host; (2) eukaryotic DNA elements that control initiation of transcription, such as a promoter; and (3) DNA elements that control the processing of transcripts, such as a transcription termination/polyadenylation sequence. As discussed above, expression vectors can also include nucleotide sequences encoding a secretory sequence that directs the heterologous polypeptide into the secretory pathway of a host cell. For example, an ZcytoR21 expression vector may comprise a ZcytoR21 gene and a secretory sequence derived from any secreted gene.

ZcytoR21 proteins of the present invention may be expressed in mammalian cells. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61; CHO DG44 (Chasin et al., Som. Cell. Molec. Genet. 12:555, 1986)), rat pituitary cells (GH1; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650) and murine embryonic cells (NIH-3T3; ATCC CRL 1658).

For a mammalian host, the transcriptional and translational regulatory signals may be derived from mammalian viral sources, for example, adenovirus, bovine papilloma virus, simian virus, or the like, in which the regulatory signals are associated with a particular gene which has a high level of expression. Suitable transcriptional and translational regulatory sequences also can be obtained from mammalian genes, for example, actin, collagen, myosin, and metallothionein genes.

Transcriptional regulatory sequences include a promoter region sufficient to direct the initiation of RNA synthesis. Suitable eukaryotic promoters include the promoter of the mouse metallothionein I gene (Hamer et al., J. Molec. Appl. Genet. 1:273 (1982)), the TK promoter of Herpes virus (McKnight, Cell 31:355 (1982)), the SV40 early promoter (Benoist et al., Nature 290:304 (1981)), the Rous sarcoma virus promoter (Gorman et al., Proc. Nat'l Acad. Sci. USA 79:6777 (1982)), the cytomegalovirus promoter (Foecking et al., Gene 45:101 (1980)), and the mouse mammary tumor virus promoter (see, generally, Etcheverry, “Expression of Engineered Proteins in Mammalian Cell Culture,” in Protein Engineering: Principles and Practice, Cleland et al. (eds.), pages 163-181 (John Wiley & Sons, Inc. 1996)).

Alternatively, a prokaryotic promoter, such as the bacteriophage T3 RNA polymerase promoter, can be used to control ZcytoR21 gene expression in mammalian cells if the prokaryotic promoter is regulated by a eukaryotic promoter (Zhou et al., Mol. Cell. Biol. 10:4529 (1990), and Kaufman et al., Nucl. Acids Res. 19:4485 (1991)).

In certain embodiments, a DNA sequence encoding a ZcytoR21 soluble receptor polypeptide, or a fragment of ZcytoR21 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers. Multiple components of a soluble receptor complex can be co-transfected on individual expression vectors or be contained in a single expression vector. Such techniques of expressing multiple components of protein complexes are well known in the art.

An expression vector can be introduced into host cells using a variety of standard techniques including calcium phosphate transfection, liposome-mediated transfection, microprojectile-mediated delivery, electroporation, and the like. The transfected cells can be selected and propagated to provide recombinant host cells that comprise the expression vector stably integrated in the host cell genome. Techniques for introducing vectors into eukaryotic cells and techniques for selecting such stable transformants using a dominant selectable marker are described, for example, by Ausubel (1995) and by Murray (ed.), Gene Transfer and Expression Protocols (Humana Press 1991).

For example, one suitable selectable marker is a gene that provides resistance to the antibiotic neomycin. In this case, selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A suitable amplifiable selectable marker is dihydrofolate reductase (DHFR), which confers resistance to methotrexate. Other drug resistance genes (e.g., hygromycin resistance, multi-drug resistance, puromycin acetyltransferase) can also be used. Alternatively, markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

ZcytoR21 polypeptides can also be produced by cultured mammalian cells using a viral delivery system. Exemplary viruses for this purpose include adenovirus, retroviruses, herpesvirus, vaccinia virus and adeno-associated virus (AAV). Adenovirus, a double-stranded DNA virus, is currently the best studied gene transfer vector for delivery of heterologous nucleic acid (for a review, see Becker et al., Meth. Cell Biol. 43:161 (1994), and Douglas and Curiel, Science & Medicine 4:44 (1997)). Advantages of the adenovirus system include the accommodation of relatively large DNA inserts, the ability to grow to high-titer, the ability to infect a broad range of mammalian cell types, and flexibility that allows use with a large number of available vectors containing different promoters.

By deleting portions of the adenovirus genome, larger inserts (up to 7 kb) of heterologous DNA can be accommodated. These inserts can be incorporated into the viral DNA by direct ligation or by homologous recombination with a co-transfected plasmid. An option is to delete the essential E1 gene from the viral vector, which results in the inability to replicate unless the E1 gene is provided by the host cell. Adenovirus vector-infected human 293 cells (ATCC Nos. CRL-1573, 45504, 45505), for example, can be grown as adherent cells or in suspension culture at relatively high cell density to produce significant amounts of protein (see Garnier et al., Cytotechnol. 15:145 (1994)).

ZcytoR21 can also be expressed in other higher eukaryotic cells, such as avian, fungal, insect, yeast, or plant cells. The baculovirus system provides an efficient means to introduce cloned ZcytoR21 genes into insect cells. Suitable expression vectors are based upon the Autographa californica multiple nuclear polyhedrosis virus (AcMNPV), and contain well-known promoters such as Drosophila heat shock protein (hsp) 70 promoter, Autographa californica nuclear polyhedrasis virus immediate-early gene promoter (ie-1) and the delayed early 39K promoter, baculovirus p10 promoter, and the Drosophila metallothionein promoter. A second method of making recombinant baculovirus utilizes a transposon-based system described by Luckow (Luckow, et al., J. Virol. 67:4566 (1993)). This system, which utilizes transfer vectors, is sold in the BAC-to-BAC kit (Life Technologies, Rockville, Md.). This system utilizes a transfer vector, PFASTBAC (Life Technologies) containing a Tn7 transposon to move the DNA encoding the ZcytoR21 polypeptide into a baculovirus genome maintained in E. coli as a large plasmid called a “bacmid.” See, Hill-Perkins and Possee, J. Gen. Virol. 71:971 (1990), Bonning, et al., J. Gen. Virol. 75:1551 (1994), and Chazenbalk, and Rapoport, J. Biol. Chem. 270:1543 (1995). In addition, transfer vectors can include an in-frame fusion with DNA encoding an epitope tag at the C- or N-terminus of the expressed ZcytoR21 polypeptide, for example, a Glu-Glu epitope tag (Grussenmeyer et al., Proc. Nat'l Acad. Sci. 82:7952 (1985)). Using a technique known in the art, a transfer vector containing a ZcytoR21 gene is transformed into E. coli, and screened for bacmids which contain an interrupted lacZ gene indicative of recombinant baculovirus. The bacmid DNA containing the recombinant baculovirus genome is then isolated using common techniques.

The illustrative PFASTBAC vector can be modified to a considerable degree. For example, the polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins (see, for example, Hill-Perkins and Possee, J. Gen. Virol. 71:971 (1990), Bonning, et al., J. Gen. Virol. 75:1551 (1994), and Chazenbalk and Rapoport, J. Biol. Chem. 270:1543 (1995). In such transfer vector constructs, a short or long version of the basic protein promoter can be used. Moreover, transfer vectors can be constructed which replace the native ZcytoR21 secretory signal sequences with secretory signal sequences derived from insect proteins. For example, a secretory signal sequence from Ecdysteroid Glucosyltransferase (EGT), honey bee Melittin (Invitrogen Corporation; Carlsbad, Calif.), or baculovirus gp67 (PharMingen: San Diego, Calif.) can be used in constructs to replace the native ZcytoR21 secretory signal sequence.

The recombinant virus or bacmid is used to transfect host cells. Suitable insect host cells include cell lines derived from IPLB-Sf-21, a Spodoptera frugiperda pupal ovarian cell line, such as Sf9 (ATCC CRL 1711), Sf21AE, and Sf21 (Invitrogen Corporation; San Diego, Calif.), as well as Drosophila Schneider-2 cells, and the HIGH FIVEO cell line (Invitrogen) derived from Trichoplusia ni (U.S. Pat. No. 5,300,435). Commercially available serum-free media can be used to grow and to maintain the cells. Suitable media are Sf900 II™ (Life Technologies) or ESF 921™ (Expression Systems) for the Sf9 cells; and Ex-cellO405™ (JRH Biosciences, Lenexa, Kans.) or Express FiveO™ (Life Technologies) for the T. ni cells. When recombinant virus is used, the cells are typically grown up from an inoculation density of approximately 2-5×10⁵ cells to a density of 1-2×10⁶ cells at which time a recombinant viral stock is added at a multiplicity of infection (MOI) of 0.1 to 10, more typically near 3.

Established techniques for producing recombinant proteins in baculovirus systems are provided by Bailey et al., “Manipulation of Baculovirus Vectors,” in Methods in Molecular Biology, Volume 7: Gene Transfer and Expression Protocols, Murray (ed.), pages 147-168 (The Humana Press, Inc. 1991), by Patel et al., “The baculovirus expression system,” in DNA Cloning 2: Expression Systems, 2nd Edition, Glover et al. (eds.), pages 205-244 (Oxford University Press 1995), by Ausubel (1995) at pages 16-37 to 16-57, by Richardson (ed.), Baculovirus Expression Protocols (The Humana Press, Inc. 1995), and by Lucknow, “Insect Cell Expression Technology,” in Protein Engineering: Principles and Practice, Cleland et al. (eds.), pages 183-218 (John Wiley & Sons, Inc. 1996).

Fungal cells, including yeast cells, can also be used to express the genes described herein. Yeast species of particular interest in this regard include Saccharomyces cerevisiae, Pichia pastoris, and Pichia methanolica. Suitable promoters for expression in yeast include promoters from GAL1 (galactose), PGK (phosphoglycerate kinase), ADH (alcohol dehydrogenase), AOX1 (alcohol oxidase), HIS4 (histidinol dehydrogenase), and the like. Many yeast cloning vectors have been designed and are readily available. These vectors include YIp-based vectors, such as YIp5, YRp vectors, such as YRp17, YEp vectors such as YEp13 and YCp vectors, such as YCp19. Methods for transforming S. cerevisiae cells with exogenous DNA and producing recombinant polypeptides therefrom are disclosed by, for example, Kawasaki, U.S. Pat. No. 4,599,311, Kawasaki et al., U.S. Pat. No. 4,931,373, Brake, U.S. Pat. No. 4,870,008, Welch et al., U.S. Pat. No. 5,037,743, and Murray et al., U.S. Pat. No. 4,845,075. Transformed cells are selected by phenotype determined by the selectable marker, commonly drug resistance or the ability to grow in the absence of a particular nutrient (e.g., leucine). A suitable vector system for use in Saccharomyces cerevisiae is the POT1 vector system disclosed by Kawasaki et al. (U.S. Pat. No. 4,931,373), which allows transformed cells to be selected by growth in glucose-containing media. Additional suitable promoters and terminators for use in yeast include those from glycolytic enzyme genes (see, e.g., Kawasaki, U.S. Pat. No. 4,599,311, Kingsman et al., U.S. Pat. No. 4,615,974, and Bitter, U.S. Pat. No. 4,977,092) and alcohol dehydrogenase genes. See also U.S. Pat. Nos. 4,990,446, 5,063,154, 5,139,936, and 4,661,454.

Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459 (1986), and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

For example, the use of Pichia methanolica as host for the production of recombinant proteins is disclosed by Raymond, U.S. Pat. No. 5,716,808, Raymond, U.S. Pat. No. 5,736,383, Raymond et al., Yeast 14:11-23 (1998), and in international publication Nos. WO 97/17450, WO 97/17451, WO 98/02536, and WO 98/02565. DNA molecules for use in transforming P. methanolica will commonly be prepared as double-stranded, circular plasmids, which are preferably linearized prior to transformation. For polypeptide production in P. methanolica, the promoter and terminator in the plasmid can be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. A suitable selectable marker for use in Pichia methanolica is a P. methanolica ADE2 gene, which encodes phosphoribosyl-5-aminoimidazole carboxylase (AIRC; EC 4.1.1.21), and which allows ade2 host cells to grow in the absence of adenine. For large-scale, industrial processes where it is desirable to minimize the use of methanol, host cells can be used in which both methanol utilization genes (AUG1 and AUG2) are deleted. For production of secreted proteins, host cells can be deficient in vacuolar protease genes (PEP4 and PRB1). Electroporation is used to facilitate the introduction of a plasmid containing DNA encoding a polypeptide of interest into P. methanolica cells. P. methanolica cells can be transformed by electroporation using an exponentially decaying, pulsed electric field having a field strength of from 2.5 to 4.5 kV/cm, preferably about 3.75 kV/cm, and a time constant (t) of from 1 to 40 milliseconds, most preferably about 20 milliseconds.

Expression vectors can also be introduced into plant protoplasts, intact plant tissues, or isolated plant cells. Methods for introducing expression vectors into plant tissue include the direct infection or co-cultivation of plant tissue with Agrobacterium tumefaciens, microprojectile-mediated delivery, DNA injection, electroporation, and the like. See, for example, Horsch et al., Science 227:1229 (1985), Klein et al., Biotechnology 10:268 (1992), and Miki et al., “Procedures for Introducing Foreign DNA into Plants,” in Methods in Plant Molecular Biology and Biotechnology, Glick et al. (eds.), pages 67-88 (CRC Press, 1993).

Alternatively, ZcytoR21 genes can be expressed in prokaryotic host cells. Suitable promoters that can be used to express ZcytoR21 polypeptides in a prokaryotic host are well-known to those of skill in the art and include promoters capable of recognizing the T4, T3, Sp6 and T7 polymerases, the P_{R} and P_{L} promoters of bacteriophage lambda, the trp, recA, heat shock, lacUV5, tac, lpp-lacSpr, phoA, and lacZ promoters of E. coli, promoters of B. subtilis, the promoters of the bacteriophages of Bacillus, Streptomyces promoters, the int promoter of bacteriophage lambda, the bla promoter of pBR322, and the CAT promoter of the chloramphenicol acetyl transferase gene. Prokaryotic promoters have been reviewed by Glick, J. Ind. Microbiol. 1:277 (1987), Watson et al., Molecular Biology of the Gene, 4th Ed. (Benjamin Cummins 1987), and by Ausubel et al. (1995).

Suitable prokaryotic hosts include E. coli and Bacillus subtilus. Suitable strains of E. coli include BL21(DE3), BL21(DE3)pLysS, BL21(DE3)pLysE, DH1, DH4I, DH5, DH5I, DH51F′, DH5IMCR, DH10B, DH10B/p3, DH11S, C600, HB101, JM101, JM105, JM109, JM110, K38, RR1, Y1088, Y1089, CSH18, ER2151, and ER1647 (see, for example, Brown (ed.), Molecular Biology Labfax (Academic Press 1991)). Suitable strains of Bacillus subtilus include BR151, YB886, MI119, MI120, and B170 (see, for example, Hardy, “Bacillus Cloning Methods,” in DNA Cloning: A Practical Approach, Glover (ed.) (IRL Press 1985)).

When expressing a ZcytoR21 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Methods for expressing proteins in prokaryotic hosts are well-known to those of skill in the art (see, for example, Williams et al., “Expression of foreign proteins in E. coli using plasmid vectors and purification of specific polyclonal antibodies,” in DNA Cloning 2: Expression Systems, 2nd Edition, Glover et al. (eds.), page 15 (Oxford University Press. 1995), Ward et al., “Genetic Manipulation and Expression of Antibodies,” in Monoclonal Antibodies: Principles and Applications, page 137 (Wiley-Liss, Inc. 1995), and Georgiou, “Expression of Proteins in Bacteria,” in Protein Engineering: Principles and Practice, Cleland et al. (eds.), page 101 (John Wiley & Sons, Inc. 1996)).

Standard methods for introducing expression vectors into bacterial, yeast, insect, and plant cells are provided, for example, by Ausubel (1995).

General methods for expressing and recovering foreign protein produced by a mammalian cell system are provided by, for example, Etcheverry, “Expression of Engineered Proteins in Mammalian Cell Culture,” in Protein Engineering: Principles and Practice, Cleland et al. (eds.), pages 163 (Wiley-Liss, Inc. 1996). Standard techniques for recovering protein produced by a bacterial system is provided by, for example, Grisshammer et al., “Purification of over-produced proteins from E. coli cells,” in DNA Cloning 2: Expression Systems, 2nd Edition, Glover et al. (eds.), pages 59-92 (Oxford University Press 1995). Established methods for isolating recombinant proteins from a baculovirus system are described by Richardson (ed.), Baculovirus Expression Protocols (The Humana Press, Inc. 1995).

As an alternative, polypeptides of the present invention can be synthesized by exclusive solid phase synthesis, partial solid phase methods, fragment condensation or classical solution synthesis. These synthesis methods are well-known to those of skill in the art (see, for example, Merrifield, J. Am. Chem. Soc. 85:2149 (1963), Stewart et al., “Solid Phase Peptide Synthesis” (2nd Edition), (Pierce Chemical Co. 1984), Bayer and Rapp, Chem. Pept. Prot. 3:3 (1986), Atherton et al., Solid Phase Peptide Synthesis: A Practical Approach (IRL Press 1989), Fields and Colowick, “Solid-Phase Peptide Synthesis,” Methods in Enzymology Volume 289 (Academic Press 1997), and Lloyd-Williams et al., Chemical Approaches to the Synthesis of Peptides and Proteins (CRC Press, Inc. 1997)). Variations in total chemical synthesis strategies, such as “native chemical ligation” and “expressed protein ligation” are also standard (see, for example, Dawson et al., Science 266:776 (1994), Hackeng et al., Proc. Nat'l Acad. Sci. USA 94:7845 (1997), Dawson, Methods Enzymol. 287: 34 (1997), Muir et al, Proc. Nat'l Acad. Sci. USA 95:6705 (1998), and Severinov and Muir, J. Biol. Chem. 273:16205 (1998)).

Peptides and polypeptides of the present invention comprise at least six, at least nine, or at least 15 contiguous amino acid residues of any of SEQ ID NOs:2, 5, 8, 11, 14, 21, 23, 107, 109, 113, 115, 117, or 119. As an illustration, polypeptides can comprise at least six, at least nine, or at least 15 contiguous amino acid residues of of any of SEQ ID NOs: 2, 5, 8, 11, 14, 21, 23, 107, 109, 113, 115, 117, or 119. Within certain embodiments of the invention, the polypeptides comprise 20, 30, 40, 50, 100, or more contiguous residues of these amino acid sequences. Nucleic acid molecules encoding such peptides and polypeptides are useful as polymerase chain reaction primers and probes.

Moreover, ZcytoR21 polypeptides and fragments thereof can be expressed as monomers, homodimers, heterodimers, or multimers within higher eukaryotic cells. Such cells can be used to produce ZcytoR21 monomeric, homodimeric, heterodimeric and multimeric receptor polypeptides that comprise at least one ZcytoR21 polypeptide (“ZcytoR21-comprising receptors” or “ZcytoR21-comprising receptor polypeptides”), or can be used as assay cells in screening systems. Within one aspect of the present invention, a polypeptide of the present invention comprising the ZcytoR21 extracellular domain is produced by a cultured cell, and the cell is used to screen for ligands for the receptor, including the natural ligand, IL-17C, or even agonists and antagonists of the natural ligand. To summarize this approach, a cDNA or gene encoding the receptor is combined with other genetic elements required for its expression (e.g., a transcription promoter), and the resulting expression vector is inserted into a host cell. Cells that express the DNA and produce functional receptor are selected and used within a variety of screening systems. Each component of the monomeric, homodimeric, heterodimeric and multimeric receptor complex can be expressed in the same cell. Moreover, the components of the monomeric, homodimeric, heterodimeric and multimeric receptor complex can also be fused to a transmembrane domain or other membrane fusion moiety to allow complex assembly and screening of transfectants as described above.

To assay the IL-17C antagonist polyepeptides and antibodies of the present invention, mammalian cells suitable for use in expressing ZcytoR21-comprising receptors or other receptors known to bind IL-17C and transducing a receptor-mediated signal include cells that express other receptor subunits that may form a functional complex with ZcytoR21. It is also preferred to use a cell from the same species as the receptor to be expressed. Within a preferred embodiment, the cell is dependent upon an exogenously supplied hematopoietic growth factor for its proliferation. Preferred cell lines of this type are the human TF-1 cell line (ATCC number CRL-2003) and the AML-193 cell line (ATCC number CRL-9589), which are GM-CSF-dependent human leukemic cell lines and BaF3 (Palacios and Steinmetz, Cell 41: 727-734, (1985)) which is an IL-3 dependent murine pre-B cell line. Other cell lines include BHK, COS-1 and CHO cells. Suitable host cells can be engineered to produce the necessary receptor subunits or other cellular component needed for the desired cellular response. This approach is advantageous because cell lines can be engineered to express receptor subunits from any species, thereby overcoming potential limitations arising from species specificity. Species orthologs of the human receptor cDNA can be cloned and used within cell lines from the same species, such as a mouse cDNA in the BaF3 cell line. Cell lines that are dependent upon one hematopoietic growth factor, such as GM-CSF or IL-3, can thus be engineered to become dependent upon another cytokine that acts through the ZcytoR21 receptor, such as IL-17C.

Cells expressing functional receptor are used within screening assays. A variety of suitable assays are known in the art. These assays are based on the detection of a biological response in a target cell. One such assay is a cell proliferation assay. Cells are cultured in the presence or absence of a test compound, and cell proliferation is detected by, for example, measuring incorporation of tritiated thymidine or by colorimetric assay based on the metabolic breakdown of 3-(4,5-dimethylthiazol-2-yl)-2,5-diphenyl tetrazolium bromide (MTT) (Mosman, J. Immunol. Meth. 65: 55-63, (1983)). An alternative assay format uses cells that are further engineered to express a reporter gene. The reporter gene is linked to a promoter element that is responsive to the receptor-linked pathway, and the assay detects activation of transcription of the reporter gene. A preferred promoter element in this regard is an NfKB responsive promoter. Additionally, one could also use a serum response element, or SRE. See, e.g., Shaw et al., Cell 56:563-572, (1989). A preferred such reporter gene is a luciferase gene (de Wet et al., Mol. Cell. Biol. 7:725, (1987)). Expression of the luciferase gene is detected by luminescence using methods known in the art (e.g., Baumgartner et al., J. Biol. Chem. 269:29094-29101, (1994); Schenborn and Goiffin, Promega_{—}Notes 41:11, 1993). Luciferase activity assay kits are commercially available from, for example, Promega Corp., Madison, Wis. Target cell lines of this type can be used to screen libraries of chemicals, cell-conditioned culture media, fungal broths, soil samples, water samples, and the like. Another alternative assay detects the phosphorylation of cell signaling pathways (transcription factors, kinases, etc) in response to ligand binding and activation of receptor. For example, a bank of cell-conditioned media samples can be assayed on a target cell to identify cells that produce ligand. Positive cells are then used to produce a cDNA library in a mammalian expression vector, which is divided into pools, transfected into host cells, and expressed. Media samples from the transfected cells are then assayed, with subsequent division of pools, re-transfection, subculturing, and re-assay of positive cells to isolate a cloned cDNA encoding the ligand.

An additional screening approach provided by the present invention includes the use of hybrid receptor polypeptides. These hybrid polypeptides fall into two general classes. Within the first class, the intracellular domain of ZcytoR21, is joined to the ligand-binding domain of a second receptor. A second class of hybrid receptor polypeptides comprise the extracellular (ligand-binding) domain of ZcytoR21 (e.g. SEQ ID NO:3, amino acid residues 24-376 of SEQ ID NO:5, amino acid residues 24-396 of SEQ ID NO:8, SEQ ID NO:12, amino acid residues 24-414 of SEQ ID NO:21, SEQ ID NO:22, SEQ ID NO:122, amino acid residues 24-414 of SEQ ID NO:109, SEQ ID NO:113, SEQ ID NO:115, SEQ ID NO:117, or SEQ ID NO:119) with an intracellular domain of a second receptor, preferably a hematopoietic cytokine receptor, and a transmembrane domain. Hybrid ZcytoR21 monomers, homodimers, heterodimers and multimers of the present invention receptors of this second class are expressed in cells known to be capable of responding to signals transduced by the second receptor. Together, these two classes of hybrid receptors enable the identification of a responsive cell type for the development of an assay for detecting IL-17C. Moreover, such cells can be used in the presence of IL-17C to assay the soluble receptor antagonists of the present invention in a competition-type assay. In such assay, a decrease in the proliferation or signal transduction activity of IL-17C in the presence of a soluble receptor of the present invention demonstrates antagonistic activity. Moreover ZcytoR21-soluble receptor binding assays, and cell-based assays, can also be used to assess whether a soluble receptor binds, blocks, inhibits, reduces, antagonizes or neutralizes IL-17C activity.

F) Production of ZcytoR21 Fusion Proteins and Conjugates

One general class of ZcytoR21 analogs are variants having an amino acid sequence that is a mutation of the amino acid sequence disclosed herein. Another general class of ZcytoR21 analogs is provided by anti-idiotype antibodies, and fragments thereof, as described below. Moreover, recombinant antibodies comprising anti-idiotype variable domains can be used as analogs (see, for example, Monfardini et al., Proc. Assoc. Am. Physicians 108:420 (1996)). Since the variable domains of anti-idiotype ZcytoR21 antibodies mimic ZcytoR21, these domains can provide ZcytoR21 binding activity. Methods of producing anti-idiotypic catalytic antibodies are known to those of skill in the art (see, for example, Joron et al., Ann. N Y Acad. Sci. 672:216 (1992), Friboulet et al., Appl. Biochem. Biotechnol. 47:229 (1994), and Avalle et al., Ann. N Y Acad. Sci. 864:118 (1998)).

Another approach to identifying ZcytoR21 analogs is provided by the use of combinatorial libraries. Methods for constructing and screening phage display and other combinatorial libraries are provided, for example, by Kay et al., Phage Display of Peptides and Proteins (Academic Press 1996), Verdine, U.S. Pat. No. 5,783,384, Kay, et. al., U.S. Pat. No. 5,747,334, and Kauffman et al., U.S. Pat. No. 5,723,323.

ZcytoR21 polypeptides have both in vivo and in vitro uses. As an illustration, a soluble form of ZcytoR21 can be added to cell culture medium to inhibit the effects of the ZcytoR21 ligand (i.e. IL-17C) produced by the cultured cells.

Fusion proteins of ZcytoR21 can be used to express ZcytoR21 in a recombinant host, and to isolate the produced ZcytoR21. As described below, particular ZcytoR21 fusion proteins also have uses in diagnosis and therapy. One type of fusion protein comprises a peptide that guides a ZcytoR21 polypeptide from a recombinant host cell. To direct a ZcytoR21 polypeptide into the secretory pathway of a eukaryotic host cell, a secretory signal sequence (also known as a signal peptide, a leader sequence, prepro sequence or pre sequence) is provided in the ZcytoR21 expression vector. While the secretory signal sequence may be derived from ZcytoR21, a suitable signal sequence may also be derived from another secreted protein or synthesized de novo. The secretory signal sequence is operably linked to a ZcytoR21-encoding sequence such that the two sequences are joined in the correct reading frame and positioned to direct the newly synthesized polypeptide into the secretory pathway of the host cell. Secretory signal sequences are commonly positioned 5′ to the nucleotide sequence encoding the polypeptide of interest, although certain secretory signal sequences may be positioned elsewhere in the nucleotide sequence of interest (see, e.g., Welch et al., U.S. Pat. No. 5,037,743; Holland et al., U.S. Pat. No. 5,143,830).

Although the secretory signal sequence of ZcytoR21 or another protein produced by mammalian cells (e.g., tissue-type plasminogen activator signal sequence, as described, for example, in U.S. Pat. No. 5,641,655) is useful for expression of ZcytoR21 in recombinant mammalian hosts, a yeast signal sequence is preferred for expression in yeast cells. Examples of suitable yeast signal sequences are those derived from yeast mating phermone α-factor (encoded by the MFα1 gene), invertase (encoded by the SUC2 gene), or acid phosphatase (encoded by the PHO5 gene). See, for example, Romanos et al., “Expression of Cloned Genes in Yeast,” in DNA Cloning 2: A Practical Approach, 2^{nd} Edition, Glover and Hames (eds.), pages 123-167 (Oxford University Press 1995).

ZcytoR21 soluble receptor polypeptides can be prepared by expressing a truncated DNA encoding the extracellular domain, for example, a polypeptide which contains SEQ ID NO:6, or the corresponding region of a non-human receptor. It is preferred that the extracellular domain polypeptides be prepared in a form substantially free of transmembrane and intracellular polypeptide segments. To direct the export of the receptor domain from the host cell, the receptor DNA is linked to a second DNA segment encoding a secretory peptide, such as a t-PA secretory peptide. To facilitate purification of the secreted receptor domain, a C-terminal extension, such as a poly-histidine tag, substance P, Flag™ peptide (Hopp et al., Biotechnology 6:1204-1210, (1988); available from Eastman Kodak Co., New Haven, Conn.) or another polypeptide or protein for which an antibody or other specific binding agent is available, can be fused to the receptor polypeptide. Moreover, ZcytoR21 antigenic epitopes from the extracellular cytokine binding domains are also prepared as described above.

In an alternative approach, a receptor extracellular domain of ZcytoR21 or other cytokine receptor component can be expressed as a fusion with immunoglobulin heavy chain constant regions, typically an Fc fragment, which contains two constant region domains and a hinge region but lacks the variable region (See, Sledziewski, A Z et al., U.S. Pat. Nos. 6,018,026 and 5,750,375). The soluble ZcytoR21 polypeptides of the present invention include such fusions. One such fusion is shown in SEQ ID NOs:100 and 102; and 123 and 124. Such fusions are typically secreted as multimeric molecules wherein the Fc portions are disulfide bonded to each other and two receptor polypeptides are arrayed in closed proximity to each other. Fusions of this type can be used to affinity purify the cognate ligand from solution, as an in vitro assay tool, to block, inhibit or reduce signals in vitro by specifically titrating out ligand, and as antagonists in vivo by administering them parenterally to bind circulating ligand and clear it from the circulation. To purify ligand, a ZcytoR21-Ig chimera is added to a sample containing the ligand (e.g., cell-conditioned culture media or tissue extracts) under conditions that facilitate receptor-ligand binding (typically near-physiological temperature, pH, and ionic strength). The chimera-ligand complex is then separated by the mixture using protein A, which is immobilized on a solid support (e.g., insoluble resin beads). The ligand is then eluted using conventional chemical techniques, such as with a salt or pH gradient. In the alternative, the chimera itself can be bound to a solid support, with binding and elution carried out as above. The chimeras may be used in vivo to regulate inflammatory responses including acute phase responses such as serum amyloid A (SAA), C-reactive protein (CRP), and the like. Chimeras with high binding affinity are administered parenterally (e.g., by intramuscular, subcutaneous or intravenous injection). Circulating molecules bind ligand and are cleared from circulation by normal physiological processes. For use in assays, the chimeras are bound to a support via the Fc region and used in an ELISA format.

To assist in isolating anti-ZcytoR21 and binding partners of the present invention, an assay system that uses a ligand-binding receptor (or an antibody, one member of a complement/anti-complement pair) or a binding fragment thereof, and a commercially available biosensor instrument (BIAcore, Pharmacia Biosensor, Piscataway, N.J.) may be advantageously employed. Such receptor, antibody, member of a complement/anti-complement pair or fragment is immobilized onto the surface of a receptor chip. Use of this instrument is disclosed by Karlsson, J. Immunol. Methods 145:229-40, 1991 and Cunningham and Wells, J. Mol. Biol. 234:554-63, 1993. A receptor, antibody, member or fragment is covalently attached, using amine or sulfhydryl chemistry, to dextran fibers that are attached to gold film within the flow cell. A test sample is passed through the cell. If a ligand, epitope, or opposite member of the complement/anti-complement pair is present in the sample, it will bind to the immobilized receptor, antibody or member, respectively, causing a change in the refractive index of the medium, which is detected as a change in surface plasmon resonance of the gold film. This system allows the determination of on- and off-rates, from which binding affinity can be calculated, and assessment of stoichiometry of binding. Alternatively, ligand/receptor binding can be analyzed using SELDI(TM) technology (Ciphergen, Inc., Palo Alto, Calif.). Moreover, BIACorE technology, described above, can be used to be used in competition experiments to determine if different monoclonal antibodies bind the same or different epitopes on the ZcytoR21 polypeptide, and as such, be used to aid in epitope mapping of neutralizing antibodies of the present invention that bind, block, inhibit, reduce, antagonize or neutralize IL-17C.

Ligand-binding receptor polypeptides can also be used within other assay systems known in the art. Such systems include Scatchard analysis for determination of binding affinity (see Scatchard, Ann. NY Acad. Sci. 51: 660-72, 1949) and calorimetric assays (Cunningham et al., Science 253:545-48, 1991; Cunningham et al., Science 245:821-25, 1991).

The present invention further provides a variety of other polypeptide fusions and related multimeric proteins comprising one or more polypeptide fusions. For example, a soluble ZcytoR21 receptor can be prepared as a fusion to a dimerizing protein as disclosed in U.S. Pat. Nos. 5,155,027 and 5,567,584. Preferred dimerizing proteins in this regard include immunoglobulin constant region domains, e.g., IgGγ1, and the human κ light chain. Immunoglobulin-soluble ZcytoR21 fusions can be expressed in genetically engineered cells to produce a variety of multimeric ZcytoR21 receptor analogs. Auxiliary domains can be fused to soluble ZcytoR21 receptor to target them to specific cells, tissues, or macromolecules (e.g., collagen, or cells expressing the ZcytoR21 ligand, IL-17C). A ZcytoR21 polypeptide can be fused to two or more moieties, such as an affinity tag for purification and a targeting domain. Polypeptide fusions can also comprise one or more cleavage sites, particularly between domains. See, Tuan et al., Connective Tissue Research 34:1-9, 1996.

In bacterial cells, it is often desirable to express a heterologous protein as a fusion protein to decrease toxicity, increase stability, and to enhance recovery of the expressed protein. For example, ZcytoR21 can be expressed as a fusion protein comprising a glutathione S-transferase polypeptide. Glutathione S-transferease fusion proteins are typically soluble, and easily purifiable from E. coli lysates on immobilized glutathione columns. In similar approaches, a ZcytoR21 fusion protein comprising a maltose binding protein polypeptide can be isolated with an amylose resin column, while a fusion protein comprising the C-terminal end of a truncated Protein A gene can be purified using IgG-Sepharose. Established techniques for expressing a heterologous polypeptide as a fusion protein in a bacterial cell are described, for example, by Williams et al., “Expression of Foreign Proteins in E. coli Using Plasmid Vectors and Purification of Specific Polyclonal Antibodies,” in DNA Cloning 2: A Practical Approach, 2^{nd} Edition, Glover and Hames (Eds.), pages 15-58 (Oxford University Press 1995). In addition, commercially available expression systems are available. For example, the PINPOINT Xa protein purification system (Promega Corporation; Madison, Wis.) provides a method for isolating a fusion protein comprising a polypeptide that becomes biotinylated during expression with a resin that comprises avidin.

Peptide tags that are useful for isolating heterologous polypeptides expressed by either prokaryotic or eukaryotic cells include polyHistidine tags (which have an affinity for nickel-chelating resin), c-myc tags, calmodulin binding protein (isolated with calmodulin affinity chromatography), substance P, the RYIRS tag (which binds with anti-RYIRS antibodies), the Glu-Glu tag, and the FLAG tag (which binds with anti-FLAG antibodies). See, for example, Luo et al., Arch. Biochem. Biophys. 329:215 (1996), Morganti et al., Biotechnol. Appl. Biochem. 23:67 (1996), and Zheng et al., Gene 186:55 (1997). Nucleic acid molecules encoding such peptide tags are available, for example, from Sigma-Aldrich Corporation (St. Louis, Mo.).

Another form of fusion protein comprises a ZcytoR21 polypeptide and an immunoglobulin heavy chain constant region, typically an F_{C} fragment, which contains two or three constant region domains and a hinge region but lacks the variable region. As an illustration, Chang et al., U.S. Pat. No. 5,723,125, describe a fusion protein comprising a human interferon and a human immunoglobulin Fc fragment. The C-terminal of the interferon is linked to the N-terminal of the Fc fragment by a peptide linker moiety. An example of a peptide linker is a peptide comprising primarily a T cell inert sequence, which is immunologically inert. An exemplary peptide linker has the amino acid sequence: GGSGG SGGGG SGGGG S (SEQ ID NO:25). In this fusion protein, an illustrative Fc moiety is a human γ4 chain, which is stable in solution and has little or no complement activating activity. Accordingly, the present invention contemplates a ZcytoR21 fusion protein that comprises a ZcytoR21 moiety and a human Fc fragment, wherein the C-terminus of the ZcytoR21 moiety is attached to the N-terminus of the Fc fragment via a peptide linker, such as a peptide comprising the amino acid sequence of SEQ ID NOs:2, 5, 8, 11, 14, 21, 23, 107, 109, 113, 115, 117, 119, or 122. The ZcytoR21 moiety can be a ZcytoR21 molecule or a fragment thereof. For example, a fusion protein can comprise the amino acid of SEQ ID NO:3 and an Fc fragment (e.g., a human Fc fragment) (SEQ ID NO:100), SEQ ID NO:6 and an Fc fragment (SEQ ID NO:102), SEQ ID NO:122 and an Fc fragment (e.g., a human Fc fragment), SEQ ID NO:109 and an Fc fragment (e.g., a human Fc fragment), SEQ ID NO:113 and an Fc fragment (e.g., a human Fc fragment) (SEQ ID NO:124), SEQ ID NO:115 and an Fc fragment (e.g., a human Fc fragment), SEQ ID NO:117 and an Fc fragment (e.g., a human Fc fragment), and SEQ ID NO:119 and an Fc fragment (e.g., a human Fc fragment).

In a preferred embodiment of the invention, an amino acid linker may be included between the soluble ZcytoR21 and the Fc domains. Additionally, an alternative secretion leader may be used in place of the native ZcytoR21 leader.

One skilled in the art would also recognize that the ZcytoR21 polypeptides disclosed herein may be fused to a number of different Fc domains (e.g. Fc4, Fc5, Fc10 or any other variation thereof).

In another variation, a ZcytoR21 fusion protein comprises an IgG sequence, a ZcytoR21 moiety covalently joined to the aminoterminal end of the IgG sequence, and a signal peptide that is covalently joined to the aminoterminal of the ZcytoR21 moiety, wherein the IgG sequence consists of the following elements in the following order: a hinge region, a CH₂ domain, and a CH₃ domain. Accordingly, the IgG sequence lacks a CH₁ domain. The ZcytoR21 moiety displays a ZcytoR21 activity, as described herein, such as the ability to bind with a ZcytoR21 ligand. This general approach to producing fusion proteins that comprise both antibody and nonantibody portions has been described by LaRochelle et al., EP 742830 (WO 95/21258).

Fusion proteins comprising a ZcytoR21 moiety and an Fc moiety can be used, for example, as an in vitro assay tool. For example, the presence of a ZcytoR21 ligand in a biological sample can be detected using a ZcytoR21-immunoglobulin fusion protein, in which the ZcytoR21 moiety is used to bind the ligand, and a macromolecule, such as Protein A or anti-Fc antibody, is used to bind the fusion protein to a solid support. Such systems can be used to identify agonists and antagonists that interfere with the binding of a ZcytoR21 ligands, e.g., IL-17C, to its receptor.

Other examples of antibody fusion proteins include polypeptides that comprise an antigen-binding domain and a ZcytoR21 fragment that contains a ZcytoR21 extracellular domain. Such molecules can be used to target particular tissues for the benefit of ZcytoR21 binding activity.

The present invention further provides a variety of other polypeptide fusions. For example, part or all of a domain(s) conferring a biological function can be swapped between ZcytoR21 of the present invention with the functionally equivalent domain(s) from another member of the cytokine receptor family. Polypeptide fusions can be expressed in recombinant host cells to produce a variety of ZcytoR21 fusion analogs. A ZcytoR21 polypeptide can be fused to two or more moieties or domains; such as an affinity tag for purification and a targeting domain. Polypeptide fusions can also comprise one or more cleavage sites, particularly between domains. See, for example, Tuan et al., Connective Tissue Research 34:1 (1996).

Fusion proteins can be prepared by methods known to those skilled in the art by preparing each component of the fusion protein and chemically conjugating them. Alternatively, a polynucleotide encoding both components of the fusion protein in the proper reading frame can be generated using known techniques and expressed by the methods described herein. General methods for enzymatic and chemical cleavage of fusion proteins are described, for example, by Ausubel (1995) at pages 16-19 to 16-25.

ZcytoR21 binding domains can be further characterized by physical analysis of structure, as determined by such techniques as nuclear magnetic resonance, crystallography, electron diffraction or photoaffinity labeling, in conjunction with mutation of putative contact site amino acids of ZcytoR21 ligand agonists. See, for example, de Vos et al., Science 255:306 (1992), Smith et al., J. Mol. Biol. 224:899 (1992), and Wlodaver et al., FEBS Lett. 309:59 (1992).

The present invention also contemplates chemically modified ZcytoR21 compositions, in which a ZcytoR21 polypeptide is linked with a polymer. Illustrative ZcytoR21 polypeptides are soluble polypeptides that lack a functional transmembrane domain, such as a polypeptide comprising any of SEQ ID NOs: 2, 5, 8, 11, 14, 21, 23, 107, 109, 113, 115, 117, 119, or 122. Typically, the polymer is water soluble so that the ZcytoR21 conjugate does not precipitate in an aqueous environment, such as a physiological environment. An example of a suitable polymer is one that has been modified to have a single reactive group, such as an active ester for acylation, or an aldehyde for alkylation. In this way, the degree of polymerization can be controlled. An example of a reactive aldehyde is polyethylene glycol propionaldehyde, or mono-(C1-C10) alkoxy, or aryloxy derivatives thereof (see, for example, Harris, et al., U.S. Pat. No. 5,252,714). The polymer may be branched or unbranched. Moreover, a mixture of polymers can be used to produce ZcytoR21 conjugates.

ZcytoR21 conjugates used for therapy can comprise pharmaceutically acceptable water-soluble polymer moieties. Suitable water-soluble polymers include polyethylene glycol (PEG), monomethoxy-PEG, mono-(C1-C10)alkoxy-PEG, aryloxy-PEG, poly-(N-vinyl pyrrolidone)PEG, tresyl monomethoxy PEG, PEG propionaldehyde, bis-succinimidyl carbonate PEG, propylene glycol homopolymers, a polypropylene oxide/ethylene oxide co-polymer, polyoxyethylated polyols (e.g., glycerol), polyvinyl alcohol, dextran, cellulose, or other carbohydrate-based polymers. Suitable PEG may have a molecular weight from about 600 to about 60,000, including, for example, 5,000, 12,000, 20,000 and 25,000. A ZcytoR21 conjugate can also comprise a mixture of such water-soluble polymers.

One example of a ZcytoR21 conjugate comprises a ZcytoR21 moiety and a polyalkyl oxide moiety attached to the N-terminus of the ZcytoR21 moiety. PEG is one suitable polyalkyl oxide. As an illustration, ZcytoR21 can be modified with PEG, a process known as “PEGylation.” PEGylation of ZcytoR21 can be carried out by any of the PEGylation reactions known in the art (see, for example, EP 0 154 316, Delgado et al., Critical Reviews in Therapeutic Drug Carrier Systems 9:249 (1992), Duncan and Spreafico, Clin. Pharmacokinet. 27:290 (1994), and Francis et al., Int J Hematol 68:1 (1998)). For example, PEGylation can be performed by an acylation reaction or by an alkylation reaction with a reactive polyethylene glycol molecule. In an alternative approach, ZcytoR21 conjugates are formed by condensing activated PEG, in which a terminal hydroxy or amino group of PEG has been replaced by an activated linker (see, for example, Karasiewicz et al., U.S. Pat. No. 5,382,657).

PEGylation by acylation typically requires reacting an active ester derivative of PEG with a ZcytoR21 polypeptide. An example of an activated PEG ester is PEG esterified to N-hydroxysuccinimide. As used herein, the term “acylation” includes the following types of linkages between ZcytoR21 and a water soluble polymer: amide, carbamate, urethane, and the like. Methods for preparing PEGylated ZcytoR21 by acylation will typically comprise the steps of (a) reacting a ZcytoR21 polypeptide with PEG (such as a reactive ester of an aldehyde derivative of PEG) under conditions whereby one or more PEG groups attach to ZcytoR21, and (b) obtaining the reaction product(s). Generally, the optimal reaction conditions for acylation reactions will be determined based upon known parameters and desired results. For example, the larger the ratio of PEG:ZcytoR21, the greater the percentage of polyPEGylated ZcytoR21 product.

The product of PEGylation by acylation is typically a polyPEGylated ZcytoR21 product, wherein the lysine ε-amino groups are PEGylated via an acyl linking group. An example of a connecting linkage is an amide. Typically, the resulting ZcytoR21 will be at least 95% mono-, di-, or tri-pegylated, although some species with higher degrees of PEGylation may be formed depending upon the reaction conditions. PEGylated species can be separated from unconjugated ZcytoR21 polypeptides using standard purification methods, such as dialysis, ultrafiltration, ion exchange chromatography, affinity chromatography, and the like.

PEGylation by alkylation generally involves reacting a terminal aldehyde derivative of PEG with ZcytoR21 in the presence of a reducing agent. PEG groups can be attached to the polypeptide via a —CH₂—NH group.

Moreover, anti-ZcytoR21 antibodies or antibody fragments of the present invention can be PEGylated using methods in the art and described herein.

Derivatization via reductive alkylation to produce a monoPEGylated product takes advantage of the differential reactivity of different types of primary amino groups available for derivatization. Typically, the reaction is performed at a pH that allows one to take advantage of the pKa differences between the ε-amino groups of the lysine residues and the α-amino group of the N-terminal residue of the protein. By such selective derivatization, attachment of a water-soluble polymer that contains a reactive group such as an aldehyde, to a protein is controlled. The conjugation with the polymer occurs predominantly at the N-terminus of the protein without significant modification of other reactive groups such as the lysine side chain amino groups. The present invention provides a substantially homogenous preparation of ZcytoR21 monopolymer conjugates.

Reductive alkylation to produce a substantially homogenous population of monopolymer ZcytoR21 conjugate molecule can comprise the steps of: (a) reacting a ZcytoR21 polypeptide with a reactive PEG under reductive alkylation conditions at a pH suitable to permit selective modification of the α-amino group at the amino terminus of the ZcytoR21, and (b) obtaining the reaction product(s). The reducing agent used for reductive alkylation should be stable in aqueous solution and able to reduce only the Schiff base formed in the initial process of reductive alkylation. Illustrative reducing agents include sodium borohydride, sodium cyanoborohydride, dimethylamine borane, trimethylamine borane, and pyridine borane.

For a substantially homogenous population of monopolymer ZcytoR21 conjugates, the reductive alkylation reaction conditions are those that permit the selective attachment of the water-soluble polymer moiety to the N-terminus of ZcytoR21. Such reaction conditions generally provide for pKa differences between the lysine amino groups and the α-amino group at the N-terminus. The pH also affects the ratio of polymer to protein to be used. In general, if the pH is lower, a larger excess of polymer to protein will be desired because the less reactive the N-terminal α-group, the more polymer is needed to achieve optimal conditions. If the pH is higher, the polymer:ZcytoR21 need not be as large because more reactive groups are available. Typically, the pH will fall within the range of 3 to 9, or 3 to 6. This method can be employed for making ZcytoR21-comprising homodimeric, heterodimeric or multimeric soluble receptor conjugates.

Another factor to consider is the molecular weight of the water-soluble polymer. Generally, the higher the molecular weight of the polymer, the fewer number of polymer molecules which may be attached to the protein. For PEGylation reactions, the typical molecular weight is about 2 kDa to about 100 kDa, about 5 kDa to about 50 kDa, or about 12 kDa to about 25 kDa. The molar ratio of water-soluble polymer to ZcytoR21 will generally be in the range of 1:1 to 100:1. Typically, the molar ratio of water-soluble polymer to ZcytoR21 will be 1:1 to 20:1 for polyPEGylation, and 1:1 to 5:1 for monoPEGylation.

General methods for producing conjugates comprising a polypeptide and water-soluble polymer moieties are known in the art. See, for example, Karasiewicz et al., U.S. Pat. No. 5,382,657, Greenwald et al., U.S. Pat. No. 5,738,846, Nieforth et al., Clin. Pharmacol. Ther. 59:636 (1996), Monkarsh et al., Anal. Biochem. 247:434 (1997)). This method can be employed for making ZcytoR21-comprising homodimeric, heterodimeric or multimeric soluble receptor conjugates.

The present invention contemplates compositions comprising a peptide or polypeptide, such as a soluble receptor or antibody described herein. Such compositions can further comprise a carrier. The carrier can be a conventional organic or inorganic carrier. Examples of carriers include water, buffer solution, alcohol, propylene glycol, macrogol, sesame oil, corn oil, and the like.

G) Isolation of ZcytoR21 Polypeptides

The polypeptides of the present invention can be purified to at least about 80% purity, to at least about 90% purity, to at least about 95% purity, or greater than 95%, such as 96%, 97%, 98%, or greater than 99% purity with respect to contaminating macromolecules, particularly other proteins and nucleic acids, and free of infectious and pyrogenic agents. The polypeptides of the present invention may also be purified to a pharmaceutically pure state, which is greater than 99.9% pure. In certain preparations, purified polypeptide is substantially free of other polypeptides, particularly other polypeptides of animal origin.

Fractionation and/or conventional purification methods can be used to obtain preparations of ZcytoR21 purified from natural sources (e.g., human tissue sources), synthetic ZcytoR21 polypeptides, and recombinant ZcytoR21 polypeptides and fusion ZcytoR21 polypeptides purified from recombinant host cells. In general, ammonium sulfate precipitation and acid or chaotrope extraction may be used for fractionation of samples. Exemplary purification steps may include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are suitable. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties.

Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Selection of a particular method for polypeptide isolation and purification is a matter of routine design and is determined in part by the properties of the chosen support. See, for example, Affinity Chromatography: Principles & Methods (Pharmacia LKB Biotechnology 1988), and Doonan, Protein Purification Protocols (The Humana Press 1996).

Additional variations in ZcytoR21 isolation and purification can be devised by those of skill in the art. For example, anti-ZcytoR21 antibodies, obtained as described below, can be used to isolate large quantities of protein by immunoaffinity purification.

The polypeptides of the present invention can also be isolated by exploitation of particular properties. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1 (1985)). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (M. Deutscher, (ed.), Meth. Enzymol. 182:529 (1990)). Within additional embodiments of the invention, a fusion of the polypeptide of interest and an affinity tag (e.g., maltose-binding protein, an immunoglobulin domain) may be constructed to facilitate purification. Moreover, the ligand-binding properties of ZcytoR21 extracellular domain can be exploited for purification, for example, of ZcytoR21-comprising soluble receptors; for example, by using affinity chromatography wherein IL-17C ligand is bound to a column and the ZcytoR21-comprising receptor is bound and subsequently eluted using standard chromatography methods.

ZcytoR21 polypeptides or fragments thereof may also be prepared through chemical synthesis, as described above. ZcytoR21 polypeptides may be monomers or multimers; glycosylated or non-glycosylated; PEGylated or non-PEGylated; and may or may not include an initial methionine amino acid residue.

H) Production of Antibodies to ZcytoR21 Proteins

Antibodies to ZcytoR21 can be obtained, for example, using the product of a ZcytoR21 expression vector or ZcytoR21 isolated from a natural source as an antigen. Particularly useful anti-ZcytoR21 antibodies “bind specifically” with ZcytoR21. Antibodies are considered to be specifically binding if the antibodies exhibit at least one of the following two properties: (1) antibodies bind to ZcytoR21 with a threshold level of binding activity, and (2) antibodies do not significantly cross-react with polypeptides related to ZcytoR21.

With regard to the first characteristic, antibodies specifically bind if they bind to a ZcytoR21 polypeptide, peptide or epitope with a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art, for example, by Scatchard analysis (Scatchard, Ann. NY Acad. Sci. 51:660 (1949)). With regard to the second characteristic, antibodies do not significantly cross-react with related polypeptide molecules, for example, if they detect ZcytoR21, but not presently known polypeptides using a standard Western blot analysis. Examples of known related polypeptides include known cytokine receptors.

Anti-ZcytoR21 antibodies can be produced using antigenic ZcytoR21 epitope-bearing peptides and polypeptides. Antigenic epitope-bearing peptides and polypeptides of the present invention contain a sequence of at least nine, or between 15 to about 30 amino acids contained within any of SEQ ID NOs: 2, 5, 8, 11, 14, 21, 23, 107, 109, 113, 115, 117, 119, or 122, or another amino acid sequence disclosed herein. However, peptides or polypeptides comprising a larger portion of an amino acid sequence of the invention, containing from 30 to 50 amino acids, or any length up to and including the entire amino acid sequence of a polypeptide of the invention, also are useful for inducing antibodies that bind with ZcytoR21. It is desirable that the amino acid sequence of the epitope-bearing peptide is selected to provide substantial solubility in aqueous solvents (i.e., the sequence includes relatively hydrophilic residues, while hydrophobic residues are typically avoided). Moreover, amino acid sequences containing proline residues may be also be desirable for antibody production.

As an illustration, potential antigenic sites in ZcytoR21 were identified using the Jameson-Wolf method, Jameson and Wolf, CABIOS 4:181, (1988), as implemented by the PROTEAN program (version 3.14) of LASERGENE (DNASTAR; Madison, Wis.). Default parameters were used in this analysis.

The Jameson-Wolf method predicts potential antigenic determinants by combining six major subroutines for protein structural prediction. Briefly, the Hopp-Woods method, Hopp et al., Proc. Nat'l Acad. Sci. USA 78:3824 (1981), was first used to identify amino acid sequences representing areas of greatest local hydrophilicity (parameter: seven residues averaged). In the second step, Emini's method, Emini et al., J. Virology 55:836 (1985), was used to calculate surface probabilities (parameter: surface decision threshold (0.6)=1). Third, the Karplus-Schultz method, Karplus and Schultz, Naturwissenschaften 72:212 (1985), was used to predict backbone chain flexibility (parameter: flexibility threshold (0.2)=1). In the fourth and fifth steps of the analysis, secondary structure predictions were applied to the data using the methods of Chou-Fasman, Chou, “Prediction of Protein Structural Classes from Amino Acid Composition,” in Prediction of Protein Structure and the Principles of Protein Confonration, Fasman (ed.), pages 549-586 (Plenum Press 1990), and Garnier-Robson, Garnier et al., J. Mol. Biol. 120:97 (1978) (Chou-Fasman parameters: conformation table=64 proteins; α region threshold=103; β region threshold=105; Garnier-Robson parameters: α and β decision constants=0). In the sixth subroutine, flexibility parameters and hydropathy/solvent accessibility factors were combined to determine a surface contour value, designated as the “antigenic index.” Finally, a peak broadening function was applied to the antigenic index, which broadens major surface peaks by adding 20, 40, 60, or 80% of the respective peak value to account for additional free energy derived from the mobility of surface regions relative to interior regions. This calculation was not applied, however, to any major peak that resides in a helical region, since helical regions tend to be less flexible. Hopp/Woods hydrophilicity profiles can be used to determine regions that have the most antigenic potential within SEQ ID NO:6 (Hopp et al., Proc. Natl. Acad. Sci. 78:3824-3828, 1981; Hopp, J. Immun. Meth. 88:1-18, 1986 and Triquier et al., Protein Engineering 11:153-169, 1998). The profile is based on a sliding six-residue window. Buried G, S, and T residues and exposed H, Y, and W residues were ignored. Moreover, ZcytoR21 antigenic epitopes within SEQ ID NO:6 as predicted by a Jameson-Wolf plot, e.g., using DNASTAR Protean program (DNASTAR, Inc., Madison, Wis.) serve as preferred antigenic epitopes, and can be determined by one of skill in the art. Such antigenic epitopes include SEQ ID NOs: 115 (“antigenic peptide 1”), 117 (“antigenic peptide 2”), 119 (“antigenic peptide 3”), and the following amino acid sequences of SEQ ID NO:6 would provide suitable antigenic peptides: amino acids 51 to 59 (“antigenic peptide 4”), amino acids 72 to 83 (“antigenic peptide 5”), 91 to 97 (“antigenic peptide 6”), amino acids 174 to 180 (“antigenic peptide 7”), and amino acids 242 to 246 (“antigenic peptide 8”). The present invention contemplates the use of any one of antigenic peptides X to Y to generate antibodies to ZcytoR21 or as a tool to screen or identify neutralizing monoclonal antibodies of the present invention. The present invention also contemplates polypeptides comprising at least one of antigenic peptides 1 to 5. The present invention contemplates the use of any antigenic peptides or epitopes described herein to generate antibodies to ZcytoR21, as well as to identify and screen anti-ZcytoR21 monoclonal antibodies that are neutralizing, and that may bind, block, inhibit, reduce, antagonize or neutralize the activity of IL-17C.

Moreover, suitable antigens also include the ZcytoR21 polypeptides comprising a ZcytoR21 cytokine binding, or extracellular domain disclosed above in combination with another cytokine extracellular domain, such as a class I or II cytokine receptor domain, such as those that may form soluble ZcytoR21 heterodimeric or multimeric polypeptides, and the like.

Polyclonal antibodies to recombinant ZcytoR21 protein or to ZcytoR21 isolated from natural sources can be prepared using methods well-known to those of skill in the art. See, for example, Green et al., “Production of Polyclonal Antisera,” in Immunochemical Protocols (Manson, ed.), pages 1-5 (Humana Press 1992), and Williams et al., “Expression of foreign proteins in E. coli using plasmid vectors and purification of specific polyclonal antibodies,” in DNA Cloning 2: Expression Systems, 2nd Edition, Glover et al. (eds.), page 15 (Oxford University Press 1995). The immunogenicity of a ZcytoR21 polypeptide can be increased through the use of an adjuvant, such as alum (aluminum hydroxide) or Freund's complete or incomplete adjuvant. Polypeptides useful for immunization also include fusion polypeptides, such as fusions of ZcytoR21 or a portion thereof with an immunoglobulin polypeptide or with maltose binding protein. The polypeptide immunogen may be a full-length molecule or a portion thereof. If the polypeptide portion is “hapten-like,” such portion may be advantageously joined or linked to a macromolecular carrier (such as keyhole limpet hemocyanin (KLH), bovine serum albumin (BSA) or tetanus toxoid) for immunization.

Although polyclonal antibodies are typically raised in animals such as horses, cows, dogs, chicken, rats, mice, rabbits, guinea pigs, goats, or sheep, an anti-ZcytoR21 antibody of the present invention may also be derived from a subhuman primate antibody. General techniques for raising diagnostically and therapeutically useful antibodies in baboons may be found, for example, in Goldenberg et al., international patent publication No. WO 91/11465, and in Losman et al., Int. J. Cancer 46:310 (1990).

Alternatively, monoclonal anti-ZcytoR21 antibodies can be generated. Rodent monoclonal antibodies to specific antigens may be obtained by methods known to those skilled in the art (see, for example, Kohler et al., Nature 256:495 (1975), Coligan et al. (eds.), Current Protocols in Immunology, Vol. 1, pages 2.5.1-2.6.7 (John Wiley & Sons 1991) [“Coligan”], Picksley et al., “Production of monoclonal antibodies against proteins expressed in E. coli,” in DNA Cloning 2: Expression Systems, 2nd Edition, Glover et al. (eds.), page 93 (Oxford University Press 1995)).

Briefly, monoclonal antibodies can be obtained by injecting mice with a composition comprising a ZcytoR21 gene product, verifying the presence of antibody production by removing a serum sample, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

In addition, an anti-ZcytoR21 antibody of the present invention may be derived from a human monoclonal antibody. Human monoclonal antibodies are obtained from transgenic mice that have been engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described, for example, by Green et al., Nature Genet. 7:13 (1994), Lonberg et al., Nature 368:856 (1994), and Taylor et al., Int. Immun. 6:579 (1994).

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, for example, Coligan at pages 2.7.1-2.7.12 and pages 2.9.1-2.9.3; Baines et al., “Purification of Immunoglobulin G (IgG),” in Methods in Molecular Biology, Vol. 10, pages 79-104 (The Humana Press, Inc. 1992)).

For particular uses, it may be desirable to prepare fragments of anti-ZcytoR21 antibodies. Such antibody fragments can be obtained, for example, by proteolytic hydrolysis of the antibody. Antibody fragments can be obtained by pepsin or papain digestion of whole antibodies by conventional methods. As an illustration, antibody fragments can be produced by enzymatic cleavage of antibodies with pepsin to provide a 5S fragment denoted F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using pepsin produces two monovalent Fab fragments and an Fc fragment directly. These methods are described, for example, by Goldenberg, U.S. Pat. No. 4,331,647, Nisonoff et al., Arch Biochem. Biophys. 89:230 (1960), Porter, Biochem. J. 73:119 (1959), Edelman et al., in Methods in Enzymology Vol. 1, page 422 (Academic Press 1967), and by Coligan at pages 2.8.1-2.8.10 and 2.10.-2.10.4.

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

For example, Fv fragments comprise an association of V_{H} and V_{L} chains. This association can be noncovalent, as described by Inbar et al., Proc. Nat'l Acad. Sci. USA 69:2659 (1972). Alternatively, the variable chains can be linked by an intermolecular disulfide bond or cross-linked by chemicals such as glutaraldehyde (see, for example, Sandhu, Crit. Rev. Biotech. 12:437 (1992)).

The Fv fragments may comprise V_{H} and V_{L} chains which are connected by a peptide linker. These single-chain antigen binding proteins (scFv) are prepared by constructing a structural gene comprising DNA sequences encoding the V_{H} and V_{L} domains which are connected by an oligonucleotide. The structural gene is inserted into an expression vector which is subsequently introduced into a host cell, such as E. coli. The recombinant host cells synthesize a single polypeptide chain with a linker peptide bridging the two V domains. Methods for producing scFvs are described, for example, by Whitlow et al., Methods: A Companion to Methods in Enzymology 2:97 (1991) (also see, Bird et al., Science 242:423 (1988), Ladner et al., U.S. Pat. No. 4,946,778, Pack et al., Bio/Technology 11:1271 (1993), and Sandhu, supra).

As an illustration, a scFV can be obtained by exposing lymphocytes to ZcytoR21 polypeptide in vitro, and selecting antibody display libraries in phage or similar vectors (for instance, through use of immobilized or labeled ZcytoR21 protein or peptide). Genes encoding polypeptides having potential ZcytoR21 polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides which interact with a known target which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409, Ladner et al., U.S. Pat. No. 4,946,778, Ladner et al., U.S. Pat. No. 5,403,484, Ladner et al., U.S. Pat. No. 5,571,698, and Kay et al., Phage Display of Peptides and Proteins (Academic Press, Inc. 1996)) and random peptide display libraries and kits for screening such libraries are available commercially, for instance from CLONTECH Laboratories, Inc. (Palo Alto, Calif.), Invitrogen Inc. (San Diego, Calif.), New England Biolabs, Inc. (Beverly, Mass.), and Pharmacia LKB Biotechnology Inc. (Piscataway, N.J.). Random peptide display libraries can be screened using the ZcytoR21 sequences disclosed herein to identify proteins which bind to ZcytoR21.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, for example, Larrick et al., Methods: A Companion to Methods in Enzymology 2:106 (1991), Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995), and Ward et al., “Genetic Manipulation and Expression of Antibodies,” in Monoclonal Antibodies: Principles and Applications, Birch et al., (eds.), page 137 (Wiley-Liss, Inc. 1995)).

Alternatively, an anti-ZcytoR21 antibody may be derived from a “humanized” monoclonal antibody. Humanized monoclonal antibodies are produced by transferring mouse complementary determining regions from heavy and light variable chains of the mouse immunoglobulin into a human variable domain. Typical residues of human antibodies are then substituted in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. USA 86:3833 (1989). Techniques for producing humanized monoclonal antibodies are described, for example, by Jones et al., Nature 321:522 (1986), Carter et al., Proc. Nat'l Acad. Sci. USA 89:4285 (1992), Sandhu, Crit. Rev. Biotech. 12:437 (1992), Singer et al., J. Immun. 150:2844 (1993), Sudhir (ed.), Antibody Engineering Protocols (Humana Press, Inc. 1995), Kelley, “Engineering Therapeutic Antibodies,” in Protein Engineering: Principles and Practice, Cleland et al. (eds.), pages 399-434 (John Wiley & Sons, Inc. 1996), and by Queen et al., U.S. Pat. No. 5,693,762 (1997).

Moreover, anti-ZcytoR21 antibodies or antibody fragments of the present invention can be PEGylated using methods in the art and described herein.

Polyclonal anti-idiotype antibodies can be prepared by immunizing animals with anti-ZcytoR21 antibodies or antibody fragments, using standard techniques. See, for example, Green et al., “Production of Polyclonal Antisera,” in Methods In Molecular Biology: Immunochemical Protocols, Manson (ed.), pages 1-12 (Humana Press 1992). Also, see Coligan at pages 2.4.1-2.4.7. Alternatively, monoclonal anti-idiotype antibodies can be prepared using anti-ZcytoR21 antibodies or antibody fragments as immunogens with the techniques, described above. As another alternative, humanized anti-idiotype antibodies or subhuman primate anti-idiotype antibodies can be prepared using the above-described techniques. Methods for producing anti-idiotype antibodies are described, for example, by Irie, U.S. Pat. No. 5,208,146, Greene, et. al., U.S. Pat. No. 5,637,677, and Varthakavi and Minocha, J. Gen. Virol. 77:1875 (1996).

An anti-ZcytoR21 antibody can be conjugated with a detectable label to form an anti-ZcytoR21 immunoconjugate. Suitable detectable labels include, for example, a radioisotope, a fluorescent label, a chemiluminescent label, an enzyme label, a bioluminescent label or colloidal gold. Methods of making and detecting such detectably-labeled immunoconjugates are well-known to those of ordinary skill in the art, and are described in more detail below.

The detectable label can be a radioisotope that is detected by autoradiography. Isotopes that are particularly useful for the purpose of the present invention are ³H, ¹²⁵I, ¹³¹I, ³⁵S and ¹⁴C.

Anti-ZcytoR21 immunoconjugates can also be labeled with a fluorescent compound. The presence of a fluorescently-labeled antibody is determined by exposing the immunoconjugate to light of the proper wavelength and detecting the resultant fluorescence. Fluorescent labeling compounds include fluorescein isothiocyanate, rhodamine, phycoerytherin, phycocyanin, allophycocyanin, o-phthaldehyde and fluorescamine.

Alternatively, anti-ZcytoR21 immunoconjugates can be detectably labeled by coupling an antibody component to a chemiluminescent compound. The presence of the chemiluminescent-tagged immunoconjugate is determined by detecting the presence of luminescence that arises during the course of a chemical reaction. Examples of chemiluminescent labeling compounds include luminol, isoluminol, an aromatic acridinium ester, an imidazole, an acridinium salt and an oxalate ester.

Similarly, a bioluminescent compound can be used to label anti-ZcytoR21 immunoconjugates of the present invention. Bioluminescence is a type of chemiluminescence found in biological systems in which a catalytic protein increases the efficiency of the chemiluminescent reaction. The presence of a bioluminescent protein is determined by detecting the presence of luminescence. Bioluminescent compounds that are useful for labeling include luciferin, luciferase and aequorin.

Alternatively, anti-ZcytoR21 immunoconjugates can be detectably labeled by linking an anti-ZcytoR21 antibody component to an enzyme. When the anti-ZcytoR21-enzyme conjugate is incubated in the presence of the appropriate substrate, the enzyme moiety reacts with the substrate to produce a chemical moiety which can be detected, for example, by spectrophotometric, fluorometric or visual means. Examples of enzymes that can be used to detectably label polyspecific immunoconjugates include β-galactosidase, glucose oxidase, peroxidase and alkaline phosphatase.

Those of skill in the art will know of other suitable labels which can be employed in accordance with the present invention. The binding of marker moieties to anti-ZcytoR21 antibodies can be accomplished using standard techniques known to the art. Typical methodology in this regard is described by Kennedy et al., Clin. Chim. Acta 70:1 (1976), Schurs et al., Clin. Chim. Acta 81:1 (1977), Shih et al., Int'l J. Cancer 46:1101 (1990), Stein et al., Cancer Res. 50:1330 (1990), and Coligan, supra.

Moreover, the convenience and versatility of immunochemical detection can be enhanced by using anti-ZcytoR21 antibodies that have been conjugated with avidin, streptavidin, and biotin (see, for example, Wilchek et al. (eds.), “Avidin-Biotin Technology,” Methods In Enzymology, Vol. 184 (Academic Press 1990), and Bayer et al., “Imunochemical Applications of Avidin-Biotin Technology,” in Methods In Molecular Biology, Vol. 10, Manson (ed.), pages 149-162 (The Humana Press, Inc. 1992).

Methods for performing immunoassays are well-established. See, for example, Cook and Self, “Monoclonal Antibodies in Diagnostic Immunoassays,” in Monoclonal Antibodies: Production, Engineering, and Clinical Application, Ritter and Ladyman (eds.), pages 180-208, (Cambridge University Press, 1995), Perry, “The Role of Monoclonal Antibodies in the Advancement of Immunoassay Technology,” in Monoclonal Antibodies: Principles and Applications, Birch and Lennox (eds.), pages 107-120 (Wiley-Liss, Inc. 1995), and Diamandis, Immunoassay (Academic Press, Inc. 1996).

The present invention also contemplates kits for performing an immunological diagnostic assay for ZcytoR21 gene expression. Such kits comprise at least one container comprising an anti-ZcytoR21 antibody, or antibody fragment. A kit may also comprise a second container comprising one or more reagents capable of indicating the presence of ZcytoR21 antibody or antibody fragments. Examples of such indicator reagents include detectable labels such as a radioactive label, a fluorescent label, a chemiluminescent label, an enzyme label, a bioluminescent label, colloidal gold, and the like. A kit may also comprise a means for conveying to the user that ZcytoR21 antibodies or antibody fragments are used to detect ZcytoR21 protein. For example, written instructions may state that the enclosed antibody or antibody fragment can be used to detect ZcytoR21. The written material can be applied directly to a container, or the written material can be provided in the form of a packaging insert.

I) Use of Anti-ZcytoR21 Antibodies to Antagonize ZcytoR21 Binding to IL-17C

Alternative techniques for generating or selecting antibodies useful herein include in vitro exposure of lymphocytes to soluble ZcytoR21 receptor polypeptides or fragments thereof, such as antigenic epitopes, and selection of antibody display libraries in phage or similar vectors (for instance, through use of immobilized or labeled soluble ZcytoR21 receptor polypeptides or fragments thereof, such as antigenic epitopes). Genes encoding polypeptides having potential binding domains such as soluble ZcytoR21 receptor polypeptides or fragments thereof, such as antigenic epitopes can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides that interact with a known target that can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially, for instance from Clontech (Palo Alto, Calif.), Invitrogen Inc. (San Diego, Calif.), New England Biolabs, Inc. (Beverly, Mass.) and Pharmacia LKB Biotechnology Inc. (Piscataway, N.J.). Random peptide display libraries can be screened using the soluble ZcytoR21 receptor polypeptides or fragments thereof, such as antigenic epitope polypeptide sequences disclosed herein to identify proteins which bind to ZcytoR21-comprising receptor polypeptides. These “binding polypeptides,” which interact with soluble ZcytoR21-comprising receptor polypeptides, can be used for tagging cells; for isolating homolog polypeptides by affinity purification; they can be directly or indirectly conjugated to drugs, toxins, radionuclides and the like. These binding polypeptides can also be used in analytical methods such as for screening expression libraries and neutralizing activity, e.g., for binding, blocking, inhibiting, reducing, antagonizing or neutralizing interaction between IL-17C and ZcytoR21, or viral binding to a receptor. The binding polypeptides can also be used for diagnostic assays for determining circulating levels of soluble ZcytoR21-comprising receptor polypeptides; for detecting or quantitating soluble or non-soluble ZcytoR21-comprising receptors as marker of underlying pathology or disease. These binding polypeptides can also act as “antagonists” to block or inhibit soluble or membrane-bound ZcytoR21 monomeric receptor or ZcytoR21 homodimeric, heterodimeric or multimeric polypeptide binding (e.g. to ligand) and signal transduction in vitro and in vivo. Again, these binding polypeptides serve as anti-ZcytoR21 monomeric receptor or anti-ZcytoR21 homodimeric, heterodimeric or multimeric polypeptides and are useful for inhibiting IL-17C activity, as well as receptor activity or protein-binding. Antibodies raised to the natural receptor complexes of the present invention, and ZcytoR21-epitope-binding antibodies, and anti-ZcytoR21 neutralizing monoclonal antibodies may be preferred embodiments, as they may act more specifically against the ZcytoR21 and can inhibit IL-17C. Moreover, the antagonistic and binding activity of the antibodies of the present invention can be assayed in an IL-17C proliferation, signal trap, luciferase, phosphoprotein, or binding assays in the presence of IL-17C, and ZcytoR21-comprising soluble receptors, and other biological or biochemical assays described herein.

Antibodies to soluble ZcytoR21 receptor polypeptides (e.g., antibodies to SEQ ID NO: 2, 5, 8, 11, 14, 21, 23, 107, 109, 113, 115, 117, 119, or 122) or fragments thereof, such as antigenic epitopes may be used for inhibiting the inflammatory effects of IL-17C in vivo, for theraputic use against inflammation and inflammatory dieases such as psoriasis, psoriatic arthritis, rheumatoid arthritis, endotoxemia, inflammatory bowel disease (IBD), colitis, asthma, allograft rejection, immune mediated renal diseases, hepatobiliary diseases, multiple sclerosis, atherosclerosis, promotion of tumor growth, or degenerative joint disease and other inflammatory conditions disclosed herein; tagging cells that express ZcytoR21 receptors; for isolating soluble ZcytoR21-comprising receptor polypeptides by affinity purification; for diagnostic assays for determining circulating levels of soluble ZcytoR21-comprising receptor polypeptides; for detecting or quantitating soluble ZcytoR21-comprising receptors as marker of underlying pathology or disease; in analytical methods employing FACS; for screening expression libraries; for generating anti-idiotypic antibodies that can act as IL-17C agonists; and as neutralizing antibodies or as antagonists to bind, block, inhibit, reduce, or antagonize ZcytoR21 receptor function, or to bind, block, inhibit, reduce, antagonize or neutralize IL-17C activity in vitro and in vivo. Suitable direct tags or labels include radionuclides, enzymes, substrates, cofactors, biotin, inhibitors, fluorescent markers, chemiluminescent markers, magnetic particles and the like; indirect tags or labels may feature use of biotin-avidin or other complement/anti-complement pairs as intermediates. Antibodies herein may also be directly or indirectly conjugated to drugs, toxins, radionuclides and the like, and these conjugates used for in vivo diagnostic or therapeutic applications. Moreover, antibodies to soluble ZcytoR21-comprising receptor polypeptides, or fragments thereof may be used in vitro to detect denatured or non-denatured ZcytoR21-comprising receptor polypeptides or fragments thereof in assays, for example, Western Blots or other assays known in the art.

Antibodies to soluble ZcytoR21 receptor or soluble ZcytoR21 homodimeric, heterodimeric or multimeric receptor polypeptides are useful for tagging cells that express the corresponding receptors and assaying their expression levels, for affinity purification, within diagnostic assays for determining circulating levels of receptor polypeptides, analytical methods employing fluorescence-activated cell sorting. Moreover, divalent antibodies, and anti-idiotypic antibodies may be used as agonists to mimic the effect of the ZcytoR21 ligand, IL-17C.

Antibodies herein can also be directly or indirectly conjugated to drugs, toxins, radionuclides and the like, and these conjugates used for in vivo diagnostic or therapeutic applications. For instance, antibodies or binding polypeptides which recognize soluble ZcytoR21 receptor or soluble ZcytoR21 homodimeric, heterodimeric or multimeric receptor polypeptides can be used to identify or treat tissues or organs that express a corresponding anti-complementary molecule (i.e., a ZcytoR21-comprising soluble or membrane-bound receptor). More specifically, antibodies to soluble ZcytoR21-comprising receptor polypeptides, or bioactive fragments or portions thereof, can be coupled to detectable or cytotoxic molecules and delivered to a mammal having cells, tissues or organs that express the ZcytoR21-comprising receptor such as ZcytoR21-expressing cancers.

Suitable detectable molecules may be directly or indirectly attached to polypeptides that bind ZcytoR21-comprising receptor polypeptides, such as “binding polypeptides,” (including binding peptides disclosed above), antibodies, or bioactive fragments or portions thereof. Suitable detectable molecules include radionuclides, enzymes, substrates, cofactors, inhibitors, fluorescent markers, chemiluminescent markers, magnetic particles and the like. Suitable cytotoxic molecules may be directly or indirectly attached to the polypeptide or antibody, and include bacterial or plant toxins (for instance, diphtheria toxin, Pseudomonas exotoxin, ricin, abrin and the like), as well as therapeutic radionuclides, such as iodine-131, rhenium-188 or yttrium-90 (either directly attached to the polypeptide or antibody, or indirectly attached through means of a chelating moiety, for instance). Binding polypeptides or antibodies may also be conjugated to cytotoxic drugs, such as adriamycin. For indirect attachment of a detectable or cytotoxic molecule, the detectable or cytotoxic molecule can be conjugated with a member of a complementary/anticomplementary pair, where the other member is bound to the binding polypeptide or antibody portion. For these purposes, biotin/streptavidin is an exemplary complementary/anticomplementary pair.

In another embodiment, binding polypeptide-toxin fusion proteins or antibody-toxin fusion proteins can be used for targeted cell or tissue inhibition or ablation (for instance, to treat cancer cells or tissues). Alternatively, if the binding polypeptide has multiple functional domains (i.e., an activation domain or a ligand binding domain, plus a targeting domain), a fusion protein including only the targeting domain may be suitable for directing a detectable molecule, a cytotoxic molecule or a complementary molecule to a cell or tissue type of interest. In instances where the fusion protein including only a single domain includes a complementary molecule, the anti-complementary molecule can be conjugated to a detectable or cytotoxic molecule. Such domain-complementary molecule fusion proteins thus represent a generic targeting vehicle for cell/tissue-specific delivery of generic anti-complementary-detectable/cytotoxic molecule conjugates.

In another embodiment, ZcytoR21 binding polypeptide-cytokine or antibody-cytokine fusion proteins can be used for enhancing in vivo killing of target tissues (for example, spleen, pancreatic, blood, lymphoid, colon, and bone marrow cancers), if the binding polypeptide-cytokine or anti-ZcytoR21 receptor antibody targets the hyperproliferative cell (See, generally, Hornick et al., Blood 89:4437-47, 1997). The described fusion proteins enable targeting of a cytokine to a desired site of action, thereby providing an elevated local concentration of cytokine. Suitable anti-ZcytoR21 monomer, homodimer, heterodimer or multimer antibodies target an undesirable cell or tissue (i.e., a tumor or a leukemia), and the fused cytokine mediates improved target cell lysis by effector cells. Suitable cytokines for this purpose include interleukin 2 and granulocyte-macrophage colony-stimulating factor (GM-CSF), for instance.

Alternatively, ZcytoR21 receptor binding polypeptides or antibody fusion proteins described herein can be used for enhancing in vivo killing of target tissues by directly stimulating a ZcytoR21 receptor-modulated apoptotic pathway, resulting in cell death of hyperproliferative cells expressing ZcytoR21-comprising receptors.

J) Therapeutic Uses of Polypeptides Having ZcytoR21 Activity or Antibodies to ZcytoR21

Amino acid sequences having soluble ZcytoR21 activity can be used to modulate the immune system by binding ZcytoR21 ligands IL-17C, and thus, preventing the binding of ZcytoR21 ligand with endogenous ZcytoR21 receptor. ZcytoR21 antagonists, such as soluble ZcytoR21 or anti-ZcytoR21 antibodies, can also be used to modulate the immune system by inhibiting the binding of ZcytoR21 ligand with the endogenous ZcytoR21 receptor. Accordingly, the present invention includes the use of proteins, polypeptides, and peptides having ZcytoR21 activity (such as soluble ZcytoR21 polypeptides, ZcytoR21 polypeptide fragments, ZcytoR21 analogs (e.g., anti-ZcytoR21 anti-idiotype antibodies), and ZcytoR21 fusion proteins) to a subject which lacks an adequate amount of this polypeptide, or which produces an excess of ZcytoR21 ligand. ZcytoR21 antagonists (e.g., anti-ZcytoR21 antibodies) can be also used to treat a subject which produces an excess of either ZcytoR21 ligand or ZcytoR21. Suitable subjects include mammals, such as humans. For example, such ZcytoR21 polypeptides and anti-ZcytoR21 antibodies are useful in binding, blocking, inhibiting, reducing, antagonizing or neutralizing IL-17C, in the treatment of inflammation and inflammatory dieases such as psoriasis, psoriatic arthritis, rheumatoid arthritis, endotoxemia, inflammatory bowel disease (IBD), colitis, asthma, allograft rejection, immune mediated renal diseases, hepatobiliary diseases, multiple sclerosis, atherosclerosis, promotion of tumor growth, or degenerative joint disease and other inflammatory conditions disclosed herein.

Within preferred embodiments, the soluble receptor form of ZcytoR21, (SEQ ID NOs:3, 6, 9, 12, 15, 21, 23, 109, 113, 115, 117, 119, or 122) is a monomer, homodimer, heterodimer, or multimer that binds to, blocks, inhibits, reduces, antagonizes or neutralizes IL-17C in vivo. Antibodies and binding polypeptides to such ZcytoR21 monomer, homodimer, heterodimer, or multimers also serve as antagonists of ZcytoR21 activity, and as IL-17C as described herein.

Thus, particular embodiments of the present invention are directed toward use of soluble ZcytoR21 and anti-ZcytoR21 antibodies as antagonists in inflammatory and immune diseases or conditions such as psoriasis, psoriatic arthritis, atopic dermatitis, inflammatory skin conditions, rheumatoid arthritis, inflammatory bowel disease (IBD), Crohn's Disease, diverticulosis, asthma, pancreatitis, type I diabetes (IDDM), pancreatic cancer, pancreatitis, Graves Disease, colon and intestinal cancer, autoimmune disease, sepsis, organ or bone marrow transplant; inflammation due to endotoxemia, trauma, sugery or infection; amyloidosis; splenomegaly; graft versus host disease; and where inhibition of inflammation, immune suppression, reduction of proliferation of hematopoietic, immune, inflammatory or lymphoid cells, macrophages, T-cells (including Th1 and Th2 cells), suppression of immune response to a pathogen or antigen, or other instances where inhibition of IL-17C or another IL-17 family member or cytokine is desired.

Moreover, antibodies or binding polypeptides such as soluble receptors that bind ZcytoR21 polypeptides described herein, and ZcytoR21 polypeptides themselves are useful to:

1) Block, inhibit, reduce, antagonize or neutralize signaling via either IL-17C or the IL-17C receptor (e.g. ZcytoR21) in the treatment of acute inflammation, inflammation as a result of trauma, tissue injury, surgery, sepsis or infection, and chronic inflammatory diseases such as asthma, inflammatory bowel disease (IBD), chronic colitis, splenomegaly, rheumatoid arthritis, recurrent acute inflammatory episodes (e.g., tuberculosis), and treatment of amyloidosis, and atherosclerosis, Castleman's Disease, asthma, and other diseases associated with the induction of acute-phase response.

2) Block, inhibit, reduce, antagonize or neutralize signaling via either IL-17C or the IL-17C receptor (e.g. ZcytoR21) in the treatment of autoimmune diseases such as IDDM, multiple sclerosis (MS), systemic Lupus erythematosus (SLE), myasthenia gravis, rheumatoid arthritis, and IBD to prevent or inhibit signaling in immune cells (e.g. lymphocytes, monocytes, leukocytes). Alternatively antibodies, such as monoclonal antibodies (MAb) to ZcytoR21-comprising receptors, can also be used as an antagonist to deplete unwanted immune cells to treat autoimmune disease. Asthma, allergy and other atopic disease may be treated with a MAb of the present invention against, for example, the ZcytoR21 binding domain (as described in any of SEQ ID NOs: 115, 117 or 119) to inhibit the immune response or to deplete offending cells. Blocking, inhibiting, reducing, or antagonizing signaling via ZcytoR21, using the soluble receptors, polypeptides and antibodies of the present invention, may also benefit diseases of the pancreas, kidney, pituitary and neuronal cells. IDDM, NIDDM, pancreatitis, and pancreatic carcinoma may benefit.

ZcytoR21 may serve as a target for MAb therapy of cancer where an antagonizing MAb inhibits cancer growth and targets immune-mediated killing. (Holliger P, and Hoogenboom, H: Nature Biotech. 16: 1015-1016, 1998). MAbs to soluble ZcytoR21 may also be useful to treat nephropathies such as glomerulosclerosis, membranous neuropathy, amyloidosis (which also affects the kidney among other tissues), renal arteriosclerosis, glomerulonephritis of various origins, fibroproliferative diseases of the kidney, as well as kidney dysfunction associated with SLE, IDDM, type II diabetes (NIDDM), renal tumors and other diseases.

3) Agonize, enhance, increase or initiate signaling via the IL-17C receptor (e.g. ZcytoR21) in the treatment of autoimmune diseases such as IDDM, MS, SLE, myasthenia gravis, rheumatoid arthritis, and IBD. Anti-ZcytoR21 neutralizing and monoclonal antibodies may signal lymphocytes or other immune cells to differentiate, alter proliferation, or change production of cytokines or cell surface proteins that ameliorate autoimmunity. Specifically, modulation of a T-helper cell response to an alternate pattern of cytokine secretion may deviate an autoimmune response to ameliorate disease (Smith J A et al., J. Immunol. 160:48414849, 1998). Similarly, agonistic anti-soluble ZcytoR21 monomers, homodimers, heterodimers and multimer monoclonal antibodies may be used to signal, deplete and deviate immune cells involved in asthma, allergy and atopoic disease. Signaling via ZcytoR21 may also benefit diseases of the pancreas, kidney, pituitary and neuronal cells. IDDM, NIDDM, pancreatitis, and pancreatic carcinoma may benefit. ZcytoR21 may serve as a target for MAb therapy of pancreatic cancer where a signaling MAb inhibits cancer growth and targets immune-mediated killing (Tutt, A L et al., J. Immunol. 161: 3175-3185, 1998). Similarly renal cell carcinoma may be treated with monoclonal antibodies to ZcytoR21-comprising soluble receptors of the present invention.

Soluble ZcytoR21 polypeptides described herein can be used to bind, block, inhibit, reduce, antagonize or neutralize IL-17C activity, in the treatment of autoimmune disease, atopic disease, NIDDM, pancreatitis and kidney dysfunction as described above. A soluble form of ZcytoR21, such as ZcytoR21s2 (SEQ ID NO:113) may be used to promote an antibody response mediated by Th cells and/or to promote the production of IL-4 or other cytokines by lymphocytes or other immune cells.

The soluble ZcytoR21-comprising receptors of the present invention are useful as antagonists of IL-17C. Such antagonistic effects can be achieved by direct neutralization or binding of IL-17C. In addition to antagonistic uses, the soluble receptors of the present invention can bind IL-17C and act as carrier proteins for IL-17C cytokine, in order to transport the ligand to different tissues, organs, and cells within the body. As such, the soluble receptors of the present invention can be fused or coupled to molecules, polypeptides or chemical moieties that direct the soluble-receptor-ligand complex to a specific site, such as a tissue, specific immune cell, or tumor. For example, in acute infection or some cancers, benefit may result from induction of inflammation and local acute phase response proteins by the action of IL-17C. Thus, the soluble receptors of the present invention can be used to specifically direct the action of IL-17C. See, Cosman, D. Cytokine 5: 95-106, 1993; and Fernandez-Botran, R. Exp. Opin. Invest. Drugs 9:497-513, 2000.

Moreover, the soluble receptors of the present invention can be used to stabilize IL-17C, to increase the bioavailability, therapeutic longevity, and/or efficacy of IL-17C by stabilizing it from degradation or clearance, or by targeting the ligand to a site of action within the body. For example the naturally occurring IL-6/soluble IL-6R complex stabilizes IL-6 and can signal through the gp130 receptor. See, Cosman, D. supra., and Fernandez-Botran, R. supra. Moreover, ZcytoR21 may be combined with a cognate ligand such as IL-17C to comprise a ligand/soluble receptor complex. Such complexes may be used to stimulate responses from cells presenting a companion receptor subunit. The cell specificity of ZcytoR21/ligand complexes may differ from that seen for the ligand administered alone. Furthermore the complexes may have distinct pharmacokinetic properties such as affecting half-life, dose/response and organ or tissue specificity. ZcytoR21/IL-17C complexes thus may have agonist activity to enhance an immune response or stimulate mesangial cells or to stimulate hepatic cells. Alternatively only tissues expressing a signaling subunit the heterodimerizes with the complex may be affected analogous to the response to IL6/IL6R complexes (Hirota H. et al., Proc. Nat'l. Acad. Sci. 92:4862-4866, 1995; Hirano, T. in Thomason, A. (Ed.) “The Cytokine Handbook”, 3^{rd} Ed., p. 208-209). Soluble receptor/cytokine complexes for IL-12 and CNTF display similar activities.

Moreover, inflammation is a protective response by an organism to fend off an invading agent. Inflammation is a cascading event that involves many cellular and humoral mediators. On one hand, suppression of inflammatory responses can leave a host immunocompromised; however, if left unchecked, inflammation can lead to serious complications including chronic inflammatory diseases (e.g., psoriasis, arthritis, rheumatoid arthritis, multiple sclerosis, inflammatory bowel disease and the like), septic shock and multiple organ failure. Importantly, these diverse disease states share common inflammatory mediators. The collective diseases that are characterized by inflammation have a large impact on human morbidity and mortality. Therefore it is clear that anti-inflammatory proteins, such as ZcytoR21, and anti-ZcytoR21 antibodies, could have crucial therapeutic potential for a vast number of human and animal diseases, from asthma and allergy to autoimmunity and septic shock.

1. Arthritis

Arthritis, including osteoarthritis, rheumatoid arthritis, arthritic joints as a result of injury, and the like, are common inflammatory conditions which would benefit from the therapeutic use of anti-inflammatory proteins, such as ZcytoR21 soluble polypeptides and MAbs of the present invention. For example, rheumatoid arthritis (RA) is a systemic disease that affects the entire body and is one of the most common forms of arthritis. It is characterized by the inflammation of the membrane lining the joint, which causes pain, stiffness, warmth, redness and swelling. Inflammatory cells release enzymes that may digest bone and cartilage. As a result of rheumatoid arthritis, the inflamed joint lining, the synovium, can invade and damage bone and cartilage leading to joint deterioration and severe pain amongst other physiologic effects. The involved joint can lose its shape and alignment, resulting in pain and loss of movement.

Rheumatoid arthritis (RA) is an immune-mediated disease particularly characterized by inflammation and subsequent tissue damage leading to severe disability and increased mortality. A variety of cytokines are produced locally in the rheumatoid joints. Numerous studies have demonstrated that IL-1 and TNF-alpha, two prototypic pro-inflammatory cytokines, play an important role in the mechanisms involved in synovial inflammation and in progressive joint destruction. Indeed, the administration of TNF-alpha and IL-1 inhibitors in patients with RA has led to a dramatic improvement of clinical and biological signs of inflammation and a reduction of radiological signs of bone erosion and cartilage destruction. However, despite these encouraging results, a significant percentage of patients do not respond to these agents, suggesting that other mediators are also involved in the pathophysiology of arthritis (Gabay, Expert. Opin. Biol. Ther. 2(2):135-149, 2002). One of those mediators could be IL-17C, and as such a molecule that binds or inhibits IL-17C activity, such as soluble ZcytoR21, ZcytoR21 polypeptides, or anti-ZcytoR21 antibodies or binding partners, could serve as a valuable therapeutic to reduce inflammation in rheumatoid arthritis, and other arthritic diseases.

There are several animal models for rheumatoid arthritis known in the art. For example, in the collagen-induced arthritis (CIA) model, mice develop chronic inflammatory arthritis that closely resembles human rheumatoid arthritis. Since CIA shares similar immunological and pathological features with RA, this makes it an ideal model for screening potential human anti-inflammatory compounds. The CIA model is a well-known model in mice that depends on both an immune response, and an inflammatory response, in order to occur. The immune response comprises the interaction of B-cells and CD4+ T-cells in response to collagen, which is given as antigen, and leads to the production of anti-collagen antibodies. The inflammatory phase is the result of tissue responses from mediators of inflammation, as a consequence of some of these antibodies cross-reacting to the mouse's native collagen and activating the complement cascade. An advantage in using the CIA model is that the basic mechanisms of pathogenesis are known. The relevant T-cell and B-cell epitopes on type II collagen have been identified, and various immunological (e.g., delayed-type hypersensitivity and anti-collagen antibody) and inflammatory (e.g., cytokines, chemokines, and matrix-degrading enzymes) parameters relating to immune-mediated arthritis have been determined, and can thus be used to assess test compound efficacy in the CIA model (Wooley, Curr. Opin. Rheum. 3:407-20, 1999; Williams et al., Immunol. 89:9784-788, 1992; Myers et al., Life Sci. 61:1861-78, 1997; and Wang et al., Immunol. 92:8955-959, 1995).

The administration of soluble ZcytoR21 comprising polypeptides (ZcytoR21), such as ZcytoR21-Fc4 or other ZcytoR21 soluble and fusion proteins to these CIA model mice is used to evaluate the use of soluble ZcytoR21 as an antagonist to IL-17C used to ameliorate symptoms and alter the course of disease. Moreover, results showing inhibition of IL-17C by a soluble ZcytoR21 polypeptide or anti-ZcytoR21 antibody of the present invention would provide proof of concept that other IL-17C antagonists, such as soluble ZcytoR21 or neutralizing antibodies thereto, can also be used to ameliorate symptoms and alter the course of disease. Furthermore, the systemic or local administration of soluble ZcytoR21 comprising polypeptides, such as ZcytoR21-Fc4 or other IL-17C soluble receptors (e.g., ZcytoR21; SEQ ID NO:3, 6, 9, 12, 15, 21, 23 109, 113, 115, 117, 119, or 122) and anti-ZcytoR21 antibodies, and fusion proteins can potentially suppress the inflammatory response in RA. By way of example and without limitation, the injection of 10-100 ug soluble ZcytoR21-Fc per mouse (one to seven times a week for up to but not limited to 4 weeks via s.c., i.p., or i.m route of administration) can significantly reduce the disease score (paw score, incident of inflammation, or disease). Depending on the initiation of ZcytoR21-Fc administration (e.g. prior to or at the time of collagen immunization, or at any time point following the second collagen immunization, including those time points at which the disease has already progressed), ZcytoR21 can be efficacious in preventing rheumatoid arthritis, as well as preventing its progression. Other potential therapeutics include ZcytoR21 polypeptides, anti-ZcytoR21 antibodies, or anti IL-17C antibodies or binding partners, and the like.

2. Endotoxemia

Endotoxemia is a severe condition commonly resulting from infectious agents such as bacteria and other infectious disease agents, sepsis, toxic shock syndrome, or in immunocompromised patients subjected to opportunistic infections, and the like. Therapeutically useful of anti-inflammatory proteins, such as ZcytoR21 polypeptides and antibodies of the present invention, could aid in preventing and treating endotoxemia in humans and animals. ZcytoR21 polypeptides, or anti-ZcytoR21 antibodies or binding partners, could serve as a valuable therapeutic to reduce inflammation and pathological effects in endotoxemia.

Lipopolysaccharide (LPS) induced endotoxemia engages many of the proinflammatory mediators that produce pathological effects in the infectious diseases and LPS induced endotoxemia in rodents is a widely used and acceptable model for studying the pharmacological effects of potential pro-inflammatory or immunomodulating agents. LPS, produced in gram-negative bacteria, is a major causative agent in the pathogenesis of septic shock (Glausner et al., Lancet 338:732, 1991). A shock-like state can indeed be induced experimentally by a single injection of LPS into animals. Molecules produced by cells responding to LPS can target pathogens directly or indirectly. Although these biological responses protect the host against invading pathogens, they may also cause harm. Thus, massive stimulation of innate immunity, occurring as a result of severe Gram-negative bacterial infection, leads to excess production of cytokines and other molecules, and the development of a fatal syndrome, septic shock syndrome, which is characterized by fever, hypotension, disseminated intravascular coagulation, and multiple organ failure (Dumitru et al. Cell 103:1071-1083, 2000).

These toxic effects of LPS are mostly related to macrophage activation leading to the release of multiple inflammatory mediators. Among these mediators, TNF appears to play a crucial role, as indicated by the prevention of LPS toxicity by the administration of neutralizing anti-TNF antibodies (Beutler et al., Science 229:869, 1985). It is well established that 1 ug injection of E. coli LPS into a C57Bl/6 mouse will result in significant increases in circulating IL-6, TNF-alpha, IL-1, and acute phase proteins (for example, SAA) approximately 2 hours post injection. The toxicity of LPS appears to be mediated by these cytokines as passive immunization against these mediators can result in decreased mortality (Beutler et al., Science 229:869, 1985). The potential immunointervention strategies for the prevention and/or treatment of septic shock include anti-TNF mAb, IL-1 receptor antagonist, LIF, IL-10, and G-CSF.

The administration of soluble ZcytoR21 comprising polypeptides, such as ZcytoR21-Fc5, ZcytoR21-Fc10 or other ZcytoR21 soluble and fusion proteins to these LPS-induced model may be used to to evaluate the use of ZcytoR21 to ameliorate symptoms and alter the course of LPS-induced disease. Moreover, results showing inhibition of IL-17C by ZcytoR21 provide proof of concept that other IL-17C antagonists, such as soluble ZcytoR21 or antibodies thereto, can also be used to ameliorate symptoms in the LPS-induced model and alter the course of disease. The model will show induction of IL-17C by LPS injection and the potential treatment of disease by ZcytoR21 polypeptides. Since LPS induces the production of pro-inflammatory factors possibly contributing to the pathology of endotoxemia, the neutralization of IL-17C activity or other pro-inflammatory factors by an antagonist ZcytoR21 polyepeptide can be used to reduce the symptoms of endotoxemia, such as seen in endotoxic shock. Other potential therapeutics include ZcytoR21 polypeptides, anti-ZcytoR21 antibodies, or binding partners, and the like.

3. Inflammatory Bowel Disease IBD

In the United States approximately 500,000 people suffer from Inflammatory Bowel Disease (IRD) which can affect either colon and rectum (Ulcerative colitis) or both, small and large intestine (Crohn's Disease). The pathogenesis of these diseases is unclear, but they involve chronic inflammation of the affected tissues. ZcytoR21 polypeptides, anti-ZcytoR21 antibodies, or binding partners, could serve as a valuable therapeutic to reduce inflammation and pathological effects in IBD and related diseases.

Ulcerative colitis (UC) is an inflammatory disease of the large intestine, commonly called the colon, characterized by inflammation and ulceration of the mucosa or innermost lining of the colon. This inflammation causes the colon to empty frequently, resulting in diarrhea. Symptoms include loosening of the stool and associated abdominal cramping, fever and weight loss. Although the exact cause of UC is unknown, recent research suggests that the body's natural defenses are operating against proteins in the body which the body thinks are foreign (an “autoimmune reaction”). Perhaps because they resemble bacterial proteins in the gut, these proteins may either instigate or stimulate the inflammatory process that begins to destroy the lining of the colon. As the lining of the colon is destroyed, ulcers form releasing mucus, pus and blood. The disease usually begins in the rectal area and may eventually extend through the entire large bowel. Repeated episodes of inflammation lead to thickening of the wall of the intestine and rectum with scar tissue. Death of colon tissue or sepsis may occur with severe disease. The symptoms of ulcerative colitis vary in severity and their onset may be gradual or sudden. Attacks may be provoked by many factors, including respiratory infections or stress.

Although there is currently no cure for UC available, treatments are focused on suppressing the abnormal inflammatory process in the colon lining. Treatments including corticosteroids immunosuppressives (eg. azathioprine, mercaptopurine, and methotrexate) and aminosalicytates are available to treat the disease. However, the long-term use of immunosuppressives such as corticosteroids and azathioprine can result in serious side effects including thinning of bones, cataracts, infection, and liver and bone marrow effects. In the patients in whom current therapies are not successful, surgery is an option. The surgery involves the removal of the entire colon and the rectum.

There are several animal models that can partially mimic chronic ulcerative colitis. Some of the most widely used models are the oxazolone and the 2,4,6-trinitrobenesulfonic acid/ethanol (TNBS) induced colitis models, which induce chronic inflammation and ulceration in the colon. When oxazolone or TNBS is introduced into the colon of susceptible mice via intra-rectal instillation, it induces T-cell mediated immune response in the colonic mucosa, in this case leading to a massive mucosal inflammation characterized by the dense infiltration of T-cells and macrophages throughout the entire wall of the large bowel. Moreover, this histopathologic picture is accompanies by the clinical picture of progressive weight loss (wasting), bloody diarrhea, rectal prolapse, and large bowel wall thickening (Neurath et al. Intern. Rev. Immunol. 19:51-62, 2000).

Another colitis model uses dextran sulfate sodium (DSS), which induces an acute colitis manifested by bloody diarrhea, weight loss, shortening of the colon and mucosal ulceration with neutrophil infiltration. DSS-induced colitis is characterized histologically by infiltration of inflammatory cells into the lamina propria, with lymphoid hyperplasia, focal crypt damage, and epithelial ulceration. These changes are thought to develop due to a toxic effect of DSS on the epithelium and by phagocytosis of lamina propria cells and production of TNF-alpha and IFN-gamma. Despite its common use, several issues regarding the mechanisms of DSS about the relevance to the human disease remain unresolved. DSS is regarded as a T cell-independent model because it is observed in T cell-deficient animals such as SCID mice.

The administration of soluble ZcytoR21 or other ZcytoR21 soluble and fusion proteins to these TNBS or DSS models can be used to evaluate the use of soluble ZcytoR21 to ameliorate symptoms and alter the course of gastrointestinal disease. Moreover, the results showing inhibition of IL-17C by ZcytoR21 provide proof of concept that other IL-17C antagonists, such as soluble ZcytoR21 or antibodies thereto, can also be used to ameliorate symptoms in the colitis/IBD models and alter the course of disease.

4. Psoriasis

Psoriasis is a chronic skin condition that affects more than seven million Americans. Psoriasis occurs when new skin cells grow abnormally, resulting in inflamed, swollen, and scaly patches of skin where the old skin has not shed quickly enough. Plaque psoriasis, the most common form, is characterized by inflamed patches of skin (“lesions”) topped with silvery white scales. Psoriasis may be limited to a few plaques or involve moderate to extensive areas of skin, appearing most commonly on the scalp, knees, elbows and trunk. Although it is highly visible, psoriasis is not a contagious disease. The pathogenesis of the diseases involves chronic inflammation of the affected tissues. ZcytoR21 polypeptides, anti-ZcytoR21 antibodies, or binding partners, could serve as a valuable therapeutic to reduce inflammation and pathological effects in psoriasis, other inflammatory skin diseases, skin and mucosal allergies, and related diseases.

Psoriasis is a T-cell mediated inflammatory disorder of the skin that can cause considerable discomfort. It is a disease for which there is no cure and affects people of all ages. Psoriasis affects approximately two percent of the populations of European and North America. Although individuals with mild psoriasis can often control their disease with topical agents, more than one million patients worldwide require ultraviolet or systemic immunosuppressive therapy. Unfortunately, the inconvenience and risks of ultraviolet radiation and the toxicities of many therapies limit their long-term use. Moreover, patients usually have recurrence of psoriasis, and in some cases rebound, shortly after stopping immunosuppressive therapy.

ZcytoR21 soluble receptor polypeptides and antibodies thereto may also be used within diagnostic systems for the detection of circulating levels of IL-17C ligand, and in the detection of IL-17C associated with acute phase inflammatory response. Within a related embodiment, antibodies or other agents that specifically bind to ZcytoR21 soluble receptors of the present invention can be used to detect circulating receptor polypeptides; conversely, ZcytoR21 soluble receptors themselves can be used to detect circulating or locally-acting IL-17C polypeptides. Elevated or depressed levels of ligand or receptor polypeptides may be indicative of pathological conditions, including inflammation or cancer. Moreover, detection of acute phase proteins or molecules such as IL-17C can be indicative of a chronic inflammatory condition in certain disease states (e.g., asthma, psoriasis, rheumatoid arthritis, colitis, IBD). Detection of such conditions serves to aid in disease diagnosis as well as help a physician in choosing proper therapy.

In addition to other disease models described herein, the activity of soluble ZcytoR21 and/or anti-ZcytoR21 antibodies on inflammatory tissue derived from human psoriatic lesions can be measured in-vivo using a severe combined immune deficient (SCID) mouse model. Several mouse models have been developed in which human cells are implanted into immunodeficient mice (collectively referred to as xenograft models); see, for example, Cattan A R, Douglas E, Leuk. Res. 18:513-22, 1994 and Flavell, D J, Hematological Oncology 14:67-82, 1996. As an in vivo xenograft model for psoriasis, human psoriatic skin tissue is implanted into the SCID mouse model, and challenged with an appropriate antagonist. Moreover, other psoriasis animal models in ther art may be used to evaluate IL-17C antagonists, such as human psoriatic skin grafts implanted into AGR129 mouse model, and challenged with an appropriate antagonist (e.g., see, Boyman, O. et al., J. Exp. Med. Online publication #20031482, 2004, incorporated hereing by reference). Soluble ZcytoR21 or anti-ZcytoR21 antibodies that bind, block, inhibit, reduce, antagonize or neutralize the activity of IL-17C are preferred antagonists, however, anti-IL-17C, soluble ZcytoR21, as well as other IL-17C antagonists can be used in this model. Similarly, tissues or cells derived from human colitis, IBD, arthritis, or other inflammatory lestions can be used in the SCID model to assess the anti-inflammatory properties of the IL-17C antagonists described herein.

Therapies designed to abolish, retard, or reduce inflammation using soluble ZcytoR21, anti-ZcytoR21 antibodies or its derivatives, agonists, conjugates or variants can be tested by administration of anti-ZcytoR21 antibodies or soluble ZcytoR21 compounds to SCID mice bearing human inflammatory tissue (e.g., psoriatic lesions and the like), or other models described herein. Efficacy of treatment is measured and statistically evaluated as increased anti-inflammatory effect within the treated population over time using methods well known in the art. Some exemplary methods include, but are not limited to measuring for example, in a psoriasis model, epidermal thickness, the number of inflammatory cells in the upper dermis, and the grades of parakeratosis. Such methods are known in the art and described herein. For example, see Zeigler, M. et al. Lab Invest 81:1253, 2001; Zollner, T. M. et al. J. Clin. Invest. 109:671, 2002; Yamanaka, N. et al. Microbiol Immunol. 45:507, 2001; Raychaudhuri, S. P. et al. Br. J. Dermatol. 144:931, 2001; Boehncke, W. H et al. Arch. Dermatol. Res. 291:104, 1999, Boehncke, W. H et al. J. Invest. Dermatol. 116:596, 2001; Nickoloff, B. J. et al. Am. J. Pathol. 146:580, 1995; Boehncke, W. H et al. J. Cutan. Pathol. 24:1, 1997; Sugai, J., M. et al. J. Dermatol. Sci. 17:85, 1998; and Villadsen L. S. et al. J. Clin. Invest. 112:1571, 2003. Inflammation may also be monitored over time using well-known methods such as flow cytometry (or PCR) to quantitate the number of inflammatory or lesional cells present in a sample, score (weight loss, diarrhea, rectal bleeding, colon length) for IBD, paw disease score and inflammation score for CIA RA model. For example, therapeutic strategies appropriate for testing in such a model include direct treatment using soluble ZcytoR21, anti-ZcytoR21 antibodies, other IL-17C antagonists or related conjugates or antagonists based on the disrupting interaction of soluble ZcytoR21 with its ligand IL-17C, or for cell-based therapies utilizing soluble ZcytoR21 or anti-ZcytoR21 antibodies or its derivatives, agonists, conjugates or variants.

Moreover, psoriasis is a chronic inflammatory skin disease that is associated with hyperplastic epidermal keratinocytes and infiltrating mononuclear cells, including CD4+ memory T cells, neutrophils and macrophages (Christophers, Int. Arch. Allergy Immunol., 110:199, 1996). It is currently believed that environmental antigens play a significant role in initiating and contributing to the pathology of the disease. However, it is the loss of tolerance to self-antigens that is thought to mediate the pathology of psoriasis. Dendritic cells and CD4⁺ T cells are thought to play an important role in antigen presentation and recognition that mediate the immune response leading to the pathology. We have recently developed a model of psoriasis based on the CD4+ CD45RB transfer model (Davenport et al., Internat. Immunopharmacol., 2:653-672). Soluble ZcytoR21 or anti-ZcytoR21 antibodies of the present invention are administered to the mice. Inhibition of disease scores (skin lesions, inflammatory cytokines) indicates the effectiveness of IL-17C antagonists in psoriasis, e.g., anti-ZcytoR21 antibodies or ZcytoR21 soluble receptors.

5. Atopic Dermatitis.

AD is a common chronic inflammatory disease that is characterized by hyperactivated cytokines of the helper T cell subset 2 (Th2). Although the exact etiology of AD is unknown, multiple factors have been implicated, including hyperactive Th2 immune responses, autoimmunity, infection, allergens, and genetic predisposition. Key features of the disease include xerosis (dryness of the skin), pruritus (itchiness of the skin), conjunctivitis, inflammatory skin lesions, Staphylococcus aureus infection, elevated blood eosinophilia, elevation of serum IgE and IgG1, and chronic dermatitis with T cell, mast cell, macrophage and eosinophil infiltration. Colonization or infection with S. aureus has been recognized to exacerbate AD and perpetuate chronicity of this skin disease.

AD is often found in patients with asthma and allergic rhinitis, and is frequently the initial manifestation of allergic disease. About 20% of the population in Western countries suffer from these allergic diseases, and the incidence of AD in developed countries is rising for unknown reasons. AD typically begins in childhood and can often persist through adolescence into adulthood. Current treatments for AD include topical corticosteroids, oral cyclosporin A, non-corticosteroid immunosuppressants such as tacrolimus (FK506 in ointment form), and interferon-gamma. Despite the variety of treatments for AD, many patients' symptoms do not improve, or they have adverse reactions to medications, requiring the search for other, more effective therapeutic agents. The soluble ZcytoR21 polypeptides and anti-ZcytoR21 antibodies of the present invention, including the neutralizing anti-human ZcytoR21 antibodies of the present invention, can be used to neutralize IL-17C in the treatment of specific human diseases such as atoptic dermatitis, inflammatory skin conditions, and other inflammatory conditions disclosed herein.

K) Pharmaceutical Use of ZcytoR21

For pharmaceutical use, the soluble ZcytoR21 or anti-ZcytoR21 antibodies of the present invention are formulated for parenteral, particularly intravenous or subcutaneous, delivery according to conventional methods. Intravenous administration will be by bolus injection, controlled release, e.g, using mini-pumps or other appropriate technology, or by infusion over a typical period of one to several hours. In general, pharmaceutical formulations will include a hematopoietic protein in combination with a pharmaceutically acceptable vehicle, such as saline, buffered saline, 5% dextrose in water or the like. Formulations may further include one or more excipients, preservatives, solubilizers, buffering agents, albumin to provent protein loss on vial surfaces, etc. When utilizing such a combination therapy, the cytokines may be combined in a single formulation or may be administered in separate formulations. Methods of formulation are well known in the art and are disclosed, for example, in Remington's Pharmaceutical Sciences, Gennaro, ed., Mack Publishing Co., Easton Pa., 1990, which is incorporated herein by reference. Therapeutic doses will generally be in the range of 0.1 to 100 mg/kg of patient weight per day, preferably 0.5-20 mg/kg per day, with the exact dose determined by the clinician according to accepted standards, taking into account the nature and severity of the condition to be treated, patient traits, etc. Determination of dose is within the level of ordinary skill in the art. The proteins will commonly be administered over a period of up to 28 days following chemotherapy or bone-marrow transplant or until a platelet count of >20,000/mm³, preferably >50,000/mm³, is achieved. More commonly, the proteins will be administered over one week or less, often over a period of one to three days. In general, a therapeutically effective amount of soluble ZcytoR21 or anti-ZcytoR21 antibodies of the present invention is an amount sufficient to produce a clinically significant increase in the proliferation and/or differentiation of lymphoid or myeloid progenitor cells, which will be manifested as an increase in circulating levels of mature cells (e.g. platelets or neutrophils). Treatment of platelet disorders will thus be continued until a platelet count of at least 20,000/mm³, preferably 50,000/mm³, is reached. The soluble ZcytoR21 or anti-ZcytoR21 antibodies of the present invention can also be administered in combination with other cytokines such as IL-3, -6 and -11; stem cell factor; erythropoietin; G-CSF and GM-CSF. Within regimens of combination therapy, daily doses of other cytokines will in general be: EPO, 150 U/kg; GM-CSF, 5-15 lg/kg; IL-3,1-5 lg/kg; and G-CSF, 1-25 lg/kg. Combination therapy with EPO, for example, is indicated in anemic patients with low EPO levels.

Generally, the dosage of administered soluble ZcytoR21 (or ZcytoR21 analog or fusion protein) or anti-ZcytoR21 antibodies will vary depending upon such factors as the patient's age, weight, height, sex, general medical condition and previous medical history. Typically, it is desirable to provide the recipient with a dosage of soluble ZcytoR21 or anti-ZcytoR21 antibodies which is in the range of from about 1 pg/kg to 10 mg/kg (amount of agent/body weight of patient), although a lower or higher dosage also may be administered as circumstances dictate.

Administration of soluble ZcytoR21 or anti-ZcytoR21 antibodies to a subject can be intravenous, intraarterial, intraperitoneal, intramuscular, subcutaneous, intrapleural, intrathecal, by perfusion through a regional catheter, or by direct intralesional injection. When administering therapeutic proteins by injection, the administration may be by continuous infusion or by single or multiple boluses.

Additional routes of administration include oral, mucosal-membrane, pulmonary, and transcutaneous. Oral delivery is suitable for polyester microspheres, zein microspheres, proteinoid microspheres, polycyanoacrylate microspheres, and lipid-based systems (see, for example, DiBase and Morrel, “Oral Delivery of Microencapsulated Proteins,” in Protein Delivery: Physical Systems, Sanders and Hendren (eds.), pages 255-288 (Plenum Press 1997)). The feasibility of an intranasal delivery is exemplified by such a mode of insulin administration (see, for example, Hinchcliffe and Illum, Adv. Drug Deliv. Rev. 35:199 (1999)). Dry or liquid particles comprising soluble ZcytoR21 or anti-ZcytoR21 antibodies can be prepared and inhaled with the aid of dry-powder dispersers, liquid aerosol generators, or nebulizers (e.g., Pettit and Gombotz, TIBTECH 16:343 (1998); Patton et al., Adv. Drug Deliv. Rev. 35:235 (1999)). This approach is illustrated by the AERX diabetes management system, which is a hand-held electronic inhaler that delivers aerosolized insulin into the lungs. Studies have shown that proteins as large as 48,000 kDa have been delivered across skin at therapeutic concentrations with the aid of low-frequency ultrasound, which illustrates the feasibility of trascutaneous administration (Mitragotri et al., Science 269:850 (1995)). Transdermal delivery using electroporation provides another means to administer a molecule having ZcytoR21 binding activity (Potts et al., Pharm. Biotechnol. 10:213 (1997)).

A pharmaceutical composition comprising a soluble ZcytoR21 or anti-ZcytoR21 antibody can be formulated according to known methods to prepare pharmaceutically useful compositions, whereby the therapeutic proteins are combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. See, for example, Gennaro (ed.), Remington's Pharmaceutical Sciences, 19th Edition (Mack Publishing Company 1995).

For purposes of therapy, soluble ZcytoR21 or anti-ZcytoR21 antibody molecules and a pharmaceutically acceptable carrier are administered to a patient in a therapeutically effective amount. A combination of a therapeutic molecule of the present invention and a pharmaceutically acceptable carrier is said to be administered in a “therapeutically effective amount” if the amount administered is physiologically significant. An agent is physiologically significant if its presence results in a detectable change in the physiology of a recipient patient. For example, an agent used to treat inflammation is physiologically significant if its presence alleviates the inflammatory response.

A pharmaceutical composition comprising ZcytoR21 (or ZcytoR21 analog or fusion protein) or neutralizing anti-ZcytoR21 antibody can be furnished in liquid form, in an aerosol, or in solid form. Liquid forms, are illustrated by injectable solutions and oral suspensions. Exemplary solid forms include capsules, tablets, and controlled-release forms. The latter form is illustrated by miniosmotic pumps and implants (Bremer et al., Pharm. Biotechnol. 10:239 (1997); Ranade, “Implants in Drug Delivery,” in Drug Delivery Systems, Ranade and Hollinger (eds.), pages 95-123 (CRC Press 1995); Bremer et al., “Protein Delivery with Infusion Pumps,” in Protein Delivery: Physical Systems, Sanders and Hendren (eds.), pages 239-254 (Plenum Press 1997); Yewey et al., “Delivery of Proteins from a Controlled Release Injectable Implant,” in Protein Delivery: Physical Systems, Sanders and Hendren (eds.), pages 93-117 (Plenum Press 1997)).

Liposomes provide one means to deliver therapeutic polypeptides to a subject intravenously, intraperitoneally, intrathecally, intramuscularly, subcutaneously, or via oral administration, inhalation, or intranasal administration. Liposomes are microscopic vesicles that consist of one or more lipid bilayers surrounding aqueous compartments (see, generally, Bakker-Woudenberg et al., Eur. J. Clin. Microbiol. Infect. Dis. 12 (Suppl. 1):S61 (1993), Kim, Drugs 46:618 (1993), and Ranade, “Site-Specific Drug Delivery Using Liposomes as Carriers,” in Drug Delivery Systems, Ranade and Hollinger (eds.), pages 3-24 (CRC Press 1995)). Liposomes are similar in composition to cellular membranes and as a result, liposomes can be administered safely and are biodegradable. Depending on the method of preparation, liposomes may be unilamellar or multilamellar, and liposomes can vary in size with diameters ranging from 0.02 μm to greater than 10 μm. A variety of agents can be encapsulated in liposomes: hydrophobic agents partition in the bilayers and hydrophilic agents partition within the inner aqueous space(s) (see, for example, Machy et al., Liposomes In Cell Biology And Pharmacology (John Libbey 1987), and Ostro et al., American J. Hosp. Pharm. 46:1576 (1989)). Moreover, it is possible to control the therapeutic availability of the encapsulated agent by varying liposome size, the number of bilayers, lipid composition, as well as the charge and surface characteristics of the liposomes.

Liposomes can adsorb to virtually any type of cell and then slowly release the encapsulated agent. Alternatively, an absorbed liposome may be endocytosed by cells that are phagocytic. Endocytosis is followed by intralysosomal degradation of liposomal lipids and release of the encapsulated agents (Scherphof et al., Ann. N.Y. Acad. Sci. 446:368 (1985)). After intravenous administration, small liposomes (0.1 to 1.0 μm) are typically taken up by cells of the reticuloendothelial system, located principally in the liver and spleen, whereas liposomes larger than 3.0 μm are deposited in the lung. This preferential uptake of smaller liposomes by the cells of the reticuloendothelial system has been used to deliver chemotherapeutic agents to macrophages and to tumors of the liver.

The reticuloendothelial system can be circumvented by several methods including saturation with large doses of liposome particles, or selective macrophage inactivation by pharmacological means (Claassen et al., Biochim. Biophys. Acta 802:428 (1984)). In addition, incorporation of glycolipid- or polyethelene glycol-derivatized phospholipids into liposome membranes has been shown to result in a significantly reduced uptake by the reticuloendothelial system (Allen et al., Biochim. Biophys. Acta 1068:133 (1991); Allen et al., Biochim. Biophys. Acta 1150:9 (1993)).

Liposomes can also be prepared to target particular cells or organs by varying phospholipid composition or by inserting receptors or ligands into the liposomes. For example, liposomes, prepared with a high content of a nonionic surfactant, have been used to target the liver (Hayakawa et al., Japanese Patent 04-244,018; Kato et al., Biol. Pharm. Bull. 16:960 (1993)). These formulations were prepared by mixing soybean phospatidylcholine, α-tocopherol, and ethoxylated hydrogenated castor oil (HCO-60) in methanol, concentrating the mixture under vacuum, and then reconstituting the mixture with water. A liposomal formulation of dipalmitoylphosphatidylcholine (DPPC) with a soybean-derived sterylglucoside mixture (SG) and cholesterol (Ch) has also been shown to target the liver (Shimizu et al., Biol. Pharm. Bull. 20:881 (1997)).

Alternatively, various targeting ligands can be bound to the surface of the liposome, such as antibodies, antibody fragments, carbohydrates, vitamins, and transport proteins. For example, liposomes can be modified with branched type galactosyllipid derivatives to target asialoglycoprotein (galactose) receptors, which are exclusively expressed on the surface of liver cells (Kato and Sugiyama, Crit. Rev. Ther. Drug Carrier Syst. 14:287 (1997); Murahashi et al., Biol. Pharm. Bull. 20:259 (1997)). Similarly, Wu et al., Hepatology 27:772 (1998), have shown that labeling liposomes with asialofetuin led to a shortened liposome plasma half-life and greatly enhanced uptake of asialofetuin-labeled liposome by hepatocytes. On the other hand, hepatic accumulation of liposomes comprising branched type galactosyllipid derivatives can be inhibited by preinjection of asialofetuin (Murahashi et al., Biol. Pharm. Bull. 20:259 (1997)). Polyaconitylated human serum albumin liposomes provide another approach for targeting liposomes to liver cells (Kamps et al., Proc. Nat'l Acad. Sci. USA 94:11681 (1997)). Moreover, Geho, et al. U.S. Pat. No. 4,603,044, describe a hepatocyte-directed liposome vesicle delivery system, which has specificity for hepatobiliary receptors associated with the specialized metabolic cells of the liver.

In a more general approach to tissue targeting, target cells are prelabeled with biotinylated antibodies specific for a ligand expressed by the target cell (Harasym et al., Adv. Drug Deliv. Rev. 32:99 (1998)). After plasma elimination of free antibody, streptavidin-conjugated liposomes are administered. In another approach, targeting antibodies are directly attached to liposomes (Harasym et al., Adv. Drug Deliv. Rev. 32:99 (1998)).

Polypeptides and antibodies can be encapsulated within liposomes using standard techniques of protein microencapsulation (see, for example, Anderson et al., Infect. Immun. 31:1099 (1981), Anderson et al., Cancer Res. 50:1853 (1990), and Cohen et al., Biochim. Biophys. Acta 1063:95 (1991), Alving et al. “Preparation and Use of Liposomes in Immunological Studies,” in Liposome Technology, 2nd Edition, Vol. III, Gregoriadis (ed.), page 317 (CRC Press 1993), Wassef et al., Meth. Enzymol. 149:124 (1987)). As noted above, therapeutically useful liposomes may contain a variety of components. For example, liposomes may comprise lipid derivatives of poly(ethylene glycol) (Allen et al., Biochim. Biophys. Acta 1150:9 (1993)).

Degradable polymer microspheres have been designed to maintain high systemic levels of therapeutic proteins. Microspheres are prepared from degradable polymers such as poly(lactide-co-glycolide) (PLG), polyanhydrides, poly (ortho esters), nonbiodegradable ethylvinyl acetate polymers, in which proteins are entrapped in the polymer (Gombotz and Pettit, Bioconjugate Chem. 6:332 (1995); Ranade, “Role of Polymers in Drug Delivery,” in Drug Delivery Systems, Ranade and Hollinger (eds.), pages 51-93 (CRC Press 1995); Roskos and Maskiewicz, “Degradable Controlled Release Systems Useful for Protein Delivery,” in Protein Delivery: Physical Systems, Sanders and Hendren (eds.), pages 45-92 (Plenum Press 1997); Bartus et al., Science 281:1161 (1998); Putney and Burke, Nature Biotechnology 16:153 (1998); Putney, Curr. Opin. Chem. Biol. 2:548 (1998)). Polyethylene glycol (PEG)-coated nanospheres can also provide carriers for intravenous administration of therapeutic proteins (see, for example, Gref et al., Pharm. Biotechnol. 10:167 (1997)).

The present invention also contemplates chemically modified polypeptides having binding ZcytoR21 activity such as ZcytoR21 monomeric, homodimeric, heterodimeric or multimeric soluble receptors, and ZcytoR21 antagonists, for example anti-ZcytoR21 antibodies or binding polypeptides, or neutralizing anti-ZcytoR21 antibodies, which a polypeptide is linked with a polymer, as discussed above.

Other dosage forms can be devised by those skilled in the art, as shown, for example, by Ansel and Popovich, Pharmaceutical Dosage Forms and Drug Delivery Systems, 5^{th} Edition (Lea & Febiger 1990), Gennaro (ed.), Remington's Pharmaceutical Sciences, 19^{th} Edition (Mack Publishing Company 1995), and by Ranade and Hollinger, Drug Delivery Systems (CRC Press 1996).

As an illustration, pharmaceutical compositions may be supplied as a kit comprising a container that comprises a polypeptide with a ZcytoR21 extracellular domain, e.g., ZcytoR21 monomeric, homodimeric, heterodimeric or multimeric soluble receptors, or a ZcytoR21 antagonist (e.g., an antibody or antibody fragment that binds a ZcytoR21 polypeptide, or neutralizing anti-ZcytoR21 antibody). Therapeutic polypeptides can be provided in the form of an injectable solution for single or multiple doses, or as a sterile powder that will be reconstituted before injection. Alternatively, such a kit can include a dry-powder disperser, liquid aerosol generator, or nebulizer for administration of a therapeutic polypeptide. Such a kit may further comprise written information on indications and usage of the pharmaceutical composition. Moreover, such information may include a statement that the ZcytoR21 composition is contraindicated in patients with known hypersensitivity to ZcytoR21.

A pharmaceutical composition comprising anti-ZcytoR21 antibodies or binding partners (or anti-ZcytoR21 antibody fragments, antibody fusions, humanized antibodies and the like), or ZcytoR21 soluble receptor, can be furnished in liquid form, in an aerosol, or in solid form. Liquid forms, are illustrated by injectable solutions, aerosols, droplets, topological solutions and oral suspensions. Exemplary solid forms include capsules, tablets, and controlled-release forms. The latter form is illustrated by miniosmotic pumps and implants (Bremer et al., Pharm. Biotechnol. 10:239 (1997); Ranade, “Implants in Drug Delivery,” in Drug Delivery Systems, Ranade and Hollinger (eds.), pages 95-123 (CRC Press 1995); Bremer et al., “Protein Delivery with Infusion Pumps,” in Protein Delivery: Physical Systems, Sanders and Hendren (eds.), pages 239-254 (Plenum Press 1997); Yewey et al., “Delivery of Proteins from a Controlled Release Injectable Implant,” in Protein Delivery: Physical Systems, Sanders and Hendren (eds.), pages 93-117 (Plenum Press 1997)). Other solid forms include creams, pastes, other topological applications, and the like.

Liposomes provide one means to deliver therapeutic polypeptides to a subject intravenously, intraperitoneally, intrathecally, intramuscularly, subcutaneously, or via oral administration, inhalation, or intranasal administration. Liposomes are microscopic vesicles that consist of one or more lipid bilayers surrounding aqueous compartments (see, generally, Bakker-Woudenberg et al., Eur. J. Clin. Microbiol. Infect. Dis. 12 (Suppl. 1):S61 (1993), Kim, Drugs 46:618 (1993), and Ranade, “Site-Specific Drug Delivery Using Liposomes as Carriers,” in Drug Delivery Systems, Ranade and Hollinger (eds.), pages 3-24 (CRC Press 1995)). Liposomes are similar in composition to cellular membranes and as a result, liposomes can be administered safely and are biodegradable. Depending on the method of preparation, liposomes may be unilamellar or multilamellar, and liposomes can vary in size with diameters ranging from 0.02 μm to greater than 10 μm. A variety of agents can be encapsulated in liposomes: hydrophobic agents partition in the bilayers and hydrophilic agents partition within the inner aqueous space(s) (see, for example, Machy et al., Liposomes In Cell Biology And Pharmacology (John Libbey 1987), and Ostro et al., American J. Hosp. Pharm. 46:1576 (1989)). Moreover, it is possible to control the therapeutic availability of the encapsulated agent by varying liposome size, the number of bilayers, lipid composition, as well as the charge and surface characteristics of the liposomes.

Liposomes can adsorb to virtually any type of cell and then slowly release the encapsulated agent. Alternatively, an absorbed liposome may be endocytosed by cells that are phagocytic. Endocytosis is followed by intralysosomal degradation of liposomal lipids and release of the encapsulated agents (Scherphof et al., Ann. N.Y. Acad. Sci. 446:368 (1985)). After intravenous administration, small liposomes (0.1 to 1.0 μm) are typically taken up by cells of the reticuloendothelial system, located principally in the liver and spleen, whereas liposomes larger than 3.0 μm are deposited in the lung. This preferential uptake of smaller liposomes by the cells of the reticuloendothelial system has been used to deliver chemotherapeutic agents to macrophages and to tumors of the liver.

The reticuloendothelial system can be circumvented by several methods including saturation with large doses of liposome particles, or selective macrophage inactivation by pharmacological means (Claassen et al., Biochim. Biophys. Acta 802:428 (1984)). In addition, incorporation of glycolipid- or polyethelene glycol-derivatized phospholipids into liposome” membranes has been shown to result in a significantly reduced uptake by the reticuloendothelial system (Allen et al., Biochim. Biophys. Acta 1068:133 (1991); Allen et al., Biochim. Biophys. Acta 1150:9 (1993)).

Liposomes can also be prepared to target particular cells or organs by varying phospholipid composition or by inserting receptors or ligands into the liposomes. For example, liposomes, prepared with a high content of a nonionic surfactant, have been used to target the liver (Hayakawa et al., Japanese Patent 04-244,018; Kato et al., Biol. Pharm. Bull. 16:960 (1993)). These formulations were prepared by mixing soybean phospatidylcholine, α-tocopherol, and ethoxylated hydrogenated castor oil (HCO-60) in methanol, concentrating the mixture under vacuum, and then reconstituting the mixture with water. A liposomal formulation of dipalmitoylphosphatidylcholine (DPPC) with a soybean-derived sterylglucoside mixture (SG) and cholesterol (Ch) has also been shown to target the liver (Shimizu et al., Biol. Pharm. Bull. 20:881 (1997)).

Alternatively, various targeting ligands can be bound to the surface of the liposome, such as antibodies, antibody fragments, carbohydrates, vitamins, and transport proteins. For example, liposomes can be modified with branched type galactosyllipid derivatives to target asialoglycoprotein (galactose) receptors, which are exclusively expressed on the surface of liver cells (Kato and Sugiyama, Crit. Rev. Ther. Drug Carrier Syst 14:287 (1997); Murahashi et al., Biol. Pharm. Bull. 20:259 (1997)). Similarly, Wu et al., Hepatology 27:772 (1998), have shown that labeling liposomes with asialofetuin led to a shortened liposome plasma half-life and greatly enhanced uptake of asialofetuin-labeled liposome by hepatocytes. On the other hand, hepatic accumulation of liposomes comprising branched type galactosyllipid derivatives can be inhibited by preinjection of asialofetuin (Murahashi et al., Biol. Pharm. Bull. 20:259 (1997)). Polyaconitylated human serum albumin liposomes provide another approach for targeting liposomes to liver cells (Kamps et al., Proc. Nat'l Acad. Sci. USA 94:11681 (1997)). Moreover, Geho, et al. U.S. Pat. No. 4,603,044, describe a hepatocyte-directed liposome vesicle delivery system, which has specificity for hepatobiliary receptors associated with the specialized metabolic cells of the liver.

In a more general approach to tissue targeting, target cells are prelabeled with biotinylated antibodies specific for a ligand expressed by the target cell (Harasym et al., Adv. Drug Deliv. Rev. 32:99 (1998)). After plasma elimination of free antibody, streptavidin-conjugated liposomes are administered. In another approach, targeting antibodies are directly attached to liposomes (Harasym et al., Adv. Drug Deliv. Rev. 32:99 (1998)).

Anti-ZcytoR21 neutralizing antibodies and binding partners with IL-17C binding activity, or ZcytoR21 soluble receptor, can be encapsulated within liposomes using standard techniques of protein microencapsulation (see, for example, Anderson et al., Infect. Immun. 31:1099 (1981), Anderson et al., Cancer Res. 50:1853 (1990), and Cohen et al., Biochim. Biophys. Acta 1063:95 (1991), Alving et al. “Preparation and Use of Liposomes in Immunological Studies,” in Liposome Technology, 2nd Edition, Vol. III, Gregoriadis (ed.), page 317 (CRC Press 1993), Wassef et al., Meth. Enzymol. 149:124 (1987)). As noted above, therapeutically useful liposomes may contain a variety of components. For example, liposomes may comprise lipid derivatives of poly(ethylene glycol) (Allen et al., Biochim. Biophys. Acta 1150:9 (1993)).

Degradable polymer microspheres have been designed to maintain high systemic levels of therapeutic proteins. Microspheres are prepared from degradable polymers such as poly(lactide-co-glycolide) (PLG), polyanhydrides, poly (ortho esters), nonbiodegradable ethylvinyl acetate polymers, in which proteins are entrapped in the polymer (Gombotz and Pettit, Bioconjugate Chem. 6:332 (1995); Ranade, “Role of Polymers in Drug Delivery,” in Drug Delivery Systems, Ranade and Hollinger (eds.), pages 51-93 (CRC Press 1995); Roskos and Maskiewicz, “Degradable Controlled Release Systems Useful for Protein Delivery,” in Protein Delivery: Physical Systems, Sanders and Hendren (eds.), pages 45-92 (Plenum Press 1997); Bartus et al., Science 281:1161 (1998); Putney and Burke, Nature Biotechnology 16:153 (1998); Putney, Curr. Opin. Chem. Biol. 2:548 (1998)). Polyethylene glycol (PEG)-coated nanospheres can also provide carriers for intravenous administration of therapeutic proteins (see, for example, Gref et al., Pharm. Biotechnol. 10:167 (1997)).

The present invention also contemplates chemically modified anti-ZcytoR21 antibody or binding partner, for example anti-ZcytoR21 antibodies or ZcytoR21 soluble receptor, linked with a polymer, as discussed above.

Other dosage forms can be devised by those skilled in the art, as shown, for example, by Ansel and Popovich, Pharmaceutical Dosage Forms and Drug Delivery Systems, 5^{th} Edition (Lea & Febiger 1990), Gennaro (ed.), Remington's Pharmaceutical Sciences, 19^{th} Edition (Mack Publishing Company 1995), and by Ranade and Hollinger, Drug Delivery Systems (CRC Press 1996).

The present invention contemplates compositions of anti-IL-17C antibodies, and methods and therapeutic uses comprising an antibody, peptide or polypeptide described herein. Such compositions can further comprise a carrier. The carrier can be a conventional organic or inorganic carrier. Examples of carriers include water, buffer solution, alcohol, propylene glycol, macrogol, sesame oil, corn oil, and the like.

J) Production of Transgenic Mice

Transgenic mice can be engineered to over-express the either IL-17C or the ZcytoR21 gene in all tissues or under the control of a tissue-specific or tissue-preferred regulatory element. These over-producers can be used to characterize the phenotype that results from over-expression, and the transgenic animals can serve as models for human disease caused by excess IL-17C or ZcytoR21. Transgenic mice that over-express any of these also provide model bioreactors for production of ZcytoR21, such as soluble ZcytoR21, in the milk or blood of larger animals. Methods for producing transgenic mice are well-known to those of skill in the art (see, for example, Jacob, “Expression and Knockout of Interferons in Transgenic Mice,” in Overexpression and Knockout of Cytokines in Transgenic Mice, Jacob (ed.), pages 111-124 (Academic Press, Ltd. 1994), Monastersky and Robl (eds.), Strategies in Transgenic Animal Science (ASM Press 1995), and Abbud and Nilson, “Recombinant Protein Expression in Transgenic Mice,” in Gene Expression Systems: Using Nature for the Art of Expression, Fernandez and Hoeffler (eds.), pages 367-397 (Academic Press, Inc. 1999)).

For example, a method for producing a transgenic mouse that expresses a ZcytoR21 gene can begin with adult, fertile males (studs) (B6C3f1, 2-8 months of age (Taconic Farms, Germantown, N.Y.)), vasectomized males (duds) (B6D2f1, 2-8 months, (Taconic Farms)), prepubescent fertile females (donors) (B6C3f1, 4-5 weeks, (Taconic Farms)) and adult fertile females (recipients) (B6D2f1, 2-4 months, (Taconic Farms)). The donors are acclimated for one week and then injected with approximately 8 IU/mouse of Pregnant Mare's Serum gonadotrophin (Sigma Chemical Company; St. Louis, Mo.) I.P., and 46-47 hours later, 8 IU/mouse of human Chorionic Gonadotropin (hCG (Sigma)) I.P. to induce superovulation. Donors are mated with studs subsequent to hormone injections. Ovulation generally occurs within 13 hours of hCG injection. Copulation is confirmed by the presence of a vaginal plug the morning following mating.

Fertilized eggs are collected under a surgical scope. The oviducts are collected and eggs are released into urinanalysis slides containing hyaluronidase (Sigma). Eggs are washed once in hyaluronidase, and twice in Whitten's W640 medium (described, for example, by Menino and O'Claray, Biol. Reprod. 77:159 (1986), and Dienhart and Downs, Zygote 4:129 (1996)) that has been incubated with 5% CO₂, 5% O₂′ and 90% N₂ at 37° C. The eggs are then stored in a 37° C./5% CO₂ incubator until microinjection.

Ten to twenty micrograms of plasmid DNA containing a ZcytoR21 encoding sequence is linearized, gel-purified, and resuspended in 10 mM Tris-HCl (pH 7.4), 0.25 mM EDTA (pH 8.0), at a final concentration of 5-10 nanograms per microliter for microinjection. For example, the ZcytoR21 encoding sequences can encode a polypeptide comprising any of SEQ ID NOs:3, 6, 9, 12, 15, 21, 23, 109, 113, 115, 117, 119, or 122.

Plasmid DNA is microinjected into harvested eggs contained in a drop of W640 medium overlaid by warm, CO₂-equilibrated mineral oil. The DNA is drawn into an injection needle (pulled from a 0.75 mm ID, 1 mm OD borosilicate glass capillary), and injected into individual eggs. Each egg is penetrated with the injection needle, into one or both of the haploid pronuclei.

Picoliters of DNA are injected into the pronuclei, and the injection needle withdrawn without coming into contact with the nucleoli. The procedure is repeated until all the eggs are injected. Successfully microinjected eggs are transferred into an organ tissue-culture dish with pre-gassed W640 medium for storage overnight in a 37° C./5% CO incubator.

The following day, two-cell embryos are transferred into pseudopregnant recipients. The recipients are identified by the presence of copulation plugs, after copulating with vasectomized duds. Recipients are anesthetized and shaved on the dorsal left side and transferred to a surgical microscope. A small incision is made in the skin and through the muscle wall in the middle of the abdominal area outlined by the ribcage, the saddle, and the hind leg, midway between knee and spleen. The reproductive organs are exteriorized onto a small surgical drape. The fat pad is stretched out over the surgical drape, and a baby serrefine (Roboz, Rockville, Md.) is attached to the fat pad and left hanging over the back of the mouse, preventing the organs from sliding back in.

With a fine transfer pipette containing mineral oil followed by alternating W640 and air bubbles, 12-17 healthy two-cell embryos from the previous day's injection are transferred into the recipient. The swollen ampulla is located and holding the oviduct between the ampulla and the bursa, a nick in the oviduct is made with a 28 g needle close to the bursa, making sure not to tear the ampulla or the bursa.

The pipette is transferred into the nick in the oviduct, and the embryos are blown in, allowing the first air bubble to escape the pipette. The fat pad is gently pushed into the peritoneum, and the reproductive organs allowed to slide in. The peritoneal wall is closed with one suture and the skin closed with a wound clip. The mice recuperate on a 37° C. slide warmer for a minimum of four hours.

The recipients are returned to cages in pairs, and allowed 19-21 days gestation. After birth, 19-21 days postpartum is allowed before weaning. The weanlings are sexed and placed into separate sex cages, and a 0.5 cm biopsy (used for genotyping) is snipped off the tail with clean scissors.

Genomic DNA is prepared from the tail snips using, for example, a QIAGEN DNEASY kit following the manufacturer's instructions. Genomic DNA is analyzed by PCR using primers designed to amplify a ZcytoR21 gene or a selectable marker gene that was introduced in the same plasmid. After animals are confirmed to be transgenic, they are back-crossed into an inbred strain by placing a transgenic female with a wild-type male, or a transgenic male with one or two wild-type female(s). As pups are born and weaned, the sexes are separated, and their tails snipped for genotyping.

To check for expression of a transgene in a live animal, a partial hepatectomy is performed. A surgical prep is made of the upper abdomen directly below the zyphoid process. Using sterile technique, a small 1.5-2 cm incision is made below the sternum and the left lateral lobe of the liver exteriorized. Using 4-0 silk, a tie is made around the lower lobe securing it outside the body cavity. An atraumatic clamp is used to hold the tie while a second loop of absorbable Dexon (American Cyanamid; Wayne, N.J.) is placed proximal to the first tie. A distal cut is made from the Dexon tie and approximately 100 mg of the excised liver tissue is placed in a sterile petri dish. The excised liver section is transferred to a 14 ml polypropylene round bottom tube and snap frozen in liquid nitrogen and then stored on dry ice. The surgical site is closed with suture and wound clips, and the animal's cage placed on a 37° C. heating pad for 24 hours post operatively. The animal is checked daily post operatively and the wound clips removed 7-10 days after surgery. The expression level of ZcytoR21 mRNA is examined for each transgenic mouse using an RNA solution hybridization assay or polymerase chain reaction.

In addition to producing transgenic mice that over-express IL-17C or ZcytoR21, it is useful to engineer transgenic mice with either abnormally low or no expression of any of these genes. Such transgenic mice provide useful models for diseases associated with a lack of IL-17C or ZcytoR21. As discussed above, ZcytoR21 gene expression can be inhibited using anti-sense genes, ribozyme genes, or external guide sequence genes. To produce transgenic mice that under-express the ZcytoR21 gene, such inhibitory sequences are targeted to ZcytoR21 mRNA. Methods for producing transgenic mice that have abnormally low expression of a particular gene are known to those in the art (see, for example, Wu et al., “Gene Underexpression in Cultured Cells and Animals by Antisense DNA and RNA Strategies,” in Methods in Gene Biotechnology, pages 205-224 (CRC Press 1997)).

An alternative approach to producing transgenic mice that have little or no ZcytoR21 gene expression is to generate mice having at least one normal ZcytoR21 allele replaced by a nonfunctional ZcytoR21 gene. One method of designing a nonfunctional ZcytoR21 gene is to insert another gene, such as a selectable marker gene, within a nucleic acid molecule that encodes ZcytoR21. Standard methods for producing these so-called “knockout mice” are known to those skilled in the art (see, for example, Jacob, “Expression and Knockout of Interferons in Transgenic Mice,” in Overexpression and Knockout of Cytokines in Transgenic Mice, Jacob (ed.), pages 111-124 (Academic Press, Ltd. 1994), and Wu et al., “New Strategies for Gene Knockout,” in Methods in Gene Biotechnology, pages 339-365 (CRC Press 1997)).

The invention is further illustrated by the following non-limiting examples.

### EXAMPLES

### Example 1

### Human ZcytoR21 Tissue Distribution in Tissue Panels Using PCR

The Human Rapid-Scan cDNA panel represents 24 adult tissues and is arrayed at 4 different concentrations called 1×, 10×, 100×, and 1000× (Origen, Rockville, Md.). The “1000× and 100x” levels were screened for ZcytoR21 transcription using PCR. The sense primer was zc39334, (5′ AGGCCCTGCCACCCACCTTC 3′) (SEQ ID NO:26) located in a cDNA area corresponding to the 5′ untranslated region. The antisense primer was zc39333, (5′-CGAGGCACCCCAAGGATTTCAG-3′) (SEQ ID NO:27) located in a cDNA area corresponding to the 3′ untranslated region. PCR was applied using pfu turbo polymerase and the manufacturer's recommendations (Stratagene, La Jolla, Calif.) except for using rediload dye, (Research Genetics, Inc., Huntsville, Ala.) a wax hot start, (Molecular Bioproducts Inc. San Diego, Calif.) and 10% (final concentration) DMSO. The amplification was carried out as follows: 1 cycle at 94° C. for 4 minutes, 40 cycles of 94° C. for 30 seconds, 51° C. for 30 seconds and 72° C. for 3 minutes, followed by 1 cycle at 72° C. for 7 minutes. About 10 μl of the PCR reaction product was subjected to standard agarose gel electrophoresis using a 1% agarose gel. Following electrophoresis, the gels were Southern blotted and the membranes hybridized by standard methods using a ³²P isotope-labeled oligonucleotide, zc40458 (5′-TCTCTGACTCTGCTGGGATTGG-3′) (SEQ ID NO:28) which maps to the cDNA area in the translated region, just downstream of the start codon. X ray film autoradiography revealed ZcytoR21-specific amplicons only in colon, lung, stomach, placenta, and bone marrow.

### Example 2

### Cloning of Human ZcytoR21x1

Human ZcytoR21x1 (SEQ ID NO:1) was cloned by PCR using 10 ng of a human hacat cell line (skin-derived) amplified plasmid cDNA library template and primers 5′CGAGGCACCCCAAGGATTTCAG 3′(SEQ ID NO:179) and 5′ AGGCCCTGCCACCCACCTTC 3′ (SEQ ID NO:180) and pfu ultra polymerase according to the manufacturer's recommendations. These primers map to the 5′ and 3′utr regions of human ZcytoR21 cDNA. The resulting products were subjected to a preparative low melt agarose TAE gel electrophoresis and the approximately 1.3-2.5 KB region size-selectively purified and then liquefied using the gelase method. (Epicenter) This template was then diluted 1:50 in sterile water and 1 uL amplified using pfu ultra polymerase by nested PCR, using 5′ CGTACGGGCCGGCCACCATGGGGAGCTCCAGACTGGCA 3′ (SEQ ID NO:181) containing a FseI restriction site and 5′ TGACGAGGCGCGCCTCAACCTAGGTCTGCAAGT 3′(SEQ ID NO:182) containing an AscI restriction site. These primers amplify just the translated region of human ZcytoR21. The resulting products were desalted and the primers eliminated utilizing a chromaspin 100 column (Clontech) and then digested with FseI and AscI restriction enzymes, size-selected on a low melt agarose gel for approximately 1.3-2.5 KB fragments. Fragments were ligated into a pZMP11 expression vector's FseI/AscI restriction sites. Clone's DNA inserts were subjected to sequencing analysis, revealing clone d2, which was designated ZcytoR21x1 (SEQ ID NO:1)

### Example 3

Cloning of Human ZcytoR21x2, ZcytoR21x3 and ZcytoR21x4

Human ZcytoR21x2 (SEQ ID NO:4), ZcytoR21x3 (SEQ ID NO:7), and ZcytoR21x4 (SEQ ID NO:10) were cloned by PCR using 1 ul of a human adult skin cDNA (clontech) template and the following primers: 5′CGAGGCACCCCAAGGATTTCAG3′ (SEQ ID NO:162) and 5′AGGCCCTGCCACCCACCTTC3′ (SEQ ID NO:163) and pfu ultra polymerase according to the manufacturer's recommendations. These primers map to the 5′ and 3′ utr regions of human ZcytoR21 cDNA. The resulting products were subjected to a preparative low melt agarose TAE gel electrophoresis and the approximately 1.3-2.5 KB region size-selectively purified and then liquefied using the gelase method. (Epicenter) This template was then diluted 1:50 in sterile water and 1 uL amplified using pfu ultra polymerase by nested PCR, using 5′CGTACGGGCCGGCCACCATGGGGAGCTCCAGACTGGCA3′ (SEQ ID NO:164) containing a FseI restriction site and 5′ TGACGAGGCGCGCCTCAACCTAGGTCTGCAAGT 3′ (SEQ ID NO:165) containing an AscI restriction site. These primers amplify just the translated region of human ZcytoR21. The resulting products were desalted and the primers eliminated utilizing a chromaspin 100 column (Clontech) and then digested with FseI and AscI restriction enzymes, size-selected on a low melt agarose gel for approximately 1.3-2.5 KB fragments. Fragments were ligated into a pZMP11 expression vector's FseI/AscI restriction sites. Clone's DNA inserts were subjected to sequencing analysis, revealing clones F1, F5, and F6, which were designated ZcytoR21x2 (SEQ ID NO:4), ZcytoR21x3 (SEQ ID NO:7), and ZcytoR21x4 (SEQ ID NO:10) respectively.

### Example 4

### Cloning of Human ZcytoR21x6 and x13

Briefly, cDNA obtained from human colon from a patient with active Crohn's disease was used as a template. One micro liter of the above template was amplified by PCR, using primers 39333 5′CGAGGCACCCCAAGGATTTCAG 3′(SEQ ID NO:53) and 39334, 5′ AGGCCCTGCCACCCACCTTC 3′ (SEQ ID NO:54) and pfu ultra polymerase according to the manufacturer's recommendations. These primers map to the 5′ and 3′ utr regions of human ZcytoR21 cDNA. The resulting products were subjected to a preparative low melt agarose TAE gel electrophoresis and the ˜1.3-2.5 KB region size-selectively purified and then liquefied using the gelase method. (Epicenter) Two micro liters of the purified fragments were amplified using pfu ultra polymerase by nested PCR, using ZC 39429, 5′CGTACGGGCCGGCCACCATGGGGAGCTCCAGACTGGCA3′ (SEQ ID NO:65) containing a FseI restriction site and zc 39433, 5′ TGACGAGGCGCGCCTCAACCTAGGTCTGCAAGT 3′ (SEQ ID NO:66) containing an AscI restriction site. These primers amplify just the translated region of human ZcytoR21. The resulting products were then digested with FseI and AscI restriction enzymes, size-selected on a low melt agarose gel for ˜1.3-2.5 KB fragments and cloned in and expression vector, pZMP11. ZcytoR21 positive clones were identified using colony lifts of the resulting colonies and hybridized to a radiolabeled oligomer, zc 39948, 5′TTTCGCCACCTGCCCCACTGGAACACCCGCTGTCC3′ (SEQ ID NO:67) One hundred human ZcytoR21 positive colonies were sent for DNA sequence determination, revealing a variety of different ZcytoR21 cDNAs including human ZcytoR21x6 (SEQ ID NOs:20 and 21) and human ZcytoR21x13 (SEQ ID NOs:106 and 107).

### Example 5

### Cloning of Murine ZcytoR21

A putative full-length mouse cDNA sequence for ZcytoR21 was identified through computational and bioinformatical methods, using homology to the sequence of human ZcytoR21 (SEQ ID NO:6). This sequence was used in a Blast query to identify potential full-length mouse clones to purchase through vendors of IMAGE consortium clones. In this manner, clones corresponding to IMAGE ID numbers 5319489, 4457159, 6311568, and 4482367 were purchased (American Type Culture Collection, Manassas, Va.) and sequenced in their entirety. Analysis of these sequences led to the identification of two isoforms of this gene designated murine ZcytoR21x5 (SEQ ID NOs: 68 and 69) and murine ZcytoR21x6 (SEQ ID NOs:13 and 14).

### Example 6

### Cloning of Murine ZcytoR21x15

To clone murine ZcytoR21x15 (SEQ ID NOs:110 and 111), total RNA was extracted from the colons of mice with artificially induced colitis (described below in Example 42) This RNA was reverse transcribed into first strand cDNA using standard methods. Approximately 50 ng cDNA was amplified by PCR using primers 51388 5′CCTGCCCCTGCCTGCGGAGTT 3′ (SEQ ID NO:70) and 51387, 5′ GTTGCTACACAGGCTGAGGCTACA 3′ (SEQ ID NO:71) and pfu ultra polymerase according to the manufacturer's recommendations. The resulting products were subjected to a preparative low melt agarose TAE gel electrophoresis and the ˜1.3-2.5 KB region size-selectively purified and then liquefied using the gelase method. (Epicenter) Approximately 0.5 uL of the purified fragments were amplified using pfu ultra polymerase by nested PCR, using the same primers described above and Advantage2 2 polymerase, (Clontech) to add 5′ T overhangs, which enabled sub-cloning them in pCR4TOPO. (Invitrogen) Amplicons were size selected as above again prior to sub-cloning. Positives were identified using colony lifts and hybridized to a radiolabeled oligomer 51602. 5′ CTACCAAGGCTCAACCAATAGTCCCTGTGGT=TC 3′. (SEQ ID NO:72) One hundred mouse ZcytoR21 positive colonies were sent for DNA sequence determination, revealing a variety of different ZcytoR21 cDNAs including murine ZcytoR21x15 (SEQ ID NOs: 110 and 111).

### Example 7

### Cloning of Human IL-17C

A fragment of a putative IL-17C cDNA was identified through computational means and the PCR primers zc18634 (5′atgaggaccgctatccacagaagc 3′) (SEQ ID NO:29) and zc18635 (5′ggacgtggatgaactcggtgtgg 3′) (SEQ ID NO:30) were synthesized and used to survey by PCR a number of potential cloning sources for IL-17C. PCR conditions were are follows: Takara ExTaq polymerase and buffer (Takara, Otsu, Shiga, Japan) were used in 50 ul PCR reactions with 5 ul marathon cDNA templates made from RNAs from salivary gland, spinal cord, MCF-7 cell line, CaCo2 cell line, T47D cell line, Molt-4 cell line, and prostate, using a Marathon cDNA Amplification Kit (Clontech, Palo Alto, Calif.) according to the manufacturer's instructions. Also, each reaction contained 2.5 ul 10×PCR buffer, 2.5 ul Redi-Load, (Invitrogen, Carlsbad, Calif.), 2 ul 2.5 mM GeneAmp dNTPs (Applied Biosystems, Foster City, Calif.) 0.5 ul ExTaq, 0.5 ul of 20 pm/ul zc18634 and zc18635, and water to 50 ul. Cycling conditions were: 94° C. 1′, 30 cycles of 94° C. 20″, 68° C. 1′, followed by one cycle of 72° C. 7′.

PCR products were subjected to agarose gel electrophoresis and the ˜200 bp fragment was excised from the gel and purified using a Qiaquick Gel extraction spin column (Qiagen, Valencia, Calif.) according to the manufacturer's directions. This fragment was then sequenced to verify it as IL-17C. Standard 5′ and 3′ nested RACE reactions were then performed on DNA from an amplified in-house fetal lung library to generate overlapping PCR fragments, the sequence of which enabled the elucidation of the complete open reading frame plus some 5′ and 3′ untranslated sequence of IL-17C.

Finally, zc21607 (5′gcacacctggcggcaccatgac3′) (SEQ ID NO:31) and zc21597 (5′ctgtcctccagacacggggaatg3′) (SEQ ID NO:32) were used to generate by PCR a cDNA containing the complete open reading frame plus some 3′ untranslated region of IL-17C from DNA of an amplified in-house fetal lung library. PCR conditions were are follows: Advantage 2 PCR reagents (Clontech, Palo Alto, Calif.) were used in a 50 ul PCR reaction with 5 ul template, 5 ul 10×PCR buffer, 5 ul Redi-Load, (Invitrogen, Carlsbad, Calif.), 4 ul 2.5 mM GeneAmp dNTPs (Applied Biosystems, Foster City, Calif.), 1 ul Advantage 2 polymerase mix, 5 ul GC-melt (Clontech, Palo Alto, Calif.), 2.5 ul DMSO, 1 ul of 20 pm/ul zc21607 and zc21597, and water to 50 ul. Cycling conditions were: 94° C. 1′, 25 cycles of 94° C. 20″, 68° C. 1′30″, followed by one cycle of 72° C. 5′. The PCR product was subjected to agarose gel electrophoresis and the ˜770 bp fragment was excised from the gel and purified using a Qiaquick Gel extraction spin column (Qiagen, Valencia, Calif.) according to the manufacturer's directions.

The fragment was subcloned into a TA cloning vector, PCR2.1 (Invitrogen, Carlsbad, Calif.), according to the manufacturer's instructions, sequenced, and compared to the sequences of the overlapping RACE products and existing human public genome sequence to identify potential PCR errors. A correct clone was archived and used for additional research applications.

### Example 8

### Identification and Cloning of Murine IL-17C

Based on the NCBI Mus musculus mRNA accession # XM_{—}146558 and in-house computational gene prediction models, the cDNA for mouse IL17C was generated by PCR of the predicted exons from mouse genomic DNA (Clonetech Cat. # 6650-1, lot # 0050310). Exon 2 PCR product was generated using primers 49910: 5′TCACTGTGATGAGTCTCCTGCTTCTAG3′ (SEQ ID NO:73) and 44991: 5′GTGTCGATGCGATATCTCCATGGTGAGA3′ (SEQ ID NO:74). Exon 3 PCR product was generated using primers 49912: 5′GAGATATCGCATCGACACAGATGAGAACC3′ (SEQ ID NO:75) and 49913: 5′TCACTGTGTAGACCTGGGAAGA3′ (SEQ ID NO:76). Exon 1 and the entire cDNA was then amplified in a cross-over PCR reaction using the PCR products for exons 2 and 3 along with primers 49959: 5′GCCACCATGGCCACCGTCACCGTCACTGTGATGAGTCTCCTGCTT3′ (SEQ ID NO:77). The resulting PCR product that encoded murine IL-17C (SEQ ID NO:19) was cloned into PCR II Blunt TOPO vector for sequence verification.

### Example 9

### Expression of IL-17C Using Adenovirus Constructs

Generation of Untagged Recombinant Adenovirus

The protein coding region of human IL-17C (SEQ ID NO:16) was amplified by PCR using primers that added FseI and AscI restriction sties at the 5′ and 3′ termini respectively. PCR primers ZC21925 (5′cacacaggccggccaccatgacgctcctccccggcctcc3′) (SEQ ID NO:37) and ZC21922 (5′cacacaggcgcgccttcacactgaacggggcagcacgc3′) (SEQ ID NO:38) were used with a pCR2.1 ta plasmid containing the full-length murine IL-17C cDNA in a PCR reaction as follows: one cycle at 95° C. for 5 minutes, followed by 18 cycles at 95° C. for 0.5 minute, 58° C. for 0.5 minute, and 72° C. for 0.5 minute, followed by 72° C. for 7 minutes, followed by a 4° C. soak. The PCR reaction product was loaded onto a 1.2% (low melt) SEAPLAQUE GTG (FMC BioProducts; Rockland, Me.) gel in TAE buffer. The IL-17C PCR product was excised from the gel, melted at 65° C., phenol extracted twice and then ethanol precipitated. The PCR product was then digested with FseI-AscI, phenol/chloroform extracted, ethanol precipitated, and rehydrated (Tris/EDTA, pH 8).

The IL-17C fragment was then ligated into the FseI-AscI sites of a modified pAdTrack CMV (He et al., Proc. Nat'l Acad. Sci. USA 95:2509 (1998)). This construct also contains the green fluorescent protein (GFP) marker gene. The CMV promoter driving GFP expression was replaced with the SV40 promoter and the SV40 polyadenylation signal was replaced with the human growth hormone polyadenylation signal. In addition, the native polylinker was replaced with FseI, EcoRV, and AscI sites. This modified form of pAdTrack CMV was named pZyTrack. Ligation was performed using the FAST-LINK DNA ligation and screening kit (EPICENTRE TECHNOLOGIES; Madison, Wis.). Clones containing the IL-17C cDNA were identified by standard mini prep procedures. In order to linearize the plasmid, approximately 5 μg of the pZyTrack IL-17C plasmid were digested with PmeI. Approximately 1 μg of the linearized plasmid was cotransformed with 200 ng of supercoiled pAdEasy (He et al., Proc. Nat'l Acad. Sci. USA 95:2509 (1998)) into BJ5183 cells. The co-transformation was performed with a BIO-RAD GENE PULSER (BIO-RAD laboratories, Inc.; Hercules, Calif.) at 2.5 kV, 200 ohms and 25mFa. The entire co-transformation was plated on four LB plates containing 25 μg/ml kanamycin. The smallest colonies were picked and expanded in LB/kanamycin and recombinant adenovirus DNA identified by standard DNA miniprep procedures. Digestion of the recombinant adenovirus DNA with FseI-AscI confirmed the presence of IL-17C. The recombinant adenovirus miniprep DNA was transformed into DH10B competent cells and DNA prepared using a QIAGEN maxi prep kit as per kit instructions.

Transfection of 293A Cells with Recombinant DNA

Approximately 5 μg of recombinant adenoviral DNA were digested with PacI enzyme for three hours at 37° C. in a reaction volume of 100 μl containing 20-30U of PacI. The digested DNA was extracted twice with an equal volume of phenol/chloroform and precipitated with ethanol. The DNA pellet was resuspended in 5 μl distilled water. A T25 flask of QBI-293A cells (Quantum Biotechnologies, Inc.; Montreal, Quebec, Canada), inoculated the day before and grown to 60-70% confluence, were transfected with the PacI digested DNA. The PacI-digested DNA was diluted up to a total volume of 50 μl with sterile HBS (150 mM NaCl, 20 mM HEPES). In a separate tube, 25 μl DOTAP (1 mg/ml; Roche Molecular Biochemicals; Indianapolis, Ind.) were diluted to a total volume of 100 μl with HBS. The DNA was added to the DOTAP, mixed gently by pipeting up and down, and left at room temperature for 15 minutes. The medium was removed from the 293A cells and washed with 5 ml serum-free MEMalpha (LIFE TECHNOLOGIES, Inc; Rockville, Md.) containing 1 mM sodium pyruvate (LIFE TECHNOLOGIES, Inc), 0.1 mM MEM non-essential amino acids (LIFE TECHNOLOGIES, Inc) and 25 mM HEPES buffer (LIFE TECHNOLOGIES, Inc). Five milliliters of serum-free MEM were added to the 293A cells and held at 37° C. The DNA/lipid mixture was added drop-wise to the T25 flask of 293A cells, mixed gently and incubated at 37° C. for 4 hours. After four hours, the medium containing the DNA/lipid mixture was aspirated off and replaced with 5 ml complete MEM containing 5% fetal bovine serum. The transfected cells were monitored for green fluorescent protein (GFP) expression and formation of foci.

Seven days after transfection of 293A cells with the recombinant adenoviral DNA, the cells expressed the GFP protein and started to form foci. These foci are viral “plaques” and the crude viral lysate was collected by using a cell scraper to collect all of the 293A cells. The lysate was transferred to a 50 ml conical tube. To release most of the virus particles from the cells, three freeze/thaw cycles were done in a dry ice/ethanol bath and a 37° C. water bath.

Amplification of Recombinant Adenovirus (rAdV)

The crude lysate was amplified (“primary amplification”) to obtain a working stock of IL-17C rAdV lysate. Two hundred milliliters of crude rAdV lysate were added to each of ten 10 cm plates of nearly confluent (80-90%) 293A cells, which had been set up 20 hours previously. The plates were monitored for 48 to 72 hours for cytopathic effect under the white light microscope and expression of GFP under the fluorescent microscope. When all of the 293A cells showed cytopathic effect, this primary amplification stock lysate was collected and freeze/thaw cycles performed as described above.

Secondary amplification of IL-17C rAdV was obtained as follows. Twenty 15 cm tissue culture dishes of 293A cells were prepared so that the cells were 80-90% confluent. All but 20 milliliters of 5% MEM media was removed, and each dish was inoculated with 300-500 ml primary amplified rAdv lysate. After 48 hours, the 293A cells were lysed from virus production and this lysate was collected into 250 ml polypropylene centrifuge bottles and the rAdV purified.

AdV/cDNA Purification

NP-40 detergent was added to a final concentration of 0.5% to the bottles of crude lysate to lyse all cells. Bottles were placed on a rotating platform for 10 minutes, agitating as fast as possible without displacing the bottles. The debris was pelleted by centrifugation at 20,000×g for 15 minutes. The supernatant was transferred to 250 ml polycarbonate centrifuge bottles, and 0.5 volume of 20% PEG8000/2.5 M NaCl solution was added. The bottles were shaken overnight on ice. The bottles were centrifuged at 20,000×g for 15 minutes and supernatant discarded into a bleach solution. The precipitated virus/PEG appeared as a white precipitate located in two vertical lines along the wall of the bottle on either side of the spin mark. Using a sterile cell scraper, the precipitate from two bottles was resuspended in 2.5 ml PBS. The virus solution was placed in 2 ml microcentrifuge tubes and centrifuged at 14,000×g in the microfuge for 10 minutes to remove any additional cell debris. The supernatant from the 2 ml microcentrifuge tubes was transferred into a 15 ml polypropylene snapcap tube and adjusted to a density of 1.34 g/ml with cesium chloride (CsCl). The volume of the virus solution was estimated and 0.55 g/ml of was CsCl added. The CsCl was dissolved and 1 ml of this solution weighed 1.34 g. The solution was transferred polycarbonate thick-walled centrifuge tubes 3.2 ml and spun at 80,000 rpm (348,000×g) for 3-4 hours at 25° C. in a Beckman Optima TLX micro-ultracentrifuge with the TLA-100.4 rotor. The virus formed a white band. Using wide-bore pipette tips, the virus band was collected.

The virus from the gradient has a large amount of CsCl which must be removed before it can be used with cells. Pharmacia PD-10 columns prepacked with SEPHADEX G-25M (Amersham Pharmacia Biotech, Inc; Piscataway, N.J.) were used to desalt the virus preparation. The column was equilibrated with 20 ml of PBS. The virus was loaded and allowed to run into the column. Five milliliters of PBS were added to the column and fractions of 8-10 drops collected. The optical densities of 1:50 dilutions of each fraction were determined at 260 nm on a spectrophotometer. A clear absorbance peak was present between fractions 7-12. These fractions were pooled and the optical density (OD) of a 1:10 dilution determined. The following formula was used to convert OD into virus concentration: (OD at 260 nm)(10)(1.1×10¹²)=virions/ml. The OD of a 1:10 dilution of the IL-17C rAdV was 0.27 giving a virus concentration of 2.8×10¹² virions/ml.

To store the virus, glycerol was added to the purified virus to a final concentration of 15%, mixed gently but effectively, and stored in aliquots at −80° C.

Tissue Culture Infectious Dose at 50% CPE (TCID 50) Viral Titration Assay

A protocol developed by Quantum Biotechnologies, Inc. (Montreal, Quebec, Canada) was followed to measure recombinant virus infectivity. Briefly, two 96-well tissue culture plates were seeded with 1×10⁴ 293A cells per well in MEM containing 2% fetal bovine serum for each recombinant virus assayed. After 24 hours, 10-fold dilutions of each virus from 1×10⁻² to 1×10⁻¹⁴ were made in MEM containing 2% fetal bovine serum. One hundred microliters of each dilution were placed in each of 20 wells. After five days at 37° C., wells were read either positive or negative for cytopathic effect, and a value for “plaque forming units/ml” (PFU) is calculated.

The TCID₅₀ formulation was produced as per Quantum Biotechnologies, Inc., above. The titer is determined from a plate where virus used is diluted from 10⁻² to 10⁻¹⁴, and read five days after the infection. At each dilution a ratio (R) of positive wells for cytopathic effect per the total number of wells is determined.

To calculate the titer of the undiluted virus sample, factor “F” was first calculated, as 1+d(S-0.5), where “S” is the sum of the ratios (R), and “d” is log10 of the dilution series (e.g., “d” is equal to one for a ten-fold dilution series). The titer of the undiluted sample is calculated as: 10^{(1+F)}=TCID₅₀/ml. To convert TCID₅₀/ml to pfu/ml, 0.7 is subtracted from the exponent in the calculation for titer (T).

Using this method, the IL-17C adenovirus had a titer of 1.3×10¹⁰ pfu/ml.

### Example 10

### Construction of Mammalian Expression Vectors that Express Human ZcytoR21

An expression vector was prepared for the expression of the soluble, extracellular domain of the human ZcytoR21 polypeptide, ZcytoR21CHIS, wherein the construct is designed to express a ZcytoR21 polypeptide comprised of the predicted initiating methionine and truncated adjacent to the predicted transmembrane domain, and with a C-terminal HIS tag: 5′GGCTCAGGATCTGGTGGCGGCCATCACCACCATCATCACTAAATCTAGA3′ (SEQ ID NO: 78).

A 1160 bp PCR generated ZcytoR21 DNA fragment was created using ZC50282: 5′GAAGAACGTCTCTCATGGGGAGCTCCAGACTGGCAGC3′ (SEQ ID NO:79) and ZC50283: 5′GAAGAACGTCTCTAGCCGTGTCTGTAAGAGACATCCGGAC3′ (SEQ ID NO:80) as PCR primers to add Esp3I restriction sites and Tgo reagents (Roche, Applied Sciences, Indianapolis, Ind.). A plasmid containing the ZcytoR21 cDNA (Clonetrack ID#100989) was used as a template. PCR amplification of the ZcytoR21 fragment was performed as follows: One cycle of 94C for 2 minutes; then fifteen cycles at 94° C. for 30 seconds, 65° C. for 30 seconds, 72° C. for 1 minute, followed by one cycle of 72° C. for 5 minutes and then a 4° C. hold. The reaction was purified using QIAquick PCR purification kit (Qiagen, Santa Clarita, Calif.) and digested with Esp3I (Fermentas, Hanover, Md.) following manufacturer's protocol. The reaction was purified using QIAquick PCR purification kit (Qiagen, Santa Clarita, Calif.) according the manufacturer's instructions.

The excised DNA was subcloned into plasmid pExpress47 which had been cut with Eco31I (Fermentas, Hanover, Md.). The pExpress47 vector uses the native ZcytoR21 signal peptide and attaches the HIS tag: 5′ GGCTCAGGATCTGGTGGCGGCCATCACCACCATCATCACTAAATCTAGA3′ (SEQ ID NO:125) to the C-terminus of the extracellular portion of the ZcytoR21 polypeptide-encoding polynucleotide sequence. Plasmid pExpress47, is a entry vector containing pDONR221 backbone, Kozak, Eco31I sites for ORF cloning, for seamless ligation to 3′ His tag and Cassette A (Invitrogen) between cloning sites. The plasmid also has a pUC origin of replication, a mammalian selectable marker expression unit.

About 10 μl of the restriction digested ZcytoR21 insert and about 75 ng of the digested vector were ligated using the Fast link ligation kit (EPICENTRE technologies (Madison, Wis.). Two microliter of the ligation reaction was transformed into One shot MAX efficiency DH10B-T1 competent cells (Invitrogen, Carlsbad, Calif.) according to manufacturer's direction and plated onto LB plates containing 25 μg/ml Kanamycin, and incubated overnight. Colonies were submitted for sequencing in 5 ml liquid cultures of individual colonies. The insert sequence of clones was verified by sequence analysis.

An LR reaction was set up using LR reaction kit (Invitrogen, Carlsbad, Calif.), about 300 ng of pExpress 4 expression vector and about 100-300 ng of ZcytoR21/pexpress47 entry clone. Plasmid pExpress4, is a expression vector made by cloning Gateway conversion cassette A into the Nru I site of pEXPRESS-01; a standard vector; modular design; Promoter (Kpn I/Mfe); poly A (Xba I/Hind III); Zeo selection marker (Hind III/Bgl II); E. coli Ori (Bgl II/Kpn I); Gene Amp cassette (Sfi I/Sap I). The reaction contained 4 μl 5×LR reaction buffer, 1 μl of Topoisomerase, 4 μl of LR Clonase enzyme mix and TE buffer for a final volume of 20. Incubated for 1 hour at 25° C., then 2 μl proteinase K added and incubated at 37° C. for 10 minutes. One microliter of the LR reaction was transformed into One shot MAX efficiency DH10B-T1 competent cells (Invitrogen, Carlsbad, Calif.) according to manufacturer's direction and plated onto LB plates containing 50 μg/ml Kanamycin, and incubated overnight. Colonies were screened by PCR and simultaneously inoculating 100 μl of LB broth.

PCR was set up using the following: Advantage 2 reagents (BD Biosciences Clontech, Palo Alto, Calif.) and ZC5020: 5′CACTGGAGTGGCAACTTCCAG3′ (SEQ ID NO:126) and ZC14063: 5′CACCAGACATAATAGCTGACAGACT3′ (SEQ ID NO:127) as PCR primers. PCR amplification of the ZcytoR21 was performed as follows: One cycle of 94C for 2 minutes; then 35 cycles at 94° C. for 30 seconds, 62° C. for 30 seconds, 72° C. for 2 minute, followed by one cycle of 72° C. for 5 minutes and then a 4° C. hold. A band of the predicted size 1468 bp was visualized by 4% agarose gel electrophoresis. 5 ml liquid culture was inoculated with the 100 μl LB clone mix and left ON at 37° C. with shaking.

A mini prep was done using a QIAprep spin Miniprep kit (Qiagen, Santa Clarita, Calif.) according the manufacturer's instructions.

### Example 11

### Construction of Mammalian Expression Vectors that Express Human IL-17C

An expression vector was prepared for the expression of human IL-17C polypeptide, IL-17CCHIS, wherein the construct is designed to express a IL-17C polypeptide comprised of the predicted initiating methionine to the last amino acid minus the stop codon and with a C-terminal HIS tag: 5′GGCTCAGGATCTGGTGGCGGCCATCACCACCATCATCACTAAATCTAGA3′ (SEQ ID NO:128).

A 594 bp PCR generated IL-17C DNA fragment was created using ZC80204″ 5′GAAGAACGTCTCTCATGACGCTCCTCCCCGGCCTCC3′ (SEQ ID NO:129) and ZC80300: 5′GAAGAACGTCTCTAGCCCACTGAACGGGGCAGCACGCAGGTG3′ (SEQ ID NO:130) as PCR primers to add Esp3I restriction sites and Tgo reagents (Roche, Applied Sciences, Indianapolis, Ind.) with or without DMSO (Sigma, ST. Louis, Mo.). A plasmid containing the IL-17C cDNA (Clonetrack ID#100527) was used as a template. PCR amplification of the IL-17C fragment was performed as follows: PCR amplification of the IL-17C fragment was performed as follows: One cycle of 94 C for 2 minutes; then three cycles at 94° C. for 15 seconds, 45° C. for 30 seconds, 72° C. for 2.5 minutes, then nine cycles at 94° C. for 15 seconds, 63° C. for 30 seconds, 72° C. for 2.5 minutes; followed by one cycle of 72° C. for 5 minutes and then a 4° C. hold. The reaction was purified using QIAquick PCR purification kit (Qiagen, Santa Clarita, Calif.) and digested with Esp3I (Fermentas, Hanover, Md.) following manufacturer's protocol. The reaction was purified using QIAquick PCR purification kit (Qiagen, Santa Clarita, Calif.) according the manufacturer's instructions.

The excised DNA was subcloned into plasmid pExpress47 which had been cut with Eco31I (Fermentas, Hanover, Md.). The pExpress47 vector uses the native IL-17C signal peptide and attaches the HIS tag: 5′ GGCTCAGGATCTGGTGGCGGCCATCACCACCATCATCACTAAATCTAGA3′ (SEQ ID NO:131) to the IL-17C polypeptide-encoding polynucleotide sequence. Plasmid pExpress47, is a entry vector containing pDONR221 backbone, Kozak, Eco311 sites for ORF cloning, for seamless ligation to 3′ His tag and Cassette A (Invitrogen) between cloning sites. The plasmid also has a pUC origin of replication, a mammalian selectable marker expression unit.

About 10 μl of the restriction digested IL-17C insert and about 75 ng of the digested vector were ligated using the Fast link ligation kit (EPICENTRE technologies (Madison, Wis.). Two microliter of the ligation reaction was transformed into One shot MAX efficiency DH10B-T1 competent cells (Invitrogen, Carlsbad, Calif.) according to manufacturer's direction and plated onto LB plates containing 25 μg/ml Kanamycin, and incubated overnight. Colonies were submitted for sequencing in 5 ml liquid cultures of individual colonies. The insert sequence of clones was verified by sequence analysis.

An LR reaction was set up using LR reaction kit (Invitrogen, Carlsbad, Calif.), about 300 ng of pExpress 4 expression vector and about 100-300 ng of IL-17C/pexpress47 entry clone. Plasmid pExpress4, is a expression vector made by cloning Gateway conversion cassette A into the Nru I site of pEXPRESS-01; a standard vector; modular design; Promoter (Kpn I/Mfe); poly A (Xba I/Hind III); Zeo selection marker (Hind III/Bgl II); E. coli Ori (Bgl II/Kpn I); Gene Amp cassette (Sfi I/Sap I). The reaction contained 4 μl 5×LR reaction buffer, 1 μl of Topoisomerase, 4 μl of LR Clonase enzyme mix and TE buffer for a final volume of 20. Incubated for 1 hour at 25° C., then 2 μl proteinase K added and incubated at 37° C. for 10 minutes. One microliter of the LR reaction was transformed into One shot MAX efficiency DH10B-T1 competent cells (Invitrogen, Carlsbad, Calif.) according to manufacturer's direction and plated onto LB plates containing 50 μg/ml Kanamycin, and incubated overnight. Colonies were screened by PCR and simultaneously inoculating 100 μl of LB broth.

PCR was set up using the following: Advantage 2 reagents (BD Biosciences Clontech, Palo Alto, Calif.) and ZC5020: 5′CACTGGAGTGGCAACTTCCAG3′ (SEQ ID NO:132) and ZC14063: 5′CACCAGACATAATAGCTGACAGACT3′ (SEQ ID NO:133) as PCR primers. PCR amplification of the IL-17C was performed as follows: One cycle of 94C for 2 minutes; then 35 cycles at 94° C. for 30 seconds, 62° C. for 30 seconds, 72° C. for 2 minute, followed by one cycle of 72° C. for 5 minutes and then a 4° C. hold. A band of the predicted size 942 bp was visualized by agarose gel electrophoresis. 5 ml liquid culture was inoculated with the 1001 μl LB clone mix and left ON at 37° C. with shaking. Glycerol stock archieved at −80° C. Plate was struck with glycerol stock and left ON at 37° C. A 5 ml liquid culture was inoculated with clone and left ON at 37° C. with shaking. 5 ml ON culture used to inoculate 500 ml of liquid culture, left ON at 37° C. with shaking.

A mega prep was done using a QIAfilter plasmid mega kit (Qiagen, Santa Clarita, Calif.) according to an optimized protocols based on manufacturer's instructions.

### Example 12

### Construction of Mammalian Expression Vector for Murine IL-17C

An expression vector was prepared for the expression of mouse IL-17C polypeptide, IL-17CCHIS, wherein the construct is designed to express a IL-17C polypeptide comprised of the predicted initiating methionine to the last amino acid minus the stop codon, and with a C-terminal HIS tag, 5′ GGCTCAGGATCTGGTGGCGGCCATCACCACCATCATCACTAAATCTAGA3′, (SEQ ID NO:134).

A 620 bp PCR generated IL-17C DNA fragment was created using ZC50745: 5′GAAGCCGAAGACTTCATGGCCACCGTCACCGTCACT3′ (SEQ ID NO:135) and ZC50743: 5′GAAGCCGAAGACTTAGCCCTGTGTAGACCTGGGAAGAA3′ (SEQ ID NO:136) as PCR primers to add BbsI restriction sites and Tgo reagents (Roche, Applied Sciences, Indianapolis, Ind.) plus 10% DMSO (Sigma, ST. Louis, Mo.). A plasmid containing the IL-17C cDNA (Clonetrack ID#101619) was used as a template. PCR amplification of the IL-17C fragment was performed as follows: One cycle of 94C for 2 minutes; then three cycles at 94° C. for 15 seconds, 45° C. for 30 seconds, 72° C. for 2.5 minutes, then nine cycles at 94° C. for 15 seconds, 63° C. for 30 seconds, 72° C. for 2.5 minutes; followed by one cycle of 72° C. for 5 minutes and then a 4° C. hold. The reaction was purified using QlAquick PCR purification kit (Qiagen, Santa Clarita, Calif.) and digested with BbsI (Fermentas, Hanover, Md.) following manufacturer's protocol. The reaction was gel extracted using QIAquick gel extraction kit (Qiagen, Santa Clarita, Calif.) according the manufacturer's instructions.

The excised DNA was subcloned into plasmid pExpress47 which had been cut with Eco31I (Fermentas, Hanover, Md.). The pExpress47 vector uses the native IL-17C signal peptide and attaches the HIS tag: 5′GGCTCAGGATCTGGTGGCGGCCATCACCACCATCATCACTAAATCTAGA3′ (SEQ ID NO:137) to the C-terminus of the IL-17C polypeptide-encoding polynucleotide sequence. Plasmid pExpress47, is a entry vector containing pDONR221 backbone, Kozak, Eco31I sites for ORF cloning, for seamless ligation to 3′ His tag and Cassette A (Invitrogen) between cloning sites. The plasmid also has a pUC origin of replication, a mammalian selectable marker expression unit.

About 10 μl of the restriction digested IL-17C insert and about 75 ng of the digested vector were ligated using the Fast link ligation kit (EPICENTRE technologies (Madison, Wis.). Two microliter of the ligation reaction was transformed into One shot MAX efficiency DH10B-T1 competent cells (Invitrogen, Carlsbad, Calif.) according to manufacturer's direction and plated onto LB plates containing 25 μg/ml Kanamycin, and incubated overnight. Colonies were submitted for sequencing in 5 ml liquid cultures of individual colonies. The insert sequence of clones was verified by sequence analysis.

An LR reaction was set up using LR reaction kit (Invitrogen, Carlsbad, Calif.), about 300 ng of pExpress 4 expression vector and about 100-300 ng of IL-17C/pexpress47 entry clone. Plasmid pExpress4, is a expression vector made by cloning Gateway conversion cassette A into the Nru I site of pEXPRESS-01; a standard vector; modular design; Promoter (Kpn I/Mfe); poly A (Xba I/Hind III); Zeo selection marker (Hind III/Bgl II); E. coli Ori (Bgl II/Kpn I); Gene Amp cassette (Sfi I/Sap I). The reaction contained 4 μl 5×LR reaction buffer, 1 μl of Topoisomerase, 4 μl of LR Clonase enzyme mix and TE buffer for a final volume of 20. Incubated for 1 hour at 25° C., then 2 μl proteinase K added and incubated at 37° C. for 10 minutes. One microliter of the LR reaction was transformed into One shot MAX efficiency DH10B-T1 competent cells (Invitrogen, Carlsbad, Calif.) according to manufacturer's direction and plated onto LB plates containing 50 μg/ml Kanamycin, and incubated overnight. Colonies were screened by PCR and simultaneously inoculating 100 μl of LB broth.

PCR was set up using the following: Advantage 2 reagents (BD Biosciences Clontech, Palo Alto, Calif.) and ZC5020: 5′CACTGGAGTGGCAACTTCCAG3′ (SEQ ID NO:138) and ZC14063: 5′CACCAGACATAATAGCTGACAGACT3′ (SEQ ID NO:139) as PCR primers. PCR amplification of the IL-17C was performed as follows: One cycle of 94C for 2 minutes; then 35 cycles at 94° C. for 30 seconds, 62° C. for 30 seconds, 72° C. for 2 minute, followed by one cycle of 72° C. for 5 minutes and then a 4° C. hold. A band of the predicted size 934 bp was visualized by agarose gel electrophoresis. 5 ml liquid culture was inoculated with the 100 μl LB clone mix and left ON at 37° C. with shaking.

A mini prep was done using a QIAprep spin Miniprep kit (Qiagen, Santa Clarita, Calif.) according the manufacturer's instructions.

### Example 13

### Transfection and Expression of Soluble Human IL-17C

On day 1, 5 L of shake flask cultured 293f cells (Invitrogen, Carlsbad, Calif. Cat# R790-07), passage 5-post thaw at 2.4e6 c/ml, were seeded into 4.5 L of Freestyle 293 Expression Medium (Invitrogen, Carlsbad, Calif. Cat# 12338-026) in a Wave Biotech reactor (Wave Biotech, Cat# cell bag 20L/O). 25 mls of a Penicillin-Streptomicin (Invitrogen, Carlsbad, Calif. Cat# 1507-063) mixture was also added at this time. The cells were cultured at 37° C. with ambient airflow @0.2 LPM supplemented with 6% CO2. The reactor was rocked 25 times per minute with an angle setting of 9.5. These settings were utilized for the entire length of the culture.

On day 4, 4.7 L of fresh Freestyle 293 media w/5 mls/L of Penicillin-Streptomicin mixture was then added to the reactor, for a final volume of 9.7L. The cells were then transfected as follows: 0.8 mg/ml mega prep. plasmid DNA (MPET construct #889, IL-17 CcH6), as described in the above Example, was obtained. Two 120 ml aliquots of Optimem media (Invitrogen, Carlsbad, Calif. Cat# 31985-070) were prewarmed to 37° C. Into one Optimem aliquot, 10 mls of the DNA prep was added and mixed. Into the other Optimem aliquot 10.5 mls of Lipofectimine 2000 (Invitrogen, Carlsbad, Calif. Cat# 11668-019) was added and mixed. The two aliquot mixtures were added together, mixed and incubated for 30 minutes at room temp., with occasional mixing. The Lipofectimine 2000/DNA mixture was then added to the reactor.

After 96 hrs post transfection, the culture was harvested, the cells spun out of the media for 10 minutes @ 4000 G's in a Beckman Coulter Avanti J-HC centrifuge. The conditioned media was then passed consecutively through a 1.2 and 0.2 um Millipore Opticap filter set (Millipore Bedford Mass. Cat#s KW1904HB3, KWSSL4HB3). The filtered media was then purified by known methods.

### Example 14

### Transfection and Expression of Soluble Murine IL-17C

On day 1, 1.25L of shake flask cultured 293f cells (Invitrogen, Carlsbad, Calif. Cat# R790-07) passage 22-post thaw at 2e6 c/mil, were seeded into 8.15 L of Freestyle 293 Expression Medium (Invitrogen, Carlsbad, Calif. Cat# 12338-026) in a Wave Biotech reactor (Wave Biotech, Cat# cell bag 20L/O). The cells were cultured at 37° C. with ambient airflow @0.2 LPM supplemented with 6% CO₂. The reactor was rocked 25 times per minute with an angle setting of 9.5. These settings were utilized for the entire length of the culture. On day 4, 700 mls of the culture was extracted and discarded. 1.4L of fresh Freestyle 293 media was then added for a final volume of 10 L. On day 5, 2.6 L of media was extracted and discarded. 1.4L of fresh Freestyle 293 media was added for a final volume of 8.8L @ 2e6 c/ml and the cells were transfected as follows: mega prep plasmid DNA (MPET construct #1280, IL-17 CmCH6) @ 1.88 mg/ml was obtained as described herein. Two 150 ml aliquots of DMEM media (Invitrogen, Carlsbad, Calif. Cat# 119092) were prewarmed to 37° C. Into one DMEM aliquot, 9.4 mls of the DNA prep was added and mixed. Into the other DMEM aliquot 17.6 mls of a 1 mg/ml solution of PEI (Polyethyleneimine, Linear 25 kDa. Cat# 23966. Polysciences, Inc. Warrington Pa.) mixture was added and mixed. The two mixtures were incubated separately at room temperature for 5 minutes, then added together, mixed and incubated for 20 minutes at room temperature with occasional mixing. The PEI/DNA mixture was then added to the reactor. Fifty mls of a Penicillin-Streptomicin mixture was also added at this time (Invitrogen, Carlsbad, Calif. Cat# 1507-063).

After 96 hrs post transfection, the culture was harvested, the cells spun out of the media for 10 minutes @ 4000 G's in a Beckman Coulter Avanti J-HC centrifuge. The conditioned media was then passed consecutively through a 1.2 and 0.2 um Millipore Opticap filter set (Millipore Bedford Mass. Cat#s KW1904HB3, KWSSL4HB3). The filtered media was then purified by known methods.

### Example 15

### ZcytoR21 Luminex Assay

Oligonucleotides specific to unique intron/exon junctions for ZcytoR21 splice variants can be designed for use in a Luminex microsphere-based assay to measure levels of splice variant specific mRNAs. However, it is not possible to design a specific oligo to ZcytoR21x1, as it contains no unique intron/exon junction that the other splice variants lack. For example, ZcytoR21x2 (SEQ ID NO:4), zc49789 (5′gcctcccacacgaggaagctgctgc 3′) (SEQ ID NO:39) is synthesized with a 5′ amine Uni-Link group and its complementary antisense oligonucleotide zc49890 (5′gcagcagcttcctcgtgtgggaggc3′) (SEQ ID NO:40) was synthesized with a 5′ biotin group for monitoring coupling efficiency later in the protocol. ZcytoR21x3 (SEQ ID NO:7) has three unique intron/exon junctions relative to the other ZcytoR21 splice variants, therefore it is necessary to design three sense oligonucleotides, zc49790 (5′tggactcacaaaggacccgagttct3′) (SEQ ID NO:41), zc49891 (5′gcctctgttattccagtctggtggg3′) (SEQ ID NO:42), and zc49892 (5′ccccgttgaagaccgtgtgggaggc3′) (SEQ ID NO:43), each with a 5′ amine Uni-Link group and their complementary antisense 5′ biotin labeled control oligonucleotides, zc49791 (5′cccaccagactggaataacagaggc3′) (SEQ ID NO:44), zc49792 (5′gcctcccacacggtcttcaacgggg3′) (SEQ ID NO:45), and zc49724 (5′agaactcgggtcctttgtgagtcca3′) (SEQ ID NO:46). ZcytoR21x4 (SEQ ID NO:10) specific sense oligonucleotide zc49793 (5′tgctgtgtcctgctccatgcttcac3′) (SEQ ID NO:47) is synthesized with a 5‘amine Uni-Link group and it’s 5′ biotin labeled antisense complement, zc49729, (5′gtgaagcatggagcaggacacagca3′) (SEQ ID NO:48) is also synthesized. To assess the efficiency of the RNA amplification step in amplifying long mRNAs, oligos are designed to the first and last exons of ZcytoR21, which are common to all known splice variants. For the first exon of ZcytoR21, zc49794 (5′tctgactctgctgggattggctttc3′) (SEQ ID NO:49) is synthesized with a 5‘amine Uni-Link group and it’s complementary antisense oligonucleotide zc49893 (5′gaaagccaatcccagcagagtcaga3′) (SEQ ID NO:50) is synthesized with a 5′ biotin group. For the last exon of ZcytoR21, zc49795 (5′tgctgctgctgtggagcggcgccga3′) (SEQ ID NO:51) is synthesized with a 5‘amine Uni-Link group and it’s complement zc49894 (5′tcggcgccgctccacagcagcagca3′) (SEQ ID NO:52). The ratio of the measurements of the first and last exons can be used to qualitatively assess the impact of measuring the levels of a sequence target that is not near the 3′ end of an mRNA, such as the unique intron/exon junction specific to ZcytoR21x2.

Each sense oligonucleotide is coupled to specific xMAP™ Multi Analysis Carboxylated Microspheres (Luminex Corporation, Austin, Tex.) as follows: stock microspheres are resuspended by vortex and sonication for approximately 20 seconds, 200 μl (2.5×10⁶ microspheres) are transferred to a microfuge tube and pelleted by microcentrifugation at >8000×g for 1-2 minutes. Supernatents are removed and the microsphere pellets are resuspended in 50 ul of 0.1M MES (2(N-Morpholino) ethanesulfonic acid, Sigma, St. Louis, Mo.), ph4.5, by vortex and sonication. A fresh solution of 10 mg/ml EDC carbodimide HCL (1-Ethyl-3- (3-dimethylaminopropyl) carbodimide HCl, Pierce, Rockford, Ill.) is prepared in dH₂O and 2.5 ul of this solution is added to the microspheres, vortexed and incubated at room temperature 30 minutes in the dark. A second fresh solution of 10 mg/ml EDC is prepared, 2.5 ul is added to the microspheres, and incubation in the dark for 30 minutes is repeated. A third iteration of the EDC addition and incubation is optional. 1 ml of 0.02% Tween20 (Polyoxyethylenesorbitan monolaurate, Sigma, St. Louis, Mo.) is added to the coupled microspheres and mixed by vortexing and pelleted by microcentrifugation. The supernatent is removed and the microsphere pellets are resuspended in 1 ml of 0.1% SDS (Lauryl Sulfate, Sigma, St. Louis, Mo.) by vortexing and pelleted by centrifugation. The supernatent is removed and pellets are resuspended in 100 μl of TE, ph 8.0 by vortexing and sonication for about 20 seconds. Microspheres are enumerated by using a hemacytometer and stored at 4° C. in the dark until use.

Coupling and hybridization efficiency of the microspheres is evaluated by mixing the coupled microspheres with the biotin labeled complementary oligonucleotide as follows: The coupled microspheres are resuspended by vortex and sonication for about 20 seconds, and a working mixture is prepared by diluting coupled microsphere stocks to 150 microspheres/ul in 1.5× TMAC hybridization buffer (4.5M TMAC (Sigma, St. Louis, Mo.) 0.15% Sarkosyl, 0.75 mM Tris-HCl, pH8 (Sigma, St. Louis, Mo.), 6 mM EDTA, pH 8.0 (Gibco, Grand Island, N.Y.). To each sample or background well in a MicroAmp optical 96 well reaction plate (Applied Biosystems, Foster City, Calif.) 33.3 ul of coupled microspheres is added, and to each background well is added 16.67 ul TE, pH 8.0. The appropriate biotinylated complementary oligonucleotide over a range from 5 to 200 femtomoles, adjusted to a final volume of 16.7 ul, is added to each sample well, the plate is sealed and reactions are mixed with a plate shaker at 400 rpm. Plates are incubated at 94° C. for 3 minutes; then 55° C. for 15 minutes. A vacuum manifold (Millipore Corporation, Billerica, Mass.) is used to remove unbound oligonucleotides and the plate is washed 3 times with 100 ul/well wash buffer (1 mM PBS, 0.01% Tween® 20), removing the buffer each time by vacuum filtration. Fresh reporter mix is prepared by diluting streptavidin-R-phycoerythrin conjugate (Molecular Probes, Eugene, Oreg.) to 4 ug/ml in wash buffer, 75 ul is added to each well, the assay plate is covered with foil and mixed on a plate shaker at 1100 rpm for 30 seconds, then incubated at room temperature for 15 minutes at 400 rpm. The plate is then washed 3× to remove unbound streptavidin-PE, and samples are resuspended in a final volume of 75 ul wash buffer. 50 ul are then analyzed on a Bio-Plex Array Reader (BioRad Laboratories, Inc, Hercules, Calif.).

Approximately 2×10⁶ U937 cells are plated and stimulated with 20 ng/ml PMA and 20 ng/ml PMA+0.5 ug/ml ionomycin for 6, 11 and 24 hours. ˜2×106 THP1 cells are stimulated with PMA at 100 ng/ml for 12, 24 and 48 hours. Cells are harvested and total RNA is purified using a Qiagen (Valencia, Calif.) RNeasy kit according to the manufacturer's instructions with the optional DNAse step incorporated into the protocol. The RNA is DNAsed using DNA-free reagents (Ambion, Inc, Austin, Tex.) according to the manufacturer's instructions. The quality of the RNA is assessed by running an aliquot on an Agilent Bioanalyzer. If the RNA is significantly degraded, it is not used for subsequent assays for ZcytoR21 mRNAs. Presence of contaminating genomic DNA is assessed by a PCR assay on an aliquot of the RNA with zc37263 (5′gaattacaccctctggagagtgg 3′) and zc37264 (5′ gaatttcggacaatccagtactc 3′), primers that amplify a single site in genomic DNA within an intron at the cathepsin Z gene locus. The PCR conditions for the contaminating genomic DNA assay are as follows: 2.5 ul 10× buffer and 0.5 ul Advantage 2 cDNA polymerase mix (BD Biosciences Clontech, Palo Alto, Calif.), 2 ul 2.5 mM dNTP mix (Applied Biosystems, Foster City, Calif.), 2.5 ul 10× Rediload (Invitrogen, Carlsbad, Calif.), and 0.5 ul 20 uM zc37263 and zc37264, in a final volume of 25 ul. Cycling parameters are 94° C. 20″, 40 cycles of 94° C. 20″ 62° C. 20″ 72° C. 1′ and one cycle of 72° C. 7′. 10 ul of each reaction is subjected to agarose gel electrophoresis and gels are examined for presence of a PCR product from contaminating genomic DNA. Only RNAs that appear to be free of contaminating genomic DNA are used in subsequent assays for ZcytoR21 splice variant mRNAs.

5 μg of each RNA to be assayed for ZcytoR21 splice variants using coupled Luminex microspheres is first amplified using an Ambion MessageAmp™ aRNA Kit (Ambion Incorporated, Austin, Tex.) according to the manufacturer's instructions, but modifying the In-Vitro transcription step synthesizing the antisense RNA such that labeled dNTPs (biotin-16-UTP and biotin-11-CTP, Perkin-Elmer Life Sciences, Boston, Mass.) are used instead of the dNTPs provided with the kit. Levels of ZcytoR21 splice variant mRNAs are determined in each amplified RNA sample as follows: appropriate housekeeping gene control oligonucleotide coupled microsphere and ZcytoR21 splice variant specific oligonucleotide coupled microspheres are used to prepare a working microsphere mixture by diluting the coupled microsphere stocks to 5000 per 33.3 ul in 1.5× TMAC hybridization buffer; the total volume being 33.3 ul multiplied by the number of sample and background wells to be tested. Mix this working microsphere solution by vortex and sonication for about 20 seconds. To each background well, add 16.7 ul TE, pH 8.0, and to each sample well add 5 ug of the amplified biotinylated RNA, which is first heated to 94° C. for 35 minutes and iced, in a volume of 16.7 ul TE, pH 8.0. To each sample and background well is added 33.3 ul of the working microsphere mixture, and wells are mixed by pipetting up and down, and shaking briefly on a plate shaker. The plate is sealed and incubated at 94° C. for 10 minutes to denature the amplified biotinylated RNA, then incubated at 60° C. in a shaking incubator for 5 hours with gentle rocking. The reactions are transferred to a microtiter plate, a vacuum manifold is used to separate the unbound nucleotides and wash the plate, and reporter mix is incubated with the samples as described above. Plates are then washed and counted in a Bio-Plex Array Reader as described above.

Results may demonstrate that in comparison to THP1, ZcytoR21 transcripts in U937 cells are expressed at a much higher level, regardless of the presence or absence of PMA. Additionally, significant expression of each splice variant ZcytoR21x2, x3 and x4 is observed. By inference the variant ZcytoR21x1 and/or possible splice variants that are as yet undescribed are also highly expressed in U937 relative to THP1; because of the high levels of expression of the last exon.

### Example 16

### Northern and Dot Blot Analysis of ZcytoR21

Northern and dot blot analyses were performed using Human Multiple Tissue Blots I, II, and III and the Human RNA Master Blot (CLONTECH Laboratories, Inc., Palo Alto, Calif.). A 1.4 kb DNA fragment was generated by digesting DNA of a ZcytoR21x1 (SEQ ID NO:1) cDNA with EcoR1 and Not, followed by gel electrophoresis and purification of the fragment using Qiaquick Gel Extraction reagents and protocol. (Qiagen, Valencia, Calif.). The DNA fragment encompassed the sequence encoding amino acids #257-690 of SEQ ID NO:2 and is predicted to hybridze to all known splice variants of ZcytoR21. The fragment was radioactively labeled using the Redi-Prime II kit (Stratagene, La Jolla, Calif.) according to the manufacturer's protocol. The probe was purified using a MicroSpin S-200 HR spin column (Amersham, Arlington Heights, Ill.) according to the manufacturer's instructions. Salmon sperm DNA (Stratagene, La Jolla, Calif.) and Cot-1 DNA (Invitrogen, Carlsbad, Calif.) were boiled 5′, snap-chilled on ice, added to ExpressHyb (CLONTECH) at 100 μg/ml and 6 μg/ml, respectively, and used as prehybridization and hybridization solutions for the blots. Prehybridization took place for 3 hours at 55 C. The radioactively labeled DNA fragment was boiled 5′, snap-chilled on ice and added to the blots at 1×10⁶ cpm/ml hybridization solution. Hybridization took place overnight at 55 C. Following hybridization, the blots were washed as follows: twice in 2×SSC, 0.1% SDS at room temperature, one time in 2×SSC, 0.1% SDS at 65 C, followed by one 20′ wash in 0.1×SSC, 0.1% SDS at 65 C. The blots were exposed to film overnight The results are illustrated in the figures below, and demonstrate ZcytoR21 mRNA is widely expressed, being most strongly expressed in stomach, pancreas and expressed to a lesser extent in prostate, thyroid, trachea, salivary gland, liver, kidney, small intestine, lung, fetal lung, fetal thymus, placenta, mammary gland, heart, cerebellum, caudate nucleus, and colon. In contrast, there is little or no expression in whole brain, skeletal muscle, spleen, thymus, testis, ovary, peripheral blood leukocytes, spinal cord, lymph node, adrenal gland, uterus, bladder, fetal whole brain, fetal heart, fetal liver, fetal spleen, and bone marrow.

### Example 17

### Northern, Dot Blot and Disease Array Analysis of IL-17C

Northern, dot blot, and disease array analyses were performed using Human Multiple Tissue Blots I and III, Human Fetal Multiple Tissue Blot II, Human RNA Master Blot, Cancer Profiling Array II, Blood Disease Profiling Array, Autoimmune Disease Profiling Array, and the Cancer Cell Line Profiling Array. (CLONTECH Laboratories, Inc., Palo Alto, Calif.). A ˜770 bp DNA fragment was generated by digesting IL-17C cDNA with EcoR1, followed by gel electrophoresis and purification of the fragment using Qiaquick Gel Extraction reagents and protocol. (Qiagen, Valencia, Calif.). The DNA fragment encompassed the sequence encoding the complete open reading frame of IL-17C. The fragment was radioactively labeled using the Redi-Prime II kit (Stratagene, La Jolla, Calif.) according to the manufacturer's protocol. The probe was purified using a MicroSpin S-200 HR spin column (Amersham, Arlington Heights, Ill.) according to the manufacturer's instructions. Salmon sperm DNA (Stratagene, La Jolla, Calif.) and Cot-1 DNA (Invitrogen, Carlsbad, Calif.) were boiled 5′, snap-chilled on ice, added to ExpressHyb (CLONTECH) at 100 ug/ml and 6 ug/ml, respectively, and used as prehybridization and hybridization solutions for the blots. Prehybridization took place overnight at 55° C. The radioactively labeled DNA fragment was boiled 5′, snap-chilled on ice and added to the blots at 1×10⁶ cpm/ml hybridization solution. Hybridization took place overnight at 55° C. Following hybridization, the blots were washed as follows: twice in 2×SSC, 0.1% SDS at room temperature, one time in 2×SSC, 0.1% SDS at 65° C., followed by one 20′ wash in 0.1×SSC, 0.1% SDS at 65° C. The blots were exposed to film with intensifying screens for six days.

The results generally demonstrate that IL-17C mRNA is not widely or highly expressed. A transcript of ˜1.4 kb is visible in fetal lung, but no IL-17C transcript is present in fetal brain, fetal liver, or fetal kidney. In adult tissues a transcript of ˜4.8 kb is visible in heart and two transcripts of ˜5 kb and 3 kb are visible in skeletal muscle. In contrast, no IL-17C transcript is observable in brain, placenta, lung, liver, kidney, pancreas, stomach, thyroid, spinal cord lymph node, trachea, adrenal gland or bone marrow. In the cancer profiling array, IL-17C is relatively absent in normal and tumor cDNAs from multiple patients with cancer of the breast, ovary, colon, stomach, lung, kidney, bladder, vulva, prostate, trachea, uterus, cervix, rectum, thyroid gland, testis, skin and pancreas cancer. However, slightly higher IL-17C hybridization is observable in the normal liver and small intestine from several patients with cancers of those same tissues. In the Autoimmune and Blood Disease profiling arrays, IL-17C mRNA can be seen to be slightly increased in the CD19 (primarily B-cell) fraction of the blood across the board in normal and diseased patients, relative to the levels of IL-17C mRNA in CD14 (primarily monocye), CD3 (primarily T cell), Mononuclear cells and Polymorphonuclear cells Interestingly, the IL-17C mRNA levels appear to be further elevated in the CD19 blood fraction in patients with Multiple Sclerosis, Von Willebrand's Disease, Lupus Anticoagulans, Takayasu's Arthritis, Idiopathic Thrombocytopenic Purpura, Hodgkin's disease, and Chronic Myelogenous Leukemia, relative to normal patient CD19 blood fraction levels of IL-17C. In the cancer cell line profiling array, IL-17C again is not highly or widely expressed, but it is visible in a scattered few cell lines under certain conditions. MDA-MB-435S stimulated with Cytochalasine D and U-87 MG stimulated with Demecolcine, Miomycine, Actinomycin D and Cyclohexamide all appear to express low levels of IL-17C mRNA while the other 24 cell lines combined with stimulation conditions express little to no IL-17C mRNA.

### Example 18

### Transient Expression of ZcytoR21

Human ZcytoR21x1 (SEQ ID NO:1) and x2 (SEQ ID NO:4) cDNAs were placed in a dicistronic expression vector, pzmp11. The cDNAs were inserted downstream of the cmv promoter, followed by an IRES site and a cDNA for the cell surface marker, human CD8. CD8 expression correlates with transcription of the inserted cDNA and can be used to facs sort for CD8 cells and ask if that population correlates with binding events, vs the non-CD8 population.

293FB suspension cells were seeded into 125 ml tissue culture erlenmeyer fermenter flasks at a density of 10⁶ cells/ml in 10 ml fresh Freestyle 293 expression medium (Invitrogen). 10 μg of ZcytoR21x1-pzmp11, ZcytoR21x2-pzmp11 and empty pzmp11 vector were transfected into these cells using lipofectamine 2000 (Invitrogen) 24-78 hours after transfection, cells were used in the binding experiments, as provided herein.

### Example 19

### Creation of a Stable nih3t3 Assay Clone Expressing ap/nfkb Transcription Factor

Murine nih3t3 cells were stably transfected with the kz142 apl/nfkb luciferase reporter construct containing a neomycin-selectible marker. The Neo resistant transfection pool was plated at clonal density. Clones were isolated using cloning rings and screened by luciferase assay using the human IL-17C ligand as an inducer. Clones with the highest mean fluorescence intensity (MFI) (via apl/NfkB luciferase) and the lowest background were selected. A stable transfectant cell line was selected and called nih3t3/kz142.8.

### Example 20

### Murine nih3t3 Cells Express ZcytoR21

Two-step PCR analysis of nih3t3 RNA demonstrated that these cells are positive for ZcytoR21 transcription, consistent with their signaling response to IL-17C being mediated through this receptor. First strand cDNA was prepared from total RNA isolated from nih3t3 cells using standard methods. PCR was applied using hot star polymerase and the manufacturer's recommendations, (Qiagen, Valencia, Calif.) except for utilizing 10% DMSO final concentration. The primers utilized included sense primer, zc40413 (5′ tgcgcccggatcctacagaagc 3′) (SEQ ID NO:55) and antisense primer, zc 40412 (5′gcacctcgggcagcaaatcaaag 3′) (SEQ ID NO:56) Agarose gel electrophoresis revealed a single, robust amplicon of the expected size.

### Example 21

### Creation of Cell Lines with Recombinant Over-Expression of ZcytoR21 Splice Variants

Stable recombinant over expression of human ZcytoR21 facilitates identification of its ligand by increasing sensitization of target cells to activation and binding by its ligand. This phenomenon has been observed for homologs of ZcytoR21. Ligand activation occurred with far lower concentrations than that seen in the same cells, lacking recombinant receptor over expression. This activation phenomenon was observed in a murine nih3t3/kz142.8 cell line, which was shown to express these receptors endogenously. Ligand binding studies were done in recombinant ZcytoR21 over expressing baby hamster kidney cells (BHK570).

Stable over Expression of Human and Mouse ZcytoR21 in the Murine Assay Cell line,nih3t3/kz142.8

Murine nih3t3/kz142.8 (Example 17) were shown to produce endogenous ZcytoR21 mRNA by PCR (Example 18). These cells were transfected with cDNAs of human ZcytoR21x1 (SEQ ID NO:1), ZcytoR21x2 (SEQ ID NO:4) ZcytoR21x3 (SEQ ID NO:7), ZcytoR21x6 (SEQ ID NO:20), ZcytoR21x13 (SEQ ID NO:106) and mouse ZcytoR21x6 (SEQ ID NO:13) in pZMP11, a dicistronic expression vector with a CMV promoter driving transcription inserted cDNA transcription, followed by an IRES, followed by a cDNA for human CD8. CD8 expressing cells can be selected for and correlated with expression of the inserted cDNAs. Pzmp11 has a methotrexate resistance gene. (dihydrofolate reductase,) Transfections were performed using a commercially available kit and the manufacturer's recommendations. (Mirus, Madison, Wis. Cat. #MIR218) Cells were placed in 1 μM mtx amended growth medium to select for the expression constructs containing the human and mouse ZcytoR21 transgenes. After selection, transfection pools were generated, and called nih3t3/kz 142.8/hcytor2 1×1, nih3t3/kz 142.8/hcytor21x2, nih3t3/kz 142.8/hcytor21x3, nih3t3/kz 142.8/hcytor21x6, nih3t3/kz 142.8/hcytor21x13 and nih3t3/kz142.8/mcytor21x6.

Stable Over Expression of Human and Mouse ZcytoR21 in the Baby Hamster Kidney Cell Line, (BHK570)

Baby Hamster Kidney cells (BHK570) were chosen for recombinant over-expression of ZcytoR21 for binding studies. These cells were transfected with cDNAs of human ZcytoR21x1 (SEQ ID NO:1), ZcytoR21x2 (SEQ ID NO:4) ZcytoR21x3 (SEQ ID NO:7), ZcytoR21x6 (SEQ ID NO:20), ZcytoR21x13 (SEQ ID NO:106) and mouse ZcytoR21x6 (SEQ ID NO:13) in pZMP11, a dicistronic expression vector with a CMV promoter driving transcription inserted cDNA transcription, followed by an IRES, followed by a cDNA for human CD8. CD8 expressing cells can be selected for and correlated with expression of the inserted cDNAs. Pzmp11 has a methotrexate resistance gene. (dihydrofolate reductase) Transfections were performed using a commercially available kit and the manufacturer's recommendations. (Mirus, Madison, Wis. Cat. #M[R218) Cells were placed in 1M mtx amended growth medium to select for the expression constructs containing the human and mouse ZcytoR21 transgenes. After selection, transfection pools were generated, and called BHK/hcytor21x1, BHK/hcytor21x2, BHK/hcytor21x3, BHK/hcytor211×6, BHK/hcytor21x13, and BHK/mcytor21x6.

### Example 22

### Distribution of ZcytoR21 mRNA in Cell Line Panels Using PCR

Total RNA was purified from resting and stimulated cell lines grown in-house and purified using a Qiagen (Valencia, Calif.) RNeasy kit according to the manufacturer's instructions, or an acid-phenol purification protocol (Chomczynski and Sacchi, Analytical Biochemistry, 162:156-9, 1987). The quality of the RNA was assessed by running an aliquot on an Agilent Bioanalyzer. If the RNA was significantly degraded, it was not used for subsequent creation of first strand cDNA. Presence of contaminating genomic DNA was assessed by a PCR assay on an aliquot of the RNA with zc41011 (5′ctctccatccttatctttcatcaac3′) (SEQ ID NO: 57) and zc41012 (5′ctctctgctggctaaacaaaacac3′) (SEQ ID NO:58), primers that amplify a single site of intergenic genomic DNA. The PCR conditions for the contaminating genomic DNA assay were as follows: 2.5 ul 10× buffer and 0.5 ul Advantage 2 cDNA polymerase mix (BD Biosciences Clontech, Palo Alto, Calif.), 2 ul 2.5 mM dNTP mix (Applied Biosystems, Foster City, Calif.), 2.5 ul 10× Rediload (Invitrogen, Carlsbad, Calif.), and 0.5 ul 20 uM zc41011 and zc41012, in a final volume of 25 ul. Cycling parameters were 94° C. 20″, 40 cycles of 94° C. 20″ 60° C. 1′20″ and one cycle of 72° C. 7′. 10 ul of each reaction was subjected to agarose gel electrophoresis and gels were examined for presence of a PCR product from contaminating genomic DNA. If contaminating genomic DNA was observed, the total RNA was DNAsed using DNA-free reagents (Ambion, Inc, Austin, Tex.) according to the manufacturer's instructions, then retested as described above. Only RNAs which appeared to be free of contaminating genomic DNA were used for subsequent creation of first strand cDNA.

20 ug total RNA from 82 human cell lines were each brought to 98 ul with H₂O, then split into two 49 ul aliquots, each containing 10 ug total RNA, and placed in two 96-well PCR plates. To each aliquot was added reagents for first strand cDNA synthesis (Invitrogen First Strand cDNA Synthesis System, Carlsbad, Calif.): 20 ul 25 mM MgCl₂, 10 ul 10× RT buffer, 10 ul 0.1M DTT, 2 ul oligo dT, 2 ul RNAseOut. Then, to one aliquot from each cell line 2 ul Superscript II Reverse Transcriptase was added, and to the corresponding cell line aliquot 2 ul H₂O was added to make a minus Reverse Transcriptase negative control. All samples were incubated as follows: 25° C. 10′, 42° C. 50′, 70° C. 15′. Samples were arranged in deep well plates and diluted to 1.7 ml with H₂O. A Multipette (Saigan) robot was used to aliquot 16.5 ul into each well of a 96-well PCR plate multiple times, generating numerous one-use PCR panels of the cell lines, which were then sealed and stored at −20° C. Each well in these panels represents first strand cDNA from approximately 100 ng total RNA. The 82 cell lines were spread across two panels, array #118A and #118B.

Quality of first strand cDNA on the panels was assessed by a multiplex PCR assay on one set of the panels using primers to two widely expressed, but only moderately abundant genes, CLTC (clathrin) and TFRC (transferrin receptor C). 0.5 ul each of Clathrin primers zc42901 (5′ctcatattgctcaactgtgtgaaaag 3′) (SEQ ID NO: 59), zc42902 (5′tagaagccacctgaacacaaatctg3′) (SEQ ID NO:60), and TFRC primers zc42599 (5′atcttgcgttgtatgttgaaaatcaatt3′) (SEQ ID NO:61), zc42600 (5′ttctccaccaggtaaacaagtctac3′) (SEQ ID NO:62), were mixed with 2.5 ul 10× buffer and 0.5 ul Advantage 2 cDNA polymerase mix (BD Biosciences Clontech, Palo Alto, Calif.), 2 ul 2.5 mM dNTP mix (Applied “Biosystems, Foster City, Calif.), 2.5 ul 10× Rediload (Invitrogen, Carlsbad, Calif.), and added to each well of a panel of array#118A and array #118B. Cycling parameters were as follows: 94° C. 20″, 35 cycles of 94° C. 20″, 67° C. 80”, and one cycle of 72° C. 7′. 10 ul of each reaction was subjected to agarose gel electrophoresis and gels were scored for the presence of a robust PCR product for each gene specific to the +RT wells for each cell line.

Expression of mRNA in the human first strand cDNA panels for ZcytoR21 was assayed by PCR with sense oligo zc40450 (5′tcctgcctctcctcctcatagtca3′) (SEQ ID NO:63) and antisense oligo zc40454 (5′ccaggatcaagagccccaggtgtc3′) (SEQ ID NO:64) under these PCR conditions per sample: 2.5 ul 10× buffer and 0.5 ul advantage 2 cDNA polymerase mix (BD Biosciences Clontech, Palo Alto, Calif.), 2 ul 2.5 mM dNTP mix (Applied Biosystems,), 2.5 ul 10× Rediload (Invitrogen, Carlsbad, Calif.), and 0.5 ul 20 uM each sense and antisense primer. Primers were predicted to pick up all known splice variants of ZcytoR21, but they did not necessarily distinguish between each variant. Cycling conditions were 94° C. 20″, 35 cycles of 94° C. 20″, 69° C. 2′30″, and one cycle of 72° C. 7′. 10 ul of each reaction was subjected to agarose gel electrophoresis and gels were scored for positive or negative expression of ZcytoR21. Results showed widespread expression of ZcytoR21mRNA in cell lines by this assay. ZcytoR21 was consistently and usually strongly positive in U-937 (unstimulated and stimulated with PMA or PMA/Ionomycin), B-lymphomas (DOHH-2 Ramos, Granta-519 and RL), and several cell lines from the digestive system (CaCO2, CaCO2 differentiated, HCT-15, and HCT-116). Overall, samples that were positive for ZcytoR21 were: L363, A375, CTB-1+PMA/Ionomycin, TF1, ARH77, G-361, MacLLC+PMA/Ionomycin, DOHH-2, REH, HaCat, Ramos, Granta-519, RL, Hs294T, HL60+butyric acid, AsPC-1, A-172. Hep G2, U937+PMA/Ionomycin, TrBMEC, HepG2+IL6, U937+PMA, ME180, ARPE, A-549, U937, CaCO2, MRC-5, PC-3, CaCO2 differentiated, DLD-1, SKLU-1, Int407, HCT116, and HCT15.

### Example 23

### Distribution of Murine ZcytoR21 mRNA in Murine Cell Line Panels Using PCR

Total RNA was purified from 60 resting and stimulated cell lines grown in-house and purified using a Qiagen (Valencia, Calif.) RNeasy kit according to the manufacturer's instructions, an acid-phenol purification protocol (Chomczynski and Sacchi, Analytical Biochemistry, 162:156-9, 1987), or a Trizol reagent protocol (Invitrogen, Carlsbad, Calif.). 5 ug of total RNA from each cell line was arranged in a deep well 96-well plate, 125 ul 3M NaOAc and 100 ul Pellet Paint (Novagen, Madison, Wis.)) were added to each well, then the final volume was adjusted to 1.25 ml with H₂O. A Multipette (Saigan) robot was used to aliquot 25 ul of the RNA mixture followed by 75 ul EtOH into each well of a 96-well PCR plate multiple times, generating numerous one-use RT PCR panels of the cell lines, each well with 100 ng total RNA in EtOH. Panels were then sealed and stored at −20° C. The arrangement and content of the samples on this array are detailed below in Table 1. RT PCR screening was performed by first centrifuging a panel in a Qiagen (Valencia, Calif.) 96-well centrifuge for 10′ at 6000 RPM. Supernatant was removed by inverting the plate onto absorbent paper. RNA pellets were washed with 100 ul 70% EtOH, followed by a 5′ centrifugation at 6000 RPM. Supernatant was again removed and plates allowed to air-dry until the remaining EtOH was evaporated.

Expression of ZcytoR21m mRNA in the mouse cell line RNA panels was assayed by RT PCR with sense oligo ZC40403 (5′ctgtgaggcgcaaaaagtgtc3′) (SEQ ID NO:81) and antisense oligo ZC48516 (5′gcaagtccacattctccaggat3′) (SEQ ID NO:82) using Superscript One-Step RT PCR reagents (Invitrogen, Carlsbad, Calif.). RNA pellets were resuspended in a total volume of 25 ul/well reaction mix that contained 2.5 ul 10× Rediload (Invitrogen, Carlsbad, Calif.), 12.5 ul 2× Reaction Mix, 0.5 ul of 20 pmol/ul sense oligo, 0.5 ul of 20 pmol/ul antisense oligo, 0.5 ul RT/Platinum Taq and 8.5 ul sterile water. Cycling conditions were: 1 cycle at 52° C. for 30 minutes, 1 cycle at 94° C. for 2 minutes, 35 cycles at 94° C. for 30 seconds, 55° C. for 30 seconds and 72° C. for 1 minute, followed by a final cycle at 72° C. for 7 minutes. 10 ul of each reaction was subjected to agarose gel electrophoresis and gels were scored for positive or negative expression of ZcytoR21m. The primers were predicted to pick up all known splice variants and not produce a product on contaminating genomic DNA. Results indicated presence of ZcytoR21 mRNA in 14 cell lines, most representing lines of pancreatic origin: pik10, pik15, pik18, pik 34, pid14, pid20 5FU-17 and 5FU-19. ZcytoR21 mRNA was also present in C2C12, a skeletal muscle myoblast cell line, RAW 264.7, a monocyte cell line, SAG-5/22-6, a salivary gland cell line, and AML, a liver cell line. In constrast, ZcytoR21m RNA was not expressed in T or B lymphocyte cell lines, embryonic cell lines, adipocyte cell lines, osteoblast and osteoclast cell lines, and hypothalamus cell lines. There were also 10 pancreatic cell lines and 4 salivary gland cell lines that did not express ZcytoR21.

### Example 24

### Construction of Mammalian Soluble ZcytoR21x1 Expression Constructs that Express ZcytoR21x1CEE, ZcytoR21x1CHIS, and ZcytoR21x1CFLAG Tagged Proteins

An expression construct containing the extracellular domain of human ZcytoR21x1 with a C-terminal tag, either Glu-Glu (CEE), six His (CHIS), or FLAG (CFLAG), is constructed via PCR and homologous recombination using a DNA fragment encoding ZcytoR21x1 (SEQ ID NO: 83) and the expression vector pZMP20.

The PCR fragment encoding ZcytoR21x1CEE contains a 5′ overlap with the pZMP20 vector sequence in the optimized tissue plasminogen activator pre-pro secretion leader sequence coding region, the ZcytoR21x1 extracellular domain coding region (SEQ ID NO: 84), the Glu-Glu tag (Glu Glu Tyr Met Pro Met Glu) coding sequence, and a 3′ overlap with the pZMP20 vector in the poliovirus internal ribosome entry site region. The PCR amplification reaction uses the following 5′ oligonucleotide (GTTTCGCTCAGCCAGGAAATCCATGCCGAGTTGAGACGCTTCCGTAGAGCT GGGATTGGCTTTCGCCAC) (SEQ ID NO:85), the following 3′ oligonucleotide (CAACCCCAGAGCTGTTTTAAGGCGCGCCTCTAGATTATTCCATGGGCATGT ATTCTTCGTAAGAGACATCTGGACACA) (SEQ ID NO:86), and a previously generated DNA clone of ZcytoR21x1 as the template (SEQ ID NO:83).

The PCR amplification reaction condition is as follows: 1 cycle, 94° C., 5 minutes; 35 cycles, 94° C., 1 minute, followed by 55° C., 2 minutes, followed by 72° C., 3 minutes; 1 cycle, 72° C., 10 minutes. The PCR reaction mixture is run on a 1% agarose gel and the DNA fragment corresponding to the expected size is extracted from the gel using a QIAquick™ Gel Extraction Kit (Qiagen, Cat. No. 28704).

Plasmid pZMP20 is a mammalian expression vector containing an expression cassette having the chimeric CMV enhancer/MPSV promoter, a BglII site for linearization prior to yeast recombination, an otPA signal peptide sequence, an internal ribosome entry element from poliovirus, the extracellular domain of CD8 truncated at the C-terminal end of the transmembrane domain; an E. coli origin of replication; a mammalian selectable marker expression unit comprising an SV40 promoter, enhancer and origin of replication, a DHFR gene, and the SV40 terminator; and URA3 and CEN-ARS sequences required for selection and replication in S. cerevisiae.

The plasmid pZMP20 is digested with BglII prior to recombination in yeast with the gel extracted ZcytoR21x1CEE PCR fragment. One hundred [l of competent yeast (S. cerevisiae) cells are combined with 10 μl of the ZcytoR21x1CEE insert DNA and 100 ng of BglII digested pZMP20 vector, and the mix is transferred to a 0.2 cm electroporation cuvette. The yeast/DNA mixture is electropulsed using power supply (BioRad Laboratories, Hercules, Calif.) settings of 0.75 kV (5 kV/cm), ∞ ohms, and 25 μF. Six hundred μl of 1.2 M sorbitol is added to the cuvette, and the yeast is plated in 100 μl and 300 ill aliquots onto two URA-D plates and incubated at 30° C. After about 72 hours, the Ura⁺ yeast transformants from a single plate are resuspended in 1 ml H₂O and spun briefly to pellet the yeast cells. The cell pellet is resuspended in 0.5 ml of lysis buffer (2% Triton X-100, 1% SDS, 100 mM NaCl, 10 mM Tris, pH 8.0, 1 mM EDTA). The five hundred μl of the lysis mixture is added to an Eppendorf tube containing 250 μl acid-washed glass beads and 300 μl phenol-chloroform, is vortexed for 3 minutes, and spun for 5 minutes in an Eppendorf centrifuge at maximum speed. Three hundred μl of the aqueous phase is transferred to a fresh tube, and the DNA is precipitated with 600 μl ethanol, followed by centrifugation for 30 minutes at maximum speed. The tube is decanted and the pellet is washed with 1 mL of 70% ethanol. The tube is decanted and the DNA pellet is resuspended in 30 μl 10 mM Tris, pH 8.0, 1 mM EDTA.

Transformation of electrocompetent E. coli host cells (DH12S) is done using 5 μl of the yeast DNA preparation and 50 μl of E. coli cells. The cells are electropulsed at 2.0 kV, 25 μF, and 400 ohms. Following electroporation, 1 ml SOC (2% Bacto™ Tryptone (Difco, Detroit, Mich.), 0.5% yeast extract (Difco), 10 mM NaCl, 2.5 mM KCl, 10 mM MgCl₂, 10 mM MgSO₄, 20 mM glucose) is added and then the cells are plated in 50 μl and 200 μl aliquots on two LB AMP plates (LB broth (Lennox), 1.8% Bacto™ Agar (Difco), 1001 mg/L Ampicillin).

The inserts of three DNA clones for the construct are subjected to sequence analysis and one clone containing the correct sequence is selected. Large-scale plasmid DNA is isolated using a commercially available kit (QIAGEN Plasmid Mega Kit, Qiagen, Valencia, Calif.) according to manufacturer's instructions.

The same process is used to prepare the ZcytoR21x1 with a C-terminal his tag, composed of Gly Ser Gly Gly His His His His His His (SEQ ID NO:87) (ZcytoR21x1CHIS) or the C-terminal FLAG tag, composed of Gly Ser Asp Tyr Lys Asp Asp Asp Asp Lys (SEQ ID NO:88) (ZcytoR21x1CFLAG). To prepare these constructs, the following 3′ oligonucleotide (CAACCCCAGAGCTGTTTTA AGGCGCGCCTCTAGATTAGTGATGGTGATGGTGATGTCCACCAGATCCGTA AGAGACATCTGGACACA) (SEQ ID NO:89) is used to generate ZcytoR21x1CHIS or the 3′ oligonucleotide (CAACCCCAGAGCTGTTTTAAGGCGCGCCTCTAGAT TACTATCATCATCATCCTTATAATCGGATCCGTAAGAGACATCTGGACACA) (SEQ ID NO: 90) is used to generate ZcytoR21x1CFLAG.

### Example 25

### Transfection and Expression of Soluble ZcytoR21x1 Receptor Expression Constructs that Express the ZcytoR21x1CEE, ZcytoR21x1CHIS, and ZcytoR21x1CFLAG C-Terminal Tagged Proteins

Three sets of 200 μg of each of the soluble ZcytoR21x1 tagged expression constructs, as described in Example 22, are separately digested with 200 units of PvuI at 37° C. for three hours, precipitated with isopropyl alcohol, and centrifuged in a 1.5 mL microfuge tube. The supernatant is decanted off the pellet, and the pellet is washed with 1 mL of 70% ethanol and allowed to incubate for 5 minutes at room temperature. The tube is spun in a microfuge for 10 minutes at 14,000 RPM and the supernatant is decanted off the pellet. The pellet is then resuspended in 750 μl of CHO cell tissue culture medium in a sterile environment, allowed to incubate at 60° C. for 30 minutes, and is allowed to cool to room temperature. Approximately 5×10⁶ CHO cells are pelleted in each of three tubes and are resuspended using the DNA-medium solution. The DNA/cell mixtures are placed in a 0.4 cm gap cuvette and electroporated using the following parameters; 950 pF, high capacitance, at 300 V. The contents of the cuvettes are then removed, pooled, and diluted to 25 mLs with CHO cell tissue culture medium and placed in a 125 mL shake flask. The flask is placed in an incubator on a shaker at 37° C., 6% CO₂ with shaking at 120 RPM.

The CHO cells are subjected to nutrient selection followed by step amplification to 200 nM methotrexate (MTX), and then to 1 μM MTX. Tagged protein expression is confirmed by Western blot, and the CHO cell pool is scaled-up for harvests for protein purification.

### Example 26

### Construction of Mammalian Soluble ZcytoR21x2 Expression Constructs that Express ZcytoR21x2CEE, ZcytoR21x2CHIS, and ZcytoR21x2CFLAG Tagged Proteins

An expression construct containing the extracellular domain of human ZcytoR21x2 with a C-terminal tag, either Glu-Glu (CEE), six His (CHIS), or FLAG (CFLAG) (Example 22), is constructed via PCR and homologous recombination using a DNA fragment encoding ZcytoR21x2 (SEQ ID NO:91) and the expression vector pZMP20.

The PCR fragment encoding ZcytoR21x2CEE contains a 5′ overlap with the pZMP20 vector sequence in the optimized tissue plasminogen activator pre-pro secretion leader sequence coding region, the ZcytoR21x2 extracellular domain coding region (SEQ ID NO: 92), the Glu-Glu tag (Glu Glu Tyr Met Pro Met Glu) coding sequence, and a 3′ overlap with the pZMP20 vector in the poliovirus internal ribosome entry site region. The PCR amplification reaction uses the 5′ oligonucleotide (GTTTCGCTCAGCCAGGAAATCCATGCCGAGTTGAGACGCTTCCGTAGAGCT GGGATTGGCTTTCGCCAC) (SEQ ID NO:93), the 3′ oligonucleotide (CAACCCCAGAGCTGTTTTAAGGCGCGCCTCTAGATrATTCCATGGGCATGT ATTCTTCGTAAGAGACATCTGGACACA) (SEQ ID NO:94), and a previously generated DNA clone of ZcytoR21x2 as the template (SEQ ID NO: 91).

The PCR amplification reaction condition is as follows: 1 cycle, 94° C., 5 minutes; 35 cycles, 94° C., 1 minute, followed by 55° C., 2 minutes, followed by 72° C., 3 minutes; 1 cycle, 72° C., 10 minutes. The PCR reaction mixture is run on a 1% agarose gel and the DNA fragment corresponding to the expected size is extracted from the gel using a QIAquick™ Gel Extraction Kit (Qiagen, Cat. No. 28704).

Plasmid pZMP20 is a mammalian expression vector containing an expression cassette having the chimeric CMV enhancer/MPSV promoter, a BglII site for linearization prior to yeast recombination, an otPA signal peptide sequence, an internal ribosome entry element from poliovirus, the extracellular domain of CD8 truncated at the C-terminal end of the transmembrane domain; an E. coli origin of replication; a mammalian selectable marker expression unit comprising an SV40 promoter, enhancer and origin of replication, a DHFR gene, and the SV40 terminator; and URA3 and CEN-ARS sequences required for selection and replication in S. cerevisiae.

The plasmid pZMP20 is digested with BglII prior to recombination in yeast with the gel extracted ZcytoR21x2CEE PCR fragment. One hundred μl of competent yeast (S. cerevisiae) cells are combined with 10 μl of the ZcytoR21x2CEE insert DNA and 100 ng of BglII digested pZMP20 vector, and the mix is transferred to a 0.2 cm electroporation cuvette. The yeast/DNA mixture is electropulsed using power supply (BioRad Laboratories, Hercules, Calif.) settings of 0.75 kV (5 kV/cm), ∞ ohms, and 25 μF. Six hundred μl of 1.2 M sorbitol is added to the cuvette, and the yeast is plated in 100 μl and 300 μl aliquots onto two URA-D plates and incubated at 30° C. After about 72 hours, the Ura⁺ yeast transformants from a single plate are resuspended in 1 ml H₂O and spun briefly to pellet the yeast cells. The cell pellet is resuspended in 0.5 ml of lysis buffer (2% Triton X-100, 1% SDS, 100 mM NaCl, 10 mM Tris, pH 8.0, 1 mM EDTA). The five hundred μl of the lysis mixture is added to an Eppendorf tube containing 250 μl acid-washed glass beads and 3001]phenol-chloroform, is vortexed for 3 minutes, and spun for 5 minutes in an Eppendorf centrifuge at maximum speed. Three hundred μl of the aqueous phase is transferred to a fresh tube, and the DNA is precipitated with 600 μl ethanol, followed by centrifugation for 30 minutes at maximum speed. The tube is decanted and the pellet is washed with 1 mL of 70% ethanol. The tube is decanted and the DNA pellet is resuspended in 30 μl 10 mM Tris, pH 8.0, 1 mM EDTA.

Transformation of electrocompetent E. coli host cells (DH12S) is done using 5 μl of the yeast DNA preparation and 50 μl of E. coli cells. The cells are electropulsed at 2.0 kV, 25° F., and 400 ohms. Following electroporation, 1 ml SOC (2% Bacto™ Tryptone (Difco, Detroit, Mich.), 0.5% yeast extract (Difco), 10 mM NaCl, 2.5 mM KCl, 10 mM MgCl₂, 10 mM MgSO₄, 20 mM glucose) is added and then the cells are plated in 50 μl and 200 μl aliquots on two LB AMP plates (LB broth (Lennox), 1.8% Bacto™ Agar (Difco), 100 mg/L Ampicillin).

The inserts of three DNA clones for the construct are subjected to sequence analysis and one clone containing the correct sequence is selected. Large-scale plasmid DNA is isolated using a commercially available kit (QIAGEN Plasmid Mega Kit, Qiagen, Valencia, Calif.) according to manufacturer's instructions.

The same process is used to prepare the ZcytoR21x2 with a C-terminal his tag, composed of Gly Ser Gly Gly His His His His His His (SEQ ID NO:95) (ZcytoR21x2CHIS) or the C-terminal FLAG tag, composed of Gly Ser Asp Tyr Lys Asp Asp Asp Asp Lys (SEQ ID NO:96) (ZcytoR21x2CFLAG). To prepare these constructs, the 3′ oligonucleotide (CAACCCCAGAGCTGTTTTAAGGCGCGCCT CTAGATTAGTGATGGTGATGGTGATGTCCACCAGATCCGTAAGAGACATCT GGACACA) (SEQ ID NO:97) is used to generate ZcytoR21x2CHIS or the 3′ oligonucleotide (CAACCCCAGAGCTGTTTTAAGGCGCGCCTCTAGATTACT TATCATCATCATCCTTATAATCGGATCCGTAAGAGACATCTGGACACA) (SEQ ID NO:98) is used to generate ZcytoR21x2CFLAG.

### Example 27

### Transfection and Expression of Soluble ZcytoR21x2 Receptor Expression Constructs that Express the ZcytoR21x2CEE, ZcytoR21x2CHIS, and ZcytoR21x2CFLAG C-Terminal Tagged Proteins

Three sets of 200 μg of each of the soluble ZcytoR21x2 tagged expression constructs, described in Example 24, are separately digested with 200 units of PvuI at 37° C. for three hours, precipitated with isopropyl alcohol, and centrifuged in a 1.5 mL microfuge tube. The supernatant is decanted off the pellet, and the pellet is washed with 1 mL of 70% ethanol and allowed to incubate for 5 minutes at room temperature. The tube is spun in a microfuge for 10 minutes at 14,000 RPM and the supernatant is decanted off the pellet. The pellet is then resuspended in 750 μl of CHO cell tissue culture medium in a sterile environment, allowed to incubate at 60° C. for 30 minutes, and is allowed to cool to room temperature. Approximately 5×10⁶ CHO cells are pelleted in each of three tubes and are resuspended using the DNA-medium solution. The DNA/cell mixtures are placed in a 0.4 cm gap cuvette and electroporated using the following parameters; 950 μF, high capacitance, at 300 V. The contents of the cuvettes are then removed, pooled, and diluted to 25 m]Ls with CHO cell tissue culture medium and placed in a 125 mL shake flask. The flask is placed in an incubator on a shaker at 37° C., 6% CO₂ with shaking at 120 RPM.

The CHO cells are subjected to nutrient selection followed by step amplification to 200 nM methotrexate (MTX), and then to 1 μM MTX. Tagged protein expression is confirmed by Western blot, and the CHO cell pool is scaled-up for harvests for protein purification.

### Example 28

### Transfection and Expression of IL-17C Expression Constructs that Express the IL-17C-CEE, IL-17C-CHIS, and IL-17C-CFLAG C-Terminal Tagged Proteins

Expression constructs of IL-17C fusion or tagged constructs were used to transfect baby hamster kidney cells (BHK) by the lipofectamine method. Specifically, 1×10⁶ BHK cells were seeded on to a 100 mm dish in Dulbeccos Modified Eagle Media (DMEM) containing 10% fetal bovine serum, 10 mM Hepes, pH 7.2 and incubated overnight at 37° C. The attached cells were rinsed with 10 ml of Serum Free Media(SFM): DMEM/F12(Ham) media(1:1) which also contained 10 mM Hepes, 1 ug/ml insulin, 4 ng/ml selenium dioxide, 25 uM ferric citrate. A 16 ug aliquot of an expression construct containing the cDNA for IL-17C-CEE was complexed with 35 ul of lipofectamine (Gibco) in 1.2 ml of SFM for 20 minutes and then following dilution with SFM, applied to the plated BHK cells. Following a 5 hr incubation at 37° C., 6.5 mls of DMEM containing 10% fetal bovine serum was added. The cells were cultured overnight at 37° C. in a humidified tissue culture incubator. Approximately 24 hrs after transfection, the cell media was replaced with fresh DMEM containing 10% fetal bovine serum and also containing 1 uM methotrexate (MTX). After 7 days in 1 uM MTX, the MTX concentration was increased to 10 uM and the cells were allowed to grow for an additional 7-10 days. The cells were maintained in culture to recover the MTX resistant clones and the media was evaluated for the expression of IL-17C-CEE by polyacrylamide gel electrophoresis (SDS-PAGE) and Western blotting using an anti-EE peptide antibody (EE peptide=Glu-Glu tag (Glu Glu Tyr Met Pro Met Glu). The IL-17C-CEE producing cells were then scaled-up for production of recombinant protein. It is well known in the art that for this process, expression contructs containing alternative fusion proteins such as Fc sequences or other tag sequences (His, Flag, etc.) may be substituted for the EE peptide sequenced described here.

### Example 29

### Purification of IL-17C CEE from BHK Cells

Conditioned media from BHK cells expressing IL-17C-CEE (Example 26) was 0.2 um sterile filtered and then loaded on to an anti-EE peptide (EE peptide=Glu-Glu tag (Glu Glu Tyr Met Pro Met Glu) antibody affinity column by loading at 4° C. Prior to loading the pH the conditioned media and the anti-EE antibody-column were adjusted to pH 7.4.

Following the loading of media on to the column, the column was washed with 10 column volumes of 20 mM Tris, 500 mM NaCl, pH7.4. Bound protein was then eluted with 3 column volumes of phosphate buffered saline containing 0.5 mg/ml of EE peptide (Glu Glu Tyr Met Pro Met Glu). Fractions were collected and were analyzed via SDS-PAGE Coomassie staining. Fractions containing IL-17C-CEE were pooled and concentrated approximately 10-fold using a 10 kD molecular weight cutoff Ultrafree-15 membrane concentrator (Millipore, Bedford, Mass.) according to the manufacturer's instructions.

The concentrated sample was then subjected to size exclusion chromatography on a Sephacryl-S100 column (16/60) (Pharmacia, Piscataway, N.J.) equilibrated in 10 mM sodium phosphate, 150 mM NaCl, pH 7.2. The eluted protein was collected in 3 ml fractions which and were analyzed via SDS-PAGE Coomassie staining. The fractions containing pure IL-17C-CEE were pooled and following 0.22 um sterile filtration, the protein was aliquoted and stored at −80° C. until use. N-terminal sequencing of the pure protein confirmed its identity as IL-17C. The analysis of the recombinant protein shows an N-terminal sequence of the mature protein, lacking the signal sequence, begins at Histidine-19 and has a molecular weight of 20663 which includes the C-terminal EE-tag.

### Example 30

### Purification of His-tagged IL-17C from 293F Transient Cells

The following procedure was used to purify both human and murine forms of IL17C having polyhistidine fused at their carboxy-termini. The purification was performed at 4° C. About 10 L of conditioned media from 293F cells transfected with His-tagged IL17C was concentrated to 1.6 L using Pellicon 2 5k filters (Millipore, Bedford, Mass.). Imidazole and NaCl were added to the 1.6 L media to a final concentration of 15 mM and 0.5 M respectively. A Talon (BD Biosciences) column with a 5 mL bed-volume was packed and equilibrated with 20 mM NaPi, 15 mM Imidazole, 0.5 M NaCl, pH 7.5. The media was loaded onto the column at a flow-rate of 1.7 mL/min then washed with 10 CV of the equilibration buffer. His-tagged IL17C was eluted from the column with 20 mM NaPi, 0.5 M NaCl, 0.5 M Imidazole, pH 7.4 at a flow-rate of 1 mL/min. 2 mL fractions were collected and analyzed for the presence of His-tagged IL17C by Coomassie-stained SDS-PAGE.

Talon column elution pool was concentrated from 12 mL to 1 mL using an Amicon Ultra 5k centrifugal filter (Millipore, Bedford, Mass.). A Superdex 75 column with a bed-volume of 121 mL was equilibrated with 50 mM NaPi, 109 mM NaCl, pH 7.3, and the 1 mL sample was injected into the column at a flow-rate of 0.5 mL/min. 2 mL fractions were collected and analyzed for the presence of His-tagged IL17C by Coomassie-stained SDS-PAGE. Fractions containing pure His-tagged IL17C were pooled and concentrated to 2 mL, sterile-filtered through a 2 μm Acrodisc filter (Pall Corporation), and stored at −80° C. Concentration of the final sample was determined by BCA (Pierce, Rockford, Ill.).

### Example 31

### ZcytoR21 Fc10 Fusion Protein Expression Constructs

Two expression plasmids containing either ZcytoR21x1-C(Fc10) (SEQ ID NO:99; SEQ ID NO:100) or ZcytoR21x2-C(Fc10) (SEQ ID NO:101; SEQ ID NO:102) were constructed via homologous recombination using DNA fragments encoding the gene of interest and the expression vector pZMP40. Fragments of polynucleotide sequence of ZcytoR21x1 (SEQ ID NO:1) and ZcytoR21x2 (SEQ ID NO:4) were generated by PCR amplification using primer zc48706 (SEQ ID NO:103), zc48707 (SEQ ID NO:104) and zc48708 (SEQ ID NO:105).

The fragments for both ZcytoR21x1 and ZcytoR21x2 both contained the extracellular domain of their respective coding regions, which was made using previously generated clones of either ZcytoR21x1 or ZcytoR21x2 as templates. The fragments both included a 5′ overlap with a partial pZMP40 vector sequence, either the ZcytoR21x1 or ZcytoR21x2 segment, a linker sequence, a Caspase-3 cleavage site, and a linker region encoding the first 5 amino acids of Fc10 followed by a 3′ overlap containing a partial pZMP40 vector sequence. PCR conditions: 1 cycle, 94° C., 5 minutes; 35 cycles, 94° C., 1 minute, followed by 55° C., 2 minutes, followed by 72° C., 3 minutes; 1 cycle, 72° C., 10 minutes.

The PCR reaction mixtures were run on a 1% agarose gel and a band corresponding to the sizes of the inserts were gel-extracted using a QIAquick™ Gel Extraction Kit (Qiagen, Cat. No. 28704).

The plasmid pZMP40, which was cut with BglII, was used in a recombination reaction using either one or the other of the PCR insert fragments. Plasmid pZMP40 is a mammalian expression vector containing an expression cassette having the MPSV promoter, multiple restriction sites for insertion of coding sequences, and an Fc9 coding region; an E. coli origin of replication; a mammalian selectable marker expression unit comprising an SV40 promoter, enhancer and origin of replication, a DHFR gene, and the SV40 terminator; and URA3 and CEN-ARS sequences required for selection and replication in S. cerevisiae. It was constructed from pZP9 (deposited at the American Type Culture Collection, 10801 University Boulevard, Manassas, Va. 20110-2209, under Accession No. 98668) with the yeast genetic elements taken from pRS316 (deposited at the American Type Culture Collection, 10801 University Boulevard, Manassas, Va. 20110-2209, under Accession No. 77145), an internal ribosome entry site (IRES) element from poliovirus, and the extracellular domain of CD8 truncated at the C-terminal end of the transmembrane domain.

One hundred microliters of competent yeast (S. cerevisiae) cells were independently combined with 10 μl of the insert DNA and 100 ng of cut pZMP40 vector, and the mix was transferred to a 0.2-cm electroporation cuvette. The yeast/DNA mixture was electropulsed using power supply (BioRad Laboratories, Hercules, Calif.) settings of 0.75 kV (5 kV/cm), ohms, and 25 μF. Six hundred μl of 1.2 M sorbitol was added to the cuvette, and the yeast was plated in a 100-μl and 300 μl aliquot onto two URA-D plates and incubated at 30° C. After about 72 hours, the Ura⁺ yeast transformants from a single plate were resuspended in 1 ml H₂O and spun briefly to pellet the yeast cells. The cell pellet was resuspended in 0.5 ml of lysis buffer (2% Triton X-100, 1% SDS, 100 mM NaCl, 10 mM Tris, pH 8.0, 1 mM EDTA). The five hundred microliters of the lysis mixture was added to an Eppendorf tube containing 250 μl acid-washed glass beads and 300 μl phenol-chloroform, was vortexed for 3 minutes, and spun for 5 minutes in an Eppendorf centrifuge at maximum speed. Three hundred microliters of the aqueous phase was transferred to a fresh tube, and the DNA was precipitated with 600 μl ethanol (EtOH) and 30 μl 3M sodium acetate, followed by centrifugation for 30 minutes at maximum speed. The tube was decanted and the pellet was washed with 1 mL of 70% ethanol. The tube was decanted and the DNA pellet was resuspended in 30 μl TE.

Transformation of electrocompetent E. coli host cells (DH12S) was done using 5 μl of the yeast DNA prep and 50 μl of cells. The cells were electropulsed at 2.0 kV, 25 μF, and 400 ohms. Following electroporation, 1 ml SOC (2% Bacto™ Tryptone (Difco, Detroit, Mich.), 0.5% yeast extract (Difco), 10 mM NaCl, 2.5 mM KCl, 10 mM MgCl₂, 10 mM MgSO₄, 20 mM glucose) was added and then the cells were plated in a 50 μl and 200 μl aliquot on two LB AMP plates (LB broth (Lennox), 1.8% Bacto™ Agar (Difco), 100 mg/L Ampicillin).

The inserts of three clones for the construct was subjected to sequence analysis and one clone for each construct, containing the correct sequence, was selected. Larger scale plasmid DNA was isolated using a commercially available kit (QIAGEN Plasmid Mega Kit, Qiagen, Valencia, Calif.) according to manufacturer's instructions.

Three sets of 200 μg of the ZcytoR21x1-C(Fc10) construct (SEQ ID NO:99) were each digested with 200 units of Pvu I at 37° C. for three hours and then were precipitated with IPA and spun down in a 1.5 mL microfuge tube. The supernatant was decanted off the pellet, and the pellet was washed with 1 mL of 70% ethanol and allowed to incubate for 5 minutes at room temperature. The tube was spun in a microfuge for 10 minutes at 14,000 RPM and the supernatant was decanted off the pellet. The pellet was then resuspended in 750 μl of PF-CHO media in a sterile environment, and allowed to incubate at 60° C. for 30 minutes. 5E6 APFDXB11 cells were spun down in each of three tubes and were resuspended using the DNA-media solution. The DNA/cell mixtures were placed in a 0.4 cm gap cuvette and electroporated using the following parameters: 950 μF, high capacitance, and 300 V. The contents of the cuvettes were then removed, pooled, and diluted to 25 mLs with PF-CHO media and placed in a 125 mL shake flask. The flask was placed in an incubator on a shaker at 37° C., 6% CO₂, and shaking at 120 RPM. Protein expression was confirmed via western blot.

The cell line was subjected to nutrient selection followed by step amplification to 100 nM methotrexate (MTX), then to 500 nM MTX. Step amplification was followed by a CD8 cell sort. The CD8 cell sort was accomplished by taking a stable 500 nM MTX amplified pool and staining approximately 5E6 cells with a monoclonal FITC anti-CD8 antibody (BD PharMingen, cat# 30324X) using manufacturers recommended concentration. The stained cells were processed and sorted on a FACS Aria (BD) flow cytometer. The top 5% of cells were collected and outgrown.

### Example 32

### Phosphoprotein Assay for Detection of Receptor-Ligand Interactions

Specific receptor-ligand binding results in activation of intracellular signaling pathways that can be detected in several different ways. Within minutes of specific receptor-ligand binding, changes occur in the phosphorylation state of kinases and transcription factors within the signaling pathways that result in activation or inactivation of downstream cellular responses including proliferation, apoptosis, cell adhesion, inflammatory responses, etc. Activation of these signaling pathways can be detected through use of antibodies that specifically recognize the phosphorylated forms of the kinases or transcription factors. The changes in phosphoprotein levels can be detected and quantitated by Western blotting, by standard ELISA methods, or in multiplexed immunoassays using commercial kits based on Luminex detection technology, such as the BioRad Bio-Plex Suspension Array System.

The BioRad Bio-Plex assay system is a bead based assay system similar to a capture sandwich immunoassay. Antibody directed against the desired target protein, (total transcription factor or kinase) is covalently coupled to internally dyed fluorescent beads. Coupled beads are allowed to react with lysate containing the target protein. After a series of washes to remove unbound protein, a biotinylated detection antibody specific for a different epitope, directed against the phosphorylated form of the target protein (phosphorylated transcription factor or kinase) is added. This results in formation of a sandwich around the target protein. Streptavidin-phycoerythrin is added to bind the biotinylated detection antibody. Antibodies coupled to beads with different fluorescent dyes can be run separately or in combination so that multiple target proteins can be measured simultaneously on the BioRad Bio-Plex Suspension Array System in combination with the BioRad Bio-Plex Manager™ 3.0 software. Up to 100 different target proteins can be assayed simultaneously in this fashion. An example of a multiplexed assay format is the simultaneous measurement of phosphorylated forms of ERK1/2, JNK, p38 MapKinase, Akt, ATF-2, STAT-3, and Iκβα.

The binding and activation of ZcytoR21 by IL-17C or other specific ligands can be detected by using cell lines endogenously expressing the receptor (as determined by RT-PCR). Alternatively, cells overexpressing a transfected ZcytoR21 receptor can be used (NIH3T3/KZ142.8 cells overexpressing a transfected ZcytoR21 receptor, as in Example 17).

Treatment of Cells

Cell lines expressing endogenous or transfected ZcytoR21 are plated at 5000 cells/well in 96 well tissue culture plates and grown overnight in complete growth medium. Cells are cultured for an additional 24 hours in serum free growth medium and then treated for 7 and 15 minutes with IL-17C at varying concentrations up to 300 ng/mL. Additionally, cells can be incubated in the presence of known cytokines or growth factors in combination with the ZcytoR21 ligand(s) (IL-17C) to look at the ability of the ZcytoR21 ligand to enhance or inhibit the signal transduction of known factors.

Lysate Preparation

Following incubation, cells are washed with 100 uL/well ice-cold wash buffer, put on ice, and 50 uL/well lysis buffer is added (BioPlex Cell Lysis Kit, Catalog# 171-304012). Lysates are pipetted up and down five times while on ice, and then agitated on a microplate platform shaker at 300 rpm at 4° C. for 20 minutes. Plates are centrifuged at 4° C. for 20 minutes at 4500 rpm. Supernatants are collected and transferred to a new microtiter plate for storage at −20° C. until time of phosphoprotein assay. The protein concentration in the lysate is determined using BioRad's DC protein assay or any standard method of determining total protein concentration. Samples are adjusted to 200-900 ug/mL total protein by addition of lysis buffer as needed.

Bio-Plex (Luminex) Phosphoprotein Assay

Capture beads (50 uL/well) (beads coupled to primary antibody for transcription factor of interest) are added to 50 uL of lysate in a microtiter plate. The aluminum foil covered plate is incubated overnight at room temperature, with shaking at 300 rpm. The plate is transferred plate to microtiter vacuum apparatus and washed three times with assay buffer. After addition of 25 uL/well detection antibody, the aluminum foil covered plate is incubated at room temperature for 30 min, at 300 rpm. The plate is filtered and washed three times with assay buffer. Streptavidin-PE (50 uL/well) is added and the aluminum foil covered plate is incubated at room temperature for 15 minutes, with shaking at 300 rpm. The plate is filtered and washed two times with bead resuspension buffer. After the final wash, beads are resuspended in 125 uL/well of bead suspension buffer, shaken for 30 seconds, and read on Bio-Plex Suspension Array System according to manufacturers instructions. Data is analyzed using Bio-Plex Manager software. Changes in the level of any of the phosphorylated transcription factors present in the lysate are indicative of a specific receptor-ligand interaction.

Western Analysis of Phosphoprotein

Lysate prepared as described above can also be analyzed using standard Western blotting protocols and probed using phosphorylation state specific antibodies. A receptor-ligand interaction between ZcytoR21 and IL-17C (or other ligands) can be demonstrated by change in the intensity of the band of phosphorylated transcription factor present on the gel.

### Example 33

### Binding of Human IL-17C to Human ZcytoR21x1 and ZcytoR21x2, and Human IL-17R

293fb cells that had been transfected with expression vectors encoding human ZcytoR21x1 and ZcytoR21x2 were assessed for their ability to bind biotinylated human IL-17C. Control transfections included 1) no DNA transfection, 2) vector only (pzmp11), and 3) human IL17R. 10⁶ cells were removed from transfected suspension cultures at day 1, day 2, day 3, and day 5. Cells were pelleted and resuspended in 100 ul of staining media (SM), which is HBSS plus 1 mg/ml bovine serum albumin (BSA), 10 mM Hepes, and 0.1% sodium azide (w/v). Biotinylated human IL-17C was incubated with the cells on ice for 45 minutes at a concentration of 1 ug/ml. An APC conjugated anti-human CD8 antibody (BD Pharmingen; cat.#555369) was also added at 1:25 dilution. After 30 minutes, excess cytokine and antibody was washed away with SM and the cells were incubated with a 1:100 dilution of streptavidin conjugated to phycoerythrin (SA-PE; BD Pharmingen; cat# 554061) for 30 minutes on ice. Excess SA-PE was washed away and cells were analyzed by flow cytometry.

The amount of cytokine binding was detected from the change in the mean fluorescence intensity of the cytokine staining relative to negative controls —1) no DNA transfection and 2) vector only. From this analysis, we find that human IL-17C binds both the human ZcytoR21x1 and ZcytoR21x2, although binding to ZcytoR21x2 is significantly greater. Binding was also seen to human IL-17R.

Baby hamster kidney cells were then transfected with expression vectors as described above, except that the cells were then subjected to methotrexate drug selection to selectively grow out only cells that had been transfected. Stable cell lines were established and these were assayed for CD8 expression and for binding of biotinylated IL-17C as above. Consistent with results obtained in analysis of transient transfections, only those BHK cell lines that expressed ZcytoR21x2 and xl forms bound to IL-17C, with x2 binding IL-17C better than the x1 form.

### Example 34

### IL-17C Binding to ZcytoR21 Variants

BHK cells stably transfected with human and mouse ZcytoR21 splice variants were plated and grown to confluency in T-75 flasks. Cells were lifted off using a non-protease reagent such as Versene (Invitrogen 15040-066), pelleted, and resuspended in a staining reagent (HBSS+1% BSA+0.1% NaAzide+10 mM HEPES) at 2×10e7 cells/ml and aliquoted to a 96-well Costar plate. IL-17C that has been labeled with biotin was independently added to cells at a concentration of 1 ug/ml. The cell/ligand mixture can be incubated for 1 hr at 4 degrees. The wells were washed 1× in staining reagent, and incubated in a secondary reagent containing staining reagent plus Streptavidin-PE (BD Pharmingen 554061) at a 1:100 ratio. The wells were incubated at 4° C. in the dark for 1 hr, followed by a 2× wash in staining media. The cells were then resuspended in a 1:1 mixture of staining media and Cytofix (BD Bioscience 554655) and incubated 10 minutes at RT. The cells were analysed by Flow Cytometry and by gating on the PE positive events for cells that bound IL-17C.

The results were as follows: ZcytoR21 splice variants that bound human IL-17C are ZcytoR21x1, ZcytoR21x2, ZcytoR21x6, ZcytoR21x13, and murine ZcytoR21x6. ZcytoR21 splice variants that bound murine IL-17C were as follows; ZcytoR21x1, ZcytoR21x2, ZcytoR21x4, ZcytoR21-S2, ZcytoR21x6, ZcytoR21x13, and murine ZcytoR21x6. Murine IL-17C also bound murine IL17-RA. Furthermore, ZcytoR21x1, ZcytoR21x2, and ZcytoR21x3 did not bind any of the following: IL-17A, IL-17B, IL-17D, and IL-17F (all biotinylated human forms).

293F cells transiently transfected with ZcytoR21 splice variants using Lipofectamine2000 (Invitrogen 11668-027) were stained as described above. The ZcytoR21 splice variants were engineered with the extra-cellular domain C-terminally linked to a Flag Tag and GPI linkage domain, as described in Examples 22 and 24. The Flag Tag was detected with an anti-Flag-FITC antibody at 1:100 (Sigma F-4049) following staining guidelines described above.

The results were as follows: ZcytoR21 splice variants that bound human IL-17C are ZcytoR21x1, ZcytoR21x2, ZcytoR21-S2, ZcytoR21x4, ZcytoR21x6, ZcytoR21x13, and murine ZcytoR21x6. To a lesser extent, ZcytoR21x3 also bound human IL-17C.

### Example 35

### Distribution of mRNA in Cell Line Panels Using PCR

Human cell lines were grown in-house, some of which were treated with various agents as follows: PMA (phorbol-12-myristate-13-acetate) at 10 ng/ml plus Ionomycin at 0.5 ug/ml for 4 hours (these cell lines are labeled as “activated”), TNF alpha 10 ng/ml for 48 hours, LPS (Lipopolysaccharide) at 100 ng/ml for 24 hours, SEB (Staphlyococcus enterotoxin B) at 1 ug/ml for 24 hours, and CTX (cholera toxin) at 50 nM for 24 hours. RNA was purified using a Qiagen (Valencia, Calif.) RNeasy kit according to the manufacturer's instructions, or an acid-phenol purification protocol (Chomczynski and Sacchi, Analytical Biochemistry, 162:156-9, 1987). The quality of the RNA was assessed by running an aliquot on an Agilent Bioanalyzer. If the RNA was significantly degraded, it was not used for subsequent creation of first strand cDNA. Presence of contaminating genomic DNA was assessed by a PCR assay on an aliquot of the RNA with zc41011: 5′CTCTCCATCCTTATCTTTCATCAAC3′(SEQ ID NO:140) and zc4102: 5′CTCTCTGCTGGCTAAACAAAACAC3′ (SEQ ID NO:141), primers that amplify a single site of intergenic genomic DNA. The PCR conditions for the contaminating genomic DNA assay were as follows: 2.5 ul 10× buffer and 0.5 ul Advantage 2 cDNA polymerase mix (BD Biosciences Clontech, Palo Alto, Calif.), 2 ul 2.5 mM dNTP mix (Applied Biosystems, Foster City, Calif.), 2.5 ul 10× Rediload (Invitrogen, Carlsbad, Calif.), and 0.5 ul 20 uM zc41011 and zc41012, in a final volume of 25 ul. Cycling parameters were 94° C. 20″, 40 cycles of 94° C. 20″ 60° C. 1′20″ and one cycle of 72° C. 7′. 10 ul of each reaction was subjected to agarose gel electrophoresis and gels were examined for presence of a PCR product from contaminating genomic DNA. If contaminating genomic DNA was observed, the total RNA was DNAsed using DNA-free reagents (Ambion, Inc, Austin, Tex.) according to the manufacturer's instructions, then retested as described above. Only RNAs which appeared to be free of contaminating genomic DNA were used for subsequent creation of first strand cDNA.

20 ug total RNA from 90 cell lines were each brought to 98 ul with H₂O, hen split into two 49 ul aliquots, each containing 10 ug total RNA, and placed in two 6-well PCR plates. To each aliquot was added reagents for first strand cDNA synthesis (Invitrogen First Strand cDNA Synthesis System, Carlsbad, Calif.): 20 ul 25 mM MgCl₂, 10 ul 10× RT buffer, 10 ul 0.1M DTT, 2 ul oligo dT, 2 ul RNAseOut. Then, to one aliquot from each cell line 2 ul Superscript II Reverse Transcriptase was added, and to the corresponding cell line aliquot 2 ul H₂O was added to make a minus Reverse Transcriptase negative control. All samples were incubated as follows: 25° C. 10′, 42° C. 50′, 70° C. 15″. Samples were arranged in deep well plates and diluted to 1.7 ml with H₂O. A Multipette (Saigan) robot was used to aliquot 16.5 ul into each well of a 96-well PCR plate multiple times, generating numerous one-use PCR panels of the cell lines, which were then sealed and stored at −20° C. Each well in these panels represents first strand cDNA from approximately 100 ng total RNA. The 180 samples are spread across two 96 well panels, array #119.01 and #119.02. Quality of first strand cDNA on the panels was assessed by a multiplex PCR assay on one set of the panels using primers to two widely expressed, but only moderately abundant genes, CLTC (clathrin) and TFRC (transferrin receptor C). 0.5 ul each of Clathrin primers zc42901: 5′CTCATATTGCTCAACTGTGTGAAAAG3′ (SEQ ID NO:142), zc42902: 5′TAGAAGCCACCTGAACACAAATCTG3′ (SEQ ID NO:143), and TFRC primers zc42599: 5′ATCTTGCGTTGTATGTTGAAAATCAATT3′ (SEQ ID NO:144), zc42600: 5′TTCTCCACCAGGTAAACAAGTCTAC3′ (SEQ ID NO:145), were mixed with 2.5 ul 10× buffer and 0.5 ul Advantage 2 cDNA polymerase mix (BD Biosciences Clontech, Palo Alto, Calif.), 2 ul 2.5 mM dNTP mix (Applied Biosystems, Foster City, Calif.), 2.5 ul 10× Rediload (Invitrogen, Carlsbad, Calif.), and added to each well of a panel of array#l 19.01 and array #119.02. Cycling parameters were as follows: 94° C. 20″, 35 cycles of 94° C. 20″, 67° C. 80″, and one cycle of 72° C. 7′. 10 ul of each reaction was subjected to agarose gel electrophoresis and gels were scored for the presence of a robust PCR product for each gene specific to the +RT wells for each cell line.

Expression of mRNA in the first strand cDNA panels for IL-17C was assayed by PCR with sense oligo zc26004: 5′cactgctactcggctgaggaactgc3′ (SEQ ID NO:146) and antisense oligo zc20996: ′5ttctgtggatagcggtcctcatc3′ (SEQ ID NO:147) under these PCR conditions per sample: 2.5ul 10× buffer and 0.5 ul advantage 2 cDNA polymerase mix (BD Biosciences Clontech, Palo Alto, Calif.), 2 ul 2.5 mM dNTP mix (Applied Biosystems,), 2.5ul 10× Rediload (Invitrogen, Carlsbad, Calif.), and 0.5 ul 20 uM each sense and antisense primer. Cycling conditions were 94° C. 2′, 35 cycles of 94° C. 30″, 68° C. 30″, 72° C. 1′, and one cycle of 72° C. 7′. 10 ul of each reaction was subjected to agarose gel electrophoresis and gels were scored for positive or negative expression of IL-17C.

Results showed some cell lines had differential expression of IL-17C depending on whether they were treated with an agent. Cell lines which were negative in the resting state and positive for IL-17C in the activated or treated state were: the bone marrow AML cell line KG-1, the NHBE (normal human bronchial epithelial primary cells) cell line treated with TNF alpha, LPS, or SEB, and the U-937 monocyte cell line. Conversely, the Tanoue ALL B-cell line and the Hodgkin's lymphoma cell line KM-H2 appeared positive in the resting state while the activated cell line RNA was negative for IL-17C.

Cell lines that were positive for IL-17C in both the stimulated and resting states were: DU-4475, U698, MN60, AML-193, DB, NK-92, Molt-4, UT-7, WeRI-Rb.1, CCRF-HSB2, and NCI-H929. Finally, the cell lines tested only in the resting state which were positive for IL-17C mRNA were: NCI-H716, NCI-H295R, MDA-MB-468, JAR, NIH: OVCAR-3, Sup-B15, NCI-H69, HEL-299, IMR-90, NIC-H292, BEAS2B, U2OS, BFLS-OA, MG-63, 5637, HK-2, Daudi, and Hut 78.

The overall results show that IL-17C is constitutively expressed in many cell lines, including several immune system-related cell lines, but there are a few cell lines that begin expressing IL-17C mRNA in response to activation by various agents. Of particular interest is the response of the bronchial primary epithelial cell line NHBE in producing IL-17C mRNA when treated with TNF alpha, LPS and SEB, which are all considered pro-inflammatory compounds. This suggests that IL-17C plays a role in the setting of inflammation.

### Example 36

### Murine ZcytoR21 mRNA is Regulated in Select Tissues in Murine Models of Disease Compared to Non-Disease Tissues

Experimental Protocol

Tissues were obtained from the following murine models of disease: Colitis, Asthma, Experimental Allergic Encephalomyelitis (EAE), Psoriasis and Collagen Induced Arthritis (CIA). Animal models were run following standard procedures and included appropriate non-diseased controls. Colitis was induced by dextran sulfate sodium (DSS) in the drinking water and the tissues isolated from the model included distal colon, proximal colon and mesenteric lymph nodes. Asthma was induced by sensitization and intranasal challenge to the antigen ovalbumin. The tissues isolated included lung, spleen and lymph node. EAE was induced by immunizing with MOG35-55 peptide in RIBI adjuvant. Tissues isolated included brain, cervical, lymph node, and spinal cord. Psoriasis was induced by adoptive transfer of naive T cells into minor histocompatibility mismatched or syngeneic immunocompromised mice. Tissues isolated included lesional skin and adjacent skin. CIA was induced by collagen injections and tissues isolated included foot and popliteal lymph node. RNA was isolated from all tissues using standard procedures. In brief, tissues were collected and immediately frozen in liquid N2 and then transferred to −80° C. until processing.

For processing, tissues were placed in Qiazol reagent (Qiagen, Valencia, Calif.) and RNA was isolated using the Qiagen RNAeasy kit according to manufacturer's recommendations. Expression of murine ZcytoR21 mRNA was measured with multiplex real-time quantitative RT-PCR method (TaqMan) and the ABI PRISM 7900 sequence detection system (PE Applied Biosystems). ZcytoR21 mRNA levels were normalized to the expression of the murine hypoxanthine guanine physphoribosyl transferase mRNA and determined by the comparative threshold cycle method (User Bulletin 2; PE Applied Biosystems). The primers and probe for murine ZcytoR21 included forward primer 5′ CCACTCACACCCTGCGAAA (SEQ ID NO:148), reverse primer 5′ GCAAGTCCACATTCTCCAGGAT (SEQ ID NO:149), and probe ACCATCCTTCTGACTCCTGTGCTGTGG (SEQ ID NO:150).

Results

Murine ZcytoR21 mRNA expression was detected in all tissues tested. Highest levels of expression were observed in the colon, skin, lung, and foot tissues. Lower levels of expression were found in brain, spinal cord, lymph node, and spleen tissues. ZcytoR21 mRNA was increased in the spinal cord tissue from animals in the EAE model compared to non-diseased controls. ZcytoR21 mRNA was increased approximately 3.75 fold in animals with mild disease score and approximately 2.8 fold in animals with severe disease scores. Murine ZcytoR21 mRNA was decreased in tissues from an acute model of DSS colitis compared to tissues from non-diseased controls. ZcytoR21 mRNA was decreased approximately 2.2 fold in the distal colon and approximately 2.8 fold in the proximal colon compared to non-diseased controls.

Accordingly, one skilled in the art would recognize that since ZcytoR21 expression is increased in such diseases, a ZcytoR21 antagonist, such as the soluble receptors and MAbs of the present invention, would be useful in the treatment of these diseases.

### Example 37

### ZcytoR21 is Regulated in Inflamed Large Intestine Sections of Patients with Ulcerative Colitis and Crohn's Disease

Human ZcytoR21 mRNA is regulated in inflamed large intestine sections of patients with ulcerative colitis and Crohn's disease compared to large intestine sections from normal control patients.

Experimental Protocol

Tissues were obtained from inflamed and un-inflamed large intestine sections of patients with Crohn's disease, ulcerative colitis or normal control patients. RNA was isolated using standard procedures. Expression of human ZcytoR21 mRNA was measured with multiplex real-time quantitative RT-PCR method (TaqMan) and the ABI PRISM 7900 sequence detection system (PE Applied Biosystems). ZcytoR21 mRNA levels were normalized to the expression of the human hypoxanthine guanine physphoribosyl transferase mRNA and determined by the comparative threshold cycle method (User Bulletin 2; PE Applied Biosystems). The primers and probe for human ZcytoR21 included forward primer 5′ TCAGCGTGCGTCTTTGTCA (SEQ ID NO:151), reverse primer 5′ GGCCCCCAGACACAATTTT (SEQ ID NO:152), and probe CATAGGGACTGCTCAGCTCTTCACACTCCA (SEQ ID NO:153).

Results

Human ZcytoR21 mRNA expression was detected in all large intestine samples tested. ZcytoR21 mRNA was decreased 2.1 fold in the large intestine of patients with ulcerative colitis compared to the large intestines from normal patients. ZcytoR21 mRNA was decreased in large intestine samples from patients with Crohn's disease. ZcytoR21 mRNA was decreased 1.5 fold compared to normal patients with no disease.

The decrease in ZcytoR21 expression may be explained by loss of ZcytoR21-expressing cells from the mucosal epithelium. For example, a rat colitis model (reference Scand J Gastroenterol. 2000 October; 35(10): 1053-9.) involving administration of dextran sulfate sodium (DSS) supports this hypothesis in demonstrating decreased epithelial cell survival 60 minutes after administration of DSS and shedding of the epithelium 2 days after administration.

### Example 38

### Murine IL-17C mRNA is Regulated in Select Tissues in Murine Models of Disease Compared to Non-Disease Tissues

Murine IL-17C mRNA is regulated in select tissues in murine models of disease compared to non-diseased controls.

Experimental Protocol

Tissues were obtained from the following murine models of disease: Colitis, Asthma, Experimental Allergic Encephalomyelitis (EAE), Psoriasis and Collagen Induced Arthritis (CIA). Animal models were run following standard procedures and included appropriate non-diseased controls. Colitis was induced by dextran sulfate sodium (DSS) in the drinking water and the tissues isolated from the model included distal colon, proximal colon and mesenteric lymph nodes. Asthma was induced by sensitization and intranasal challenge to the antigen ovalbumin. The tissues isolated included lung, spleen and lymph node. EAE was induced by immunizing with MOG35-55 peptide in RIBI adjuvant. Tissues isolated included brain, cervical, lymph node, and spinal cord. Psoriasis was induced by adoptive transfer of naive T cells into minor histocompatibility mismatched or syngeneic immunocompromised mice. Tissues isolated included lesional skin and adjacent skin. CIA was induced by collagen injections and tissues isolated included foot and popliteal lymph node. RNA was isolated from all tissues using standard procedures. In brief, tissues were collected and immediately frozen in liquid N2 and then transferred to −80° C. until processing. For processing, tissues were placed in Qiazol reagent (Qiagen, Valencia, Calif.) and RNA was isolated using the Qiagen Rneasy kit according to manufacturer's recommendations. Expression of murine IL-17C mRNA was measured with multiplex real-time quantitative RT-PCR method (TaqMan) and the ABI PRISM 7900 sequence detection system (PE Applied Biosystems). IL-17C mRNA levels were normalized to the expression of the murine hypoxanthine guanine physphoribosyl transferase mRNA and determined by the comparative threshold cycle method (User Bulletin 2; PE Applied Biosystems). The primers and probe for murine IL-17C included forward primer 5′ TGGAGATATCGCATCGACACA (SEQ ID NO:154), reverse primer 5′ GCATCCACGACACAAGCATT (SEQ ID NO:155), and probe CCGCTACCCACAGAAGCTGGCG (SEQ ID NO:156).

Results

Murine IL-17C mRNA expression was detected in all tissues tested. Highest levels of expression were observed in the lymph node, colon, skin, lung, foot and spleen tissues. Lower levels of expression were found in brain and spinal cord tissues. IL-17C mRNA was increased in whole foot tissue from mice in the CIA model of arthritis compared to foot tissue from non-diseased controls. IL-17C mRNA was increased approximately 6.6 fold in animals scored with mild disease, approximately 9.1 fold in animals scored with mid level disease and approximately 5 fold in animals with severe disease. IL-17C mRNA was increased in the spinal cord tissue from animals in the EAE model compared to non-diseased controls. IL-17C mRNA was increased approximately 2.05 fold in animals with mild disease score and approximately 2.9 fold in animals with severe disease scores. Murine IL-17C mRNA was increased in tissues from a acute model of DSS colitis compared to tissues from non-diseased controls. IL-17C mRNA was increased approximately 2.8 fold in the distal colon and approximately 1.9 fold in the proximal colon compared to non-diseased controls.

### Example 39

### IL-17C is Regulated in Inflamed Large Intestine Sections of Patients with Ulcerative Colitis and Crohn's Disease

Human IL-17C mRNA is regulated in inflamed large intestine sections of patients with Crohn's disease.

Experimental Protocol

Tissues were obtained from inflamed and un-inflamed large intestine sections of patients with Crohn's disease, Ulcerative Colitis or normal control patients. RNA was isolated using standard procedures. Expression of human IL-17C mRNA was measured with multiplex real-time quantitative RT-PCR method (TaqMan) and the ABI PRISM 7900 sequence detection system (PE Applied Biosystems). IL-17C mRNA levels were normalized to the expression of the human hypoxanthine guanine physphoribosyl transferase mRNA and determined by the comparative threshold cycle method (User Bulletin 2; PE Applied Biosystems). The primers and probe for human IL-17C included forward primer: 5′ atg agg acc gct atc cac aga 3′ (SEQ ID NO:157), reverse primer: 5′ ccc gtc cgt gca tcg a3′ (SEQ ID NO:158), and probe: tgg cct tcg ccg agt gcc tg (SEQ ID NO:159).

Results

Human IL-17C mRNA expression was detected in all large intestine samples tested. IL-17C mRNA was increased in large intestine samples from patients with Crohn's disease. IL-17C mRNA was increased approximately 7.7 fold compared to normal patients with no disease. IL-17C mRNA was increased in the large intestine of some but not all patients with Ulcerative colitis compared to the large intestines from normal patients.

### Example 40

### IL-17C Functional Response on ZcytoR21 Transfectants

NIH-3T3/KZ142 cells were stably transfected with human ZcytoR21x1, human ZcytoR21x2, and human ZcytoR21x6 receptor splice variants as describe din Example 19. As described in Example 32, each cell line was treated for 7 and 15 minutes with a dose response of human IL-17C (SEQ ID NO:17), mouse IL-17C (SEQ ID NO:19), and appropriate controls. The human ZcytoR21x1 transfectants were analyzed with only human IL-17C. Human and mouse IL-17C induced a dose dependent response in phosphorylated IκB-α in the lines overexpressing human ZcytoR21x1 (n=3), human ZcytoR21x2 (n=3), and human ZcytoR21x6 (n=2) splice variants and gave no response in untransfected NIH-3T3/KZ142 cells (n=3). At the 7 minute time point human IL-17C gave a maximum response of 4.68 fold at 300 ng/mL while mouse IL-17C gave a maximum response of 5.22 fold at 300 ng/mL on the NIH-3T3/humanZcytoR21x2 line. Similarly human IL-17C gave a maximum response of 3.04 fold while mouse IL-17C gave a maximum response of 2.92 fold on the NIH-3T3/humanZcytoR21x6 line. At the 15 minute time point human IL-17C (A903G) gave a maximum response of 2.54 fold at 100 ng/mL on the NIH-3T3/humanZcytoR21x1 line. Thus, one skilled in the art would recognize that the binding and cellular signaling produced by IL-17C, that occurs only in cells where ZcytoR21 receptor splice variants are over-expressed, is evidence of a specific receptor-ligand interaction between IL-17C and ZcytoR21.

### Example 41

### Luciferase Reporter Assay To Determine IL-17C Activity on NIH3T3/KZ142.8 Cells and NIH3T3/KZ142.8 Tranfected with human ZcytoRR21x1 ZcytoRR21x2 Splice Variants

Day 1: NIH3T3/KZ142.8 (NIH3T3 cells stably transfected with a inducible NFκB/AP1 luciferase reporter), and these same cells additionally stably transfected with ZcytoR21 receptor splice variants human ZcytoR21x1, ZcytoR21x2, or ZcytoR21X6 were plated at 5000 cells/well in solid white tissue culture 96 well plates (Cat. #3917. Costar) in DMEM high glucose, 5% FBS, 1 mM Na Pyruvate, 1×G418, and 1 uM MTX. (MTX is omitted in the NIH3T3/KZ142.8 parental cell line growth medium). Plates were cultured overnight at 37° C., 5% CO₂.

Day 2: Growth media was replaced with DMEM high glucose, 1 mM Na Pyruvate, 0.1% BSA, and 25 mM Hepes (Assay medium) and plates were incubated overnight at 37° C., 5% CO₂ overnight.

Day 3: Human IL-17C, mouse IL-17C, and appropriate control proteins were serially diluted in assay medium. The human ZcytoR21x1 transfectants were analyzed with only human IL-17C. Spent medium was removed from cells, and each concentration of test ligand or control protein was added to triplicate wells for final assay concentrations of 0, 0.1, 1, 10 and 100 ng/ml. Following incubation for 4 hr at 37° C., 5% CO₂, assay medium was removed and 25 ul/well of 1× lysis buffer (Promega cat #E1531) was added. Plates were incubated for 10 minutes at room temperature then read on a Berthold microplate luminometer using 3 seconds of integration and 40 ul of luciferase substrate (Promega cat #E4550).

Both human and mouse IL-17C induced luciferase reporter gene expression by 2-fold or greater in cells over expressing the ZcytoR21X2 and ZcytoR21X6 splice variants. No induction was observed in the parental NIH3T3/KZ142.8 cells. Thus, one skilled in the art would recognize that the binding and cellular signaling produced by IL-17C, that occurs only in cells where ZcytoR21 receptor splice variants are over-expressed, is evidence of a specific receptor-ligand interaction between IL-17C and ZcytoR21.

### Example 42

### Efficacy of Soluble ZcytoR21 in Disease Models

Based on the expression patterns for IL-17C and ZcytoR21, one skilled in the art would recognize that modulation of the interaction between these two molecules would have biological activity in the following disease models. Such modulation could be facilitated using an Fc fusion protein with an ZcytoR21 polypeptide disclosed herein (e.g. any of SEQ ID NOs: 100, 102 or 124).

Soluble ZcytoR21 Efficacy in a Murine Model of Asthma

A murine model of asthma is induced by sensitization and challenge with the DerP1 antigen or with ovalbumin. Mice can be sensitized by intra-peritoneal injection with antigen in alum and then challenged by intra-nasal administration of antigen.

To demonstrate efficacy of soluble ZcytoR21 mice can be treated at challenge with recombinant ZcytoR21. Lung inflammation can be assessed at various time points post challenge by quantitation of inflammatory cells in lavage fluid, by measurement of airway hyper responsiveness and by pathological analysis. In vivo efficacy of ZcytoR21 will be demonstrated by a reduction in the migration of inflammatory cells into the lung and by alterations in lung pathology and airway hyper responsiveness.

Soluble ZcytoR21 Efficacy in a Murine Model of Collagen Induced Arthritis

The model can be used to investigate mechanisms of disease and potential therapeutics for rheumatoid arthritis. Mice can be immunized with chick type II collagen in Complete Freunds Adjuvant on day —21 and with chick type II collagen in Incomplete Freunds Adjuvant on day 0 in the base of the tail. Disease progression can be scored daily after the second immunization and is assessed by collecting qualitative clinical scores (scale 0-3) and caliper measurements of paw thickness. Clinical scores can be assessed as follows:

0—normal toes and paw

0.5—a single toe is inflamed

1—Two or more toes are inflamed or the top of the foot is inflamed

2—The Top of the foot and the arch (till the ankle) are inflamed (excluding the ankle)

3—The whole foot including the ankle is inflamed

To demonstrate efficacy of soluble ZcytoR21 mice can be treated with recombinant ZcytoR21 by intraperitoneal, intramuscular, subcutaneous, or intravenous injection prior to immunization or during the progression of disease. In vivo efficacy of ZcytoR21 can be demonstrated by a reduction in the progression of disease as judged by a decrease in clinical symptoms, a reduction in paw swelling, a reduction in inflammatory infiltrates as measured by histopathology, and/or reductions in bone/cartilage degradation in the leg as measured by histopathology.

Soluble ZcytoR21 Efficacy in a Murine Model of EAE

EAE is used to investigate mechanisms of disease and potential therapeutics for multiple sclerosis in animal models. It can be induced in C57BL/6 mice using rMOG protein or MOG35-55 peptide, or SJL mice with proteolipid protein peptide(s). To induce EAE mice can be immunized subcutaneously on day 0 with a rMOG/complete Freund's adjuvant (CFA), MOG35-55 peptide/RIBI, or PLP/CFA emulsion, followed by treatment on day 0 and/or day 2 with an intra-venous injection of pertussis toxin. Disease progression can be monitored by clinical score and by weight loss starting after pertussis toxin injection. Clinical scores are based on the animals tail tone, posture and gait as follows: 0—healthy, 1—tail weakness (tip of tail does not curl), 2—tail paralysis (unable to hold tail upright), 3—tail paralysis and mild waddle, 4—tail paralysis and severe waddle, 5—tail paralysis and paralysis of one limb, 6—tail paralysis and paralysis of ANY 2 limbs, 7—tetrapareisis (all 4 limbs paralyzed), 8—moribund or dead.

To demonstrate efficacy of soluble ZcytoR21 mice can be treated with recombinant ZcytoR21 prior to immunization or during the progression of disease. In vivo efficacy of ZcytoR21 can be demonstrated by a reduction in the progression of disease as judged by a decrease in clinical symptoms, by an amelioration of weight loss and by a reduction in inflammatory infiltrates in the brain as measured by histopathology.

Soluble ZcytoR21 Efficacy in a Murine Model of Experimental Colitis

Colitis models can be induced in the mouse and used to evaluate the mechanisms of efficacy of therapeutics in human disease.

Mice can be treated with a solution of dextran sulfate sodium (DSS) administered ad libitum in drinking water. DSS can be administered in such a way as to induce either acute or chronic disease. Disease progression can be monitored by loss of weight and by disease activity index (DAI) scores, composed of percent body weight loss, stool consistency (where 0=normal, 2=soft stool, 4=diarrhea) and hemocult (where 0=normal, 2=no visible blood on anus or in feces, but blue color on Hemocult slide, 4=visible blood on anus or in feces). In the chronic form of this model progression and regression of disease can be measured using these criteria. In vivo efficacy of ZcytoR21 can be demonstrated by a reduction in the progression of disease using the above criteria and by a reduction in inflammatory infiltrates in the gut as measured by pathology.

A hapten induced model of colitis can be used to study Th2 mediated colitis. In this model mice are sensitized by topical application of oxazalone or TNBS on day 0 and challenged by intrarectal administration of oxazalone or TNBS on day 6. Disease progression can be monitored by loss of weight and by disease activity index (DAI) scores, composed of percent body weight loss, stool consistency (where 0=normal, 2=soft stool, 4=diarrhea) and hemocult (where 0=normal, 2=no visible blood on anus or in feces, but blue color on Hemocult slide, 4=visible blood on anus or in feces). In vivo efficacy of ZcytoR21 can be demonstrated by a reduction in the progression of disease using the above criteria and by a reduction in inflammatory infiltrates in the gut as measured by histopathology.

### Example 43

### Construction of ZcytoR21 Variant Extracellular Domains in a Vector that Allowing Expression of a Carboxy-Terminal Epitope Tag and GPI Mediated, Plasma Membrane Anchorage

Expression of ZcytoR21 extracellular (ECD) domains fused to a carboxy-terminal FLAG epitope tag and anchored to cell plasma membranes via a GPI linker allows ligand binding studies to be normalized to protein expression levels. The commercial mammalian expression vector pVAC2 (Invivogen, SanDiego, Calif.) allows for the fusion of ECD's to the 32 amino acid carboxy-terminal domain of human placental alkaline phosphatase (PLAP). During processing of the pro-peptide as it transits the Golgi, a transaminase cleaves this PLAP domain and simultaneously adds a GPI tail thus providing a hydrophobic anchor for the ECD in the cell membrane. Each of the following ZcytoR21 ECD splice variants was cloned into the commercial mammalian expression vector pVAC2 utilizing the vector's BamH1 and EcoR1 sites such that the PLAP fragment was kept in frame. The FLAG epitope sequence is commonly used and there are monoclonal antibodies commercially available. The epitope sequence was coded for in the each antisense oligonucleotide utilized in the PCR reactions that generated the ECD's. The fragments for human ZcytoR21x1, human ZcytoR21x2, human ZcytoR21x3, human ZcytoR21x6, human ZcytoR21x13 and murine ZcytoR21x6 were generated by PCR using previously generated clones as templates. The regions of difference between these clones lay internal to the oligos thus all PCR reactions utilized the same oligonucleotide pair as shown in SEQ ID NO:166 and SEQ ID NO:167. The human ZcytoR21-S2 clone was generated using human ZcytoR21x2 as template and a different sense oligonucleotide as shown by SEQ ID NO:168 but the same antisense primer. A murine version of ZcytoR21x6 was generated using a previously cloned template and the primers as shown in SEQ ID NO:169 and SEQ ID NO:170. Due to the presence of an internal EcoR1 site, PCR products were digested with the restriction enzyme Esp3I that left cohesive ends matching EcoR1 and BamH1. The digested and purified products were successfully ligated into pVAC2 and sequenced yielding: pVAC2-human ZcytoR21x1, (SEQ ID NO:171), pVAC2-ZcytoR21x2, (SEQ ID NO:172), pVAC2-hZcytoR21x3, (SEQ ID NO:173), pVAC2-hZcytoR21x6, (SEQ ID NO:174), pVAC2-hZcytoR21x13, (SEQ ID NO:175), pVAC2-mZcytoR21x6, (SEQ ID NO:176), pVAC2-hcytor21-S2, (SEQ ID NO:177).

### Example 44

### ZcytoR21 Fc10 Fusion Protein Expression Constructs

An expression plasmid containing ZcytoR21x2-C(Fc10) with a native leader was constructed from a previously described, optimized TPA leader version (Example 29; SEQ ID NO:101 and SEQ ID NO:102) This was accomplished by exchanging an approximately 530 bp EcoRI fragment from the TPA leader version, for an approximately 480 bp EcoRI fragment from a full length human ZcytoR21x2 pzmp11 dicistronic expression construction described in Example 16. The two expression constructions in question share a vector-derived EcoR1 site just upstream of the insert, on one hand, and a ZcytoR21 insert-derived EcoRI site, on the other hand. Several clones resulting from this genetic engineering event were sequenced and a clone with a correctly oriented EcoRI fragment was selected for expression. This native leader version of ZcytoR21x2-C(Fc10) is called mpet 1330 (SEQ ID NO:178).

### Example 45

### Expression of GPI anchored, ZcytoR21 Variant Extracellular Domains in Mammalian Cells

The assessment of the ligand binding characteristics of cytokine receptors can be facilitated through the expression of their extracellular domains tethered to the surface of cells via a GPI linker. The following constructs, previously described in Example 41, were transiently expressed in 293f cells (Invitrogen, Carlsbad, Calif.) for 48-96 hours, harvested by centrifugation and utilized for ligand binding analysis by FACS: pVAC2-hZcytoR21x1, pVAC2-hZcytoR21x2, pVAC2-hZcytoR21x3, pVAC2-hZcytoR21x6, pVAC2-hZcytoR21x13, pVAC2-hZcytoR21-S2 and pVAC2-mZcytoR21x6.

On day 1; 25 ml of shake flask cultured, low passage 293f cells were seeded into 100 ml of Freestye Expression Medium (Invitrogen, Carlsbad, Calif.) in a 500 ml Erlenmeyer, polycarbonate TC flask (Corning, Corning N.Y.) at a density of approximately 0.7e6 cells/ml. The cells were cultured at 37° C. with ambient airflow @ 0.2 LPM supplemented with 6% CO₂, affixed to an orbital shaker rotating at 90 rpm. These settings were utilized for the entire length of the culture. On day 2, the cells were counted using a haemocytometer, centrifuged at 800 g, resuspended in fresh Freestyle media to 1.0e6 cells/ml and divided into 20 125 ml Erlenmeyer, polycarbonate TC flask (Corning, Corning N.Y.) at 10 ml/flask and transfected as follows. 10 ug of plasmid DNA prepared using either a miniprep or maxiprep Qiagen kit (Valencia, Calif.) following the manufacturer's suggested procedures was diluted into 200 microliters of Optimem media (Invitrogen, Carlsbad, Calif.). Simultaneously, 12.5 microliters of Lipofectamine2000 transfection reagent (Invitrogen, Carlsbad, Calif.) was mixed with 200 microliters of Optimem. After both mixtures had incubated for 5 minutes at room temperature they were mixed by pipetting and incubated at room temperature an additional 30 minutes. Each DNA-lipid mixture was then added to a 125 ml flask of cells. Thus transfected cells were incubated for 48-96, harvested and washed into PBS+azide/BSA by centrifugation and utilized for FACS based binding studies. Receptor expression levels were assessed by measurement of a FLAG epitope specific antibody and biotinylated IL17C binding compared to the nonspecific binding seen in cells transfected with an unmodified pVAC2 “empty” vector.

### Example 46

### Human ZcytoR21 Polyclonal Antibodies

Anti-ZcytoR21 polyclonal antibodies are prepared by immunizing 2 female New Zealand white rabbits with either: the purified mature recombinant human ZcytoR21 polypeptide produced from 293 cells (ZytoR1-293), purified recombinant human ZcytoR21s2, or subdomains thereof, including SEQ ID NOs:113, 115, 117 or 119 containing a C-terminal tag fusion to facilitate purification (e.g. His, FLAG, EE, Fc).

Alternatively, a ZcytoR21-MBP fusion protein, produced in E. coli, which utilizes the extracellular domain sequence of ZcytoR21 fused to the Maltose-binding protein (MBP), or synthetic peptides containing a portion of the peptide sequence found in the extracellular domain of human ZcytoR21 with an additional Cys added to the N-teminus or C-terminus of the peptides to facilitate conjugation. The peptides and fusion proteins are conjugated by methods known in the art (e.g. Maleimide Activated Supercarrier System, No 77656, or Pharmalink Kit No 77158, Pierce Biotechnology, Rockland Ill.) to a carrier protein such as BSA and KLH to increase the antigenicity of the peptide or fusion-protein. The rabbits were each given an initial intraperitoneal (ip) injection of 200 μg of purified protein in Complete Freund's Adjuvant followed by booster IP injections of 100 μg peptide in Incomplete Freund's Adjuvant every three weeks. Seven to ten days after the administration of the second booster injection (3 total injections), the animals were bled and the serum was collected. The animals were then boosted and bled every three weeks.

The human ZcytoR21-specific polyclonal antibodies are affinity purified from the immune rabbit serum using a CNBr-SEPHAROSE 4B protein column (Pharmacia LKB) that was prepared using 10 mg of the specific antigen purified recombinant protein human ZcytoR21-293 or peptide per gram of CNBr-SEPHAROSE, followed by 20× dialysis in PBS overnight. Human ZcytoR21-specific antibodies are characterized by ELISA using 500 ng/ml of the purified recombinant protein human ZcytoR21-293 as antibody target. The lower limit of detection (LID) of the rabbit anti-human ZcytoR21 affinity purified antibody is usually 10-500 pg/ml on its specific purified recombinant antigen human ZcytoR21-293. Alternatively, the serum can be processed to isolate the IgG fraction by Protein A-affinity chromatography or other methods known in the art.

The human ZcytoR21-specific polyclonal antibodies are characterized for their ability to bind the ZcytoR21-Fc protein in an ELISA format or to specifically bind ZcytoR21 transfected NIH3T3, 293 or BHK cells or to block the induction of luciferase in IL-17C treated NIH3T3 cells which contain an NFkB-sensitive luciferase reporter construct and have also been transfected with ZcytoR21. The ability of ZcytoR21 directed polyclonal antibodies to inhibit the binding of purified recombinant human IL-17C to ZcytoR21-Fc protein or ZcytoR21 transfected NIH3T3, 293 or BHK cells or to inhibit the bioactivity of IL-17C in the NIH3T3/ZcytoR21/NFkB-luciferase bioassay would be evidence of the ability of the ZcytoR21 specific antibody to antagonize the bioactivity of human IL-17C.

### Example 47

### Generation of Murine Anti-Human ZcytoR21 Monoclonal Antibodies

A. Immunization for Generation of Anti-ZcytoR21 Antibodies

1. Soluble ZcytoR21-Fc

Six to twelve week old intact or ZcytoR21 knockout mice are immunized by intraperitoneal injection with 50-100 ug of soluble human ZcytoR21-mFc protein mixed 1:1 (v:v) with Ribi adjuvant (Sigma) on a biweekly schedule. Seven to ten days following the third immunization, blood samples are taken via retroorbital bleed, the serum harvested and evaluated for its ability to inhibit the binding of IL-17C to ZcytoR21 in neutralization assays and to stain ZcytoR21 transfected versus transfected P815 or NIH3T3 cells in a FACS staining assay or on a FMAT system. Mice are continued to be immunized and blood samples taken and evaluated as described above until neutralization titers reached a plateau. At that time, mice with the highest neutralization titers are injected intravenously with 25-50 ug of soluble ZcytoR21-Fc protein in PBS. Three days later, the spleen and lymph nodes from these mice are harvested and used for hybridoma generation, for example using mouse myeloma (P3-X63-Ag8.653.3.12.11) cells or other appropriate cell lines in the art, using standard methods known in the art (e.g. see Kearney, J. F. et al., J Immunol. 123:1548-50, 1979; and Lane, R. D. J Immunol Methods 81:223-8. 1985.

2. Soluble ZcytoR21, ZcytoR21-CEE, ZcytoR21-His, ZcytoR21-FLAG

Six to twelve week old intact or ZcytoR21 knockout mice are immunized by intraperitoneal injection with 50-100 ug of soluble human soluble ZcytoR21, ZcytoR21-CEE, ZcytoR21-His, ZcytoR21-FLAG protein mixed 1:1 (v:v) with Ribi adjuvant (Sigma) on a biweekly schedule. Seven to ten days following the third immunization, blood samples were taken via retroorbital bleed, the serum harvested and evaluated for its ability to inhibit the binding of IL-17C to ZcytoR21-Fc, human soluble ZcytoR21, ZcytoR21-CEE, ZcytoR21-His, or ZcytoR21-FLAG in neutralization assays and to stain ZcytoR21 transfected versus transfected P815 or NIH3T3 cells in a FACS staining assay or on a FMAT system. Mice are continued to be immunized and blood samples taken an evaluated as described above until neutralization titers reached a plateau. At that time, mice with the highest neutralization titers were injected intravenously with 25-50 ug of soluble ZcytoR21-Fc protein in PBS. Three days later, the spleen and lymph nodes from these mice are harvested and used for hybridoma generation, for example using mouse myeloma (P3-X63-Ag8.653.3.12.11) cells or other appropriate cell lines in the art, using standard methods known in the art (e.g. see Kearney, J. F. et al., J Immunol. 123:1548-50, 1979; and Lane, R. D. J Immunol Methods 81:223-8. 1985.

3. Soluble ZcytoR21Domains

Six to twelve week old intact or ZcytoR21 knockout mice are immunized by intraperitoneal injection with 50-100 ug of soluble purified recombinant human ZcytoR21 domain HUZCYTOR21s2 (SEQ ID NO:113), or the subdomains thereof (e.g. SEQ ID NOs: 115, 117 or 119) containing a C-terminal tag fusion to facilitate purification (e.g. His, FLAG, EE, Fc) conjugated by methods known in the art (e.g. Pharmalink Immunogen Kit No 77158, Pierce Biotechnology, Rockland Ill.) to a carrier protein such as BSA and KLH to increase the antigenicity. The pure protein is mixed 1:1 (v:v) with Ribi adjuvant (Sigma) on a biweekly schedule. Seven to ten days following the third immunization, blood samples were taken via retroorbital bleed, the serum harvested and evaluated for its ability to inhibit the binding of IL-17C to ZcytoR21-Fc, human soluble ZcytoR21, ZcytoR21-CEE, ZcytoR21-His, or ZcytoR21-FLAG in neutralization assays and to stain ZcytoR21 transfected versus transfected P815 or 293 cells in a FACS staining assay or on a FMAT system. Mice are continued to be immunized and blood samples taken an evaluated as described above until neutralization titers reached a plateau. At that time, mice with the highest neutralization titers were injected intravenously with 25-50 ug of soluble ZcytoR21 protein antigen in PBS. Three days later, the spleen and lymph nodes from these mice are harvested and used for hybridoma generation, for example using mouse myeloma (P3-X63-Ag8.653.3.12.11) cells or other appropriate cell lines in the art, using standard methods known in the art (e.g. see Kearney, J. F. et al., J Immunol. 123:1548-50, 1979; and Lane, R. D. J Immunol Methods 81:223-8. 1985.

4. P815 Transfectants that Express ZcytoR21

Six to ten week old female DBA/2 mice are immunized by intraperitoneal injection 1-5×10⁶ irradiated, transfected cells every 2-3 weeks. In this approach, no animals develop and die of ascites tumor. Instead, animals are monitored for a neutralizing immune response to ZcytoR21 in their serum as outlined above, starting with a bleed after the second immunization. Once neutralization titers have reached a maximal level, the mice with highest titers are given a pre-fusion, intraperitoneal injection of 5×10⁶ irradiated cells and four days later, the spleen and lymph nodes from these mice are harvested and used for hybridoma generation, for example using mouse myeloma (P3-X63-Ag8.653.3.12.11) cells or other appropriate cell lines in the art, using standard methods known in the art (e.g. see Kearney, J. F. et al., J Immunol. 123:1548-50, 1979; and Lane, R. D. J Immunol Methods 81:223-8. 1985.

B. Screening the Hybridoma Fusions for Antibodies that Bind ZcytoR21 and Inhibit the Binding of IL-17C to ZcytoR21

Four different primary screens are performed on the hybridoma supernatants at 8-10 days post-fusion. For the first assay, antibodies in supernatants were tested for their ability to bind to plate bound soluble ZcytoR21-Fc, human soluble ZcytoR21, ZcytoR21-CEE, ZcytoR21-His, or ZcytoR21-FLAG protein by ELISA using HRP-conjugated goat anti mouse kappa and anti-lambda light chain second step reagents to identify bound mouse antibodies. To demonstrate specificity for the ZcytoR21 portion of the ZcytoR21 fusion proteins, positive supernatants in the initial assay are evaluated on an irrelevant protein fused to the same murine Fc region (mG2a), EE sequence, His sequence, or FLAG sequence. Antibody in those supernatants that bound to ZcytoR21-fusion protein and not he irrelevant muFc or other proteins containing fusion protein sequence were deemed to be specific for ZcytoR21. For the second assay, antibodies in all hybridoma supernatants were evaluated by ELISA for their ability to inhibit the binding of biotinylated human IL-17C to plate bound ZcytoR21-Fc or other ZcytoR21-fusion proteins.

All supernatants containing antibodies that bound specifically to ZcytoR21, whether they inhibited the binding of IL-17C to ZcytoR21 or not in the ELISA assay, are subsequently tested for their ability to inhibit the binding of IL-17C to ZcytoR21 transfected NIH3T3, 293 or BHK cells or normal human epithelial cells. All supernatants that are neutralization positive in the IL-17C neutralization assays are subsequently evaluated for their ability to stain ZcytoR21 transfected NIH3T3, 293 or BHK cells by FACS analysis. This analysis is designed to confirm that inhibition of IL-17C binding to ZcytoR21, was indeed due to the antibody that specifically binds the ZcytoR21 receptor. Additionally, since the FACS analysis in performed with an anti-IgG second step reagent, specific FACS positive results indicate that the neutralizing antibody is likely to be of the IgG class. By these means, a master well is identified that binds ZcytoR21 in a plate bound ELISA, inhibits the binding of IL-17C to ZcytoR21 in the ELISA based inhibition assay, blocks the interaction of IL-17C with ZcytoR21 transfected NIH3T3, 293 or BHK cells, respectively, and is positive for the staining of ZcytoR21 transfected NIH3T3, 293 or BHK cells with an anti-mouse IgG second step reagent.

The third assay consists of NIH/3T3 cells containing an NFkB sensitive luciferase reporter construct and which have also been transfected with ZcytoR21 and can therefore respond to IL-17C treatment. These cells respond to IL-17C treatment by increasing the expression of luciferase which can then be assayed by standard methods known in the art. The specific monoclonal antibody to ZcytoR21 is assayed by its ability to, for example, inhibit IL17C— stimulated luciferase production by these cells.

The fourth assay consists of primary human epithelial cells or cell lines of human origin such as U937, HCT15, DLD-1 or Caco2 cells which express ZcytoR21 and respond to IL-17C treatment. The specific monoclonal antibody is assayed by its ability to, for example, inhibit IL17C stimulated chemokine or cytokine production by these cells. Chemokine or cytokine production is assayed in response to IL-17C using commercially available ELISA assay kits (e.g. R&D Systems, Minneapolis, Minn.). Alternatively, the phospho-IkB levels in the IL-17C responsive cells can be monitored using phosphorylation specific antibodies available for this purpose (BioRad, Richmond, Calif.). The inhibition of IL-17C mediated phospho-IkB production would be a measure of ZcytoR21 antagonist activity by the monoclonal antibody.

C. Cloning Anti-ZcytoR21 Specific Antibody Producing Hybridomas

Hybridoma cell lines producing a specific anti-ZcytoR21 mAb that neutralizes the binding of IL-17C to appropriately transfected BaF3 or BHK cells are cloned by a standard low-density dilution (less than 1 cell per well) approach. Approximately 5-7 days after plating, the clones are screened by ELISA on, for example, plate bound human ZcytoR21-Fc followed by a retest of positive wells by ELIDA on irrelevant Fc containing fusion protein as described above. Selected clones, whose supernatants bind to ZcytoR21-Fc and not the irrelevant Fc containing fusion protein, are further confirmed for specific antibody activity by repeated both neutralization assays as well as the FACS analysis. All selected ZcytoR21 antibody positive clones are cloned a minimum of two times to help insure clonality and to assess stability of antibody production. Further rounds of cloning are performed and screened as described until, preferably, at least 95% of the resulting clones are positive for neutralizing anti-ZcytoR21 antibody production.

D. Biochemical Characterization of the Molecule Recognized by Anti-ZcytoR21 mAbs

Biochemical confirmation that the target molecule, ZcytoR21, recognized by the putative anti-ZcytoR21 mAbs is indeed ZcytoR21 is performed by standard immunoprecipitation followed by SDS-PAGE analysis or western blotting procedures, both employing soluble membrane preparations from ZcytoR21 transfected versus untransfected Baf3 or BHK cells. Moreover, soluble membrane preparations of non-transfected cell lines that express ZcytoR21 are used to show that mAbs recognize the native receptor chain as well as the transfected one. Alternatively, the mAbs are tested for their ability to specifically immunoprecipitate or western blot the souble ZcytoR21-Fc protein.

### Example 48

### Neutalization of Human ZcytoR21 by Sera from Mice Injected with P815 Cells Transfected with Human ZcytoR21

Using a cell based neutralization assay, serum from mice injected with human ZcytoR21 transfected P815 cells, as described herein, is added as a serial dilution at 1%, 0.5%, 0.25%, 0.13%, 0.06%, 0.03%, 0.02% and 0% The assay plates are incubated at 370C, 5% CO₂ for 4 days at which time Alamar Blue (Accumed, Chicago, Ill.) is added at 20 μl/well. Plates are again incubated at 37° C., 5% CO₂ for 4-16 hours. Differences in Alamar Blue conversion shows that serum from the animals can neutralize the signaling of Il-17C through human ZcytoR21.

Results from this assay can provide additional evidence that effectively blocking ZcytoR21 binding, blocking, inhibiting, reducing, antagonizing or neutralizing IL-17C activity, for example via a neutralizing monoclonal antibody to ZcytoR21 of the present invention, could be advantageous in reducing the effects of IL-17C in vivo and may reduce IL-17C associated inflammation, such as that seen in, psoriasis, IBD, colitis, chronic obstructive pulmonary disease, cystic fibrosis, arthritis, asthma, psoriatic arthritis, atopic dermatitis or other inflammatory diseases.

Peptide Synthesis

Peptide ZcytoR21-1.1 [CIEASYLQEDTVRRKK-amide] and peptide ZcytoR21-2.1[ISHKGLRSKRTQPSDPETWESC] were synthesized with Fmoc chemistry on a model 433A Peptide Synthesizer (Applied Biosystems). Fmoc-Amide or Fmoc-Cys (Trt)-Wang resin (AnaSpec) (0.25 mmol) was used as the initial support resins, respectively. A mixture of 2-(1H-Benzotriazol-1-yl)-1,1,3,3-Tetramethyluronium hexafluorophosphate (HBTU), 1-Hydroxybenzotriazole (HOBt), N,N-Diisopropylethyamine, N-Methylpyrrolidone, Dichloromethane (Applied Biosystems and Piperidine (Aldrich Chemical Co.) were used as synthesis reagents. The peptide was cleaved from the solid support wit 95% trifluoroacetic acid(TFA). Purification of the peptide was performed by RP-HBLC using a Vydac C18, 10-15 micron, 50×250 mm preparative column with water/acetonitrile/TFA gradients. Eluted fractions from the column were collected and analyzed for purity by analytical RP-HPLC. Pooled fractions were lyophilized to dryness and resuspended in 10% acetonitrile, 1% acetic acid, then re-lyophilized to dryness in a Falcon tube of known weight. Analytical HPLC and mass spectrometry (MS) were performed before the final dry-down. Overall synthesis yields were 30-33%

### Example 49

Construction of Mammalian Soluble ZcytoR21-S2 Expression Constructs

Mutagenesis, protein engineering and binding studies have suggested that a ZcytoR21x2 extracellular domain without amino acids 24-96 of SEQ ID NO:5, designated ZcytoR21-S2 (SEQ ID NO:113) has high ligand binding affinity. Expression constructs containing the extracellular domains of human or mouse ZcytoR21-S2 with a carboxy-terminal Fc type tag placed into the mammalian expression vector pZMP40 (SEQ ID NO:183>are constructed using PCR and homologous recombination in yeast as follows. To construct human pZMP40-hZcytoR21-S2-FC10, (SEQ ID NO:184) a PCR product is obtained by combining the sense oligonucleotide 5′CATGCCGAGTTGAGACGCTTCCGTAGA GGACCCGAGTTCTCCTTTTGATTT3′ (SEQ ID NO:185) and the antisense oligonucleotide 5′ctctgatccatcaacttcatcagatccGTGTCTGTAAGAGACATCCGGACA3′ (SEQ ID NO:186) in a PCR reaction with a previously generated human ZcytoR21x2 (SEQ ID NO: 91) plasmid as template. Briefly, the PCR reaction is run utilizing Expand T DNA polymerase (Roche Applied Science, Indianapolis, Ind.) following the manufacturer's suggested reagent concentrations using the following cycling parameters: 1 cycle, 94° C., 5 minutes; 30 cycles, 94° C., 30 seconds, followed by approximately 62° C., 30 seconds, followed by 72° C., 1 minute; 1 cycle, 72° C., 10 minutes. The PCR reaction mixture is run on a 1% agarose gel and the DNA fragment corresponding to the expected size is extracted from the gel using a QIAquick™ Gel Extraction Kit (Qiagen, Cat. No. 28704) yielding a purified DNA fragment.

This PCR fragment contains a 5′ overlap with the MPET 1122 vector sequence (SEQ ID NO:187) in the optimized tissue plasminogen activator pre-pro secretion leader sequence coding region, the ZcytoR21-S2 extracellular domain coding region contained within pZMP40-hZcytoR21-S2-Fc10 (SEQ ID NO:184), and a 3′ overlap with the MPET 1122 vector in the Fc10 carboxy terminal tag.

Plasmid pZMP40 is a mammalian expression vector containing an expression cassette having the chimeric CMV enhancer/MPSV promoter, a BglII site for linearization prior to yeast recombination, an otPA signal peptide sequence, an internal ribosome entry element from poliovirus, the extracellular domain of CD8 truncated at the C-terminal end of the transmembrane domain; an E. coli origin of replication; a mammalian selectable marker expression unit comprising an SV40 promoter, enhancer and origin of replication, a DHFR gene, and the SV40 terminator; and URA3 and CEN-ARS sequences required for selection and replication in S. cerevisiae, and is the scaffolding for the MPET 1122 construct.

The plasmid MPET 1122 is digested with Srf1 prior to recombination in yeast with the gel extracted ZcytoR21-S2 PCR fragment. 100 ul of competent yeast (S. cerevisiae) cells are combined with 10 ul of the ZcytoR21-S2 insert DNA and 100 ng of BglII digested pZMP20 vector, and the mix is transferred to a 0.2 cm electroporation cuvette. The yeast/DNA mixture is electropulsed using power supply (BioRad Laboratories, Hercules, Calif.) settings of 0.75 kV (5 kV/cm), ∞ ohms, and 25 uF. Six hundred ul of 1.2 M sorbitol is added to the cuvette, and the yeast is plated in 100 ul and 300 μl aliquots onto two URA-D plates and incubated at 30° C. After about 72 hours, the Ura⁺ yeast transformants from a single plate are resuspended in 1 ml H₂O and spun briefly to pellet the yeast cells. The cell pellet is resuspended in 0.5 ml of lysis buffer (2% Triton X-100, 1% SDS, 100 mM NaCl, 10 mM Tris, pH 8.0, 1 mM EDTA). The five hundred ul of the lysis mixture is added to an Eppendorf tube containing 250 ul acid-washed glass beads and 300 ul phenol-chloroform, is vortexed or 1 minutes, and spun for 5 minutes in an Eppendorf centrifuge at maximum speed. Three hundred ul of the aqueous phase is transferred to a fresh tube, and the DNA is precipitated with 600 ul ethanol, followed by centrifugation for 30 minutes at maximum speed. The tube is decanted and the pellet is washed with 1 mL of 70% ethanol. The tube is decanted and the DNA pellet is resuspended in 30 ul 10 mM Tris, pH 8.0, 1 mM EDTA.

Transformation of electrocompetent E. coli host cells (DH10b, Invitrogen, Carlsbad, Calif.) is done using 5 ul of the yeast DNA preparation and 50 ul of E. coli cells. The cells are electropulsed at 2.0 kV, 25 μF, and 400 ohms. Following electroporation, 1 ml SOC (2% Bacto™ Tryptone (Difco, Detroit, Mich.), 0.5% yeast extract (Difco), 10 mM NaCl, 2.5 mM KCl, 10 mM MgCl₂, 10 mM MgSO₄, 20 mM glucose) is added and then the cells are plated in 50 ul and 200 ul aliquots on two LB AMP plates (LB broth (Lennox), 1.8% Bacto™ Agar (Difco), 100 mg/L Ampicillin).

The inserts of three DNA clones for the construct are subjected to sequence analysis and one clone containing the correct sequence is selected. Large-scale plasmid DNA is isolated using a commercially available kit (QIAGEN Plasmid Mega Kit, Qiagen, Valencia, Calif.) according to manufacturer's instructions. The soluble protein is produced by common mammalian production cells such as CHO or BHK following standard transfection procedures as previously described in examples 25 and 26.

Variations of this ZcytoR21-S2 construct may be built utilizing other secretion leaders, epitope tags or fusion partners conferring different useful properties on the soluble protein or placed in different expression vectors useful in improving protein expression.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. An isolated ZcytoR21 soluble receptor comprising SEQ ID NO:9.

2. An isolated ZcytoR21 soluble receptor comprising SEQ ID NO:12.

3. An isolated ZcytoR21 soluble receptor comprising amino acid residues 136-414 of SEQ ID NO:21.

4. An isolated ZcytoR21 soluble receptor comprising amino acid residues 24-414 of SEQ ID NO:21.

5. An isolated ZcytoR21 soluble receptor comprising SEQ ID NO:23.

6. An isolated ZcytoR21 soluble receptor comprising amino acid residues 159-437 of SEQ ID NO:107.

7. An isolated ZcytoR21 soluble receptor comprising SEQ ID NO:122.

8. An isolated ZcytoR21 soluble receptor comprising amino acid residues 136-414 of SEQ ID NO:109.

9. An isolated ZcytoR21 soluble receptor comprising amino acid residues 24-414 of SEQ ID NO:109.

10. An isolated ZcytoR21 soluble receptor comprising SEQ ID NO:113.

11. An isolated ZcytoR21 soluble receptor comprising SEQ ID NO:115.

12. An isolated ZcytoR21 soluble receptor comprising SEQ ID NO:117.

13. An isolated ZcytoR21 soluble receptor comprising SEQ ID NO:119.

14. An isolated ZcytoR21 soluble receptor comprising a first polypeptide comprising SEQ ID NO:115 and a second polypeptide comprising a polypeptide from the group consisting of: SEQ ID NOs: 117 and 119.

15. The isolated ZcytoR21 soluble receptor of claim 14, wherein the second polypeptide comprises SEQ ID NO: 117.

16. The isolated ZcytoR21 soluble receptor of claim 14, wherein the second polypeptide comprises SEQ ID NO:119.

17. An isolated ZcytoR21 soluble receptor comprising a first polypeptide comprising SEQ ID NO:117 and a second polypeptide comprising a polypeptide from the group consisting of: SEQ ID NOs: 115 and 119.

18. The isolated ZcytoR21 soluble receptor of claim 17, wherein the second polypeptide comprises SEQ ID NO: 115.

19. The isolated ZcytoR21 soluble receptor of claim 17, wherein the second polypeptide comprises SEQ ID NO: 119.

20. An isolated ZcytoR21 soluble receptor comprising a first polypeptide comprising SEQ ID NO:119 and a second polypeptide comprising a polypeptide from the group consisting of: SEQ ID NOs: 115 and 117.

18. The isolated ZcytoR21 soluble receptor of claim 20, wherein the second polypeptide comprises SEQ ID NO: 115.

21. The isolated ZcytoR21 soluble receptor of claim 17, wherein the second polypeptide comprises SEQ ID NO: 117.

22. An isolated soluble receptor comprising ZcytoR21, wherein ZcytoR21 comprises a polypeptide having a sequence of amino acid residues selected from the group consisting of: SEQ ID NOs:9, 12, 23, 122, 113, 115, 117, 119, amino acid residues 136-414 of SEQ ID NO:21, amino acid residues 24-414 of SEQ ID NO:21, amino acid residues 159-437 of SEQ ID NO:107, amino acid residues 136-414 of SEQ ID NO:109, and amino acid residues 24-414 of SEQ ID NO:109, and wherein said soluble receptor reduces the activity of IL-17C (SEQ ID NO:17).

23. An antibody or antibody fragment that binds to a polypeptide having a sequence of amino acid residues selected from the group consisting of: SEQ ID NOs:9, 12, 23, 122, 113, 115, 117, 119, amino acid residues 136-414 of SEQ ID NO:21, amino acid residues 24-414 of SEQ ID NO:21, amino acid residues 159-437 of SEQ ID NO:107, amino acid residues 136-414 of SEQ ID NO:109, and amino acid residues 24-414 of SEQ ID NO:109, and wherein said soluble receptor reduces the activity of IL-17C (SEQ ID NO:17).

24. The antibody or antibody fragment according to claim 23, wherein the or antibody fragment is (a) a polyclonal antibody, (b) a murine monoclonal antibody, (c) a humanized antibody derived from (b), (d) an antibody fragment, or (e) a human monoclonal antibody.

25. The antibody or antibody fragment according to claim 23, wherein the antibody further comprises a radionuclide, enzyme, substrate, cofactor, fluorescent marker, chemiluminescent marker, peptide tag, magnetic particle, drug, or toxin.

26. The antibody of claim 24, wherein the antibody further comprises PEGylation.

27. A method for treatment of an immune-mediated disease in a patient in need of such treatment comprising the step of administering a pharmaceutical composition comprising a soluble ZcytoR21 receptor.

28. The method of claim 27, wherein the soluble ZcytoR21 receptor is a polypeptide having a sequence of amino acid residues selected from the group consisting of: SEQ ID NOs:9, 12, 23, 122, 113, 115, 117, 119, amino acid residues 136-414 of SEQ ID NO:21, amino acid residues 24-414 of SEQ ID NO:21, amino acid residues 159-437 of SEQ ID NO:107, amino acid residues 136-414 of SEQ ID NO:109, and amino acid residues 24-414 of SEQ ID NO:109

29. A method of reducing IL-17C-mediated inflammation comprising administering to a mammal with inflammation an amount of a composition of an antibody according to claim 23 sufficient to reduce inflammation.

30. A method of reducing IL-17C-mediated inflammation comprising administering to a mammal with inflammation an amount of a composition comprising a ZcytoR21 soluble receptor sufficient to reduce inflammation, wherein said ZcytoR21 soluble receptor comprises a a polypeptide having a sequence of amino acid residues selected from the group consisting of: SEQ ID NOs:9, 12, 23, 122, 113, 115, 117, 119, amino acid residues 136-414 of SEQ ID NO:21, amino acid residues 24-414 of SEQ ID NO:21, amino acid residues 159-437 of SEQ ID NO:107, amino acid residues 136-414 of SEQ ID NO:109, and amino acid residues 24-414 of SEQ ID NO:109.

31. A method of treating a mammal afflicted with an inflammatory disease in which IL-17C plays a role, comprising:
administering an antagonist of ZcytoR21 to the mammal such that he inflammation is reduced, wherein the antagonist comprises (i) an antibody, antibody fragment, or binding polypeptide that specifically binds a polypeptide or polypeptide fragment of ZcytoR21, or (ii) a polypeptide or polypeptide fragment of ZcytoR21; and
wherein the inflammatory activity of IL-17C is reduced.

32. The method of claim 31, wherein the disease is asthma.

33. The method of claim 31, wherein the disease is a chronic inflammatory disease.

34. The method of claim 33, wherein the disease is a chronic inflammatory disease comprising inflammatory bowel disease, ulcerative colitis, Crohn's disease, arthritis, atopic dermatitis, or psoriasis.

35. The method of claim 31, wherein the disease is an acute inflammatory disease.

36. The method of claim 35, wherein the disease is an acute inflammatory disease comprising endotoxemia, septicemia, toxic shock syndrome or infectious disease.

37. The method of claim 31, wherein the antibody, antibody fragment, or binding polypeptide further comprises a radionuclide, enzyme, substrate, cofactor, fluorescent marker, chemiluminescent marker, peptide tag, magnetic particle, drug, or toxin.

38. A method of treating a pathological condition in a subject associated with ZcytoR21 activity comprising administering an effective amount of a ZcytoR21 soluble receptor, wherein said ZcytoR21 soluble receptor comprises a a polypeptide having a sequence of amino acid residues selected from the group consisting of: SEQ ID NOs:9, 12, 23, 122, 113, 115, 117, 119, amino acid residues 136-414 of SEQ ID NO:21, amino acid residues 24-414 of SEQ ID NO:21, amino acid residues 159-437 of SEQ ID NO:107, amino acid residues 136-414 of SEQ ID NO:109, and amino acid residues 24-414 of SEQ ID NO:109, thereby treating said pathological condition.

39. The method of claim 38, wherein said pathological condition is asthma.

40. The method of claim 38, wherein said pathological condition is a chronic inflammatory condition.

41. The method of claim 40 wherein said chronic inflammatory condition comprising inflammatory bowel disease, ulcerative colitis, Crohn's disease, arthritis, atopic dermatitis, or psoriasis.

42. The method of claim 38, wherein said pathological condition is an acute inflammatory condition.

43. The method of claim 42, wherein said acute inflammatory condition comprises endotoxemia, septicemia, toxic shock syndrome, or infectious disease.

44. A method of treating a mammal afflicted with an inflammatory disease in which ZcytoR21 plays a role, comprising:
administering an antagonist of ZcytoR21 to the mammal such that the inflammation is reduced, wherein the antagonist comprises an antibody, antibody fragment, ZcytoR21 soluble receptor or binding polypeptide that specifically binds a polypeptide or polypeptide fragment of ZcytoR21, and
wherein the inflammatory activity is reduced.

45. The method of claim 44, wherein the disease is asthma.

46. The method of claim 44, wherein the disease is a chronic inflammatory disease.

47. The method of claim 46, wherein the disease is a chronic inflammatory disease comprising inflammatory bowel disease, ulcerative colitis, Crohn's disease, arthritis, atopic dermatitis, or psoriasis.

48. The method of claim 44, wherein the disease is an acute inflammatory disease.

49. The method of claim 48, wherein the disease is an acute inflammatory disease comprising endotoxemia, septicemia, toxic shock syndrome or infectious disease.

50. The method of claim 44, wherein the antibody, antibody fragment, or binding polypeptide further comprises a radionuclide, enzyme, substrate, cofactor, fluorescent marker, chemiluminescent marker, peptide tag, magnetic particle, drug, or toxin.

51. The method of claim 44, wherein the antibody, antibody fragment, or binding polypeptide further comprises PEGylation.


SEQUENCE LISTING (186 sequences)

SEQ ID NO: 1 - DNA, 2172 bp, Homo sapiens
  CDS (66)...(2069)
aggccctgccacccaccttcaggccatgcagccatgttccgggagccctaattgcacagaagcccatggggagctccagactggcagccctgctcctgcctctcctcctcatagtcatcgacctctctgactctgctgggattggctttcgccacctgccccactggaacacccgctgtcctctggcctcccacacggatgacagtttcactggaagttctgcctatatcccttgccgcacctggtgggccctcttctccacaaagccttggtgtgtgcgagtctggcactgttcccgctgtttgtgccagcatctgctgtcaggtggctcaggtcttcaacggggcctcttccacctcctggtgcagaaatccaaaaagtcttccacattcaagttctataggagacacaagatgccagcacctgctcagaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacggctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccagatgtctcttacagacacctggggctcttgatcctggcactgctggccctcctcaccctactgggtgttgttctggccctcacctgccggcgcccacagtcaggcccgggcccagcgcggccagtgctcctcctgcacgcggcggactcggaggcgcagcggcgcctggtgggagcgctggctgaactgctacgggcagcgctgggcggcgggcgcgacgtgatcgtggacctgtgggaggggaggcacgtggcgcgcgtgggcccgctgccgtggctctgggcggcgcggacgcgcgtagcgcgggagcagggcactgtgctgctgctgtggagcggcgccgaccttcgcccggtcagcggccccgacccccgcgccgcgcccctgctcgccctgctccacgctgccccgcgcccgctgctgctgctcgcttacttcagtcgcctctgcgccaagggcgacatccccccgccgctgcgcgccctgccgcgctaccgcctgctgcgcgacctgccgcgtctgctgcgggcgctggacgcgcggcctttcgcagaggccaccagctggggccgccttggggcgcggcagcgcaggcagagccgcctagagctgtgcagccggcttgaacgagaggccgcccgacttgcagacctaggttgagcagagctccaccgcagtcccgggtgtctgcggccgcaacgcaacggacactggctggaaccccggaatgagccttcgaccctgaaatccttggggtgcctcg

SEQ ID NO: 2 - protein, 667 aa, Homo sapiens
MGSSRLAALLLPLLLIVIDLSDSAGIGFRHLPHWNTRCPLASHTDDSFTGSSAYIPCRTWWALFSTKPWCVRVWHCSRCLCQHLLSGGSGLQRGLFHLLVQKSKKSSTFKFYRRHKMPAPAQRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLVWRSDVQFAWKHLLCPDVSYRHLGLLILALLALLTLLGVVLALTCRRPQSGPGPARPVLLLHAADSEAQRRLVGALAELLRAALGGGRDVIVDLWEGRHVARVGPLPWLWAARTRVAREQGTVLLLWSGADLRPVSGPDPRAAPLLALLHAAPRPLLLLAYFSRLCAKGDIPPPLRALPRYRLLRDLPRLLRALDARPFAEATSWGRLGARQRRQSRLELCSRLEREAARLADLG

SEQ ID NO: 3 - protein, 454 aa, Homo sapiens
MGSSRLAALLLPLLLIVIDLSDSAGIGFRHLPHWNTRCPLASHTDDSFTGSSAYIPCRTWWALFSTKPWCVRVWHCSRCLCQHLLSGGSGLQRGLFHLLVQKSKKSSTFKFYRRHKMPAPAQRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLVWRSDVQFAWKHLLCPDVSYRH

SEQ ID NO: 4 - DNA, 1938 bp, Homo sapiens
  CDS (66)...(1835)
aggccctgccacccaccttcaggccatgcagccatgttccgggagccctaattgcacagaagcccatggggagctccagactggcagccctgctcctgcctctcctcctcatagtcatcgacctctctgactctgctgggattggctttcgccacctgccccactggaacacccgctgtcctctggcctcccacacgaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacagctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccggatgtctcttacagacacctggggctcttgatcctggcactgctggccctcctcaccctactgggtgttgttctggccctcacctgccggcgcccacagtcaggcccgggcccagcgcggccagtgctcctcctgcacgcggcggactcggaggcgcagcggcgcctggtgggagcgctggctgaactgctacgggcagcgctgggcggcgggcgcgacgtgatcgtggacctgtgggaggggaggcacgtggcgcgcgtgggcccgctgccgtggctctgggcggcgcggacgcgcgtagcgcgggagcagggcactgtgctgctgctgtggagcggcgccgaccttcgcccggtcagcggccccgacccccgcgccgcgcccctgctcgccctgctccacgctgccccgcgcccgctgctgctgctcgcttacttcagtcgcctctgcgccaagggcgacatccccccgccgctgcgcgccctgccgcgctaccgcctgctgcgcgacctgccgcgtctgctgcgggcgctggacgcgcggcctttcgcagaggccaccagctggggccgccttggggcgcggcagcgcaggcagagccgcctagagctgtgcagccggctcgaacgagaggccgcccgacttgcagacctaggttgagcagagctccaccacagtcccgggtgtctgcggccgcaacgcaacggacactggctggaaccccggaatgagccttcgaccctgaaatccttggggtgcctcg

SEQ ID NO: 5 - protein, 589 aa, Homo sapiens
MGSSRLAALLLPLLLIVIDLSDSAGIGFRHLPHWNTRCPLASHTRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLVWRSDVQFAWKHLLCPDVSYRHLGLLILALLALLTLLGVVLALTCRRPQSGPGPARPVLLLHAADSEAQRRLVGALAELLRAALGGGRDVIVDLWEGRHVARVGPLPWLWAARTRVAREQGTVLLLWSGADLRPVSGPDPRAAPLLALLHAAPRPLLLLAYFSRLCAKGDIPPPLRALPRYRLLRDLPRLLRALDARPFAEATSWGRLGARQRRQSRLELCSRLEREAARLADLG

SEQ ID NO: 6 - protein, 376 aa, Homo sapiens
MGSSRLAALLLPLLLIVIDLSDSAGIGFRHLPHWNTRCPLASHTRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLVWRSDVQFAWKHLLCPDVSYRH

SEQ ID NO: 7 - DNA, 1998 bp, Homo sapiens
  CDS (66)...(1892)
aggccctgccacccaccttcaggccatgcagccatgttccgggagccctaattgcacagaagcccatggggagctccagactggcagccctgctcctgcctctcctcctcatagtcatcgacctctctgactctgctgggattggctttcgccacctgccccactggaacacccgctgtcctctggcctcccacacggtcttcaacggggcctcttccacctcctggtgcagaaatccaaaaagtcttccacattcaagttctataggagacacaagatgccagcacctgctcagaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacagctcgagagtcagatgggtggtatgttttggagaaggtggacctgcacccccagctctgcttcaagttctcttttggaaacagcagccatgttgaatgcccccaccagactggaataacagaggcaagggactggccctcccacattcaggtgtcctgtagcccaggggtcccaatccgtgagccgcagaccagtaactgtctgtggtttgtgagaaacgaggccacacagcaggaggcccggggctcaagcccagtgtcactagacctcatcattcccttcctgaggccagggtgctgtgtcctggtgtggcggtcagatgtccagtttgcctggaagcacctcttgtgtccggatgtctcttacagacacctggggctcttgatcctggcactgctggccctcctcaccctactgggtgttgttctggccctcacctgccggcgcccacagtcaggcccgggcccagcgcggccagtgctcctcctgcacgcggcggactcggaggcgcagcggcgcctggtgggagcgctggctgaactgctacgggcagcgctgggcggcgggcgcgacgtgatcgtggacctgtgggaggggaggcacgtggcgcgcgtgggcccgctgccgtggctctgggcggcgcggacgcgcgtagcgcgggagcagggcactgtgctgctgctgtggagcggcgccgaccttcgcccggtcagcggccccgacccccgcgccgcgcccctgctcgccctgctccacgctgccccgcgcccgctgctgctgctcgcttacttcagtcgcctctgcgccaagggcgacatccccccgccgctgcgcgccctgccgcgctaccgcctgctgcgcgacctgccgcgtctgctgcgggcgctggacgcgcggcctttcgcagaggccaccagctggggccgccttggggcgcggcagcgcaggcagagccgcctagagctgtgcagccggctcgaacgagaggccgcccgacttgcagacctaggttgagcagagctccaccgcagtcccgggtgtctgcggccgcaacgcaacggacactggctggaaccccggaatgagccttcgaccctgaaatccttggggtgcctcg

SEQ ID NO: 8 - protein, 609 aa, Homo sapiens
MGSSRLAALLLPLLLIVIDLSDSAGIGFRHLPHWNTRCPLASHTVFNGASSTSWCRNPKSLPHSSSIGDTRCQHLLRGSCCLVVTCLRRAITFPSPPQTSPTRDFALKGPNLRIQRHGKVFPDWTHKGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGITEARDWPSHIQVSCSPGVPIREPQTSNCLWFVRNEATQQEARGSSPVSLDLIIPFLRPGCCVLVWRSDVQFAWKHLLCPDVSYRHLGLLILALLALLTLLGVVLALTCRRPQSGPGPARPVLLLHAADSEAQRRLVGALAELLRAALGGGRDVIVDLWEGRHVARVGPLPWLWAARTRVAREQGTVLLLWSGADLRPVSGPDPRAAPLLALLHAAPRPLLLLAYFSRLCAKGDIPPPLRALPRYRLLRDLPRLLRALDARPFAEATSWGRLGARQRRQSRLELCSRLEREAARLADLG

SEQ ID NO: 9 - protein, 373 aa, Homo sapiens
AGIGFRHLPHWNTRCPLASHTVFNGASSTSWCRNPKSLPHSSSIGDTRCQHLLRGSCCLVVTCLRRAITFPSPPQTSPTRDFALKGPNLRIQRHGKVFPDWTHKGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGITEARDWPSHIQVSCSPGVPIREPQTSNCLWFVRNEATQQEARGSSPVSLDLIIPFLRPGCCVLVWRSDVQFAWKHLLCPDVSYRH

SEQ ID NO: 10 - DNA, 2245 bp, Homo sapiens
  CDS (66)...(1664)
aggccctgccacccaccttcaggccatgcagccatgttccgggagccctaattgcacagaagcccatggggagctccagactggcagccctgctcctgcctctcctcctcatagtcatcgacctctctgactctgctgggattggctttcgccacctgccccactggaacacccgctgtcctctggcctcccacacggatgacagtttcactggaagttctgcctatatcccttgccgcacctggtgggccctcttctccacaaagccttggtgtgtgcgagtctggcactgttcccgctgtttgtgccagcatctgctgtcaggtggctcaggtcttcaacggggcctcttccacctcctggtgcagaaatccaaaaagtcttccacattcaagttctataggagacacaagatgccagcacctgctcagaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacagctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctgctccatgcttcactcagctccccgggaggagaagatgcctggctcataggggtggggggctctgtgccctcaggtgtggcggtcagatgtccagtttgcctggaagcacctcttgtgtccggatgtctcttacagacacctggggctcttgatcctggcactgctggccctcctcaccctactgggtgttgttctggccctcacctgccggcgcccacagtcaggcccgggcccagcgcggccagtgctcctcctgcacgcggcggactcggaggcgcagcggcgcctggtgggagcgctggctgaactgctacgggcagcgctgggcggcgggcgcgacgtgatcgtggacctgtgggaggggaggcacgtggcgcgcgtgggcccgctgccgtggctctgggcggcgcggacgcgcgtagcgcgggagcagggcactgtgctgctgctgtggagcggcgccgaccttcgcccggtcagcggccccgacccccgcgccgcgcccctgctcgccctgctccacgctgccccgcgcccgctgctgctgctcgcttacttcagtcgcctctgcgccaagggcgacatccccccgccgctgcgcgccctgccgcgctaccgcctgctgcgcgacctgccgcgtctgctgcgggcgctggacgcgcggcctttcgcagaggccaccagctggggccgccttggggcgcggcagcgcaggcagagccgcctagagctgtgcagccggctcgaacgagaggccgcccgacttgcagacctaggttgagcagagctccaccgcagtcccgggtgtctgcggccgcaacgcaacggacactggctggaaccccggaatgagccttcgaccctgaaatccttggggtgcctcg

SEQ ID NO: 11 - protein, 533 aa, Homo sapiens
MGSSRLAALLLPLLLIVIDLSDSAGIGFRHLPHWNTRCPLASHTDDSFTGSSAYIPCRTWWALFSTKPWCVRVWHCSRCLCQHLLSGGSGLQRGLFHLLVQKSKKSSTFKFYRRHKMPAPAQRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLLHASLSSPGGEDAWLIGVGGSVPSGVAVRCPVCLEAPLVSGCLLQTPGALDPGTAGPPHPTGCCSGPHLPAPTVRPGPSAASAPPARGGLGGAAAPGGSAG

SEQ ID NO: 12 - protein, 510 aa, Homo sapiens
AGIGFRHLPHWNTRCPLASHTDDSFTGSSAYIPCRTWWALFSTKPWCVRVWHCSRCLCQHLLSGGSGLQRGLFHLLVQKSKKSSTFKFYRRHKMPAPAQRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLLHASLSSPGGEDAWLIGVGGSVPSGVAVRCPVCLEAPLVSGCLLQTPGALDPGTAGPPHPTGCCSGPHLPAPTVRPGPSAASAPPARGGLGGAAAPGGSAG

SEQ ID NO: 13 - DNA, 2221 bp, Mus musculus
  CDS (76)...(1989)
ctccagggccaggccctgctgccctcttgcagacaggaaagacatggtctctgcgcccggatcctacagaagctcatggggagccccagactggcagccttgctcctgtctctcccgctactgctcatcggcctcgctgtgtctgctcgggttgcctgcccctgcctgcggagttggaccagccactgtctcctggcctaccgtgtggataaacgttttgctggccttcagtggggctggttccctctcttggtgaggaaatctaaaagtcctcctaaatttgaagactattggaggcacaggacaccagcatccttccagaggaagctgctaggcagcccttccctgtctgaggaaagccatcgaatttccatcccctcctcagccatctcccacagaggccaacgcaccaaaagggcccagccttcagctgcagaaggaagagaacatctccctgaagcagggtcacaaaagtgtggaggacctgaattctcctttgatttgctgcccgaggtgcaggctgttcgggtgactattcctgcaggccccaaggccagtgtgcgcctttgttatcagtgggcactggaatgtgaagacttgagtagcccttttgatacccagaaaattgtgtctggaggccacactgtagacctgccttatgaattccttctgccctgcatgtgcatagaggcctcctacctgcaagaggacactgtgaggcgcaaaaagtgtcccttccagagctggcctgaagcttatggctcagacttctggcagtcaatacgcttcactgactacagccagcacaatcagatggtcatggctctgacactccgctgcccactgaaactggaggcctccctctgctggaggcaggacccactcacaccctgcgaaacccttcccaacgccacagcacaggagtcagaaggatggtatatcctggagaatgtggacttgcacccccagctctgctttaagttctcatttgaaaacagcagccacgttgaatgtccccaccagagtggctctctcccatcctggactgtgagcatggatacccaggcccagcagctgacgcttcacttttcttcgaggacatatgccaccttcagtgctgcctggagtgacccaggtttggggccggatacccccatgcctcctgtgtacagcatcagccagacccagggctcagtcccagtgacgctagacctcatcatccccttcctgaggcaggagaattgcatcctggtgtggaggtcagatgtccattttgcctggaagcacgtcttgtgtcctgatgtctcccatagacacctcgggctcttgatcctggcactgctggctctcaccgctctagtgggtgtagttctggtcctcctcggccggcgcctactgccaggctccggtcgaacaaggccagttttactcctacatgcagcggactcagaggcacagcgacgcctggtgggagctttggccgaactgctgcggacggcgctgggaggtggacgcgacgtgatcgtggatctctgggaagggacgcacgtagcacgcattggaccactgccgtggctttgggcagcgcgggagcgcgtggcgcgggagcagggcacagtgctgctcctgtggaactgtgcgggtcccagcaccgcctgcagcggtgacccgcaggctgcgtcccttcgcaccttgttgtgcgctgctccacgtccgctgctgctcgcctacttcagtcgcctctgcgccaaaggtgacatcccccggccgctgcgcgctctgccacgctaccgcctgcttcgtgacctgccgcgcctgctgagagcactggatgctcagcctgccaccctagcctccagctggagtcaccttggggctaagcggtgcttgaaaaaccgtctggagcagtgtcacctgctggaacttgaggctgccaaagatgactaccaaggctcaaccaatagtccctgtggtttcagctgtctgtagcctcagcctgtgtagcaacagcaggaactccagaatgaggcctcacacatgtactctttgggggtgcttcttgtcccccaaaccgtaagactcaccttaagtcccacacttgaccaacctccctcacatttgctccctcttagagttcctgagaggaacttgggctttcctgataggtcctcagccctttctgagaaggagggacgatttttccatttcttttcaaaactga

SEQ ID NO: 14 - protein, 637 aa, Mus musculus
MGSPRLAALLLSLPLLLIGLAVSARVACPCLRSWTSHCLLAYRVDKRFAGLQWGWFPLLVRKSKSPPKFEDYWRHRTPASFQRKLLGSPSLSEESHRISIPSSAISHRGQRTKRAQPSAAEGREHLPEAGSQKCGGPEFSFDLLPEVQAVRVTIPAGPKASVRLCYQWALECEDLSSPFDTQKIVSGGHTVDLPYEFLLPCMCIEASYLQEDTVRRKKCPFQSWPEAYGSDFWQSIRFTDYSQHNQMVMALTLRCPLKLEASLCWRQDPLTPCETLPNATAQESEGWYILENVDLHPQLCFKFSFENSSHVECPHQSGSLPSWTVSMDTQAQQLTLHFSSRTYATFSAAWSDPGLGPDTPMPPVYSISQTQGSVPVTLDLIIPFLRQENCILVWRSDVHFAWKHVLCPDVSHRHLGLLILALLALTALVGVVLVLLGRRLLPGSGRTRPVLLLHAADSEAQRRLVGALAELLRTALGGGRDVIVDLWEGTHVARIGPLPWLWAARERVAREQGTVLLLWNCAGPSTACSGDPQAASLRTLLCAAPRPLLLAYFSRLCAKGDIPRPLRALPRYRLLRDLPRLLRALDAQPATLASSWSHLGAKRCLKNRLEQCHLLELEAAKDDYQGSTNSPCGFSCL

SEQ ID NO: 15 - protein, 391 aa, Mus musculus
ARVACPCLRSWTSHCLLAYRVDKRFAGLQWGWFPLLVRKSKSPPKFEDYWRHRTPASFQRKLLGSPSLSEESHRISIPSSAISHRGQRTKRAQPSAAEGREHLPEAGSQKCGGPEFSFDLLPEVQAVRVTIPAGPKASVRLCYQWALECEDLSSPFDTQKIVSGGHTVDLPYEFLLPCMCIEASYLQEDTVRRKKCPFQSWPEAYGSDFWQSIRFTDYSQHNQMVMALTLRCPLKLEASLCWRQDPLTPCETLPNATAQESEGWYILENVDLHPQLCFKFSFENSSHVECPHQSGSLPSWTVSMDTQAQQLTLHFSSRTYATFSAAWSDPGLGPDTPMPPVYSISQTQGSVPVTLDLIIPFLRQENCILVWRSDVHFAWKHVLCPDVSHRH

SEQ ID NO: 16 - DNA, 1048 bp, Homo sapiens
  CDS (50)...(643)
gccaggtgtgcaggccgctccaagcccagcctgccccgctgccgccaccatgacgctcctccccggcctcctgtttctgacctggctgcacacatgcctggcccaccatgacccctccctcagggggcacccccacagtcacggtaccccacactgctactcggctgaggaactgcccctcggccaggcccccccacacctgctggctcgaggtgccaagtgggggcaggctttgcctgtagccctggtgtccagcctggaggcagcaagccacagggggaggcacgagaggccctcagctacgacccagtgcccggtgctgcggccggaggaggtgttggaggcagacacccaccagcgctccatctcaccctggagataccgtgtggacacggatgaggaccgctatccacagaagctggccttcgccgagtgcctgtgcagaggctgtatcgatgcacggacgggccgcgagacagctgcgctcaactccgtgcggctgctccagagcctgctggtgctgcgccgccggccctgctcccgcgacggctcggggctccccacacctggggcctttgccttccacaccgagttcatccacgtccccgtcggctgcacctgcgtgctgccccgttcagtgtgaccgccgaggccgtggggcccctagactggacacgtgtgctccccagagggcaccccctatttatgtgtatttattgttatttatatgcctcccccaacactacccttggggtctgggcattccccgtgtctggaggacagccccccactgttctcctcatctccagcctcagtagttgggggtagaaggagctcagcacctcttccagcccttaaagctgcagaaaaggtgtcacacggctgcctgtaccttggctccctgtcctgctcccggcttcccttaccctatcactggcctcaggcccccgcaggctgcctcttcccaacctccttggaagtacccctgtttcttaaacaattatttaagtgtacgtgtattattaaactgatgaacacatccccaaaa

SEQ ID NO: 17 - protein, 197 aa, Homo sapiens
MTLLPGLLFLTWLHTCLAHHDPSLRGHPHSHGTPHCYSAEELPLGQAPPHLLARGAKWGQALPVALVSSLEAASHRGRHERPSATTQCPVLRPEEVLEADTHQRSISPWRYRVDTDEDRYPQKLAFAECLCRGCIDARTGRETAALNSVRLLQSLLVLRRRPCSRDGSGLPTPGAFAFHTEFIHVPVGCTCVLPRSV

SEQ ID NO: 18 - DNA, 609 bp, Mus musculus
  CDS (1)...(609)
atggccaccgtcaccgtcactgtgatgagtctcctgcttctaggctggttgcctactgggatgacccaccaagatcccccgtcctgggggaaaccccgaagccataggaccctgcggtgctactctgctgaggaattatctcacggccaggctcctccacacctgctaactcgaagtgccaggtgggagcaggccctccctgtggccctggtggccagtttggaggccacgggccacaggagacagcatgaaggacctctagctggaacacagtgccccgtgctgcggccggaggaggtgctggaagctgacactcacgagcgctccatctcaccatggagatatcgcatcgacacagatgagaaccgctacccacagaagctggcggtggcagaatgcttgtgtcgtggatgcatcaacgccaagacaggccgtgagacagctgccctgaactcggtgcagctgctgcagagcctgctggtactacggcgacagccctgctcccgagacggcacggcggaccctacaccaggatccttcgccttccacaccgagttcatccgcgtgcctgtcggctgcacctgcgttcttcccaggtctacacagtga

SEQ ID NO: 19 - protein, 202 aa, Mus musculus
MATVTVTVMSLLLLGWLPTGMTHQDPPSWGKPRSHRTLRCYSAEELSHGQAPPHLLTRSARWEQALPVALVASLEATGHRRQHEGPLAGTQCPVLRPEEVLEADTHERSISPWRYRIDTDENRYPQKLAVAECLCRGCINAKTGRETAALNSVQLLQSLLVLRRQPCSRDGTADPTPGSFAFHTEFIRVPVGCTCVLPRSTQ

SEQ ID NO: 20 - DNA, 1884 bp, Homo sapiens
  CDS (1)...(1884)
atggggagctccagactggcagccctgctcctgcctctcctcctcatagtcatcgacctctctgactctgctgggattggctttcgccacctgccccactggaacacccgctgtcctctggcctcccacacggatgacagtttcactggtcttcaacggggcctcttccacctcctggtgcagaaatccaaaaagtcttccacattcaagttctataggagacacaagatgccagcacctgctcagaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacggctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccagatgtctcttacagacacctggggctcttgatcctggcactgctggccctcctcaccctactgggtgttgttctggccctcacctgccggcgcccacagtcaggcccgggcccagcgcggccagtgctcctcctgcacgcggcggactcggaggcgcagcggcgcctggtgggagcgctggctgaactgctacgggcagcgctgggcggcgggcgcgacgtgatcgtggacctgtgggaggggaggcacgtggcgcgcgtgggcccgctgccgtggctctgggcggcgcggacgcgcgtagcgcgggagcagggcactgtgctgctgctgtggagcggcgccgaccttcgcccggtcagcggccccgacccccgcgccgcgcccctgctcgccctgctccacgctgccccgcgcccgctgctgctgctcgcttacttcagtcgcctctgcgccaagggcgacatccccccgccgctgcgcgccctgccgcgctaccgcctgctgcgcgacctgccgcgtctgctgcgggcgctggacgcgcggcctttcgcagaggccaccagctggggccgccttggggcgcggcagcgcaggcagagccgcctagagctgtgcagccggcttgaacgagaggccgcccgacttgcagacctaggttga

SEQ ID NO: 21 - protein, 627 aa, Homo sapiens
MGSSRLAALLLPLLLIVIDLSDSAGIGFRHLPHWNTRCPLASHTDDSFTGLQRGLFHLLVQKSKKSSTFKFYRRHKMPAPAQRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLVWRSDVQFAWKHLLCPDVSYRHLGLLILALLALLTLLGVVLALTCRRPQSGPGPARPVLLLHAADSEAQRRLVGALAELLRAALGGGRDVIVDLWEGRHVARVGPLPWLWAARTRVAREQGTVLLLWSGADLRPVSGPDPRAAPLLALLHAAPRPLLLLAYFSRLCAKGDIPPPLRALPRYRLLRDLPRLLRALDARPFAEATSWGRLGARQRRQSRLELCSRLEREAARLADLG

SEQ ID NO: 22 - DNA, 1650 bp, Homo sapiens
  CDS (1)...(1266)
atggggagctccagactggcagccctgctcctgcctctcctcctcatagtcatcgacctctctgactctgctgggattggctttcgccacctgccccactggaacacccgctgtcctctggcctcccacacggatgacagtttcactggaagttctgcctatatcccttgccgcacctggtgggccctcttctccacaaagccttggtgtgtgcgagtctggcactgttcccgctgtttgtgccagcatctgctgtcaggtggctcaggtcttcaacggggcctcttccacctcctggtgcagaaatccaaaaagtcttccacattcaagttctataggagacacaagatgccagcacctgctcagaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacggctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccagggcttggcacagaggacacactcagagtctgtctgttgacaaatacctgactgcagcaggagctgagctctgggaaagatcagtgggagccggagtgactggggaagccttcttgtcagaggcccggggctcaagcccagtgtcactagacctcatcattcccttcctgaggccagggtgctgtgtcctggtgtggcggtcagatgtccagtttgcctggaagcacctcttgtgtccagatgtctcttacagacacctggggctcttgatcctggcactgctggccctcctcaccctactgggtgttgttctggccctcacctgccggcgcccacagtcaggcccgggcccagcgcggccagtgctcctcctgcacgcggcggactcggaggcgcagcggcgcctggtgggagcgctggctga

SEQ ID NO: 23 - protein, 421 aa, Homo sapiens
MGSSRLAALLLPLLLIVIDLSDSAGIGFRHLPHWNTRCPLASHTDDSFTGSSAYIPCRTWWALFSTKPWCVRVWHCSRCLCQHLLSGGSGLQRGLFHLLVQKSKKSSTFKFYRRHKMPAPAQRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQGLAQRTHSESVC

SEQ ID NO: 24 - protein, 320 aa, Homo sapiens
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLW

SEQ ID NO: 25 - protein, 16 aa, Artificial Sequence
  peptide linker
GGSGGSGGGGSGGGGS

SEQ ID NO: 26 - DNA, 20 bp, Artificial Sequence
  primer
aggccctgccacccaccttc

SEQ ID NO: 27 - DNA, 22 bp, Artificial Sequence
  primer
cgaggcaccccaaggatttcag

SEQ ID NO: 28 - DNA, 22 bp, Artificial Sequence
  primer
tctctgactctgctgggattgg

SEQ ID NO: 29 - DNA, 24 bp, Artificial Sequence
  primer
atgaggaccgctatccacagaagc

SEQ ID NO: 30 - DNA, 23 bp, Artificial Sequence
  primer
ggacgtggatgaactcggtgtgg

SEQ ID NO: 31 - DNA, 22 bp, Artificial Sequence
  primer
gcacacctggcggcaccatgac

SEQ ID NO: 32 - DNA, 23 bp, Artificial Sequence
  primer
ctgtcctccagacacggggaatg

SEQ ID NO: 33 - DNA, 28 bp, Artificial Sequence
  primer
cagccagaattcacctgttacagcactg

SEQ ID NO: 34 - DNA, 29 bp, Artificial Sequence
  primer
tacacagaattcgaaggtgtcctcctctg

SEQ ID NO: 35 - DNA, 39 bp, Artificial Sequence
  primer
cacacaggccggccaccatgacgctcctccccggcctcc

SEQ ID NO: 36 - DNA, 38 bp, Artificial Sequence
  primer
cacacaggcgcgccttcacactgaacggggcagcacgc

SEQ ID NO: 37 - DNA, 39 bp, Artificial Sequence; identical to SEQ ID NO: 35
  primer

SEQ ID NO: 38 - DNA, 38 bp, Artificial Sequence; identical to SEQ ID NO: 36
  primer

SEQ ID NO: 39 - DNA, 25 bp, Artificial Sequence
  primer
gcctcccacacgaggaagctgctgc

SEQ ID NO: 40 - DNA, 25 bp, Artificial Sequence
  primer
gcagcagcttcctcgtgtgggaggc

SEQ ID NO: 41 - DNA, 25 bp, Artificial Sequence
  primer
tggactcacaaaggacccgagttct

SEQ ID NO: 42 - DNA, 25 bp, Artificial Sequence
  primer
gcctctgttattccagtctggtggg

SEQ ID NO: 43 - DNA, 25 bp, Artificial Sequence
  primer
ccccgttgaagaccgtgtgggaggc

SEQ ID NO: 44 - DNA, 25 bp, Artificial Sequence
  primer
cccaccagactggaataacagaggc

SEQ ID NO: 45 - DNA, 25 bp, Artificial Sequence
  primer
gcctcccacacggtcttcaacgggg

SEQ ID NO: 46 - DNA, 25 bp, Artificial Sequence
  primer
agaactcgggtcctttgtgagtcca

SEQ ID NO: 47 - DNA, 25 bp, Artificial Sequence
  primer
tgctgtgtcctgctccatgcttcac

SEQ ID NO: 48 - DNA, 25 bp, Artificial Sequence
  primer
gtgaagcatggagcaggacacagca

SEQ ID NO: 49 - DNA, 25 bp, Artificial Sequence
  primer
tctgactctgctgggattggctttc

SEQ ID NO: 50 - DNA, 25 bp, Artificial Sequence
  primer
gaaagccaatcccagcagagtcaga

SEQ ID NO: 51 - DNA, 25 bp, Artificial Sequence
  primer
tgctgctgctgtggagcggcgccga

SEQ ID NO: 52 - DNA, 25 bp, Artificial Sequence
  primer
tcggcgccgctccacagcagcagca

SEQ ID NO: 53 - DNA, 22 bp, Artificial Sequence
  primer
cgaggcaccccaaggatttcag

SEQ ID NO: 54 - DNA, 20 bp, Artificial Sequence
  primer
aggccctgccacccaccttc

SEQ ID NO: 55 - DNA, 22 bp, Artificial Sequence
  primer
tgcgcccggatcctacagaagc

SEQ ID NO: 56 - DNA, 23 bp, Artificial Sequence
  primer
gcacctcgggcagcaaatcaaag

SEQ ID NO: 57 - DNA, 25 bp, Artificial Sequence
  primer
ctctccatccttatctttcatcaac

SEQ ID NO: 58 - DNA, 24 bp, Artificial Sequence
  primer
ctctctgctggctaaacaaaacac

SEQ ID NO: 59 - DNA, 26 bp, Artificial Sequence
  primer
ctcatattgctcaactgtgtgaaaag

SEQ ID NO: 60 - DNA, 25 bp, Artificial Sequence
  primer
tagaagccacctgaacacaaatctg

SEQ ID NO: 61 - DNA, 28 bp, Artificial Sequence
  primer
atcttgcgttgtatgttgaaaatcaatt

SEQ ID NO: 62 - DNA, 25 bp, Artificial Sequence
  primer
ttctccaccaggtaaacaagtctac

SEQ ID NO: 63 - DNA, 24 bp, Artificial Sequence
  primer
tcctgcctctcctcctcatagtca

SEQ ID NO: 64 - DNA, 24 bp, Artificial Sequence
  primer
ccaggatcaagagccccaggtgtc

SEQ ID NO: 65 - DNA, 38 bp, Artificial Sequence
  primer
cgtacgggccggccaccatggggagctccagactggca

SEQ ID NO: 66 - DNA, 33 bp, Artificial Sequence
  primer
tgacgaggcgcgcctcaacctaggtctgcaagt

SEQ ID NO: 67 - DNA, 35 bp, Artificial Sequence
  primer
tttcgccacctgccccactggaacacccgctgtcc

SEQ ID NO: 68 - DNA, 2095 bp, Mus musculus
  CDS (89)...(1864)
gtgcttctcacagctccagggccaggccctgctgccctcttgcagacaggaaagacatggtctctgcgcccggatcctacagaagctcatggggagccccagactggcagccttgctcctgtctctcccgctactgctcatcggcctcgctgtgtctgctcgggttgcctgcccctgcctgcggagttggaccagccactgtctcctggcctaccgtgtggataaacgttttgctggccttcagtggggctggttccctctcttggtgaggaaatctaaaagtcctcctaaatttgaagactattggaggcacaggacaccagcatccttccagaggaagctgctaggcagcccttccctgtctgaggaaagccatcgaatttccatcccctcctcagccatctcccacagaggccaacgcaccaaaagggcccagccttcagctgcagaaggaagagaacatctccctgaagcagggtcacaaaagtgtggaggacctgaattctcctttgatttgctgcccgaggtgcaggctgttcgggtgactattcctgcaggccccaaggccagtgtgcgcctttgttatcagtgggcactggaatgtgaagacttgagtagcccttttgatacccagaaaattgtgtctggaggccacactgtagacctgccttatgaattccttctgccctgcatgtgcatagaggcctcctacctgcaagaggacactgtgaggcgcaaaaagtgtcccttccagagctggcctgaagcttatggctcagacttctggcagtcaatacgcttcactgactacagccagcacaatcagatggtcatggctctgacactccgctgcccactgaaactggaggcctccctctgctggaggcaggacccactcacaccctgcgaaacccttcccaacgccacagcacaggagtcagaaggatggtatatcctggagaatgtggacttgcacccccagctctgctttaagttctcatttgaaaacagcagccacgttgaatgtccccaccagagtggctctctcccatcctggactgtgagcatggatacccaggcccagcagctgacgcttcacttttcttcgaggacatatgccaccttcagtgctgcctggagtgacccaggtttggggccggatacccccatgcctcctgtgtacagcatcagccagacccagggctcagtcccagtgacgctagacctcatcatccccttcctgaggcaggagaattgcatcctggtgtggaggtcagatgtccattttgcctggaagcacgtcttgtgtcctgatgcggactcagaggcacagcgacgcctggtgggagctttggccgaactgctgcggacggcgctgggaggtggacgcgacgtgatcgtggatctctgggaagggacgcacgtagcacgcattggaccactgccgtggctttgggcagcgcgggagcgcgtggcgcgggagcagggcacagtgctgctcctgtggaactgtgcgggtcccagcaccgcctgcagcggtgacccgcaggctgcgtcccttcgcaccttgttgtgcgctgctccacgtccgctgctgctcgcctacttcagtcgcctctgcgccaaaggtgacatcccccggccgctgcgcgctctgccacgctaccgcctgcttcgtgacctgccgcgcctgctgagagcactggatgctcagcctgccaccctagcctccagctggagtcaccttggggctaagcggtgcttgaaaaaccgtctggagcagtgtcacctgctggaacttgaggctgccaaagatgactaccaaggctcaaccaatagtccctgtggtttcagctgtctgtagcctcagcctgtgtagcaacagcaggaactccagaatgaggcctcacacatgtactctttgggggtgcttcttgtcccccaaaccgtaagactcaccttaagtcccacacttgaccaacctccctcacatttgctccctcttagagttcctgagaggaacttgggctttcctgataggtcctcagccctttctgagaaggagggacgatttttccatttcttttcaaaactg

SEQ ID NO: 69 - protein, 21 aa, Mus musculus
CCTGCCCCTGCCTGCGGAGTT

SEQ ID NO: 70 - DNA, 21 bp, Artificial Sequence
  primer
cctgcccctgcctgcggagtt

SEQ ID NO: 71 - DNA, 24 bp, Artificial Sequence
  primer
gttgctacacaggctgaggctaca

SEQ ID NO: 72 - DNA, 34 bp, Artificial Sequence
  primer
ctaccaaggctcaaccaatagtccctgtggtttc

SEQ ID NO: 73 - DNA, 27 bp, Artificial Sequence
  primer
tcactgtgatgagtctcctgcttctag

SEQ ID NO: 74 - DNA, 28 bp, Artificial Sequence
  primer
gtgtcgatgcgatatctccatggtgaga

SEQ ID NO: 75 - DNA, 29 bp, Artificial Sequence
  primer
gagatatcgcatcgacacagatgagaacc

SEQ ID NO: 76 - DNA, 22 bp, Artificial Sequence
  primer
tcactgtgtagacctgggaaga

SEQ ID NO: 77 - DNA, 45 bp, Artificial Sequence
  primer
gccaccatggccaccgtcaccgtcactgtgatgagtctcctgctt

SEQ ID NO: 78 - DNA, 49 bp, Artificial Sequence
  C-terminal HIS tag
ggctcaggatctggtggcggccatcaccaccatcatcactaaatctaga

SEQ ID NO: 79 - DNA, 37 bp, Artificial Sequence
  primer
gaagaacgtctctcatggggagctccagactggcagc

SEQ ID NO: 80 - DNA, 40 bp, Artificial Sequence
  primer
gaagaacgtctctagccgtgtctgtaagagacatccggac

SEQ ID NO: 81 - DNA, 21 bp, Artificial Sequence
  primer
ctgtgaggcgcaaaaagtgtc

SEQ ID NO: 82 - DNA, 22 bp, Artificial Sequence
  primer
gcaagtccacattctccaggat

SEQ ID NO: 83 - DNA, 1287 bp, Homo sapiens
gctgggattggctttcgccacctgccccactggaacacccgctgtcctctggcctcccacacggatgacagtttcactggaagttctgcctatatcccttgccgcacctggtgggccctcttctccacaaagccttggtgtgtgcgagtctggcactgttcccgctgtttgtgccagcatctgctgtcaggtggctcaggtcttcaacggggcctcttccacctcctggtgcagaaatccaaaaagtcttccacattcaagttctataggagacacaagatgccagcacctgctcagaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacggctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccagatgtctcttac

SEQ ID NO: 84 - protein, 429 aa, Homo sapiens
AGIGFRHLPHWNTRCPLASHTDDSFTGSSAYIPCRTWWALFSTKPWCVRVWHCSRCLCQHLLSGGSGLQRGLFHLLVQKSKKSSTFKFYRRHKMPAPAQRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLVWRSDVQFAWKHLLCPDVSY

SEQ ID NO: 85 - DNA, 69 bp, Artificial Sequence
  primer
gtttcgctcagccaggaaatccatgccgagttgagacgcttccgtagagctgggattggctttcgccac

SEQ ID NO: 86 - DNA, 78 bp, Artificial Sequence
  primer
caaccccagagctgttttaaggcgcgcctctagattattccatgggcatgtattcttcgtaagagacatctggacaca

SEQ ID NO: 87 - protein, 10 aa, Artificial Sequence
  C-terminal HIS tag
GSGGHHHHHH

SEQ ID NO: 88 - protein, 10 aa, Artificial Sequence
  C-terminal FLAG tag
GSDYKDDDDK

SEQ ID NO: 89 - DNA, 87 bp, Artificial Sequence
  primer
caaccccagagctgttttaaggcgcgcctctagattagtgatggtgatggtgatgtccaccagatccgtaagagacatctggacaca

SEQ ID NO: 90 - DNA, 86 bp, Artificial Sequence
  primer
caaccccagagctgttttaaggcgcgcctctagattactatcatcatcatccttataatcggatccgtaagagacatctggacaca

SEQ ID NO: 91 - DNA, 1053 bp, Homo sapiens
gctgggattggctttcgccacctgccccactggaacacccgctgtcctctggcctcccacacgaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacagctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccagatgtctcttac

SEQ ID NO: 92 - protein, 351 aa, Homo sapiens
AGIGFRHLPHWNTRCPLASHTRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLVWRSDVQFAWKHLLCPDVSY

SEQ ID NO: 93 - DNA, 69 bp, Artificial Sequence; identical to SEQ ID NO: 85
  primer

SEQ ID NO: 94 - DNA, 78 bp, Artificial Sequence; identical to SEQ ID NO: 86
  primer

SEQ ID NO: 95 - protein, 10 aa, Artificial Sequence
  C-terminal His tag
GSGGHHHHHH

SEQ ID NO: 96 - protein, 10 aa, Artificial Sequence
  C-terminal FLAG tag
GSDYKDDDDK

SEQ ID NO: 97 - DNA, 87 bp, Artificial Sequence; identical to SEQ ID NO: 89
  primer

SEQ ID NO: 98 - DNA, 87 bp, Artificial Sequence
  primer
caaccccagagctgttttaaggcgcgcctctagattacttatcatcatcatccttataatcggatccgtaagagacatctggacaca

SEQ ID NO: 99 - DNA, 2145 bp, Homo sapiens
atggatgcaatgaagagagggctctgctgtgtgctgctgctgtgtggcgccgtcttcgtttcgctcagccaggaaatccatgccgagttgagacgcttccgtagagctgggattggctttcgccacctgccccactggaacacccgctgtcctctggcctcccacacggatgacagtttcactggaagttctgcctatatcccttgccgcacctggtgggccctcttctccacaaagccttggtgtgtgcgagtctggcactgttcccgctgtttgtgccagcatctgctgtcaggtggctcaggtcttcaacggggcctcttccacctcctggtgcagaaatccaaaaagtcttccacattcaagttctataggagacacaagatgccagcacctgctcagaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacggctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccagatgtctcttacgggggttcgggtggctcaggcggaggatccggatctgatgaagttgatggatcagagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 100 - protein, 714 aa, Homo sapiens [sequence omitted]

SEQ ID NO: 101 - DNA, 1911 bp, Homo sapiens
atggatgcaatgaagagagggctctgctgtgtgctgctgctgtgtggcgccgtcttcgtttcgctcagccaggaaatccatgccgagttgagacgcttccgtagagctgggattggctttcgccacctgccccactggaacacccgctgtcctctggcctcccacacgaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacagctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccagatgtctcttacgggggttcgggtggctcaggcggaggatccggatctgatgaagttgatggatcagagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 102 - protein, 636 aa, Homo sapiens
MDAMKRGLCCVLLLCGAVFVSLSQEIHAELRRFRRAGIGFRHLPHWNTRCPLASHTRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLVWRSDVQFAWKHLLCPDVSYGGSGGSGGGSGSDEVDGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - DNA, 63 bp, Artificial Sequence
  primer
agccaggaaatccatgccgagttgagacgcttccgtagagctgggattggctttcgccacctg

SEQ ID NO: 104 - DNA, 68 bp, Artificial Sequence
  primer
acttcatcagatccggatcctccgcctgagccacccgaacccccgtaagagacatctggacacaagag

SEQ ID NO: 105 - DNA, 86 bp, Artificial Sequence
  primer
tcaggtgctgggcacggtgggcatgtgtgagttttgtctgaagatttgggctctgatccatcaacttcatcagatccggatcctcc

SEQ ID NO: 106 - DNA, 1953 bp, Homo sapiens
  CDS (1)...(1953)
atggggagctccagactggcagccctgctcctgcctctcctcctcatagtcatcgacctctctgactctgctgggattggctttcgccacctgccccactggaacacccgctgtcctctggcctcccacacggaagttctgcctatatcccttgccgcacctggtgggccctcttctccacaaagccttggtgtgtgcgagtctggcactgttcccgctgtttgtgccagcatctgctgtcaggtggctcagaaatccaaaaagtcttccacattcaagttctataggagacacaagatgccagcacctgctcagaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacggctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccagatgtctcttacagacacctggggctcttgatcctggcactgctggccctcctcaccctactgggtgttgttctggccctcacctgccggcgcccacagtcaggcccgggcccagcgcggccagtgctcctcctgcacgcggcggactcggaggcgcagcggcgcctggtgggagcgctggctgaactgctacgggcagcgctgggcggcgggcgcgacgtgatcgtggacctgtgggaggggaggcacgtggcgcgcgtgggcccgctgccgtggctctgggcggcgcggacgcgcgtagcgcgggagcagggcactgtgctgctgctgtggagcggcgccgaccttcgcccggtcagcggccccgacccccgcgccgcgcccctgctcgccctgctccacgctgccccgcgcccgctgctgctgctcgcttacttcagtcgcctctgcgccaagggcgacatccccccgccgctgcgcgccctgccgcgctaccgcctgctgcgcgacctgccgcgtctgctgcgggcgctggacgcgcggcctttcgcagaggccaccagctggggccgccttggggcgcggcagcgcaggcagagccgcctagagctgtgcagccggcttgaacgagaggccgcccgacttgcagacctaggttga

SEQ ID NO: 107 - protein, 650 aa, Homo sapiens
MGSSRLAALLLPLLLIVIDLSDSAGIGFRHLPHWNTRCPLASHTEVLPISLAAPGGPSSPQSLGVCESGTVPAVCASICCQVAQKSKKSSTFKFYRRHKMPAPAQRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLVWRSDVQFAWKHLLCPDVSYRHLGLLILALLALLTLLGVVLALTCRRPQSGPGPARPVLLLHAADSEAQRRLVGALAELLRAALGGGRDVIVDLWEGRHVARVGPLPWLWAARTRVAREQGTVLLLWSGADLRPVSGPDPRAAPLLALLHAAPRPLLLLAYFSRLCAKGDIPPPLRALPRYRLLRDLPRLLRALDARPFAEATSWGRLGARQRRQSRLELCSRLEREAARLADLG

SEQ ID NO: 108 - DNA, 1977 bp, Homo sapiens
  CDS (1)...(1245)
atggggagctccagactggcagccctgctcctgcctctcctcctcatagtcatcgacctctctgactctgctgggattggctttcgccacctgccccactggaacacccgctgtcctctggcctcccacacggatgacagtttcactggtcttcaacggggcctcttccacctcctggtgcagaaatccaaaaagtcttccacattcaagttctataggagacacaagatgccagcacctgctcagaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacggctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccagatgacacctggggctcttgatcctggcactgctggccctcctcaccctactgggtgttgttctggccctcacctgccggcgcccacagtcaggcccgggcccagcgcggccagtgctcctcctgcacgcggcggactcggaggcgcagcggcgcctggtgggagcgctggctgaactgctacgggcagcgctgggcggcgggcgcgacgtgatcgtggacctgtgggaggggaggcacgtggcgcgcgtgggcccgctgccgtggctctgggcggcgcggacgcgcgtagcgcgggagcagggcactgtgctgctgctgtggagcggcgccgaccttcgcccggtcagcggccccgacccccgcgccgcgcccctgctcgccctgctccacgctgccccgcgcccgctgctgctgctcgcttacttcagtcgcctctgcgccaagggcgacatccccccgccgctgcgcgccctgccgcgctaccgcctgctgcgcgacctgccgcgtctgctgcgggcgctggacgcgcggcctttcgcagaggccaccagctggggccgccttggggcgcggcagcgcaggcagagccgcctagagctgtgcagccggcttgaacgagaggccgcccgacttgcagacctaggttgagcagagctccaccgcagtcccgggtgtctgcggccgcaacgcaacggacactggctggaaccccggaatgagccttcgaccctgaaatccttggggtgcctcg

SEQ ID NO: 109 - protein, 414 aa, Homo sapiens
MGSSRLAALLLPLLLIVIDLSDSAGIGFRHLPHWNTRCPLASHTDDSFTGLQRGLFHLLVQKSKKSSTFKFYRRHKMPAPAQRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLVWRSDVQFAWKHLLCPDDTWGS

SEQ ID NO: 110 - DNA, 1986 bp, Mus musculus
  CDS (1)...(1986)
atggggagccccagactggcagccttgctcctgtctctcccgctactgctcatcggcctcgctgtgtctgctcgggttgcctgcccctgcctgcggagttggaccagccactgtctcctggcctaccgtgtggataaacgttttgctggccttcagtggggctggttccctctcttggtgaggaaatctaaaagtcctcctaaatttgaagactattggaggcacaggacaccagcatccttccagaggaagctgctaggcagcccttccctgtctgaggaaagccatcgaatttccatcccctcctcagccatctcccacagaggccaacgcaccaaaagggcccagccttcagctgcagaaggaagagaacatctccctgaagcagggtcacaaaagtgtggaggacctgaattctcctttgatttgctgcccgaggtgcaggctgttcgggtgactattcctgcaggccccaaggccagtgtgcgcctttgttatcagtgggcactggaatgtgaagacttgagtagcccttttgatacccagaaaattgtgtctggaggccacactgtagacctgccttatgaattccttctgccctgcatgtgcatagaggcctcctacctgcaagaggacactgtgaggcgcaaaaagtgtcccttccagagctggcctgaagcttatggctcagacttctggcagtcaatacgcttcactgactacagccagcacaatcagatggtcatggctctgacactccgctgcccactgaaactggaggcctccctctgctggaggcaggacccactcacaccctgcgaaacccttcccaacgccacagcacaggagtcagaaggatggtatatcctggagaatgtggacttgcacccccagctctgctttaagttctcatttgaaaacagcagccacgttgaatgtccccaccagagtggctctctcccatcctggactgtgagcatggatacccaggcccagcagctgacgcttcacttttcttcgaggacatatgccaccttcagtgctgcctggagtgacccaggtttggggccggatacccccatgcctcctgtgtacagcatcagccagacccagggctcagtcccagtgacgctagacctcatcatccccttcctgaggcaggagaattgcatcctggtgtggaggtcagatgtccattttgcctggaagcacgtcttgtgtcctgatggtgagttcttgagtgaggagggggggagcatgggcactcgagagatggctcgcctttacctgcttgattcagtctcccatagacacctcgggctcttgatcctggcactgctggctctcaccgctctagtgggtgtagttctggtcctcctcggccggcgcctactgccaggctccggtcgaacaaggccagttttactcctacatgcagcggactcagaggcacagcgacgcctggtgggagctttggccgaactgctgcggacggcgctgggaggtggacgcgacgtgatcgtggatctctgggaagggacgcacgtagcacgcattggaccactgccgtggctttgggcagcgcgggagcgcgtggcgcgggagcagggcacagtgctgctcctgtggaactgtgcgggtcccagcaccgcctgcagcggtgacccgcaggctgcgtcccttcgcaccttgttgtgcgctgctccacgtccgctgctgctcgcctacttcagtcgcctctgcgccaaaggtgacatcccccggccgctgcgcgctctgccacgctaccgcctgcttcgtgacctgccgcgcctgctgagagcactggatgctcagcctgccaccctagcctccagctggagtcaccttggggctaagcggtgcttgaaaaaccgtctggagcagtgtcacctgctggaacttgaggctgccaaagatgactaccaaggctcaaccaatagtccctgtggtttcagctgtctgtag

SEQ ID NO: 111 - protein, 661 aa, Mus musculus
MGSPRLAALLLSLPLLLIGLAVSARVACPCLRSWTSHCLLAYRVDKRFAGLQWGWFPLLVRKSKSPPKFEDYWRHRTPASFQRKLLGSPSLSEESHRISIPSSAISHRGQRTKRAQPSAAEGREHLPEAGSQKCGGPEFSFDLLPEVQAVRVTIPAGPKASVRLCYQWALECEDLSSPFDTQKIVSGGHTVDLPYEFLLPCMCIEASYLQEDTVRRKKCPFQSWPEAYGSDFWQSIRFTDYSQHNQMVMALTLRCPLKLEASLCWRQDPLTPCETLPNATAQESEGWYILENVDLHPQLCFKFSFENSSHVECPHQSGSLPSWTVSMDTQAQQLTLHFSSRTYATFSAAWSDPGLGPDTPMPPVYSISQTQGSVPVTLDLIIPFLRQENCILVWRSDVHFAWKHVLCPDGEFLSEEGGSMGTREMARLYLLDSVSHRHLGLLILALLALTALVGVVLVLLGRRLLPGSGRTRPVLLLHAADSEAQRRLVGALAELLRTALGGGRDVIVDLWEGTHVARIGPLPWLWAARERVAREQGTVLLLWNCAGPSTACSGDPQAASLRTLLCAAPRPLLLAYFSRLCAKGDIPRPLRALPRYRLLRDLPRLLRALDAQPATLASSWSHLGAKRCLKNRLEQCHLLELEAAKDDYQGSTNSPCGFSCL

SEQ ID NO: 112 - DNA, 837 bp, Homo sapiens
  CDS (1)...(837)
ggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacagctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccggatgtctcttacagacac

SEQ ID NO: 113 - protein, 279 aa, Homo sapiens
GPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLVWRSDVQFAWKHLLCPDVSYRH

SEQ ID NO: 114 - DNA, 276 bp, Homo sapiens
  CDS (1)...(276)
ggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcc

SEQ ID NO: 115 - protein, 92 aa, Homo sapiens
GPEFSFDLLPEARAIRVTISSGPEVSVRLCHQWALECEELSSPYDVQKIVSGGHTVELPYEFLLPCLCIEASYLQEDTVRRKKCPFQSWPEA

SEQ ID NO: 116 - DNA, 270 bp, Homo sapiens
  CDS (1)...(270)
tatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacagctcgagagtcagatgggtggtatgttttggagaaggtggacctgcacccccagctctgcttcaagttctcttttggaaacagcagccatgttgaatgcccccaccagact

SEQ ID NO: 117 - protein, 90 aa, Homo sapiens
YGSDFWKSVHFTDYSQHTQMVMALTLRCPLKLEAALCQRHDWHTLCKDLPNATARESDGWYVLEKVDLHPQLCFKFSFGNSSHVECPHQT

SEQ ID NO: 118 - DNA, 291 bp, Homo sapiens
  CDS (1)...(291)
gggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccggatgtctcttacagacac

SEQ ID NO: 119 - protein, 97 aa, Homo sapiens
GSLTSWNVSMDTQAQQLILHFSSRMHATFSAAWSLPGLGQDTLVPPVYTVSQARGSSPVSLDLIIPFLRPGCCVLVWRSDVQFAWKHLLCPDVSYRH

SEQ ID NO: 120 - DNA, 837 bp, Mus musculus
  CDS (1)...(837)
ggacctgaattctcctttgatttgctgcccgaggtgcaggctgttcgggtgactattcctgcaggccccaaggccagtgtgcgcctttgttatcagtgggcactggaatgtgaagacttgagtagcccttttgatacccagaaaattgtgtctggaggccacactgtagacctgccttatgaattccttctgccctgcatgtgcatagaggcctcctacctgcaagaggacactgtgaggcgcaaaaagtgtcccttccagagctggcctgaagcttatggctcagacttctggcagtcaatacgcttcactgactacagccagcacaatcagatggtcatggctctgacactccgctgcccactgaaactggaggcctccctctgctggaggcaggacccactcacaccctgcgaaacccttcccaacgccacagcacaggagtcagaaggatggtatatcctggagaatgtggacttgcacccccagctctgctttaagttctcatttgaaaacagcagccacgttgaatgtccccaccagagtggctctctcccatcctggactgtgagcatggatacccaggcccagcagctgacgcttcacttttcttcgaggacatatgccaccttcagtgctgcctggagtgacccaggtttggggccggatacccccatgcctcctgtgtacagcatcagccagacccagggctcagtcccagtgacgctagacctcatcatccccttcctgaggcaggagaattgcatcctggtgtggaggtcagatgtccattttgcctggaagcacgtcttgtgtcctgatgtctcccatagacac

SEQ ID NO: 121 - protein, 279 aa, Mus musculus
GPEFSFDLLPEVQAVRVTIPAGPKASVRLCYQWALECEDLSSPFDTQKIVSGGHTVDLPYEFLLPCMCIEASYLQEDTVRRKKCPFQSWPEAYGSDFWQSIRFTDYSQHNQMVMALTLRCPLKLEASLCWRQDPLTPCETLPNATAQESEGWYILENVDLHPQLCFKFSFENSSHVECPHQSGSLPSWTVSMDTQAQQLTLHFSSRTYATFSAAWSDPGLGPDTPMPPVYSISQTQGSVPVTLDLIIPFLRQENCILVWRSDVHFAWKHVLCPDVSHRH

SEQ ID NO: 122 - protein, 414 aa, Homo sapiens
AGIGFRHLPHWNTRCPLASHTEVLPISLAAPGGPSSPQSLGVCESGTVPAVCASICCQVAQKSKKSSTFKFYRRHKMPAPAQRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLVWRSDVQFAWKHLLCPDVSYRH

SEQ ID NO: 123 - DNA, 1605 bp, Homo sapiens
  CDS (1)...(1602)
atggggagctccagactggcagccctgctcctgcctctcctcctcatagtcatcgacctctctgactctggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacagctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccggatgtctcttacagacacgagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 124 - protein, 534 aa, Homo sapiens
MGSSRLAALLLPLLLIVIDLSDSGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLVWRSDVQFAWKHLLCPDVSYRHEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - DNA, 49 bp, Artificial Sequence; identical to SEQ ID NO: 78
  C-terminal HIS tag

SEQ ID NO: 126 - DNA, 21 bp, Artificial Sequence
  primer
cactggagtggcaacttccag

SEQ ID NO: 127 - DNA, 25 bp, Artificial Sequence
  primer
caccagacataatagctgacagact

SEQ ID NO: 128 - DNA, 49 bp, Artificial Sequence; identical to SEQ ID NO: 78
  C-terminal HIS tag

SEQ ID NO: 129 - DNA, 36 bp, Artificial Sequence
  primer
gaagaacgtctctcatgacgctcctccccggcctcc

SEQ ID NO: 130 - DNA, 42 bp, Artificial Sequence
  primer
gaagaacgtctctagcccactgaacggggcagcacgcaggtg

SEQ ID NO: 131 - DNA, 49 bp, Artificial Sequence; identical to SEQ ID NO: 78
  C-terminal HIS tag

SEQ ID NO: 132 - DNA, 21 bp, Artificial Sequence
  primer
cactggagtggcaacttccag

SEQ ID NO: 133 - DNA, 25 bp, Artificial Sequence
  primer
caccagacataatagctgacagact

SEQ ID NO: 134 - DNA, 49 bp, Artificial Sequence; identical to SEQ ID NO: 78
  primer

SEQ ID NO: 135 - DNA, 36 bp, Artificial Sequence
  primer
gaagccgaagacttcatggccaccgtcaccgtcact

SEQ ID NO: 136 - DNA, 38 bp, Artificial Sequence
  primer
gaagccgaagacttagccctgtgtagacctgggaagaa

SEQ ID NO: 137 - DNA, 49 bp, Artificial Sequence; identical to SEQ ID NO: 78
  primer

SEQ ID NO: 138 - DNA, 21 bp, Artificial Sequence
  primer
cactggagtggcaacttccag

SEQ ID NO: 139 - DNA, 25 bp, Artificial Sequence
  primer
caccagacataatagctgacagact

SEQ ID NO: 140 - DNA, 25 bp, Artificial Sequence
  primer
ctctccatccttatctttcatcaac

SEQ ID NO: 141 - DNA, 24 bp, Artificial Sequence
  primer
ctctctgctggctaaacaaaacac

SEQ ID NO: 142 - DNA, 26 bp, Artificial Sequence
  primer
ctcatattgctcaactgtgtgaaaag

SEQ ID NO: 143 - DNA, 25 bp, Artificial Sequence
  primer
tagaagccacctgaacacaaatctg

SEQ ID NO: 144 - DNA, 28 bp, Artificial Sequence
  primer
atcttgcgttgtatgttgaaaatcaatt

SEQ ID NO: 145 - DNA, 25 bp, Artificial Sequence
  primer
ttctccaccaggtaaacaagtctac

SEQ ID NO: 146 - DNA, 25 bp, Artificial Sequence
  primer
cactgctactcggctgaggaactgc

SEQ ID NO: 147 - DNA, 23 bp, Artificial Sequence
  primer
ttctgtggatagcggtcctcatc

SEQ ID NO: 148 - DNA, 19 bp, Artificial Sequence
  primer
ccactcacaccctgcgaaa

SEQ ID NO: 149 - DNA, 22 bp, Artificial Sequence
  primer
gcaagtccacattctccaggat

SEQ ID NO: 150 - DNA, 27 bp, Artificial Sequence
  primer
accatccttctgactcctgtgctgtgg

SEQ ID NO: 151 - DNA, 19 bp, Artificial Sequence
  primer
tcagcgtgcgtctttgtca

SEQ ID NO: 152 - DNA, 19 bp, Artificial Sequence
  primer
ggcccccagacacaatttt

SEQ ID NO: 153 - DNA, 30 bp, Artificial Sequence
  primer
catagggactgctcagctcttcacactcca

SEQ ID NO: 154 - DNA, 21 bp, Artificial Sequence
  primer
tggagatatcgcatcgacaca

SEQ ID NO: 155 - DNA, 20 bp, Artificial Sequence
  primer
gcatccacgacacaagcatt

SEQ ID NO: 156 - DNA, 22 bp, Artificial Sequence
  primer
ccgctacccacagaagctggcg

SEQ ID NO: 157 - DNA, 21 bp, Artificial Sequence
  primer
atgaggaccgctatccacaga

SEQ ID NO: 158 - DNA, 16 bp, Artificial Sequence
  primer
cccgtccgtgcatcga

SEQ ID NO: 159 - DNA, 20 bp, Artificial Sequence
  primer
tggccttcgccgagtgcctg

SEQ ID NO: 160 - DNA, 2095 bp, Mus musculus; identical to SEQ ID NO: 68
  CDS (89)...(1864)

SEQ ID NO: 161 - protein, 591 aa, Mus musculus
MGSPRLAALLLSLPLLLIGLAVSARVACPCLRSWTSHCLLAYRVDKRFAGLQWGWFPLLVRKSKSPPKFEDYWRHRTPASFQRKLLGSPSLSEESHRISIPSSAISHRGQRTKRAQPSAAEGREHLPEAGSQKCGGPEFSFDLLPEVQAVRVTIPAGPKASVRLCYQWALECEDLSSPFDTQKIVSGGHTVDLPYEFLLPCMCIEASYLQEDTVRRKKCPFQSWPEAYGSDFWQSIRFTDYSQHNQMVMALTLRCPLKLEASLCWRQDPLTPCETLPNATAQESEGWYILENVDLHPQLCFKFSFENSSHVECPHQSGSLPSWTVSMDTQAQQLTLHFSSRTYATFSAAWSDPGLGPDTPMPPVYSISQTQGSVPVTLDLIIPFLRQENCILVWRSDVHFAWKHVLCPDADSEAQRRLVGALAELLRTALGGGRDVIVDLWEGTHVARIGPLPWLWAARERVAREQGTVLLLWNCAGPSTACSGDPQAASLRTLLCAAPRPLLLAYFSRLCAKGDIPRPLRALPRYRLLRDLPRLLRALDAQPATLASSWSHLGAKRCLKNRLEQCHLLELEAAKDDYQGSTNSPCGFSCL

SEQ ID NO: 162 - DNA, 22 bp, Artificial Sequence
  primer
cgaggcaccccaaggatttcag

SEQ ID NO: 163 - DNA, 20 bp, Artificial Sequence
  primer
aggccctgccacccaccttc

SEQ ID NO: 164 - DNA, 38 bp, Artificial Sequence; identical to SEQ ID NO: 65
  primer

SEQ ID NO: 165 - DNA, 33 bp, Artificial Sequence; identical to SEQ ID NO: 66
  primer

SEQ ID NO: 166 - DNA, 43 bp, Artificial Sequence
  primer
ttttcgtctcagatccgccaccatggggagctccagactggca

SEQ ID NO: 167 - DNA, 75 bp, Artificial Sequence
  primer
tttcgtctcaaattcggatcccttatcatcatcatccttataatctccggagtgtctgtaagagacatccggaca

SEQ ID NO: 168 - DNA, 116 bp, Artificial Sequence
  primer
ttttcgtctcagatccgccaccatggggagctccagactggcagccctgctcctgcctctcctcctcatagtcatcgacctctctgactctggacccgagttctcctttgatttgc

SEQ ID NO: 169 - DNA, 43 bp, Artificial Sequence
  primer
aaaacgtctcagatccgccaccatggggagccccagactggca

SEQ ID NO: 170 - DNA, 73 bp, Artificial Sequence
  primer
aaacgtctcaaattcggatcccttatcatcatcatccttataatctccggagtgtctatgggagacatcagga

SEQ ID NO: 171 - DNA, 5055 bp, Artificial Sequence
  pVAC expression construct
cctgcagggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagtggagccgagagtaattcatacaaaaggactcgcccctgccttggggaatcccagggaccgtcgttaaactcccactaacctagaacccagagatcgctgcgttcccgccccctcacacgcccgctctcgtcatcaccaaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcccttttcccgagggtgggggagaaccgtatataagtgcagtagtcgctgtgaacgttctttttcgcaacgggtttgccgccagaacacaggtaagtactgtgtgtggctcctgcgggcctggcctctttacgggctatggccctcgcgtgccttttattacttacacgcccatggccgctgtacgtgattcttgatcccgagcttcgggttggaagtgggtgggagaggtcgaggccttgcacttaaggagtcccttcgcctcgtgcttgagtcgaggcctggcttgggctctggggctgccgcgtgcgaatctggtagcaccttcgcgcctgccccgctgctttcactaagtttctagccatttaaaatttttgatgaccagctgcaacgccttttttctggcgagataatcttataaatgcggaccaggatctgcacactgatattggggttttgggggccgcgggctgcgacggggctcgtgcgtcccagcgcacatgttcggcgaggcggggcctgcgagcgcggccaccgagagtcggacggggggagtctcaagctggccgtcctgctctggtgccgggcctcgcgccgcggtgtgtcgccccgccctggtcggcaagcctggcccggtcggcaccagttgcgtgagcggaaagatggccgcttcccggccctgccgcagggagctcaaaatggaggacgcggcgcccgggagagcgggcgggtgagtcacccacacaaaggaaaagggcctttccctcctcggtcgccgcttcatgtgaccccacggagtaccgggcgccgtccaggcacctcgattagttctccgagcttttggagtacgtcttccttaggtttgggggaggggttttgtgcggtggagtttccccacacttggtgggtggagactgaagagttaggccagcttggcgctcgatgtaattctccttggaatttgcccttttcgaatttggatcttggcttattctcaagcttcagacagtggttcaaagttttttttctcccatttcaggtgtcgtgaaaactacccctaaaagccatcggatccgccaccatggggagctccagactggcagccctgctcctgcctctcctcctcatagtcatcgacctctctgactctgctgggattggctttcgccacctgccccactggaacacccgctgtcctctggcctcccacacggatgacagtttcactggaagttctgcctatatcccttgccgcacctggtgggccctcttctccacaaagccttggtgtgtgcgagtctggcactgttcccgctgtttgtgccagcatctgctgtcaggtggctcaggtcttcaacggggcctcttccacctcctggtgcagaaatccaaaaagtcttccacattcaagttctataggagacacaagatgccagcacctgctcagaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacggctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccggatgtctcttacagacactccggagattataaggatgatgatgataagggatccgaattcaccactgatgctgcccatcctggaaggtctgtggtgcctgccttgctgcctctgctggctggcactctgctgctgctggagactgccactgctccctaaacctgagctagcattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggtttctttgattacaacactggagagaaatgcagcatgttgctgattgcctgtcactaaaacaggccaaaaactgagtccttgggttgcatagaaagcttcatgttgctaaaccaatgttaagtgaatctttggaaacaaaatgtttccaaattactgggatgtgcatgttgaaacgtgggttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaaatttttcaaaagtagttgacaattaatcatcggcatagtatatcggcatagtataatacgactcactataggagggccatcatggccaagttgaccagtgctgtcccagtgctcacagccagggatgtggctggagctgttgagttctggactgacaggttggggttctccagagattttgtggaggatgactttgcaggtgtggtcagagatgatgtcaccctgttcatctcagcagtccaggaccaggtggtgcctgacaacaccctggcttgggtgtgggtgagaggactggatgagctgtatgctgagtggagtgaggtggtctccaccaacttcagggatgccagtggccctgccatgacagagattggagagcagccctgggggagagagtttgccctgagagacccagcaggcaactgtgtgcactttgtggcagaggagcaggactgaggataagaattgtaacaaaaaaccccgccccggcggggttttttgttaattaa

SEQ ID NO: 172 - DNA, 4821 bp, Artificial Sequence
  pVAC expression construct
cctgcagggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagtggagccgagagtaattcatacaaaaggactcgcccctgccttggggaatcccagggaccgtcgttaaactcccactaacctagaacccagagatcgctgcgttcccgccccctcacacgcccgctctcgtcatcaccaaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcccttttcccgagggtgggggagaaccgtatataagtgcagtagtcgctgtgaacgttctttttcgcaacgggtttgccgccagaacacaggtaagtactgtgtgtggctcctgcgggcctggcctctttacgggctatggccctcgcgtgccttttattacttacacgcccatggccgctgtacgtgattcttgatcccgagcttcgggttggaagtgggtgggagaggtcgaggccttgcacttaaggagtcccttcgcctcgtgcttgagtcgaggcctggcttgggctctggggctgccgcgtgcgaatctggtagcaccttcgcgcctgccccgctgctttcactaagtttctagccatttaaaatttttgatgaccagctgcaacgccttttttctggcgagataatcttataaatgcggaccaggatctgcacactgatattggggttttgggggccgcgggctgcgacggggctcgtgcgtcccagcgcacatgttcggcgaggcggggcctgcgagcgcggccaccgagagtcggacggggggagtctcaagctggccgtcctgctctggtgccgggcctcgcgccgcggtgtgtcgccccgccctggtcggcaagcctggcccggtcggcaccagttgcgtgagcggaaagatggccgcttcccggccctgccgcagggagctcaaaatggaggacgcggcgcccgggagagcgggcgggtgagtcacccacacaaaggaaaagggcctttccctcctcggtcgccgcttcatgtgaccccacggagtaccgggcgccgtccaggcacctcgattagttctccgagcttttggagtacgtcttccttaggtttgggggaggggttttgtgcggtggagtttccccacacttggtgggtggagactgaagagttaggccagcttggcgctcgatgtaattctccttggaatttgcccttttcgaatttggatcttggcttattctcaagcttcagacagtggttcaaagttttttttctcccatttcaggtgtcgtgaaaactacccctaaaagccatcggatccgccaccatggggagctccagactggcagccctgctcctgcctctcctcctcatagtcatcgacctctctgactctgctgggattggctttcgccacctgccccactggaacacccgctgtcctctggcctcccacacgaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacagctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccggatgtctcttacagacacggatcctccggagattataaggatgatgatgataaggaattcaccactgatgctgcccatcctggaaggtctgtggtgcctgccttgctgcctctgctggctggcactctgctgctgctggagactgccactgctccctaaacctgagctagcattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggtttctttgattacaacactggagagaaatgcagcatgttgctgattgcctgtcactaaaacaggccaaaaactgagtccttgggttgcatagaaagcttcatgttgctaaaccaatgttaagtgaatctttggaaacaaaatgtttccaaattactgggatgtgcatgttgaaacgtgggttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaaatttttcaaaagtagttgacaattaatcatcggcatagtatatcggcatagtataatacgactcactataggagggccatcatggccaagttgaccagtgctgtcccagtgctcacagccagggatgtggctggagctgttgagttctggactgacaggttggggttctccagagattttgtggaggatgactttgcaggtgtggtcagagatgatgtcaccctgttcatctcagcagtccaggaccaggtggtgcctgacaacaccctggcttgggtgtgggtgagaggactggatgagctgtatgctgagtggagtgaggtggtctccaccaacttcagggatgccagtggccctgccatgacagagattggagagcagccctgggggagagagtttgccctgagagacccagcaggcaactgtgtgcactttgtggcagaggagcaggactgaggataagaattgtaacaaaaaaccccgccccggcggggttttttgttaattaa

SEQ ID NO: 173 - DNA, 4881 bp, Artificial Sequence
  pVAC expression construct
cctgcagggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagtggagccgagagtaattcatacaaaaggactcgcccctgccttggggaatcccagggaccgtcgttaaactcccactaacctagaacccagagatcgctgcgttcccgccccctcacacgcccgctctcgtcatcaccaaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcccttttcccgagggtgggggagaaccgtatataagtgcagtagtcgctgtgaacgttctttttcgcaacgggtttgccgccagaacacaggtaagtactgtgtgtggctcctgcgggcctggcctctttacgggctatggccctcgcgtgccttttattacttacacgcccatggccgctgtacgtgattcttgatcccgagcttcgggttggaagtgggtgggagaggtcgaggccttgcacttaaggagtcccttcgcctcgtgcttgagtcgaggcctggcttgggctctggggctgccgcgtgcgaatctggtagcaccttcgcgcctgccccgctgctttcactaagtttctagccatttaaaatttttgatgaccagctgcaacgccttttttctggcgagataatcttataaatgcggaccaggatctgcacactgatattggggttttgggggccgcgggctgcgacggggctcgtgcgtcccagcgcacatgttcggcgaggcggggcctgcgagcgcggccaccgagagtcggacggggggagtctcaagctggccgtcctgctctggtgccgggcctcgcgccgcggtgtgtcgccccgccctggtcggcaagcctggcccggtcggcaccagttgcgtgagcggaaagatggccgcttcccggccctgccgcagggagctcaaaatggaggacgcggcgcccgggagagcgggcgggtgagtcacccacacaaaggaaaagggcctttccctcctcggtcgccgcttcatgtgaccccacggagtaccgggcgccgtccaggcacctcgattagttctccgagcttttggagtacgtcttccttaggtttgggggaggggttttgtgcggtggagtttccccacacttggtgggtggagactgaagagttaggccagcttggcgctcgatgtaattctccttggaatttgcccttttcgaatttggatcttggcttattctcaagcttcagacagtggttcaaagttttttttctcccatttcaggtgtcgtgaaaactacccctaaaagccatcggatccgccaccatggggagctccagactggcagccctgctcctgcctctcctcctcatagtcatcgacctctctgactctgctgggattggctttcgccacctgccccactggaacacccgctgtcctctggcctcccacacggtcttcaacggggcctcttccacctcctggtgcagaaatccaaaaagtcttccacattcaagttctataggagacacaagatgccagcacctgctcagaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacagctcgagagtcagatgggtggtatgttttggagaaggtggacctgcacccccagctctgcttcaagttctcttttggaaacagcagccatgttgaatgcccccaccagactggaataacagaggcaagggactggccctcccacattcaggtgtcctgtagcccaggggtcccaatccgtgagccgcagaccagtaactgtctgtggtttgtgagaaacgaggccacacagcaggaggcccggggctcaagcccagtgtcactagacctcatcattcccttcctgaggccagggtgctgtgtcctggtgtggcggtcagatgtccagtttgcctggaagcacctcttgtgtccggatgtctcttacagacactccggagattataaggatgatgatgataagggatccgaattcaccactgatgctgcccatcctggaaggtctgtggtgcctgccttgctgcctctgctggctggcactctgctgctgctggagactgccactgctccctaaacctgagctagcattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggtttctttgattacaacactggagagaaatgcagcatgttgctgattgcctgtcactaaaacaggccaaaaactgagtccttgggttgcatagaaagcttcatgttgctaaaccaatgttaagtgaatctttggaaacaaaatgtttccaaattactgggatgtgcatgttgaaacgtgggttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaaatttttcaaaagtagttgacaattaatcatcggcatagtatatcggcatagtataatacgactcactataggagggccatcatggccaagttgaccagtgctgtcccagtgctcacagccagggatgtggctggagctgttgagttctggactgacaggttggggttctccagagattttgtggaggatgactttgcaggtgtggtcagagatgatgtcaccctgttcatctcagcagtccaggaccaggtggtgcctgacaacaccctggcttgggtgtgggtgagaggactggatgagctgtatgctgagtggagtgaggtggtctccaccaacttcagggatgccagtggccctgccatgacagagattggagagcagccctgggggagagagtttgccctgagagacccagcaggcaactgtgtgcactttgtggcagaggagcaggactgaggataagaattgtaacaaaaaaccccgccccggcggggttttttgttaattaa

SEQ ID NO: 174 - DNA, 4935 bp, Artificial Sequence
  pVAC expression construct
cctgcagggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagtggagccgagagtaattcatacaaaaggactcgcccctgccttggggaatcccagggaccgtcgttaaactcccactaacctagaacccagagatcgctgcgttcccgccccctcacacgcccgctctcgtcatcaccaaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcccttttcccgagggtgggggagaaccgtatataagtgcagtagtcgctgtgaacgttctttttcgcaacgggtttgccgccagaacacaggtaagtactgtgtgtggctcctgcgggcctggcctctttacgggctatggccctcgcgtgccttttattacttacacgcccatggccgctgtacgtgattcttgatcccgagcttcgggttggaagtgggtgggagaggtcgaggccttgcacttaaggagtcccttcgcctcgtgcttgagtcgaggcctggcttgggctctggggctgccgcgtgcgaatctggtagcaccttcgcgcctgccccgctgctttcactaagtttctagccatttaaaatttttgatgaccagctgcaacgccttttttctggcgagataatcttataaatgcggaccaggatctgcacactgatattggggttttgggggccgcgggctgcgacggggctcgtgcgtcccagcgcacatgttcggcgaggcggggcctgcgagcgcggccaccgagagtcggacggggggagtctcaagctggccgtcctgctctggtgccgggcctcgcgccgcggtgtgtcgccccgccctggtcggcaagcctggcccggtcggcaccagttgcgtgagcggaaagatggccgcttcccggccctgccgcagggagctcaaaatggaggacgcggcgcccgggagagcgggcgggtgagtcacccacacaaaggaaaagggcctttccctcctcggtcgccgcttcatgtgaccccacggagtaccgggcgccgtccaggcacctcgattagttctccgagcttttggagtacgtcttccttaggtttgggggaggggttttgtgcggtggagtttccccacacttggtgggtggagactgaagagttaggccagcttggcgctcgatgtaattctccttggaatttgcccttttcgaatttggatcttggcttattctcaagcttcagacagtggttcaaagttttttttctcccatttcaggtgtcgtgaaaactacccctaaaagccatcggatccgccaccatggggagctccagactggcagccctgctcctgcctctcctcctcatagtcatcgacctctctgactctgctgggattggctttcgccacctgccccactggaacacccgctgtcctctggcctcccacacggatgacagtttcactggtcttcaacggggcctcttccacctcctggtgcagaaatccaaaaagtcttccacattcaagttctataggagacacaagatgccagcacctgctcagaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacggctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccggatgtctcttacagacactccggagattataaggatgatgatgataagggatccgaattcaccactgatgctgcccatcctggaaggtctgtggtgcctgccttgctgcctctgctggctggcactctgctgctgctggagactgccactgctccctaaacctgagctagcattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggtttctttgattacaacactggagagaaatgcagcatgttgctgattgcctgtcactaaaacaggccaaaaactgagtccttgggttgcatagaaagcttcatgttgctaaaccaatgttaagtgaatctttggaaacaaaatgtttccaaattactgggatgtgcatgttgaaacgtgggttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaaatttttcaaaagtagttgacaattaatcatcggcatagtatatcggcatagtataatacgactcactataggagggccatcatggccaagttgaccagtgctgtcccagtgctcacagccagggatgtggctggagctgttgagttctggactgacaggttggggttctccagagattttgtggaggatgactttgcaggtgtggtcagagatgatgtcaccctgttcatctcagcagtccaggaccaggtggtgcctgacaacaccctggcttgggtgtgggtgagaggactggatgagctgtatgctgagtggagtgaggtggtctccaccaacttcagggatgccagtggccctgccatgacagagattggagagcagccctgggggagagagtttgccctgagagacccagcaggcaactgtgtgcactttgtggcagaggagcaggactgaggataagaattgtaacaaaaaaccccgccccggcggggttttttgttaattaa

SEQ ID NO: 175 - DNA, 5004 bp, Artificial Sequence
  pVAC expression construct
cctgcagggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagtggagccgagagtaattcatacaaaaggactcgcccctgccttggggaatcccagggaccgtcgttaaactcccactaacctagaacccagagatcgctgcgttcccgccccctcacacgcccgctctcgtcatcaccaaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcccttttcccgagggtgggggagaaccgtatataagtgcagtagtcgctgtgaacgttctttttcgcaacgggtttgccgccagaacacaggtaagtactgtgtgtggctcctgcgggcctggcctctttacgggctatggccctcgcgtgccttttattacttacacgcccatggccgctgtacgtgattcttgatcccgagcttcgggttggaagtgggtgggagaggtcgaggccttgcacttaaggagtcccttcgcctcgtgcttgagtcgaggcctggcttgggctctggggctgccgcgtgcgaatctggtagcaccttcgcgcctgccccgctgctttcactaagtttctagccatttaaaatttttgatgaccagctgcaacgccttttttctggcgagataatcttataaatgcggaccaggatctgcacactgatattggggttttgggggccgcgggctgcgacggggctcgtgcgtcccagcgcacatgttcggcgaggcggggcctgcgagcgcggccaccgagagtcggacggggggagtctcaagctggccgtcctgctctggtgccgggcctcgcgccgcggtgtgtcgccccgccctggtcggcaagcctggcccggtcggcaccagttgcgtgagcggaaagatggccgcttcccggccctgccgcagggagctcaaaatggaggacgcggcgcccgggagagcgggcgggtgagtcacccacacaaaggaaaagggcctttccctcctcggtcgccgcttcatgtgaccccacggagtaccgggcgccgtccaggcacctcgattagttctccgagcttttggagtacgtcttccttaggtttgggggaggggttttgtgcggtggagtttccccacacttggtgggtggagactgaagagttaggccagcttggcgctcgatgtaattctccttggaatttgcccttttcgaatttggatcttggcttattctcaagcttcagacagtggttcaaagttttttttctcccatttcaggtgtcgtgaaaactacccctaaaagccatcggatccgccaccatggggagctccagactggcagccctgctcctgcctctcctcctcatagtcatcgacctctctgactctgctgggattggctttcgccacctgccccactggaacacccgctgtcctctggcctcccacacggaagttctgcctatatcccttgccgcacctggtgggccctcttctccacaaagccttggtgtgtgcgagtctggcactgttcccgctgtttgtgccagcatctgctgtcaggtggctcagaaatccaaaaagtcttccacattcaagttctataggagacacaagatgccagcacctgctcagaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacggctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccggatgtctcttacagacactccggagattataaggatgatgatgataagggatccgaattcaccactgatgctgcccatcctggaaggtctgtggtgcctgccttgctgcctctgctggctggcactctgctgctgctggagactgccactgctccctaaacctgagctagcattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggtttctttgattacaacactggagagaaatgcagcatgttgctgattgcctgtcactaaaacaggccaaaaactgagtccttgggttgcatagaaagcttcatgttgctaaaccaatgttaagtgaatctttggaaacaaaatgtttccaaattactgggatgtgcatgttgaaacgtgggttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaaatttttcaaaagtagttgacaattaatcatcggcatagtatatcggcatagtataatacgactcactataggagggccatcatggccaagttgaccagtgctgtcccagtgctcacagccagggatgtggctggagctgttgagttctggactgacaggttggggttctccagagattttgtggaggatgactttgcaggtgtggtcagagatgatgtcaccctgttcatctcagcagtccaggaccaggtggtgcctgacaacaccctggcttgggtgtgggtgagaggactggatgagctgtatgctgagtggagtgaggtggtctccaccaacttcagggatgccagtggccctgccatgacagagattggagagcagccctgggggagagagtttgccctgagagacccagcaggcaactgtgtgcactttgtggcagaggagcaggactgaggataagaattgtaacaaaaaaccccgccccggcggggttttttgttaattaa

SEQ ID NO: 176 - DNA, 4935 bp, Artificial Sequence
  pVAC expression construct
cctgcagggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagtggagccgagagtaattcatacaaaaggactcgcccctgccttggggaatcccagggaccgtcgttaaactcccactaacctagaacccagagatcgctgcgttcccgccccctcacacgcccgctctcgtcatcaccaaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcccttttcccgagggtgggggagaaccgtatataagtgcagtagtcgctgtgaacgttctttttcgcaacgggtttgccgccagaacacaggtaagtactgtgtgtggctcctgcgggcctggcctctttacgggctatggccctcgcgtgccttttattacttacacgcccatggccgctgtacgtgattcttgatcccgagcttcgggttggaagtgggtgggagaggtcgaggccttgcacttaaggagtcccttcgcctcgtgcttgagtcgaggcctggcttgggctctggggctgccgcgtgcgaatctggtagcaccttcgcgcctgccccgctgctttcactaagtttctagccatttaaaatttttgatgaccagctgcaacgccttttttctggcgagataatcttataaatgcggaccaggatctgcacactgatattggggttttgggggccgcgggctgcgacggggctcgtgcgtcccagcgcacatgttcggcgaggcggggcctgcgagcgcggccaccgagagtcggacggggggagtctcaagctggccgtcctgctctggtgccgggcctcgcgccgcggtgtgtcgccccgccctggtcggcaagcctggcccggtcggcaccagttgcgtgagcggaaagatggccgcttcccggccctgccgcagggagctcaaaatggaggacgcggcgcccgggagagcgggcgggtgagtcacccacacaaaggaaaagggcctttccctcctcggtcgccgcttcatgtgaccccacggagtaccgggcgccgtccaggcacctcgattagttctccgagcttttggagtacgtcttccttaggtttgggggaggggttttgtgcggtggagtttccccacacttggtgggtggagactgaagagttaggccagcttggcgctcgatgtaattctccttggaatttgcccttttcgaatttggatcttggcttattctcaagcttcagacagtggttcaaagttttttttctcccatttcaggtgtcgtgaaaactacccctaaaagccatcggatccgccaccatggggagccccagactggcagccttgctcctgtctctcccgctactgctcatcggcctcgctgtgtctgctcgggttgcctgcccctgcctgcggagttggaccagccactgtctcctggcctaccgtgtggataaacgttttgctggccttcagtggggctggttccctctcttggtgaggaaatctaaaagtcctcctaaatttgaagactattggaggcacaggacaccagcatccttccagaggaagctgctaggcagcccttccctgtctgaggaaagccatcgaatttccatcccctcctcagccatctcccacagaggccaacgcaccaaaagggcccagccttcagctgcagaaggaagagaacatctccctgaagcagggtcacaaaagtgtggaggacctgaattctcctttgatttgctgcccgaggtgcaggctgttcgggtgactattcctgcaggccccaaggccagtgtgcgcctttgttatcagtgggcactggaatgtgaagacttgagtagcccttttgatacccagaaaattgtgtctggaggccacactgtagacctgccttatgaattccttctgccctgcatgtgcatagaggcctcctacctgcaagaggacactgtgaggcgcaaaaagtgtcccttccagagctggcctgaagcttatggctcagacttctggcagtcaatacgcttcactgactacagccagcacaatcagatggtcatggctctgacactccgctgcccactgaaactggaggcctccctctgctggaggcaggacccactcacaccctgcgaaacccttcccaacgccacagcacaggagtcagaaggatggtatatcctggagaatgtggacttgcacccccagctctgctttaagttctcatttgaaaacagcagccacgttgaatgtccccaccagagtggctctctcccatcctggactgtgagcatggatacccaggcccagcagctgacgcttcacttttcttcgaggacatatgccaccttcagtgctgcctggagtgacccaggtttggggccggatacccccatgcctcctgtgtacagcatcagccagacccagggctcagtcccagtgacgctagacctcatcatccccttcctgaggcaggagaattgcatcctggtgtggaggtcagatgtccattttgcctggaagcacgtcttgtgtcctgatgtctcccatagacactccggagattataaggatgatgatgataagggatccgaattcaccactgatgctgcccatcctggaaggtctgtggtgcctgccttgctgcctctgctggctggcactctgctgctgctggagactgccactgctccctaaacctgagctagcattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggtttctttgattacaacactggagagaaatgcagcatgttgctgattgcctgtcactaaaacaggccaaaaactgagtccttgggttgcatagaaagcttcatgttgctaaaccaatgttaagtgaatctttggaaacaaaatgtttccaaattactgggatgtgcatgttgaaacgtgggttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaaatttttcaaaagtagttgacaattaatcatcggcatagtatatcggcatagtataatacgactcactataggagggccatcatggccaagttgaccagtgctgtcccagtgctcacagccagggatgtggctggagctgttgagttctggactgacaggttggggttctccagagattttgtggaggatgactttgcaggtgtggtcagagatgatgtcaccctgttcatctcagcagtccaggaccaggtggtgcctgacaacaccctggcttgggtgtgggtgagaggactggatgagctgtatgctgagtggagtgaggtggtctccaccaacttcagggatgccagtggccctgccatgacagagattggagagcagccctgggggagagagtttgccctgagagacccagcaggcaactgtgtgcactttgtggcagaggagcaggactgaggataagaattgtaacaaaaaaccccgccccggcggggttttttgttaattaa

SEQ ID NO: 177 - DNA, 4599 bp, Artificial Sequence
  pVAC expression construct
cctgcagggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagtggagccgagagtaattcatacaaaaggactcgcccctgccttggggaatcccagggaccgtcgttaaactcccactaacctagaacccagagatcgctgcgttcccgccccctcacacgcccgctctcgtcatcaccaaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcccttttcccgagggtgggggagaaccgtatataagtgcagtagtcgctgtgaacgttctttttcgcaacgggtttgccgccagaacacaggtaagtactgtgtgtggctcctgcgggcctggcctctttacgggctatggccctcgcgtgccttttattacttacacgcccatggccgctgtacgtgattcttgatcccgagcttcgggttggaagtgggtgggagaggtcgaggccttgcacttaaggagtcccttcgcctcgtgcttgagtcgaggcctggcttgggctctggggctgccgcgtgcgaatctggtagcaccttcgcgcctgccccgctgctttcactaagtttctagccatttaaaatttttgatgaccagctgcaacgccttttttctggcgagataatcttataaatgcggaccaggatctgcacactgatattggggttttgggggccgcgggctgcgacggggctcgtgcgtcccagcgcacatgttcggcgaggcggggcctgcgagcgcggccaccgagagtcggacggggggagtctcaagctggccgtcctgctctggtgccgggcctcgcgccgcggtgtgtcgccccgccctggtcggcaagcctggcccggtcggcaccagttgcgtgagcggaaagatggccgcttcccggccctgccgcagggagctcaaaatggaggacgcggcgcccgggagagcgggcgggtgagtcacccacacaaaggaaaagggcctttccctcctcggtcgccgcttcatgtgaccccacggagtaccgggcgccgtccaggcacctcgattagttctccgagcttttggagtacgtcttccttaggtttgggggaggggttttgtgcggtggagtttccccacacttggtgggtggagactgaagagttaggccagcttggcgctcgatgtaattctccttggaatttgcccttttcgaatttggatcttggcttattctcaagcttcagacagtggttcaaagttttttttctcccatttcaggtgtcgtgaaaactacccctaaaagccatcggatccgccaccatggggagctccagactggcagccctgctcctgcctctcctcctcatagtcatcgacctctctgactctggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacagctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccggatgtctcttacagacactccggagattataaggatgatgatgataagggatccgaattcaccactgatgctgcccatcctggaaggtctgtggtgcctgccttgctgcctctgctggctggcactctgctgctgctggagactgccactgctccctaaacctgagctagcattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggtttctttgattacaacactggagagaaatgcagcatgttgctgattgcctgtcactaaaacaggccaaaaactgagtccttgggttgcatagaaagcttcatgttgctaaaccaatgttaagtgaatctttggaaacaaaatgtttccaaattactgggatgtgcatgttgaaacgtgggttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaaatttttcaaaagtagttgacaattaatcatcggcatagtatatcggcatagtataatacgactcactataggagggccatcatggccaagttgaccagtgctgtcccagtgctcacagccagggatgtggctggagctgttgagttctggactgacaggttggggttctccagagattttgtggaggatgactttgcaggtgtggtcagagatgatgtcaccctgttcatctcagcagtccaggaccaggtggtgcctgacaacaccctggcttgggtgtgggtgagaggactggatgagctgtatgctgagtggagtgaggtggtctccaccaacttcagggatgccagtggccctgccatgacagagattggagagcagccctgggggagagagtttgccctgagagacccagcaggcaactgtgtgcactttgtggcagaggagcaggactgaggataagaattgtaacaaaaaaccccgccccggcggggttttttgttaattaa

SEQ ID NO: 178 - DNA, 10937 bp, Artificial Sequence
  zcytor21-fc10 fusion protein
ggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaatgaaagaccccacctgtaggtttggcaagctagcttaagtaacgccatttgcaaggcatggaaaaatacataactgagaatagagaagttcagatcaaggtcaggaacagagaaacaggagaatatgggccaaacaggatatctgtggtaagcagttcctgccccgctcagggccaagaacagttggaacaggagaatatgggccaaacaggatatctgtggtaagcagttcctgccccgctcagggccaagaacagatggtccccagatcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccggggctagcggctcgtggatctcagctacaggtaaggggctcacagtagcaggcttgaggtctggccatatacatcggtgacattgacatccactttgcctttctctccacaggtgtcctcgagaattcatataggccggccaccatggggagctccagactggcagccctgctcctgcctctcctcctcatagtcatcgacctctctgactctgctgggattggctttcgccacctgccccactggaacacccgctgtcctctggcctcccacacgaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacagctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccagatgtctcttacgggggttcgggtggctcaggcggaggatccggatctgatgaagttgatggatcagagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataaaaataatctagaggcgcgccttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattgcggccgcccaagcttgggcggccgcccattatgtgatcaggggagcgcgtcatggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaaccacaggtaaggatctggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttaagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaacgttctgcagacgagccgtcgagatccgtgccatcatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggctttagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggagagagactagtagagagtatcgatagagagcgatcgaattaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccaccttttcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgagtgacgttgcattagttattaatagtaatcaattacggggtcattagttcatagcccatatatggagttccgcgttacataacttacggtaaatggcccgcct

SEQ ID NO: 179 - DNA, 22 bp, Artificial Sequence
  primer
cgaggcaccccaaggatttcag

SEQ ID NO: 180 - DNA, 20 bp, Artificial Sequence
  primer
aggccctgccacccaccttc

SEQ ID NO: 181 - DNA, 38 bp, Artificial Sequence; identical to SEQ ID NO: 65
  primer

SEQ ID NO: 182 - DNA, 33 bp, Artificial Sequence; identical to SEQ ID NO: 66
  primer

SEQ ID NO: 183 - DNA, 9159 bp, Artificial Sequence
  mammalian expression vector
cagtcacgacgttgtaaaacgacggccagtgagtgacgttgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaatgaaagaccccacctgtaggtttggcaagctagcttaagtaacgccatttgcaaggcatggaaaaatacataactgagaatagagaagttcagatcaaggtcaggaacagagaaacaggagaatatgggccaaacaggatatctgtggtaagcagttcctgccccgctcagggccaagaacagttggaacaggagaatatgggccaaacaggatatctgtggtaagcagttcctgccccgctcagggccaagaacagatggtccccagatcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccggggctagcggctcgtggatctcagctacaggtaaggggctcacagtagcaggcttgaggtctggccatatacatcggtgacattgacatccactttgcctttctctccacaggtgtcctcgagaattcatataggccggccaccatggatgcaatgaagagagggctctgctgtgtgctgctgctgtgtggcgccgtcttcgtttcgctcagccaggaaatccatgccgagttgagacgcttccgtagaaaataatctagaggcgcgccttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattgcggccgcccaagcttgggcggccgcccattatgtgatcaggggagcgcgtcatggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaaccacaggtaaggatctggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttaagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaacgttctgcagacgagccgtcgagatccgtgccatcatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttctaagaccatgggacttttgctggctttagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctagatatcgatgaattaattcgtaatcatgtcatagctgtttcctgtgtgaaattgttatccgctcacaattccacacaacatacgagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtcthabvrrtnrmattcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcc

SEQ ID NO: 184 - DNA, 10719 bp, Artificial Sequence
  mammalian expression vector
cagtcacgacgttgtaaaacgacggccagtgagtgacgttgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaatgaaagaccccacctgtaggtttggcaagctagcttaagtaacgccatttgcaaggcatggaaaaatacataactgagaatagagaagttcagatcaaggtcaggaacagagaaacaggagaatatgggccaaacaggatatctgtggtaagcagttcctgccccgctcagggccaagaacagttggaacaggagaatatgggccaaacaggatatctgtggtaagcagttcctgccccgctcagggccaagaacagatggtccccagatcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccggggctagcggctcgtggatctcagctacaggtaaggggctcacagtagcaggcttgaggtctggccatatacatcggtgacattgacatccactttgcctttctctccacaggtgtcctcgagaattcatataggccggccaccatggatgcaatgaagagagggctctgctgtgtgctgctgctgtgtggcgccgtcttcgtttcgctcagccaggaaatccatgccgagttgagacgcttccgtagaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacagctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccggatgtctcttacagacacggatctgatgaagttgatggatcagagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataaaaataatctagaggcgcgccttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattgcggccgcccaagcttgggcggccgcccattatgtgatcaggggagcgcgtcatggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaaccacaggtaaggatctggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttaagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaacgttctgcagacgagccgtcgagatccgtgccatcatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttctaagaccatgggacttttgctggctttagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctagatatcgatgaattaattcgtaatcatgtcatagctgtttcctgtgtgaaattgttatccgctcacaattccacacaacatacgagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtcthabvrrtnrmattcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcc

SEQ ID NO: 185 - DNA, 51 bp, Artificial Sequence
  primer
ctctgatccatcaacttcatcagatccgtgtctgtaagagacatccggaca

SEQ ID NO: 186 - DNA, 11199 bp, Artificial Sequence
  expression vector
cagtcacgacgttgtaaaacgacggccagtgagtgacgttgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaatgaaagaccccacctgtaggtttggcaagctagcttaagtaacgccatttgcaaggcatggaaaaatacataactgagaatagagaagttcagatcaaggtcaggaacagagaaacaggagaatatgggccaaacaggatatctgtggtaagcagttcctgccccgctcagggccaagaacagttggaacaggagaatatgggccaaacaggatatctgtggtaagcagttcctgccccgctcagggccaagaacagatggtccccagatcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccggggctagcggctcgtggatctcagctacaggtaaggggctcacagtagcaggcttgaggtctggccatatacatcggtgacattgacatccactttgcctttctctccacaggtgtcctcgagaattcatataggccggccaccatggatgcaatgaagagagggctctgctgtgtgctgctgctgtgtggcgccgtcttcgtttcgctcagccaggaaatccatgccgagttgagacgcttccgtagagctgggattggctttcgccacctgccccactggaacacccgctgtcctctggcctcccacacggatgacagtttcactggaagttctgcctatatcccttgccgcacctggtgggccctcttctccacaaagccttggtgtgtgcgagtctggcactgttcccgctgtttgtgccagcatctgctgtcaggtggctcaggtcttcaacggggcctcttccacctcctggtgcagaaatccaaaaagtcttccacattcaagttctataggagacacaagatgccagcacctgctcagaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacggctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccagatgtctcttacgggggttcgggtggctcaggcggaggatccggatctgatgaagttgatggatcagagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataaaaataatctagaggcgcgccttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattgcggccgcccaagcttgggcggccgcccattatgtgatcaggggagcgcgtcatggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaaccacaggtaaggatctggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttaagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaacgttctgcagacgagccgtcgagatccgtgccatcatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttctaagaccatgggacttttgctggctttagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctagatatcgatgaattaattcgtaatcatgtcatagctgtttcctgtgtgaaattgttatccgctcacaattccacacaacatacgagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtcthabvrrtnrmattcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcc